SEQUENCE LISTING (144 sequences)

SEQ ID NO: 1 - protein, 21 aa, Homo sapiens
MALPVTALLLPLALLLHAARP

SEQ ID NO: 2 - protein, 248 aa, Artificial Sequence
  Made in Lab - anti-CD3 scFv
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSAAAKP

SEQ ID NO: 3 - protein, 45 aa, Homo sapiens
TTTPAPRPPTPAPTIASQPLSLRPEASRPAAGGAVHTRGLDFASD

SEQ ID NO: 4 - protein, 53 aa, Artificial Sequence
  Made in Lab - CD4 TM, cytoplasmic tail and T2A linker construct
MALIVLGGVAGLLLFIGLGIFFCVRCRHRRRQGSGEGRGSLLTCGDVEENPGP

SEQ ID NO: 5 - protein, 511 aa, Cocal virus
NFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 6 - DNA, 63 bp, Artificial Sequence
  Made in Lab - polynucleotide encoding CD8 signal peptide
atggcactgcctgtgacagccctgctgctgccactggccctgctgctgcacgcagcacgccca

SEQ ID NO: 7 - DNA, 744 bp, Artificial Sequence
  Made in Lab - polynucleotide encoding anti-cd3 scFv construct
gatatccagatgacccagtccccaagctccctgagcgcctccgtgggcgaccgggtgacaatcacctgcagcgcctctagctccgtgtcctacatgaactggtatcagcagacacctggcaaggccccaaagagatggatctacgataccagcaagctggcctccggcgtgccttctaggttttctggcagcggctccggcacagattatacattcaccatctctagcctgcagccagaggacatcgccacctactattgccagcagtggtcctctaatccctttacattcggccagggcaccaagctgcagatcacaagaacctctggaggaggaggaagcggaggaggaggatccggcggcggcggctctcaggtgcagctggtgcagagcggaggaggagtggtgcagccaggcagaagcctgaggctgtcctgtaaggcctctggctacacattcaccagatatacaatgcactgggtgaggcaggcaccaggcaagggactggagtggatcggctacatcaacccctccaggggctacaccaactataatcagaaggtgaaggatcggttcaccatcagcagggacaactccaagaataccgccttcctgcagatggacagcctgaggccagaggataccggcgtgtacttttgcgcccggtactatgacgatcactactgtctggattattggggccagggaacaccagtgaccgtgagctccgccgcagcaaagcct

SEQ ID NO: 8 - DNA, 135 bp, Artificial Sequence
  Made in Lab - polynucleotide encoding cd8 hinge
accacaacccctgccccaaggccacctacacccgcccctaccatcgcctctcagccactgagcctgaggccagaggcatccaggcctgccgcagggggggccgtgcacacccggggcctggactttgcctctgat

SEQ ID NO: 9 - DNA, 159 bp, Artificial Sequence
  Made in Lab - polynucleotide encoding cd tm, cytoplasmic tail and T2a linker construct
atggcactgatcgtgctgggaggagtggcaggactgctgctgttcatcggactgggcatcttcttttgcgtgcgctgtaggcaccggagaaggcagggatctggagagggaaggggaagcctgctgacatgcggcgacgtggaggagaacccaggacca

SEQ ID NO: 10 - DNA, 1533 bp, Artificial Sequence
  Made in Lab - polynucleotide encoding cocal env
aattttctgctgctgaccttcatcgtgctgcctctgtgcagccacgccaagttttccatcgtgttcccacagtcccagaagggcaactggaagaatgtgccctctagctaccactattgcccttcctctagcgaccagaactggcacaatgatctgctgggcatcacaatgaaggtgaagatgcccaagacccacaaggccatccaggcagatggatggatgtgccacgcagccaagtggatcacaacctgtgactttcggtggtacggccccaagtatatcacacactccatccactctatccagcctacctccgagcagtgcaaggagtctatcaagcagacaaagcagggcacctggatgagccctggcttcccaccccagaactgtggctacgccacagtgaccgactccgtggcagtggtggtgcaggcaacacctcaccacgtgctggtggatgagtataccggcgagtggatcgacagccagtttccaaacggcaagtgcgagacagaggagtgtgagaccgtgcacaattctacagtgtggtacagcgattataaggtgacaggcctgtgcgacgccaccctggtggatacagagatcaccttcttttctgaggacggcaagaaggagagcatcggcaagcccaacaccggctacagatccaattacttcgcctatgagaagggcgataaggtgtgcaagatgaattattgtaagcacgccggggtgcggctgcctagcggcgtgtggtttgagttcgtggaccaggacgtgtacgcagcagcaaagctgcctgagtgcccagtgggagcaaccatctccgccccaacacagacctccgtggacgtgtctctgatcctggatgtggagcgcatcctggactacagcctgtgccaggagacctggagcaagatccggtccaagcagcccgtgtcccctgtggacctgtcttacctggcaccaaagaacccaggaaccggaccagcctttacaatcatcaatggcaccctgaagtacttcgagacccgctatatccggatcgacatcgataaccctatcatcagcaagatggtgggcaagatctctggcagccagacagagagagagctgtggaccgagtggttcccttacgagggcgtggagatcggcccaaatggcatcctgaagacaccaaccggctataagtttcccctgttcatgatcggccacggcatgctggacagcgatctgcacaagacctcccaggccgaggtgtttgagcacccacacctggcagaggcaccaaagcagctgcctgaggaggagacactgttctttggcgataccggcatctctaagaaccccgtggagctgatcgagggctggttttcctcttggaagagcacagtggtgaccttctttttcgccatcggcgtgttcatcctgctgtacgtggtggccagaatcgtgatcgccgtgagatacaggtatcagggctccaacaataagaggatctataatgacatcgagatgtctcgcttccggaag

SEQ ID NO: 11 - protein, 17 aa, Unknown
  Gaussia species luciferase
MGVKVLFALICIAVAEA

SEQ ID NO: 12 - protein, 245 aa, Artificial Sequence
  Made in Lab - anti-cd3 scFv construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSAS

SEQ ID NO: 13 - protein, 62 aa, Cocal virus
GVELIEGWFSSWKSTVVTFFFAIGVFILLYVVARIVIAVRYRYQGSNNKRIYNDIEMSRFRK

SEQ ID NO: 14 - DNA, 51 bp, Artificial Sequence
  Made in Lab - polynucleotide encoding a Gaussia species luciferase signal peptide
atgggcgtgaaagtgctgttcgccctgatctgcatcgcagttgctgaagcc

SEQ ID NO: 15 - DNA, 735 bp, Artificial Sequence
  Made in Lab - polynucleotide encoding anti-cd3 scFv construct
gacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgcctcc

SEQ ID NO: 16 - DNA, 186 bp, Artificial Sequence
  Made in Lab - polynucleotide encoding short hinge-TM-CT from Cocal Env
ggagtggaactgatcgagggctggttcagcagctggaaaagcaccgtggttacattctttttcgccatcggcgtgttcatcctgctgtacgtggtcgccagaattgtgatcgccgtgcggtatagataccagggcagcaacaacaagcggatctacaacgacatcgagatgagcagattcagaaag

SEQ ID NO: 17-18 - skipped (2 entries)

SEQ ID NO: 19 - protein, 93 aa, Cocal virus
SGFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 20-21 - skipped (2 entries)

SEQ ID NO: 22 - DNA, 279 bp, Artificial Sequence
  Made in Lab - polynucleotide encoding long hinge-TM-CT from Cocal Env
tccggattcgagcacccccacctggccgaggcccctaagcagctgcctgaagaagagacactgtttttcggagataccggcatcagcaaaaaccccgtggagctgatcgagggctggttcagctcttggaagagcaccgtggtcacattctttttcgccatcggcgtctttatcctgctgtacgtggtagccagaatcgtgatcgccgtgcggtacagataccagggcagcaacaacaagcggatctacaacgacatcgagatgagccggttcagaaag

SEQ ID NO: 23-24 - skipped (2 entries)

SEQ ID NO: 25 - protein, 113 aa, Artificial Sequence
  Made in Lab - human Glycophorin A ecto-TM_HIV Env CT construct
SGGSTSGSGKPGSGEGSTKGPEITLIIFGVMAGVIGTILLISYGIRRLALKYWWNLLQYWSQELKNSAVSLLNATAIAVAEGTDRVIEVVQGACRAIRHIPRRIRQGLERILL

SEQ ID NO: 26-27 - skipped (2 entries)

SEQ ID NO: 28 - DNA, 339 bp, Artificial Sequence
  Made in Lab - polynucleotide encoding human Glycophorin A ecto-TM_HIV Env CT construct
tccggaggatctacaagcggctctggcaagcctggcagcggagaaggcagcaccaagggccctgagatcacactgatcatcttcggcgtgatggccggcgtcatcggcaccatcctgctgatcagctacggcatcagaagactggctctgaagtactggtggaatctgctgcaatactggagccaggagctgaaaaacagcgccgtgtccctgctcaacgccaccgccatcgccgtggccgagggcaccgacagagtgatcgaggtggtgcagggagcctgcagagctattcggcacatccccagacggatcaggcagggcctggaaagaatcctgctg

SEQ ID NO: 29-30 - skipped (2 entries)

SEQ ID NO: 31 - protein, 119 aa, Artificial Sequence
  Made in Lab - 218 linker_HIV Env-TM-CT construct
SGGSTSGSGKPGSGEGSTKGNWLWYIRIFIIIVGSLIGLRIVFAVLSLVNRGWEALKYWWNLLQYWSQELKNSAVSLLNATAIAVAEGTDRVIEVVQGACRAIRHIPRRIRQGLERILL

SEQ ID NO: 32-33 - skipped (2 entries)

SEQ ID NO: 34 - DNA, 357 bp, Artificial Sequence
  Made in Lab - polynucleotide encoding 218 linker_HIV Env-TM-CT construct
tccggaggaagcaccagcggctctggcaagcctggcagcggcgagggctctaccaagggcaattggctgtggtacatcagaatcttcatcatcatcgtgggcagcctgatcggcctgagaatcgtgttcgccgtgctgagcctggtgaaccggggctgggaagctctgaagtactggtggaacctgctgcaatactggtcccaggagctgaaaaacagcgctgtgtccctgctcaacgccaccgccatcgccgtcgccgagggaacagacagagtgatcgaggtggtgcagggagcctgcagagccattcggcacatccccagacgcatcagacagggcctggaaagaatcctgctg

SEQ ID NO: 35 - DNA, 353 bp, Artificial Sequence
  Synthetic MND promoter
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgctagc

SEQ ID NO: 36 - skipped

SEQ ID NO: 37 - protein, 112 aa, Artificial Sequence
  Made in Lab - G4Sx3 linker_HIV Env-TM-CT construct
SGGGGGSGGGGSGGGGSYIRIFIIIVGSLIGLRIVFAVLSLVNRGWEALKYWWNLLQYWSQELKNSAVSLLNATAIAVAEGTDRVIEVVQGACRAIRHIPRRIRQGLERILL

SEQ ID NO: 38-39 - skipped (2 entries)

SEQ ID NO: 40 - DNA, 336 bp, Artificial Sequence
  Made in Lab - polynucleotide encoding G4Sx3 linker_HIV Env-TM-CT construct
tccggaggcggtggaggctctggtggcggagggagcggtggcggaggcagctacatcagaatcttcatcatcatcgtgggcagcctgatcggcctgagaatcgtgttcgccgttctgagcctggtgaaccggggctgggaagccctgaagtactggtggaatctgctccagtactggtctcaggagctgaagaacagcgccgtgtccctgctgaacgctacagctatcgccgtcgccgagggcaccgacagagtgatcgaggtggtgcagggcgcctgcagagccatccggcacatccctagaaggattcggcaaggcctggaaagaatcctgctg

SEQ ID NO: 41-42 - skipped (2 entries)

SEQ ID NO: 43 - protein, 83 aa, Artificial Sequence
  Made in Lab - Short hinge-TM-CT from Cocal Env_T2A construct
GVELIEGWFSSWKSTVVTFFFAIGVFILLYVVARIVIAVRYRYQGSNNKRIYNDIEMSRFRKGSGEGRGSLLTCGDVEENPGP

SEQ ID NO: 44-46 - skipped (3 entries)

SEQ ID NO: 47 - DNA, 249 bp, Artificial Sequence
  Made in Lab - polynucleotide encoding Short hinge-TM-CT from Cocal Env_T2A construct
ggagtggaactgatcgagggctggttcagcagctggaaaagcaccgtggttacattctttttcgccatcggcgtgttcatcctgctgtacgtggtcgccagaattgtgatcgccgtgcggtatagataccagggcagcaacaacaagcggatctacaacgacatcgagatgagcagattcagaaagggatctggagagggaaggggaagcctgctgacatgcggcgacgtggaggagaacccaggacca

SEQ ID NO: 48 - skipped

SEQ ID NO: 49 - protein, 1296 aa, Artificial Sequence
  Made in Lab - CD19 CAR_FRB_RACR lentiviral vector construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPEMWHEGLEEASRLYFGERNVKGMFEVLEPLHAMMERGPQTLKETSFNQAYGRDLMEAQEWCRKYMKSGNVKDLLQAWDLYYHVFRRISKGSGATNFSLLKQAGDVEENPGPMPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 50 - protein, 22 aa, Homo sapiens
MLLLVTSLLLCELPHPAFLLIP

SEQ ID NO: 51 - protein, 245 aa, Artificial Sequence
  Made in Lab - VL-linker-VH construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 52 - protein, 40 aa, Artificial Sequence
  Made in Lab - IgG4 linker-CD28 TM construct
ESKYGPPCPPCPMFWVLVVVGGVLACYSLLVTVAFIIFWV

SEQ ID NO: 53 - protein, 42 aa, Homo sapiens
KRGRKKLLYIFKQPFMRPVQTTQEEDGCSCRFPEEEEGGCEL

SEQ ID NO: 54 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 22 aa, Artificial Sequence
  P2A synthetic construct
GSGATNFSLLKQAGDVEENPGP

SEQ ID NO: 56 - protein, 89 aa, Homo sapiens
EMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 57 - protein, 126 aa, Artificial Sequence
  Signal peptide-FKBP12 synthetic construct
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKL

SEQ ID NO: 58 - protein, 125 aa, Artificial Sequence
  IL-2R gamma TM-Cytoplasmic domain synthetic construct
GEGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 59 - protein, 114 aa, Artificial Sequence
  Signal peptide-FRB synthetic construct
MALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 60 - protein, 315 aa, Homo sapiens
GKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 61 - DNA, 3891 bp, Artificial Sequence
  Polynucleotide encoding CD19 CAR_FRB_RACR lentiviral vector construct
atgctgctgctggtgacctccctgctgctgtgcgagctgcctcacccagcctttctgctgatccccgacatccagatgacacagaccacaagctccctgtctgccagcctgggcgacagagtgaccatctcctgtagggcctctcaggatatcagcaagtacctgaactggtatcagcagaagccagatggcacagtgaagctgctgatctaccacacctccaggctgcactctggagtgccaagccggttctccggatctggaagcggcaccgactattccctgacaatctctaacctggagcaggaggatatcgccacatacttttgccagcagggcaataccctgccatatacattcggcggaggaaccaagctggagatcaccggatccacatctggaagcggcaagccaggaagcggagagggatccacaaagggagaggtgaagctgcaggagagcggaccaggactggtggcaccatcccagtctctgagcgtgacctgtacagtgtccggcgtgtctctgcctgactacggcgtgtcctggatcaggcagccacctaggaagggactggagtggctgggcgtgatctggggctctgagaccacatactataattctgccctgaagagccgcctgaccatcatcaaggacaactccaagtctcaggtgtttctgaagatgaatagcctgcagaccgacgatacagccatctactattgcgccaagcactactattacggcggctcctacgccatggattattggggccagggcacctccgtgacagtgtctagcgagtctaagtatggcccaccctgccctccatgtccaatgttctgggtgctggtggtggtgggaggcgtgctggcctgttactccctgctggtgaccgtggcctttatcatcttctgggtgaagagaggcaggaagaagctgctgtatatctttaagcagcccttcatgcgccctgtgcagaccacacaggaggaggacggctgcagctgtcggtttccagaggaggaggagggaggatgcgagctgcgcgtgaagttcagccggtccgccgatgcccctgcctaccagcagggccagaaccagctgtataacgagctgaatctgggccggagagaggagtacgacgtgctggataagaggaggggaagggacccagagatgggaggcaagcctcggagaaagaacccacaggagggcctgtacaatgagctgcagaaggacaagatggccgaggcctattctgagatcggcatgaagggagagaggcgccggggcaagggacacgatggcctgtaccagggcctgagcaccgccacaaaggacacatatgatgccctgcacatgcaggccctgccacctaggggatctggagccaccaactttagcctgctgaagcaggcaggcgatgtggaggagaatccaggacctgagatgtggcacgagggactggaggaggcaagcaggctgtactttggcgagcggaatgtgaagggcatgttcgaggtgctggagccactgcacgcaatgatggagaggggaccacagaccctgaaggagacatccttcaaccaggcatacggaagggacctgatggaggcacaggagtggtgccggaagtatatgaagtctggcaatgtgaaggacctgctgcaggcctgggatctgtattaccacgtgtttagaaggatcagcaagggctccggcgccaccaacttctccctgctgaagcaggccggcgatgtggaagaaaatccaggaccaatgcctctgggactgctgtggctgggactggccctgctgggcgccctgcacgcccaggccggcgtgcaggtggagacaatcagccctggcgacggcagaacctttccaaagaggggccagacatgcgtggtgcactacaccggcatgctggaggatggcaagaagttcgactcctctcgcgatcggaacaagccctttaagttcatgctgggcaagcaggaagtgatcagaggctgggaggagggcgtggcccagatgtctgtgggccagagggccaagctgacaatcagcccagactatgcatacggagcaaccggacaccctggaatcatcccaccacacgccacactggtgttcgatgtggagctgctgaagctgggcgagggctctaacaccagcaaggagaatccatttctgttcgcactggaggcagtggtcatctccgtgggctctatgggcctgatcatctccctgctgtgcgtgtacttttggctggagagaacaatgccaaggatccccaccctgaagaacctggaggacctggtgaccgagtaccacggcaatttcagcgcctggtccggcgtgtctaagggactggcagagtccctgcagccagattattctgagcggctgtgcctggtgagcgagatccctccaaagggaggcgccctgggagagggaccaggagccagcccctgcaaccagcactccccttactgggcccccccttgttataccctgaagccagagacaggctctggcgccaccaacttcagcctgctgaagcaagccggcgacgtggaagaaaacccaggaccaatggcactgccagtgaccgccctgctgctgcctctggccctgctgctgcacgcagccagacccatcctgtggcacgaaatgtggcatgaaggcctggaggaggcaagcagactgtactttggcgagagaaatgtgaaaggaatgtttgaggtgctggagcctctgcacgccatgatggagaggggccctcagaccctgaaggagacatcctttaaccaggcctacggcagagacctgatggaggcccaggagtggtgcaggaagtatatgaagagcggaaatgtgaaagacctgctgcaggcctgggatctgtactaccacgtgttccgccggatctctaagggcaaggatacaatcccttggctgggacacctgctggtgggactgagcggagcctttggcttcatcatcctggtgtatctgctgatcaactgcagaaatacaggcccatggctgaagaaggtgctgaagtgtaacacccctgacccatccaagttcttttctcagctgagctccgagcacggcggcgatgtgcagaagtggctgtctagcccctttccttcctctagcttcagccctggaggactggcacctgagatctccccactggaggtgctggagagggacaaggtgacccagctgctgctgcagcaggataaggtgccagagcccgcctccctgtcctctaaccacagcctgacctcctgctttacaaatcagggctacttctttttccacctgccagacgcactggagatcgaggcatgtcaggtgtatttcacatacgatccctatagcgaggaggaccctgatgagggagtggccggcgccccaaccggaagctcccctcagccactgcagccactgagcggagaggacgatgcatattgtacatttccttcccgcgacgatctgctgctgttctctccaagcctgctgggaggaccatctccacccagcaccgcacctggaggatccggggcaggggaggagcggatgcctccatctctgcaggagagagtgccaagggactgggatccacagcctctgggaccacctacccctggagtgccagacctggtggatttccagccaccccctgagctggtgctgcgggaggcaggagaggaggtgccagacgcaggacctagagagggcgtgagctttccctggtccaggccaccaggacagggagagttccgcgccctgaacgcccggctgcccctgaatacagacgcctacctgtctctgcaggagctgcagggccaggatcctacccacctggtgtga

SEQ ID NO: 62 - DNA, 66 bp, Artificial Sequence
  Made in Lab - polynucleotide encoding signal peptide (human CSF2r)
atgctgctgctggtgacctccctgctgctgtgcgagctgcctcacccagcctttctgctgatcccc

SEQ ID NO: 63 - DNA, 735 bp, Artificial Sequence
  Made in Lab - polynucleotide encoding CD19 scFv (VL-linker-VH)
gacatccagatgacacagaccacaagctccctgtctgccagcctgggcgacagagtgaccatctcctgtagggcctctcaggatatcagcaagtacctgaactggtatcagcagaagccagatggcacagtgaagctgctgatctaccacacctccaggctgcactctggagtgccaagccggttctccggatctggaagcggcaccgactattccctgacaatctctaacctggagcaggaggatatcgccacatacttttgccagcagggcaataccctgccatatacattcggcggaggaaccaagctggagatcaccggatccacatctggaagcggcaagccaggaagcggagagggatccacaaagggagaggtgaagctgcaggagagcggaccaggactggtggcaccatcccagtctctgagcgtgacctgtacagtgtccggcgtgtctctgcctgactacggcgtgtcctggatcaggcagccacctaggaagggactggagtggctgggcgtgatctggggctctgagaccacatactataattctgccctgaagagccgcctgaccatcatcaaggacaactccaagtctcaggtgtttctgaagatgaatagcctgcagaccgacgatacagccatctactattgcgccaagcactactattacggcggctcctacgccatggattattggggccagggcacctccgtgacagtgtctagc

SEQ ID NO: 64 - DNA, 120 bp, Artificial Sequence
  Made in Lab - polynucleotide encoding IgG4 linker-CD28 TM part of vector construct
gagtctaagtatggcccaccctgccctccatgtccaatgttctgggtgctggtggtggtgggaggcgtgctggcctgttactccctgctggtgaccgtggcctttatcatcttctgggtg

SEQ ID NO: 65 - DNA, 126 bp, Artificial Sequence
  Made in Lab - polynucleotide encoding “4-1BB signal domain part of vector construct
aagagaggcaggaagaagctgctgtatatctttaagcagcccttcatgcgccctgtgcagaccacacaggaggaggacggctgcagctgtcggtttccagaggaggaggagggaggatgcgagctg

SEQ ID NO: 66 - DNA, 336 bp, Artificial Sequence
  Made in Lab - polynucleotide encoding CD3zeta part of vector construct
cgcgtgaagttcagccggtccgccgatgcccctgcctaccagcagggccagaaccagctgtataacgagctgaatctgggccggagagaggagtacgacgtgctggataagaggaggggaagggacccagagatgggaggcaagcctcggagaaagaacccacaggagggcctgtacaatgagctgcagaaggacaagatggccgaggcctattctgagatcggcatgaagggagagaggcgccggggcaagggacacgatggcctgtaccagggcctgagcaccgccacaaaggacacatatgatgccctgcacatgcaggccctgccacctagg

SEQ ID NO: 67 - DNA, 66 bp, Artificial Sequence
  Made in Lab - polynucloetide encoding P@A self-cleaving peptide part of vector construct
ggatctggagccaccaactttagcctgctgaagcaggcaggcgatgtggaggagaatccaggacct

SEQ ID NO: 68 - DNA, 267 bp, Artificial Sequence
  Made in Lab - polynucleotide enconding Naked FRB part of vector construct
gagatgtggcacgagggactggaggaggcaagcaggctgtactttggcgagcggaatgtgaagggcatgttcgaggtgctggagccactgcacgcaatgatggagaggggaccacagaccctgaaggagacatccttcaaccaggcatacggaagggacctgatggaggcacaggagtggtgccggaagtatatgaagtctggcaatgtgaaggacctgctgcaggcctgggatctgtattaccacgtgtttagaaggatcagcaag

SEQ ID NO: 69 - DNA, 66 bp, Artificial Sequence
  Made in Lab - polynucleotide encoding P2A self-cleaving peptide part of vector construct
ggctccggcgccaccaacttctccctgctgaagcaggccggcgatgtggaagaaaatccaggacca

SEQ ID NO: 70 - DNA, 378 bp, Artificial Sequence
  Made in Lab - polynucleotide encoding signal peptide FKBP12 part of vector construct
atgcctctgggactgctgtggctgggactggccctgctgggcgccctgcacgcccaggccggcgtgcaggtggagacaatcagccctggcgacggcagaacctttccaaagaggggccagacatgcgtggtgcactacaccggcatgctggaggatggcaagaagttcgactcctctcgcgatcggaacaagccctttaagttcatgctgggcaagcaggaagtgatcagaggctgggaggagggcgtggcccagatgtctgtgggccagagggccaagctgacaatcagcccagactatgcatacggagcaaccggacaccctggaatcatcccaccacacgccacactggtgttcgatgtggagctgctgaagctg

SEQ ID NO: 71 - DNA, 375 bp, Artificial Sequence
  Made in Lab - polynucleotide encoding ”IL-2R gamma TM-Cytoplasmic domain part of vector construct
ggcgagggctctaacaccagcaaggagaatccatttctgttcgcactggaggcagtggtcatctccgtgggctctatgggcctgatcatctccctgctgtgcgtgtacttttggctggagagaacaatgccaaggatccccaccctgaagaacctggaggacctggtgaccgagtaccacggcaatttcagcgcctggtccggcgtgtctaagggactggcagagtccctgcagccagattattctgagcggctgtgcctggtgagcgagatccctccaaagggaggcgccctgggagagggaccaggagccagcccctgcaaccagcactccccttactgggcccccccttgttataccctgaagccagagaca

SEQ ID NO: 72 - DNA, 66 bp, Artificial Sequence
  Made in Lab - polynucleotide encoding P2A self-cleaving peptide part of vector construct
ggctctggcgccaccaacttcagcctgctgaagcaagccggcgacgtggaagaaaacccaggacca

SEQ ID NO: 73 - DNA, 342 bp, Artificial Sequence
  Made in Lab - polynucleotide encoding signal peptideFRB part of vector construct
atggcactgccagtgaccgccctgctgctgcctctggccctgctgctgcacgcagccagacccatcctgtggcacgaaatgtggcatgaaggcctggaggaggcaagcagactgtactttggcgagagaaatgtgaaaggaatgtttgaggtgctggagcctctgcacgccatgatggagaggggccctcagaccctgaaggagacatcctttaaccaggcctacggcagagacctgatggaggcccaggagtggtgcaggaagtatatgaagagcggaaatgtgaaagacctgctgcaggcctgggatctgtactaccacgtgttccgccggatctctaag

SEQ ID NO: 74 - DNA, 945 bp, Artificial Sequence
  Made in Lab - polynucleotide encoding “IL-2R beta TM-Cytoplasmic domain part of vector construct
ggcaaggatacaatcccttggctgggacacctgctggtgggactgagcggagcctttggcttcatcatcctggtgtatctgctgatcaactgcagaaatacaggcccatggctgaagaaggtgctgaagtgtaacacccctgacccatccaagttcttttctcagctgagctccgagcacggcggcgatgtgcagaagtggctgtctagcccctttccttcctctagcttcagccctggaggactggcacctgagatctccccactggaggtgctggagagggacaaggtgacccagctgctgctgcagcaggataaggtgccagagcccgcctccctgtcctctaaccacagcctgacctcctgctttacaaatcagggctacttctttttccacctgccagacgcactggagatcgaggcatgtcaggtgtatttcacatacgatccctatagcgaggaggaccctgatgagggagtggccggcgccccaaccggaagctcccctcagccactgcagccactgagcggagaggacgatgcatattgtacatttccttcccgcgacgatctgctgctgttctctccaagcctgctgggaggaccatctccacccagcaccgcacctggaggatccggggcaggggaggagcggatgcctccatctctgcaggagagagtgccaagggactgggatccacagcctctgggaccacctacccctggagtgccagacctggtggatttccagccaccccctgagctggtgctgcgggaggcaggagaggaggtgccagacgcaggacctagagagggcgtgagctttccctggtccaggccaccaggacagggagagttccgcgccctgaacgcccggctgcccctgaatacagacgcctacctgtctctgcaggagctgcagggccaggatcctacccacctggtg

SEQ ID NO: 75 - protein, 1297 aa, Artificial Sequence
  FRB_RACR_CD19 CAR lentiviral vector Construct
MEMWHEGLEEASRLYFGERNVKGMFEVLEPLHAMMERGPQTLKETSFNQAYGRDLMEAQEWCRKYMKSGNVKDLLQAWDLYYHVFRRISKGSGATNFSLLKQAGDVEENPGPMPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVGSGATNFSLLKQAGDVEENPGPMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 112 aa, Artificial Sequence
  Made in Lab - Naked FRB_P2A part of construct
MEMWHEGLEEASRLYFGERNVKGMFEVLEPLHAMMERGPQTLKETSFNQAYGRDLMEAQEWCRKYMKSGNVKDLLQAWDLYYHVFRRISKGSGATNFSLLKQAGDVEENPGP

SEQ ID NO: 77 - protein, 273 aa, Artificial Sequence
  Made in Lab - RACRg (FKBP12_IL-2R gamma)_P2A part of construct
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGP

SEQ ID NO: 78 - protein, 451 aa, Artificial Sequence
  Made in Lab - RACRb (FRB_IL-2R beta)_P2A part of construct
MALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVGSGATNFSLLKQAGDVEENPGP

SEQ ID NO: 79 - protein, 267 aa, Artificial Sequence
  Made in Lab - CD19 scFv part of construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 80 - protein, 194 aa, Artificial Sequence
  Made in Lab - IgG4 linker-CD28 TM_4-1BB-CD3zeta part of construct
ESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - DNA, 3894 bp, Artificial Sequence
  Made in Lab - polynucleotide encoding FRB_RACR_CD19 CAR lentiviral vector construct
atggagatgtggcacgagggactggaggaggcaagcagactgtactttggcgagaggaacgtgaagggcatgttcgaggtgctggagccactgcacgcaatgatggagaggggaccacagaccctgaaggagacatctttcaaccaggcatacggaagggacctgatggaggcacaggagtggtgccggaagtatatgaagagcggcaatgtgaaggacctgctgcaggcctgggatctgtactatcacgtgtttcggagaatctccaagggctctggcgccaccaacttctccctgctgaagcaggccggcgatgtggaggagaatcctggaccaatgccactgggactgctgtggctgggactggccctgctgggcgccctgcacgcccaggccggcgtgcaggtggagacaatcagccctggcgacggacgcacctttccaaagaggggacagacatgcgtggtgcactacaccggcatgctggaggatggcaagaagttcgacagctccagagataggaataagccctttaagttcatgctgggcaagcaggaagtgatcaggggatgggaggagggagtggcacagatgtctgtgggacagcgggccaagctgacaatcagcccagactatgcatacggagcaaccggacaccctggaatcatcccacctcacgccacactggtgtttgatgtggagctgctgaagctgggcgagggcagcaacacctccaaggagaatccatttctgttcgccctggaggccgtggtcatctctgtgggcagcatgggcctgatcatctccctgctgtgcgtgtacttttggctggagcgcacaatgccacggatccccaccctgaagaacctggaggacctggtgaccgagtaccacggcaatttctccgcctggtctggcgtgagcaagggactggcagagtctctgcagccagattatagcgagcggctgtgcctggtgagcgagatcccacccaagggaggcgccctgggagagggaccaggagcctccccttgcaaccagcactctccttactgggcccctccatgttataccctgaagccagagacaggcagcggagctactaacttctccctgctgaagcaagcaggcgacgtggaagaaaatcctggaccaatggcactgccagtgaccgccctgctgctgcctctggccctgctgctgcacgcagccagacccatcctgtggcacgaaatgtggcatgaaggcctggaggaggcaagcaggctgtactttggcgagcggaatgtgaaaggaatgtttgaagtgctggagcctctgcacgccatgatggagaggggccctcagaccctgaaggagacatcctttaaccaggcctacggcagagacctgatggaggcccaggagtggtgcaggaagtatatgaagtctggaaatgtgaaagacctgctgcaggcctgggatctgtattatcacgtgttcaggcgcatctctaagggcaaggatacaatcccttggctgggacacctgctggtgggactgagcggagcctttggcttcatcatcctggtgtatctgctgatcaactgccgcaatacaggcccatggctgaagaaggtgctgaagtgtaacacccccgacccttccaagttcttttctcagctgtctagcgagcacggcggcgatgtgcagaagtggctgtcctctccatttcccagctcctctttcagcccaggaggactggcaccagagatctccccactggaggtgctggagagggacaaggtgacccagctgctgctgcagcaggataaggtgcctgagccagcctccctgagctccaaccactccctgacctcttgctttacaaatcagggctacttctttttccacctgccagacgcactggagatcgaggcatgtcaggtgtatttcacatacgatccctatagcgaggaggaccctgatgagggagtggccggcgccccaaccggatctagcccacagcctctgcagccactgagcggagaggacgatgcatattgtacatttccttcccgcgacgatctgctgctgttctctccaagcctgctgggaggaccaagcccaccttccaccgcaccaggcggctccggggcaggggaggagcggatgccaccctctctgcaggagagagtgccaagggactgggatccacagccactgggacctccaacccctggagtgccagacctggtggatttccagccccctccagagctggtgctgagagaggcaggagaggaggtgcctgacgcaggaccaagagagggcgtgagctttccttggtccaggccacctggacagggagagttcagagccctgaacgccaggctgcccctgaatacagacgcctacctgtctctgcaggagctgcagggccaggatcctacacacctggtcggatctggcgccaccaactttagcctgctgaagcaggcaggcgacgtggaagagaaccctggaccaatgctgctgctggtgaccagcctgctgctgtgcgagctgccacaccctgccttcctgctgatcccagatatccagatgacacagaccacatcctctctgtccgcctctctgggcgacagagtgaccatctcttgtagggccagccaggatatctccaagtacctgaactggtatcagcagaagcctgacggcacagtgaagctgctgatctaccacacctctaggctgcacagcggagtgccatcccggttcagcggatccggatctggaacagactattctctgaccatcagcaacctggagcaggaggatatcgccacatacttttgccagcagggcaataccctgccatatacattcggcggaggaaccaagctggagatcaccggaagcacatccggatctggcaagccaggatccggagagggatctacaaagggagaggtgaagctgcaggagagcggaccaggactggtggcacccagccagtccctgtctgtgacctgtacagtgtctggcgtgagcctgcccgattacggcgtgtcctggatcagacagccaccaaggaagggactggagtggctgggcgtgatctggggctctgagaccacatactataatagcgccctgaagtcccggctgaccatcatcaaggacaacagcaagtcccaggtgtttctgaagatgaatagcctgcagaccgacgatacagccatctactattgcgccaagcactactattacggcggctcctacgccatggattattggggccagggcacctccgtgacagtgagctccgagtctaagtatggccctccatgccccccttgtcctatgttctgggtgctggtggtggtgggaggcgtgctggcctgttactccctgctggtgaccgtggcctttatcatcttctgggtgaagcgcggccggaagaagctgctgtatatctttaagcagcccttcatgagacctgtgcagaccacacaggaggaggacggctgcagctgtaggtttccagaggaggaggagggaggatgcgagctgcgcgtgaagttctctcggagcgccgatgcccctgcctaccagcagggacagaaccagctgtataacgagctgaatctgggccggagagaggagtacgacgtgctggataagaggaggggaagagacccagagatgggaggcaagcctcggagaaagaacccacaggagggcctgtacaatgagctgcagaaggacaagatggccgaggcctattccgagatcggcatgaagggagagaggcgccggggcaagggacacgatggcctgtaccagggcctgagcaccgccacaaaggacacctatgatgccctgcacatgcaggccctgccacccaggtga

SEQ ID NO: 82 - DNA, 336 bp, Artificial Sequence
  Made in Lab - polynucleotide encoding Naked FRB_P2A part of vector construct
atggagatgtggcacgagggactggaggaggcaagcagactgtactttggcgagaggaacgtgaagggcatgttcgaggtgctggagccactgcacgcaatgatggagaggggaccacagaccctgaaggagacatctttcaaccaggcatacggaagggacctgatggaggcacaggagtggtgccggaagtatatgaagagcggcaatgtgaaggacctgctgcaggcctgggatctgtactatcacgtgtttcggagaatctccaagggctctggcgccaccaacttctccctgctgaagcaggccggcgatgtggaggagaatcctggacca

SEQ ID NO: 83 - DNA, 819 bp, Artificial Sequence
  Made in Lab - polynucleotide encoding RACRg (FKBP12_IL-2R gamma)_P2A part of vector construct
atgccactgggactgctgtggctgggactggccctgctgggcgccctgcacgcccaggccggcgtgcaggtggagacaatcagccctggcgacggacgcacctttccaaagaggggacagacatgcgtggtgcactacaccggcatgctggaggatggcaagaagttcgacagctccagagataggaataagccctttaagttcatgctgggcaagcaggaagtgatcaggggatgggaggagggagtggcacagatgtctgtgggacagcgggccaagctgacaatcagcccagactatgcatacggagcaaccggacaccctggaatcatcccacctcacgccacactggtgtttgatgtggagctgctgaagctgggcgagggcagcaacacctccaaggagaatccatttctgttcgccctggaggccgtggtcatctctgtgggcagcatgggcctgatcatctccctgctgtgcgtgtacttttggctggagcgcacaatgccacggatccccaccctgaagaacctggaggacctggtgaccgagtaccacggcaatttctccgcctggtctggcgtgagcaagggactggcagagtctctgcagccagattatagcgagcggctgtgcctggtgagcgagatcccacccaagggaggcgccctgggagagggaccaggagcctccccttgcaaccagcactctccttactgggcccctccatgttataccctgaagccagagacaggcagcggagctactaacttctccctgctgaagcaagcaggcgacgtggaagaaaatcctggacca

SEQ ID NO: 84 - DNA, 1353 bp, Artificial Sequence
  Made in Lab - polynucleotide encoding RACRb (FRB_IL-2R beta)_P2A part of vector construct
atggcactgccagtgaccgccctgctgctgcctctggccctgctgctgcacgcagccagacccatcctgtggcacgaaatgtggcatgaaggcctggaggaggcaagcaggctgtactttggcgagcggaatgtgaaaggaatgtttgaagtgctggagcctctgcacgccatgatggagaggggccctcagaccctgaaggagacatcctttaaccaggcctacggcagagacctgatggaggcccaggagtggtgcaggaagtatatgaagtctggaaatgtgaaagacctgctgcaggcctgggatctgtattatcacgtgttcaggcgcatctctaagggcaaggatacaatcccttggctgggacacctgctggtgggactgagcggagcctttggcttcatcatcctggtgtatctgctgatcaactgccgcaatacaggcccatggctgaagaaggtgctgaagtgtaacacccccgacccttccaagttcttttctcagctgtctagcgagcacggcggcgatgtgcagaagtggctgtcctctccatttcccagctcctctttcagcccaggaggactggcaccagagatctccccactggaggtgctggagagggacaaggtgacccagctgctgctgcagcaggataaggtgcctgagccagcctccctgagctccaaccactccctgacctcttgctttacaaatcagggctacttctttttccacctgccagacgcactggagatcgaggcatgtcaggtgtatttcacatacgatccctatagcgaggaggaccctgatgagggagtggccggcgccccaaccggatctagcccacagcctctgcagccactgagcggagaggacgatgcatattgtacatttccttcccgcgacgatctgctgctgttctctccaagcctgctgggaggaccaagcccaccttccaccgcaccaggcggctccggggcaggggaggagcggatgccaccctctctgcaggagagagtgccaagggactgggatccacagccactgggacctccaacccctggagtgccagacctggtggatttccagccccctccagagctggtgctgagagaggcaggagaggaggtgcctgacgcaggaccaagagagggcgtgagctttccttggtccaggccacctggacagggagagttcagagccctgaacgccaggctgcccctgaatacagacgcctacctgtctctgcaggagctgcagggccaggatcctacacacctggtcggatctggcgccaccaactttagcctgctgaagcaggcaggcgacgtggaagagaaccctggacca

SEQ ID NO: 85 - DNA, 801 bp, Artificial Sequence
  Made in Lab - polynucleotide encoding CD19 scFv part of vector construct
atgctgctgctggtgaccagcctgctgctgtgcgagctgccacaccctgccttcctgctgatcccagatatccagatgacacagaccacatcctctctgtccgcctctctgggcgacagagtgaccatctcttgtagggccagccaggatatctccaagtacctgaactggtatcagcagaagcctgacggcacagtgaagctgctgatctaccacacctctaggctgcacagcggagtgccatcccggttcagcggatccggatctggaacagactattctctgaccatcagcaacctggagcaggaggatatcgccacatacttttgccagcagggcaataccctgccatatacattcggcggaggaaccaagctggagatcaccggaagcacatccggatctggcaagccaggatccggagagggatctacaaagggagaggtgaagctgcaggagagcggaccaggactggtggcacccagccagtccctgtctgtgacctgtacagtgtctggcgtgagcctgcccgattacggcgtgtcctggatcagacagccaccaaggaagggactggagtggctgggcgtgatctggggctctgagaccacatactataatagcgccctgaagtcccggctgaccatcatcaaggacaacagcaagtcccaggtgtttctgaagatgaatagcctgcagaccgacgatacagccatctactattgcgccaagcactactattacggcggctcctacgccatggattattggggccagggcacctccgtgacagtgagctcc

SEQ ID NO: 86 - DNA, 582 bp, Artificial Sequence
  Made in Lab - polynucleotide encoding IgG4 linker-CD28 TM_4-1BB-CD3zeta part of vector construct
gagtctaagtatggccctccatgccccccttgtcctatgttctgggtgctggtggtggtgggaggcgtgctggcctgttactccctgctggtgaccgtggcctttatcatcttctgggtgaagcgcggccggaagaagctgctgtatatctttaagcagcccttcatgagacctgtgcagaccacacaggaggaggacggctgcagctgtaggtttccagaggaggaggagggaggatgcgagctgcgcgtgaagttctctcggagcgccgatgcccctgcctaccagcagggacagaaccagctgtataacgagctgaatctgggccggagagaggagtacgacgtgctggataagaggaggggaagagacccagagatgggaggcaagcctcggagaaagaacccacaggagggcctgtacaatgagctgcagaaggacaagatggccgaggcctattccgagatcggcatgaagggagagaggcgccggggcaagggacacgatggcctgtaccagggcctgagcaccgccacaaaggacacctatgatgccctgcacatgcaggccctgccacccagg

SEQ ID NO: 87 - protein, 794 aa, Artificial Sequence
  FRB_CD19 CAR_TGFbDN lentiviral vector Construct
MEMWHEGLEEASRLYFGERNVKGMFEVLEPLHAMMERGPQTLKETSFNQAYGRDLMEAQEWCRKYMKSGNVKDLLQAWDLYYHVFRRISKGSGATNFSLLKQAGDVEENPGPMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKRRR

SEQ ID NO: 88 - protein, 112 aa, Artificial Sequence; identical to SEQ ID NO: 76
  Made in Lab - Naked FRB_P2A part of construct

SEQ ID NO: 89 - protein, 267 aa, Artificial Sequence; identical to SEQ ID NO: 79
  Made in Lab - CD19 scFv part of construct

SEQ ID NO: 90 - protein, 216 aa, Artificial Sequence
  Made in Lab - IgG4 linker-CD28 TM_4-1BB-CD3zeta_P2A part of construct
ESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGP

SEQ ID NO: 91 - protein, 199 aa, Artificial Sequence
  Made in Lab - TGF beta DN part of construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKRRR

SEQ ID NO: 92 - DNA, 2385 bp, Artificial Sequence
  Made in Lab - polynucleotide encoding FRB_CD19 CAR_TGFbDN lentiviral vector construct
atggagatgtggcacgagggactggaggaggcatccagactgtacttcggcgagaggaacgtgaagggcatgtttgaggtgctggagccactgcacgccatgatggagagaggcccccagaccctgaaggagacatctttcaaccaggcctatggaagggacctgatggaggcacaggagtggtgccggaagtacatgaagagcggcaatgtgaaggacctgctgcaggcctgggatctgtactatcacgtgttccggagaatcagcaagggctccggcgccaccaactttagcctgctgaagcaggcaggcgacgtggaggagaatccaggacctatgctgctgctggtgacatccctgctgctgtgcgagctgccacacccagccttcctgctgatccccgatatccagatgacccagaccacaagctccctgagcgcctccctgggcgacagggtgacaatctcttgtcgggccagccaggatatctccaagtatctgaattggtaccagcagaagcccgacggcaccgtgaagctgctgatctatcacacatctagactgcacagcggcgtgccttccaggttttctggcagcggctccggcaccgactactctctgacaatcagcaacctggagcaggaggatatcgccacctatttctgccagcagggcaataccctgccttacacatttggcggcggcacaaagctggagatcaccggctctacaagcggatccggcaagccaggatccggagagggatctaccaagggagaggtgaagctgcaggagagcggacctggactggtggcaccatctcagagcctgtccgtgacctgtacagtgtctggcgtgagcctgccagattatggcgtgagctggatcaggcagccacctaggaagggactggagtggctgggcgtgatctggggctccgagaccacatactataacagcgccctgaagtcccgcctgaccatcatcaaggacaactctaagagccaggtgttcctgaagatgaattccctgcagaccgacgatacagccatctactattgcgccaagcactactattacggcggctcttatgccatggattactggggccagggcaccagcgtgacagtgtctagcgagtccaagtacggcccaccctgccctccatgtcccatgttttgggtgctggtggtggtgggaggcgtgctggcctgttattccctgctggtgaccgtggccttcatcatcttttgggtgaagcgcggccggaagaagctgctgtacatcttcaagcagcccttcatgagacccgtgcagaccacacaggaggaggacggctgcagctgtaggttcccagaggaggaggagggaggatgcgagctgagggtgaagttttcccggtctgccgatgcccctgcctatcagcagggccagaatcagctgtacaacgagctgaatctgggcaggcgcgaggagtacgacgtgctggataagaggagaggaagggaccctgagatgggaggcaagccaaggcgcaagaaccctcaggagggcctgtataatgagctgcagaaggacaagatggccgaggcctactccgagatcggcatgaagggagagcggagaaggggcaagggacacgatggcctgtatcagggcctgagcaccgccacaaaggacacctacgatgcactgcacatgcaggccctgccacctagaggatctggagccacaaacttcagcctgctgaagcaggccggcgatgtggaggagaatcctggaccaatgggaagaggactgctgaggggactgtggccactgcacatcgtgctgtggaccaggatcgcctctacaatcccaccccacgtgcagaagagcgtgaacaatgacatgatcgtgaccgataacaatggcgccgtgaagtttccccagctgtgcaagttctgtgacgtgcgcttttccacctgtgataaccagaagtcctgcatgtctaattgtagcatcacatccatctgcgagaagcctcaggaggtgtgcgtggccgtgtggcggaagaacgacgagaatatcaccctggagacagtgtgccacgatcccaagctgccttatcacgacttcatcctggaggatgccgcctctcctaagtgtatcatgaaggagaagaagaagccaggcgagaccttctttatgtgcagctgttcctctgacgagtgcaacgataatatcatcttctccgaggagtacaacacctctaatcctgacctgctgctggtcatctttcaggtgacaggcatctccctgctgcctccactgggcgtggccatctctgtgatcatcatcttttattgttacagagtgaacaggcagcagaagcgccggcgctag

SEQ ID NO: 93 - DNA, 336 bp, Artificial Sequence
  Made in Lab - polynucleotide encoding Naked FRB_P2A part of vector construct
atggagatgtggcacgagggactggaggaggcatccagactgtacttcggcgagaggaacgtgaagggcatgtttgaggtgctggagccactgcacgccatgatggagagaggcccccagaccctgaaggagacatctttcaaccaggcctatggaagggacctgatggaggcacaggagtggtgccggaagtacatgaagagcggcaatgtgaaggacctgctgcaggcctgggatctgtactatcacgtgttccggagaatcagcaagggctccggcgccaccaactttagcctgctgaagcaggcaggcgacgtggaggagaatccaggacct

SEQ ID NO: 94 - DNA, 801 bp, Artificial Sequence
  Made in Lab - polynucleotide encoding CD19 scFv part of vector construct
atgctgctgctggtgacatccctgctgctgtgcgagctgccacacccagccttcctgctgatccccgatatccagatgacccagaccacaagctccctgagcgcctccctgggcgacagggtgacaatctcttgtcgggccagccaggatatctccaagtatctgaattggtaccagcagaagcccgacggcaccgtgaagctgctgatctatcacacatctagactgcacagcggcgtgccttccaggttttctggcagcggctccggcaccgactactctctgacaatcagcaacctggagcaggaggatatcgccacctatttctgccagcagggcaataccctgccttacacatttggcggcggcacaaagctggagatcaccggctctacaagcggatccggcaagccaggatccggagagggatctaccaagggagaggtgaagctgcaggagagcggacctggactggtggcaccatctcagagcctgtccgtgacctgtacagtgtctggcgtgagcctgccagattatggcgtgagctggatcaggcagccacctaggaagggactggagtggctgggcgtgatctggggctccgagaccacatactataacagcgccctgaagtcccgcctgaccatcatcaaggacaactctaagagccaggtgttcctgaagatgaattccctgcagaccgacgatacagccatctactattgcgccaagcactactattacggcggctcttatgccatggattactggggccagggcaccagcgtgacagtgtctagc

SEQ ID NO: 95 - DNA, 648 bp, Artificial Sequence
  Made in Lab - polynucleotide encoding IgG4 linker-CD28 TM_4-1BB-CD3zeta_P2A part of vector construct
gagtccaagtacggcccaccctgccctccatgtcccatgttttgggtgctggtggtggtgggaggcgtgctggcctgttattccctgctggtgaccgtggccttcatcatcttttgggtgaagcgcggccggaagaagctgctgtacatcttcaagcagcccttcatgagacccgtgcagaccacacaggaggaggacggctgcagctgtaggttcccagaggaggaggagggaggatgcgagctgagggtgaagttttcccggtctgccgatgcccctgcctatcagcagggccagaatcagctgtacaacgagctgaatctgggcaggcgcgaggagtacgacgtgctggataagaggagaggaagggaccctgagatgggaggcaagccaaggcgcaagaaccctcaggagggcctgtataatgagctgcagaaggacaagatggccgaggcctactccgagatcggcatgaagggagagcggagaaggggcaagggacacgatggcctgtatcagggcctgagcaccgccacaaaggacacctacgatgcactgcacatgcaggccctgccacctagaggatctggagccacaaacttcagcctgctgaagcaggccggcgatgtggaggagaatcctggacca

SEQ ID NO: 96 - DNA, 600 bp, Artificial Sequence
  Made in Lab - polynucleotide encoding TGF beta DNpart of vector construct
atgggaagaggactgctgaggggactgtggccactgcacatcgtgctgtggaccaggatcgcctctacaatcccaccccacgtgcagaagagcgtgaacaatgacatgatcgtgaccgataacaatggcgccgtgaagtttccccagctgtgcaagttctgtgacgtgcgcttttccacctgtgataaccagaagtcctgcatgtctaattgtagcatcacatccatctgcgagaagcctcaggaggtgtgcgtggccgtgtggcggaagaacgacgagaatatcaccctggagacagtgtgccacgatcccaagctgccttatcacgacttcatcctggaggatgccgcctctcctaagtgtatcatgaaggagaagaagaagccaggcgagaccttctttatgtgcagctgttcctctgacgagtgcaacgataatatcatcttctccgaggagtacaacacctctaatcctgacctgctgctggtcatctttcaggtgacaggcatctccctgctgcctccactgggcgtggccatctctgtgatcatcatcttttattgttacagagtgaacaggcagcagaagcgccggcgctag

SEQ ID NO: 97 - protein, 67 aa, Artificial Sequence
  human Glycophorin A TM_ CT
SGHFSEPEITLIIFGVMAGVIGTILLISYGIRRLIKKSPSDVKPLPSPDTDVPLSSVEIENPETSDQ

SEQ ID NO: 98 - DNA, 201 bp, Artificial Sequence
  218 linker_human Glycophorin A ecto-TM_HIV Env CT
tccggacacttcagcgagcctgagatcaccctgatcatcttcggcgtgatggccggagtgatcggcacaatcctgctgatcagctacggcatcagaagactgattaagaaatccccatctgatgtgaagcctctgccttctcctgacaccgacgtccccctgagcagcgtggaaatcgagaaccccgaaaccagcgaccag

SEQ ID NO: 99 - protein, 500 aa, Artificial Sequence
  gag protein
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTHNPPIPVGEIYKRWIILGLNKIVRMYSPTSILDIRQGPKEPFRDYVDRFYKTLRAEQASQEVKNWMTETLLVQNANPDCKTILKALGPGATLEEMMTACQGVGGPGHKARVLAEAMSQVTNPATIMIQKGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLASLRSLFGSDPSSQ

SEQ ID NO: 100 - protein, 1003 aa, Artificial Sequence
  Pol protein
FFREDLAFPQGKAREFSSEQTRANSPTRRELQVWGRDNNSLSEAGADRQGTVSFSFPQITLWQRPLVTIKIGGQLKEALLDTGADDTVLEEMNLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKQKKSVTVLDVGDAYFSVPLDKDFRKYTAFTIPSINNETPGIRYQYNVLPQGWKGSPAIFQCSMTKILEPFRKQNPDIVIYQYMDDLYVGSDLEIGQHRTKIEELRQHLLRWGFTTPDKKHQKEPPFLWMGYELHPDKWTVQPIVLPEKDSWTVNDIQKLVGKLNWASQIYAGIKVRQLCKLLRGTKALTEVVPLTEEAELELAENREILKEPVHGVYYDPSKDLIAEIQKQGQGQWTYQIYQEPFKNLKTGKYARMKGAHTNDVKQLTEAVQKIATESIVIWGKTPKFKLPIQKETWEAWWTEYWQATWIPEWEFVNTPPLVKLWYQLEKEPIIGAETFYVDGAANRETKLGKAGYVTDRGRQKVVPLTDTTNQKTELQAIHLALQDSGLEVNIVTDSQYALGIIQAQPDKSESELVSQIIEQLIKKEKVYLAWVPAHKGIGGNEQVDKLVSAGIRKVLFLDGIDKAQEEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNFKRKGGIGGYSAGERIVDIIATDIQTKELQKQITKIQNFRVYYRDSRDPVWKGPAKLLWKGEGAVVIQDNSDIKVVPRRKAKIIRDYGKQMAGDDCVASRQDED

SEQ ID NO: 101 - DNA, 4307 bp, Artificial Sequence
  gag-pol nucleic acid
atgggcgcccgcgccagcgtgctttctggcggcgagctggacaggtgggagaagattcgcctgcggcctggaggaaagaaaaagtacaagctgaagcacatcgtgtgggcttctcgggaactggaaagattcgccgtgaaccctggactgctagagacctccgaaggctgcagacagatcctgggacagctgcaacctagcctgcagaccggcagcgaggagctgagaagcctgtacaacaccgtcgccaccctgtattgtgtgcaccaaagaatcgagatcaaggacaccaaggaggctctggataagatcgaggaagagcagaacaagagcaagaaaaaagcccagcaggccgccgctgataccggccattctaatcaggtgtcccagaactaccccattgtgcaaaatatccagggccagatggtccaccaggccatcagccctagaaccctgaatgcctgggtgaaggtggtggaagagaaggccttttctccagaggtgatccctatgttcagcgccctgagcgagggcgctacccctcaggacctgaacacaatgctgaacaccgtgggcggccaccaggccgccatgcagatgctgaaagaaaccatcaacgaggaggccgccgaatgggaccgggtgcaccccgttcacgccggcccaatcgcccctggccagatgcgggaacctagaggcagcgacatcgccggcacaaccagcacactgcaagagcagatcggatggatgacacacaacccccccatccccgtgggcgaaatctacaagcggtggatcattctgggactgaacaaaatcgttagaatgtacagccctaccagcatcctggatatcagacagggcccaaaggagcctttccgggactacgtggacagattttacaagaccctgagagccgaacaggcctcccaagaggtgaagaactggatgacagagaccctgctggtgcagaatgccaaccctgattgtaagacaatcctgaaggccctcggacctggcgctacactggaagaaatgatgaccgcctgccagggcgtgggcggccccggccacaaggccagagtgctggccgaggctatgagccaggtgacaaaccccgccacaatcatgatccagaagggcaacttcagaaaccagcggaagaccgtgaaatgcttcaactgcggcaaggaaggccacatcgcaaagaactgcagagcccctaggaagaaaggctgttggaagtgcggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagatcggcggacagctgaaagaggccttgctggacaccggagccgatgacaccgtgctggaagaaatgaacctgcctggaagatggaaacctaagatgatcggtggcatcggcggatttatcaaagtgcgacagtatgaccagatcctgatcgagatttgcggccacaaagctatcggaacagtgctggtcggcccgacccccgtgaacatcattggccgcaacctgctgacacagatcggttgtacactgaactttcctatcagccctatcgaaaccgtgccggtcaagctgaagcccggcatggatggccctaaggtgaagcagtggcccctgacagaggaaaagatcaaggcactggtggaaatctgcacagaaatggaaaaagagggcaagatttctaaaatcggcccagagaacccctacaacacccctgttttcgccatcaagaagaaagattccaccaagtggaggaagctggtggactttcgggaactgaacaagcggacccaggatttctgggaggtgcagctgggcatcccccaccctgccggcctgaaacaaaaaaaaagcgtgaccgtgctggacgtgggcgacgcctatttcagcgtgcctctggataaggacttccggaaatacaccgcctttaccatccctagcatcaacaacgagacccctggcatccggtaccagtacaacgtgctcccacagggctggaagggctcacccgccatcttccagtgcagcatgaccaagatcctggagcctttcagaaagcagaatcctgacatcgtgatctaccagtacatggacgacctgtacgtgggctctgatctggagatcggacagcacagaacaaagatcgaagagctgagacagcatctgctgagatggggtttcaccacccccgacaagaagcaccagaaggaacctccttttctgtggatgggctacgagctgcaccccgataagtggacagtgcagcccatcgtgctgcccgagaaggactcctggaccgtgaacgacattcagaagctggtcggaaagctgaattgggcttcccaaatctacgccggcatcaaggtgcggcagctgtgcaagcttctgcgcggcacaaaggccctgacggaagtcgtgccactgaccgaggaagccgaattagagctggccgaaaacagagaaattctgaaagaacctgtgcacggcgtttactacgacccttctaaggacctgatcgccgaaatccagaaacaaggccagggccagtggacttaccaaatctaccaggagcctttcaaaaacctcaagaccggcaagtacgccagaatgaagggagcccatacaaacgacgtgaagcagctgacagaggctgttcagaagatcgccacagaaagcatcgtgatctggggcaagaccccaaagttcaagctgcctatccaaaaggaaacctgggaggcctggtggaccgagtactggcaggccacctggattcctgaatgggagttcgtgaacacaccacctcttgtgaagctgtggtaccagctggaaaaggagccaatcatcggcgccgagacattctacgtggacggcgccgccaaccgggagaccaaactgggaaaggccggatatgtgaccgacagaggcagacaaaaggtggtgcctctgaccgatacaactaaccagaaaacagagctgcaggccattcacctggccctgcaagacagcggcctggaagtgaatatcgtgacagacagtcagtacgccctgggcatcatccaggctcagcctgacaagagcgagagcgagctggtgtcccagatcatcgagcagctgatcaaaaaggagaaggtttatctggcctgggtgcccgcccacaagggcatcggaggcaacgagcaggtggacaagctggtcagcgccggcatccggaaggtgctgttcctggacggcatcgacaaggcccaggaggaacacgagaagtaccacagcaactggcgggccatggccagcgacttcaacctgccacctgtggttgctaaggagatcgtcgcctcttgtgataagtgccagctgaagggcgaggccatgcacggccaagtggattgcagccctgggatctggcagctagactgtacccacctggagggcaaggtgatcctggtggcagtgcacgtggccagcggctacatcgaggctgaggtgatccccgccgaaacgggccaggagaccgcctactttctgctgaagctagccggccggtggcctgtgaagaccgtgcacaccgataacggcagcaatttcaccagcacaaccgtgaaggctgcctgctggtgggctggaatcaagcaggagttcggcatcccatacaatcctcagtctcagggcgtgatcgagagcatgaacaaggaactgaagaagatcattggtcaggtcagagatcaggccgagcacctgaaaaccgccgttcaaatggctgtgttcatccataacttcaaaagaaaaggcggcatcggcggctacagcgccggcgaaagaatcgtggatatcatcgcgaccgacatccaaacaaaagagctgcaaaagcaaatcaccaagatccagaacttcagagtgtactacagagatagcagagatcctgtgtggaagggacctgccaagctgctgtggaagggcgagggcgccgtggtgatccaggacaatagcgacatcaaggtcgtgcccagaagaaaggctaaaatcattagagactacggcaaacagatggccggagatgattgcgtggcttctagacaggacgaggactga

SEQ ID NO: 102 - protein, 116 aa, Artificial Sequence
  Rev protein
MAGRSGDSDEDLLKAVRLIKFLYQSNPPPNPEGTRQARRNRRRRWRERQRQIHSISERILSTYLGRSAEPVPLQLPPLERLTLDCNEDCGTSGTQGVGSPQILVESPTILESGAKE

SEQ ID NO: 103 - DNA, 351 bp, Artificial Sequence
  Rev nucleic acid
atggccggcagaagcggcgacagcgacgaggatctgctgaaagccgtgcggctgatcaagttcctgtaccagagcaaccctcctcctaaccccgagggcaccagacaggctagacggaaccgcagaagaaggtggcgggaacggcaaagacagatccactctatcagcgagagaatcctgagcacctacctgggaagatccgccgagcctgtccccctgcagctgcctccactggaaagactgaccctggattgtaatgaggactgcggcacaagcggaacccagggcgtgggcagcccccagattctggtggaatcccctacaatcctcgagtctggcgccaaggaatga

SEQ ID NO: 104 - DNA, 1539 bp, Artificial Sequence
  Cocal glycoprotein
atgaactttctgctgctgaccttcatcgtgctgcctctgtgcagccacgccaagttttccatcgtgttcccacagtcccagaagggcaactggaagaatgtgcccagctcctaccactattgtccttctagctccgaccagaactggcacaatgatctgctgggcatcaccatgaaggtgaagatgcctaagacacacaaggccatccaggcagatggatggatgtgccacgcagccaagtggatcaccacatgtgactttcggtggtacggccccaagtatatcacccacagcatccactccatccagcctacaagcgagcagtgcaaggagtccatcaagcagaccaagcagggcacatggatgtctcccggcttcccccctcagaactgtggctacgccaccgtgacagatagcgtggcagtggtggtgcaggcaaccccacaccacgtgctggtggatgagtatacaggcgagtggatcgacagccagtttcccaacggcaagtgcgagaccgaggagtgtgagacagtgcacaattctaccgtgtggtacagcgattataaggtgaccggcctgtgcgacgccacactggtggataccgagatcacattcttttccgaggacggcaagaaggagtctatcggcaagcccaacaccggctacaggtctaattacttcgcctatgagaagggcgataaggtgtgcaagatgaattattgtaagcacgccggggtgcggctgccaagcggcgtgtggtttgagttcgtggaccaggacgtgtacgcagcagcaaagctgccagagtgcccagtgggagcaaccatcagcgcccccacccagacatctgtggacgtgagcctgatcctggatgtggagagaatcctggactactccctgtgccaggagacatggtccaagatccgctctaagcagcccgtgagcccagtggacctgtcttacctggcaccaaagaaccctggaacaggacctgcctttaccatcatcaatggcacactgaagtacttcgagacccggtatatcagaatcgacatcgataacccaatcatctccaagatggtgggcaagatctccggctctcagaccgagagagagctgtggacagagtggttcccatacgagggcgtggagatcggccccaatggcatcctgaagacccctacaggctataagtttccactgttcatgatcggccacggcatgctggactctgatctgcacaagaccagccaggccgaggtgtttgagcacccacacctggcagaggcaccaaagcagctgcccgaggaggagaccctgttctttggcgatacaggcatctccaagaaccctgtggagctgatcgagggctggttttctagctggaagtctaccgtggtgacattctttttcgccatcggcgtgttcatcctgctgtacgtggtggcaaggatcgtgatcgccgtgcggtacagatatcagggcagcaacaataagagaatctataatgacatcgagatgtccaggttccgcaagtga

SEQ ID NO: 105 - protein, 65 aa, Artificial Sequence
  human Glycophorin A hinge-TM-CT
HFSEPEITLIIFGVMAGVIGTILLISYGIRRLIKKSPSDVKPLPSPDTDVPLSSVEIENPETSDQ

SEQ ID NO: 106 - DNA, 195 bp, Artificial Sequence
  human Glycophorin A hinge-TM-CT - nucleic acid
cacttcagcgagcctgagatcaccctgatcatcttcggcgtgatggccggagtgatcggcacaatcctgctgatcagctacggcatcagaagactgattaagaaatccccatctgatgtgaagcctctgccttctcctgacaccgacgtccccctgagcagcgtggaaatcgagaaccccgaaaccagcgaccag

SEQ ID NO: 107 - protein, 324 aa, Artificial Sequence
  anti-CD3scFv_short hinge-TM-CT
MGVKVLFALICIAVAEADIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASGVELIEGWFSSWKSTVVTFFFAIGVFILLYVVARIVIAVRYRYQGSNNKRIYNDIEMSRFRK

SEQ ID NO: 108 - DNA, 972 bp, Artificial Sequence
  anti-CD3scFv_short hinge-TM-CT - nucleic acid
atgggcgtgaaagtgctgttcgccctgatctgcatcgcagttgctgaagccgacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgcctccggagtggaactgatcgagggctggttcagcagctggaaaagcaccgtggttacattctttttcgccatcggcgtgttcatcctgctgtacgtggtcgccagaattgtgatcgccgtgcggtatagataccagggcagcaacaacaagcggatctacaacgacatcgagatgagcagattcagaaag

SEQ ID NO: 109 - protein, 355 aa, Artificial Sequence
  anti-CD3scFv_long hinge_TM_CT
MGVKVLFALICIAVAEADIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASSGFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 110 - DNA, 1062 bp, Artificial Sequence
  anti-CD3scFv_long hinge_TM_CT - nucleic acid
atgggcgtgaaagtgctgttcgccctgatctgcatcgcagttgctgaagccgacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgcctccggattcgagcacccccacctggccgaggcccctaagcagctgcctgaagaagagacactgtttttcggagataccggcatcagcaaaaaccccgtggagctgatcgagggctggttcagctcttggaagagcaccgtggtcacattctttttcgccatcggcgtctttatcctgctgtacgtggtagccagaatcgtgatcgccgtgcggtacagataccagggcagcaacaacaagcggatctacaacgacatcgagatgagccggttcagaaag

SEQ ID NO: 111 - protein, 374 aa, Artificial Sequence
  anti-CD3scFv_ hGlycophorinA_TM_HIV Env CT
MGVKVLFALICIAVAEADIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASGGSTSGSGKPGSGEGSTKGPEITLIIFGVMAGVIGTILLISYGIRRLALKYWWNLLQYWSQELKNSAVSLLNATAIAVAEGTDRVIEVVQGACRAIRHIPRRIRQGLERILL

SEQ ID NO: 112 - DNA, 1122 bp, Artificial Sequence
  anti-CD3scFv_ hGlycophorinA_TM_HIV Env CT
atgggcgtgaaagtgctgttcgccctgatctgcatcgcagttgctgaagccgacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgcctccggaggatctacaagcggctctggcaagcctggcagcggagaaggcagcaccaagggccctgagatcacactgatcatcttcggcgtgatggccggcgtcatcggcaccatcctgctgatcagctacggcatcagaagactggctctgaagtactggtggaatctgctgcaatactggagccaggagctgaaaaacagcgccgtgtccctgctcaacgccaccgccatcgccgtggccgagggcaccgacagagtgatcgaggtggtgcagggagcctgcagagctattcggcacatccccagacggatcaggcagggcctggaaagaatcctgctg

SEQ ID NO: 113 - protein, 380 aa, Artificial Sequence
  anti-CD3scFv_ 218 linker_HIV Env ecto-TM-CT
MGVKVLFALICIAVAEADIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASGGSTSGSGKPGSGEGSTKGNWLWYIRIFIIIVGSLIGLRIVFAVLSLVNRGWEALKYWWNLLQYWSQELKNSAVSLLNATAIAVAEGTDRVIEVVQGACRAIRHIPRRIRQGLERILL

SEQ ID NO: 114 - DNA, 1140 bp, Artificial Sequence
  anti-CD3scFv_ 218 linker_HIV Env ecto-TM-CT - nucleic acid
atgggcgtgaaagtgctgttcgccctgatctgcatcgcagttgctgaagccgacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgcctccggaggaagcaccagcggctctggcaagcctggcagcggcgagggctctaccaagggcaattggctgtggtacatcagaatcttcatcatcatcgtgggcagcctgatcggcctgagaatcgtgttcgccgtgctgagcctggtgaaccggggctgggaagctctgaagtactggtggaacctgctgcaatactggtcccaggagctgaaaaacagcgctgtgtccctgctcaacgccaccgccatcgccgtcgccgagggaacagacagagtgatcgaggtggtgcagggagcctgcagagccattcggcacatccccagacgcatcagacagggcctggaaagaatcctgctg

SEQ ID NO: 115 - protein, 373 aa, Artificial Sequence
  anti-CD3scFv_ G4S linker_HIV Env ecto-TM-CT
MGVKVLFALICIAVAEADIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASGGGGGSGGGGSGGGGSYIRIFIIIVGSLIGLRIVFAVLSLVNRGWEALKYWWNLLQYWSQELKNSAVSLLNATAIAVAEGTDRVIEVVQGACRAIRHIPRRIRQGLERILL

SEQ ID NO: 116 - DNA, 1119 bp, Artificial Sequence
  anti-CD3scFv_ G4S linker_HIV Env ecto-TM-CT - nucleic acid
atgggcgtgaaagtgctgttcgccctgatctgcatcgcagttgctgaagccgacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgcctccggaggcggtggaggctctggtggcggagggagcggtggcggaggcagctacatcagaatcttcatcatcatcgtgggcagcctgatcggcctgagaatcgtgttcgccgttctgagcctggtgaaccggggctgggaagccctgaagtactggtggaatctgctccagtactggtctcaggagctgaagaacagcgccgtgtccctgctgaacgctacagctatcgccgtcgccgagggcaccgacagagtgatcgaggtggtgcagggcgcctgcagagccatccggcacatccctagaaggattcggcaaggcctggaaagaatcctgctg

SEQ ID NO: 117 - protein, 856 aa, Artificial Sequence
  anti-CD3scFv_short hinge_TM_CT _T2A_ Cocal envelope
MGVKVLFALICIAVAEADIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASGVELIEGWFSSWKSTVVTFFFAIGVFILLYVVARIVIAVRYRYQGSNNKRIYNDIEMSRFRKGSGEGRGSLLTCGDVEENPGPNFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 118 - DNA, 2568 bp, Artificial Sequence
  anti-CD3scFv_short hinge_TM_CT _T2A_ Cocal envelope - nucleic acid
atgggcgtgaaagtgctgttcgccctgatctgcatcgcagttgctgaagccgacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgcctccggagtggaactgatcgagggctggttcagcagctggaaaagcaccgtggttacattctttttcgccatcggcgtgttcatcctgctgtacgtggtcgccagaattgtgatcgccgtgcggtatagataccagggcagcaacaacaagcggatctacaacgacatcgagatgagcagattcagaaagggatctggagagggaaggggaagcctgctgacatgcggcgacgtggaggagaacccaggaccaaattttctgctgctgaccttcatcgtgctgcctctgtgcagccacgccaagttttccatcgtgttcccacagtcccagaagggcaactggaagaatgtgccctctagctaccactattgcccttcctctagcgaccagaactggcacaatgatctgctgggcatcacaatgaaggtgaagatgcccaagacccacaaggccatccaggcagatggatggatgtgccacgcagccaagtggatcacaacctgtgactttcggtggtacggccccaagtatatcacacactccatccactctatccagcctacctccgagcagtgcaaggagtctatcaagcagacaaagcagggcacctggatgagccctggcttcccaccccagaactgtggctacgccacagtgaccgactccgtggcagtggtggtgcaggcaacacctcaccacgtgctggtggatgagtataccggcgagtggatcgacagccagtttccaaacggcaagtgcgagacagaggagtgtgagaccgtgcacaattctacagtgtggtacagcgattataaggtgacaggcctgtgcgacgccaccctggtggatacagagatcaccttcttttctgaggacggcaagaaggagagcatcggcaagcccaacaccggctacagatccaattacttcgcctatgagaagggcgataaggtgtgcaagatgaattattgtaagcacgccggggtgcggctgcctagcggcgtgtggtttgagttcgtggaccaggacgtgtacgcagcagcaaagctgcctgagtgcccagtgggagcaaccatctccgccccaacacagacctccgtggacgtgtctctgatcctggatgtggagcgcatcctggactacagcctgtgccaggagacctggagcaagatccggtccaagcagcccgtgtcccctgtggacctgtcttacctggcaccaaagaacccaggaaccggaccagcctttacaatcatcaatggcaccctgaagtacttcgagacccgctatatccggatcgacatcgataaccctatcatcagcaagatggtgggcaagatctctggcagccagacagagagagagctgtggaccgagtggttcccttacgagggcgtggagatcggcccaaatggcatcctgaagacaccaaccggctataagtttcccctgttcatgatcggccacggcatgctggacagcgatctgcacaagacctcccaggccgaggtgtttgagcacccacacctggcagaggcaccaaagcagctgcctgaggaggagacactgttctttggcgataccggcatctctaagaaccccgtggagctgatcgagggctggttttcctcttggaagagcacagtggtgaccttctttttcgccatcggcgtgttcatcctgctgtacgtggtggccagaatcgtgatcgccgtgagatacaggtatcagggctccaacaataagaggatctataatgacatcgagatgtctcgcttccggaag

SEQ ID NO: 119 - protein, 327 aa, Artificial Sequence
  anti-CD3scFv_human Glycophorin A hinge-TM-CT
MGVKVLFALICIAVAEADIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASHFSEPEITLIIFGVMAGVIGTILLISYGIRRLIKKSPSDVKPLPSPDTDVPLSSVEIENPETSDQ

SEQ ID NO: 120 - DNA, 981 bp, Artificial Sequence
  anti-CD3scFv_human Glycophorin A hinge-TM-CT - nucleic acid
atgggcgtgaaagtgctgttcgccctgatctgcatcgcagttgctgaagccgacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgcctcccacttcagcgagcctgagatcaccctgatcatcttcggcgtgatggccggagtgatcggcacaatcctgctgatcagctacggcatcagaagactgattaagaaatccccatctgatgtgaagcctctgccttctcctgacaccgacgtccccctgagcagcgtggaaatcgagaaccccgaaaccagcgaccag

SEQ ID NO: 121 - DNA, 7145 bp, Artificial Sequence
  payload plasmid 142 - CAR comprising anti-CD19, cytosolic FRB, RACR
tgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcggccgcggggtacctaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaagcctgcagggccgccaccatgctgctgctggtgacctccctgctgctgtgcgagctgcctcacccagcctttctgctgatccccgacatccagatgacacagaccacaagctccctgtctgccagcctgggcgacagagtgaccatctcctgtagggcctctcaggatatcagcaagtacctgaactggtatcagcagaagccagatggcacagtgaagctgctgatctaccacacctccaggctgcactctggagtgccaagccggttctccggatctggaagcggcaccgactattccctgacaatctctaacctggagcaggaggatatcgccacatacttttgccagcagggcaataccctgccatatacattcggcggaggaaccaagctggagatcaccggatccacatctggaagcggcaagccaggaagcggagagggatccacaaagggagaggtgaagctgcaggagagcggaccaggactggtggcaccatcccagtctctgagcgtgacctgtacagtgtccggcgtgtctctgcctgactacggcgtgtcctggatcaggcagccacctaggaagggactggagtggctgggcgtgatctggggctctgagaccacatactataattctgccctgaagagccgcctgaccatcatcaaggacaactccaagtctcaggtgtttctgaagatgaatagcctgcagaccgacgatacagccatctactattgcgccaagcactactattacggcggctcctacgccatggattattggggccagggcacctccgtgacagtgtctagcgagtctaagtatggcccaccctgccctccatgtccaatgttctgggtgctggtggtggtgggaggcgtgctggcctgttactccctgctggtgaccgtggcctttatcatcttctgggtgaagagaggcaggaagaagctgctgtatatctttaagcagcccttcatgcgccctgtgcagaccacacaggaggaggacggctgcagctgtcggtttccagaggaggaggagggaggatgcgagctgcgcgtgaagttcagccggtccgccgatgcccctgcctaccagcagggccagaaccagctgtataacgagctgaatctgggccggagagaggagtacgacgtgctggataagaggaggggaagggacccagagatgggaggcaagcctcggagaaagaacccacaggagggcctgtacaatgagctgcagaaggacaagatggccgaggcctattctgagatcggcatgaagggagagaggcgccggggcaagggacacgatggcctgtaccagggcctgagcaccgccacaaaggacacatatgatgccctgcacatgcaggccctgccacctaggggatctggagccaccaactttagcctgctgaagcaggcaggcgatgtggaggagaatccaggacctgagatgtggcacgagggactggaggaggcaagcaggctgtactttggcgagcggaatgtgaagggcatgttcgaggtgctggagccactgcacgcaatgatggagaggggaccacagaccctgaaggagacatccttcaaccaggcatacggaagggacctgatggaggcacaggagtggtgccggaagtatatgaagtctggcaatgtgaaggacctgctgcaggcctgggatctgtattaccacgtgtttagaaggatcagcaagggctccggcgccaccaacttctccctgctgaagcaggccggcgatgtggaagaaaatccaggaccaatgcctctgggactgctgtggctgggactggccctgctgggcgccctgcacgcccaggccggcgtgcaggtggagacaatcagccctggcgacggcagaacctttccaaagaggggccagacatgcgtggtgcactacaccggcatgctggaggatggcaagaagttcgactcctctcgcgatcggaacaagccctttaagttcatgctgggcaagcaggaagtgatcagaggctgggaggagggcgtggcccagatgtctgtgggccagagggccaagctgacaatcagcccagactatgcatacggagcaaccggacaccctggaatcatcccaccacacgccacactggtgttcgatgtggagctgctgaagctgggcgagggctctaacaccagcaaggagaatccatttctgttcgcactggaggcagtggtcatctccgtgggctctatgggcctgatcatctccctgctgtgcgtgtacttttggctggagagaacaatgccaaggatccccaccctgaagaacctggaggacctggtgaccgagtaccacggcaatttcagcgcctggtccggcgtgtctaagggactggcagagtccctgcagccagattattctgagcggctgtgcctggtgagcgagatccctccaaagggaggcgccctgggagagggaccaggagccagcccctgcaaccagcactccccttactgggcccccccttgttataccctgaagccagagacaggctctggcgccaccaacttcagcctgctgaagcaagccggcgacgtggaagaaaacccaggaccaatggcactgccagtgaccgccctgctgctgcctctggccctgctgctgcacgcagccagacccatcctgtggcacgaaatgtggcatgaaggcctggaggaggcaagcagactgtactttggcgagagaaatgtgaaaggaatgtttgaggtgctggagcctctgcacgccatgatggagaggggccctcagaccctgaaggagacatcctttaaccaggcctacggcagagacctgatggaggcccaggagtggtgcaggaagtatatgaagagcggaaatgtgaaagacctgctgcaggcctgggatctgtactaccacgtgttccgccggatctctaagggcaaggatacaatcccttggctgggacacctgctggtgggactgagcggagcctttggcttcatcatcctggtgtatctgctgatcaactgcagaaatacaggcccatggctgaagaaggtgctgaagtgtaacacccctgacccatccaagttcttttctcagctgagctccgagcacggcggcgatgtgcagaagtggctgtctagcccctttccttcctctagcttcagccctggaggactggcacctgagatctccccactggaggtgctggagagggacaaggtgacccagctgctgctgcagcaggataaggtgccagagcccgcctccctgtcctctaaccacagcctgacctcctgctttacaaatcagggctacttctttttccacctgccagacgcactggagatcgaggcatgtcaggtgtatttcacatacgatccctatagcgaggaggaccctgatgagggagtggccggcgccccaaccggaagctcccctcagccactgcagccactgagcggagaggacgatgcatattgtacatttccttcccgcgacgatctgctgctgttctctccaagcctgctgggaggaccatctccacccagcaccgcacctggaggatccggggcaggggaggagcggatgcctccatctctgcaggagagagtgccaagggactgggatccacagcctctgggaccacctacccctggagtgccagacctggtggatttccagccaccccctgagctggtgctgcgggaggcaggagaggaggtgccagacgcaggacctagagagggcgtgagctttccctggtccaggccaccaggacagggagagttccgcgccctgaacgcccggctgcccctgaatacagacgcctacctgtctctgcaggagctgcagggccaggatcctacccacctggtgtgacgccggcgctagtgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 122 - DNA, 6926 bp, Artificial Sequence
  payload plasmid - 201 comprising an anti-CD19 CAR, cytosolic FRB, and RACR
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgattgcctgacgcgtaatgaaagaccccacctgtaggtttggcaagctaggatcaaggtcaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagagcccacaacccctcactcggcgcgtgaacacaattctgcagtcgaaggcgtaccgtcacttacgagtcggtagcctgcagggccgccaccatgctgctgctggtgacctccctgctgctgtgcgagctgcctcacccagcctttctgctgatccccgacatccagatgacacagaccacaagctccctgtctgccagcctgggcgacagagtgaccatctcctgtagggcctctcaggatatcagcaagtacctgaactggtatcagcagaagccagatggcacagtgaagctgctgatctaccacacctccaggctgcactctggagtgccaagccggttctccggatctggaagcggcaccgactattccctgacaatctctaacctggagcaggaggatatcgccacatacttttgccagcagggcaataccctgccatatacattcggcggaggaaccaagctggagatcaccggatccacatctggaagcggcaagccaggaagcggagagggatccacaaagggagaggtgaagctgcaggagagcggaccaggactggtggcaccatcccagtctctgagcgtgacctgtacagtgtccggcgtgtctctgcctgactacggcgtgtcctggatcaggcagccacctaggaagggactggagtggctgggcgtgatctggggctctgagaccacatactataattctgccctgaagagccgcctgaccatcatcaaggacaactccaagtctcaggtgtttctgaagatgaatagcctgcagaccgacgatacagccatctactattgcgccaagcactactattacggcggctcctacgccatggattattggggccagggcacctccgtgacagtgtctagcgagtctaagtatggcccaccctgccctccatgtccaatgttctgggtgctggtggtggtgggaggcgtgctggcctgttactccctgctggtgaccgtggcctttatcatcttctgggtgaagagaggcaggaagaagctgctgtatatctttaagcagcccttcatgcgccctgtgcagaccacacaggaggaggacggctgcagctgtcggtttccagaggaggaggagggaggatgcgagctgcgcgtgaagttcagccggtccgccgatgcccctgcctaccagcagggccagaaccagctgtataacgagctgaatctgggccggagagaggagtacgacgtgctggataagaggaggggaagggacccagagatgggaggcaagcctcggagaaagaacccacaggagggcctgtacaatgagctgcagaaggacaagatggccgaggcctattctgagatcggcatgaagggagagaggcgccggggcaagggacacgatggcctgtaccagggcctgagcaccgccacaaaggacacatatgatgccctgcacatgcaggccctgccacctaggggatctggagccaccaactttagcctgctgaagcaggcaggcgatgtggaggagaatccaggacctgagatgtggcacgagggactggaggaggcaagcaggctgtactttggcgagcggaatgtgaagggcatgttcgaggtgctggagccactgcacgcaatgatggagaggggaccacagaccctgaaggagacatccttcaaccaggcatacggaagggacctgatggaggcacaggagtggtgccggaagtatatgaagtctggcaatgtgaaggacctgctgcaggcctgggatctgtattaccacgtgtttagaaggatcagcaagggctccggcgccaccaacttctccctgctgaagcaggccggcgatgtggaagaaaatccaggaccaatgcctctgggactgctgtggctgggactggccctgctgggcgccctgcacgcccaggccggcgtgcaggtggagacaatcagccctggcgacggcagaacctttccaaagaggggccagacatgcgtggtgcactacaccggcatgctggaggatggcaagaagttcgactcctctcgcgatcggaacaagccctttaagttcatgctgggcaagcaggaagtgatcagaggctgggaggagggcgtggcccagatgtctgtgggccagagggccaagctgacaatcagcccagactatgcatacggagcaaccggacaccctggaatcatcccaccacacgccacactggtgttcgatgtggagctgctgaagctgggcgagggctctaacaccagcaaggagaatccatttctgttcgcactggaggcagtggtcatctccgtgggctctatgggcctgatcatctccctgctgtgcgtgtacttttggctggagagaacaatgccaaggatccccaccctgaagaacctggaggacctggtgaccgagtaccacggcaatttcagcgcctggtccggcgtgtctaagggactggcagagtccctgcagccagattattctgagcggctgtgcctggtgagcgagatccctccaaagggaggcgccctgggagagggaccaggagccagcccctgcaaccagcactccccttactgggcccccccttgttataccctgaagccagagacaggctctggcgccaccaacttcagcctgctgaagcaagccggcgacgtggaagaaaacccaggaccaatggcactgccagtgaccgccctgctgctgcctctggccctgctgctgcacgcagccagacccatcctgtggcacgaaatgtggcatgaaggcctggaggaggcaagcagactgtactttggcgagagaaatgtgaaaggaatgtttgaggtgctggagcctctgcacgccatgatggagaggggccctcagaccctgaaggagacatcctttaaccaggcctacggcagagacctgatggaggcccaggagtggtgcaggaagtatatgaagagcggaaatgtgaaagacctgctgcaggcctgggatctgtactaccacgtgttccgccggatctctaagggcaaggatacaatcccttggctgggacacctgctggtgggactgagcggagcctttggcttcatcatcctggtgtatctgctgatcaactgcagaaatacaggcccatggctgaagaaggtgctgaagtgtaacacccctgacccatccaagttcttttctcagctgagctccgagcacggcggcgatgtgcagaagtggctgtctagcccctttccttcctctagcttcagccctggaggactggcacctgagatctccccactggaggtgctggagagggacaaggtgacccagctgctgctgcagcaggataaggtgccagagcccgcctccctgtcctctaaccacagcctgacctcctgctttacaaatcagggctacttctttttccacctgccagacgcactggagatcgaggcatgtcaggtgtatttcacatacgatccctatagcgaggaggaccctgatgagggagtggccggcgccccaaccggaagctcccctcagccactgcagccactgagcggagaggacgatgcatattgtacatttccttcccgcgacgatctgctgctgttctctccaagcctgctgggaggaccatctccacccagcaccgcacctggaggatccggggcaggggaggagcggatgcctccatctctgcaggagagagtgccaagggactgggatccacagcctctgggaccacctacccctggagtgccagacctggtggatttccagccaccccctgagctggtgctgcgggaggcaggagaggaggtgccagacgcaggacctagagagggcgtgagctttccctggtccaggccaccaggacagggagagttccgcgccctgaacgcccggctgcccctgaatacagacgcctacctgtctctgcaggagctgcagggccaggatcctacccacctggtgtgacgccggcgctagtgtcgacgtagtggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaatgtgtgtgttggttttttgtgtgt

SEQ ID NO: 123 - DNA, 3020 bp, Artificial Sequence
  Cocal envelope plasmid - 209
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggcgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgtctgacatcctgcagggccgccaccatgaactttctgctgctgaccttcatcgtgctgcctctgtgcagccacgccaagttttccatcgtgttcccacagtcccagaagggcaactggaagaatgtgcccagctcctaccactattgtccttctagctccgaccagaactggcacaatgatctgctgggcatcaccatgaaggtgaagatgcctaagacacacaaggccatccaggcagatggatggatgtgccacgcagccaagtggatcaccacatgtgactttcggtggtacggccccaagtatatcacccacagcatccactccatccagcctacaagcgagcagtgcaaggagtccatcaagcagaccaagcagggcacatggatgtctcccggcttcccccctcagaactgtggctacgccaccgtgacagatagcgtggcagtggtggtgcaggcaaccccacaccacgtgctggtggatgagtatacaggcgagtggatcgacagccagtttcccaacggcaagtgcgagaccgaggagtgtgagacagtgcacaattctaccgtgtggtacagcgattataaggtgaccggcctgtgcgacgccacactggtggataccgagatcacattcttttccgaggacggcaagaaggagtctatcggcaagcccaacaccggctacaggtctaattacttcgcctatgagaagggcgataaggtgtgcaagatgaattattgtaagcacgccggggtgcggctgccaagcggcgtgtggtttgagttcgtggaccaggacgtgtacgcagcagcaaagctgccagagtgcccagtgggagcaaccatcagcgcccccacccagacatctgtggacgtgagcctgatcctggatgtggagagaatcctggactactccctgtgccaggagacatggtccaagatccgctctaagcagcccgtgagcccagtggacctgtcttacctggcaccaaagaaccctggaacaggacctgcctttaccatcatcaatggcacactgaagtacttcgagacccggtatatcagaatcgacatcgataacccaatcatctccaagatggtgggcaagatctccggctctcagaccgagagagagctgtggacagagtggttcccatacgagggcgtggagatcggccccaatggcatcctgaagacccctacaggctataagtttccactgttcatgatcggccacggcatgctggactctgatctgcacaagaccagccaggccgaggtgtttgagcacccacacctggcagaggcaccaaagcagctgcccgaggaggagaccctgttctttggcgatacaggcatctccaagaaccctgtggagctgatcgagggctggttttctagctggaagtctaccgtggtgacattctttttcgccatcggcgtgttcatcctgctgtacgtggtggcaaggatcgtgatcgccgtgcggtacagatatcagggcagcaacaataagagaatctataatgacatcgagatgtccaggttccgcaagtgacgccggcgtcgacgctcaacagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 124 - DNA, 5398 bp, Artificial Sequence
  helper plasmid 300 - gag/pol
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcactagaagcttagctgcgaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctaggatccgccgccaccatgggcgcccgcgccagcgtgctttctggcggcgagctggacaggtgggagaagattcgcctgcggcctggaggaaagaaaaagtacaagctgaagcacatcgtgtgggcttctcgggaactggaaagattcgccgtgaaccctggactgctagagacctccgaaggctgcagacagatcctgggacagctgcaacctagcctgcagaccggcagcgaggagctgagaagcctgtacaacaccgtcgccaccctgtattgtgtgcaccaaagaatcgagatcaaggacaccaaggaggctctggataagatcgaggaagagcagaacaagagcaagaaaaaagcccagcaggccgccgctgataccggccattctaatcaggtgtcccagaactaccccattgtgcaaaatatccagggccagatggtccaccaggccatcagccctagaaccctgaatgcctgggtgaaggtggtggaagagaaggccttttctccagaggtgatccctatgttcagcgccctgagcgagggcgctacccctcaggacctgaacacaatgctgaacaccgtgggcggccaccaggccgccatgcagatgctgaaagaaaccatcaacgaggaggccgccgaatgggaccgggtgcaccccgttcacgccggcccaatcgcccctggccagatgcgggaacctagaggcagcgacatcgccggcacaaccagcacactgcaagagcagatcggatggatgacacacaacccccccatccccgtgggcgaaatctacaagcggtggatcattctgggactgaacaaaatcgttagaatgtacagccctaccagcatcctggatatcagacagggcccaaaggagcctttccgggactacgtggacagattttacaagaccctgagagccgaacaggcctcccaagaggtgaagaactggatgacagagaccctgctggtgcagaatgccaaccctgattgtaagacaatcctgaaggccctcggacctggcgctacactggaagaaatgatgaccgcctgccagggcgtgggcggccccggccacaaggccagagtgctggccgaggctatgagccaggtgacaaaccccgccacaatcatgatccagaagggcaacttcagaaaccagcggaagaccgtgaaatgcttcaactgcggcaaggaaggccacatcgcaaagaactgcagagcccctaggaagaaaggctgttggaagtgcggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagatcggcggacagctgaaagaggccttgctggacaccggagccgatgacaccgtgctggaagaaatgaacctgcctggaagatggaaacctaagatgatcggtggcatcggcggatttatcaaagtgcgacagtatgaccagatcctgatcgagatttgcggccacaaagctatcggaacagtgctggtcggcccgacccccgtgaacatcattggccgcaacctgctgacacagatcggttgtacactgaactttcctatcagccctatcgaaaccgtgccggtcaagctgaagcccggcatggatggccctaaggtgaagcagtggcccctgacagaggaaaagatcaaggcactggtggaaatctgcacagaaatggaaaaagagggcaagatttctaaaatcggcccagagaacccctacaacacccctgttttcgccatcaagaagaaagattccaccaagtggaggaagctggtggactttcgggaactgaacaagcggacccaggatttctgggaggtgcagctgggcatcccccaccctgccggcctgaaacaaaaaaaaagcgtgaccgtgctggacgtgggcgacgcctatttcagcgtgcctctggataaggacttccggaaatacaccgcctttaccatccctagcatcaacaacgagacccctggcatccggtaccagtacaacgtgctcccacagggctggaagggctcacccgccatcttccagtgcagcatgaccaagatcctggagcctttcagaaagcagaatcctgacatcgtgatctaccagtacatggacgacctgtacgtgggctctgatctggagatcggacagcacagaacaaagatcgaagagctgagacagcatctgctgagatggggtttcaccacccccgacaagaagcaccagaaggaacctccttttctgtggatgggctacgagctgcaccccgataagtggacagtgcagcccatcgtgctgcccgagaaggactcctggaccgtgaacgacattcagaagctggtcggaaagctgaattgggcttcccaaatctacgccggcatcaaggtgcggcagctgtgcaagcttctgcgcggcacaaaggccctgacggaagtcgtgccactgaccgaggaagccgaattagagctggccgaaaacagagaaattctgaaagaacctgtgcacggcgtttactacgacccttctaaggacctgatcgccgaaatccagaaacaaggccagggccagtggacttaccaaatctaccaggagcctttcaaaaacctcaagaccggcaagtacgccagaatgaagggagcccatacaaacgacgtgaagcagctgacagaggctgttcagaagatcgccacagaaagcatcgtgatctggggcaagaccccaaagttcaagctgcctatccaaaaggaaacctgggaggcctggtggaccgagtactggcaggccacctggattcctgaatgggagttcgtgaacacaccacctcttgtgaagctgtggtaccagctggaaaaggagccaatcatcggcgccgagacattctacgtggacggcgccgccaaccgggagaccaaactgggaaaggccggatatgtgaccgacagaggcagacaaaaggtggtgcctctgaccgatacaactaaccagaaaacagagctgcaggccattcacctggccctgcaagacagcggcctggaagtgaatatcgtgacagacagtcagtacgccctgggcatcatccaggctcagcctgacaagagcgagagcgagctggtgtcccagatcatcgagcagctgatcaaaaaggagaaggtttatctggcctgggtgcccgcccacaagggcatcggaggcaacgagcaggtggacaagctggtcagcgccggcatccggaaggtgctgttcctggacggcatcgacaaggcccaggaggaacacgagaagtaccacagcaactggcgggccatggccagcgacttcaacctgccacctgtggttgctaaggagatcgtcgcctcttgtgataagtgccagctgaagggcgaggccatgcacggccaagtggattgcagccctgggatctggcagctagactgtacccacctggagggcaaggtgatcctggtggcagtgcacgtggccagcggctacatcgaggctgaggtgatccccgccgaaacgggccaggagaccgcctactttctgctgaagctagccggccggtggcctgtgaagaccgtgcacaccgataacggcagcaatttcaccagcacaaccgtgaaggctgcctgctggtgggctggaatcaagcaggagttcggcatcccatacaatcctcagtctcagggcgtgatcgagagcatgaacaaggaactgaagaagatcattggtcaggtcagagatcaggccgagcacctgaaaaccgccgttcaaatggctgtgttcatccataacttcaaaagaaaaggcggcatcggcggctacagcgccggcgaaagaatcgtggatatcatcgcgaccgacatccaaacaaaagagctgcaaaagcaaatcaccaagatccagaacttcagagtgtactacagagatagcagagatcctgtgtggaagggacctgccaagctgctgtggaagggcgagggcgccgtggtgatccaggacaatagcgacatcaaggtcgtgcccagaagaaaggctaaaatcattagagactacggcaaacagatggccggagatgattgcgtggcttctagacaggacgaggactgataagaattccatgtcgacgctcaacagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 125 - DNA, 1153 bp, Artificial Sequence
  helper plasmid 246 - Rev
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggccaccatggccggcagaagcggcgacagcgacgaggatctgctgaaagccgtgcggctgatcaagttcctgtaccagagcaaccctcctcctaaccccgagggcaccagacaggctagacggaaccgcagaagaaggtggcgggaacggcaaagacagatccactctatcagcgagagaatcctgagcacctacctgggaagatccgccgagcctgtccccctgcagctgcctccactggaaagactgaccctggattgtaatgaggactgcggcacaagcggaacccagggcgtgggcagcccccagattctggtggaatcccctacaatcctcgagtctggcgccaaggaatgataactagcacctgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 126 - DNA, 2454 bp, Artificial Sequence
  anti-CD3 plasmid 224
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggcgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgccaccatgggcgtgaaagtgctgttcgccctgatctgcatcgcagttgctgaagccgacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgcctccggagtggaactgatcgagggctggttcagcagctggaaaagcaccgtggttacattctttttcgccatcggcgtgttcatcctgctgtacgtggtcgccagaattgtgatcgccgtgcggtatagataccagggcagcaacaacaagcggatctacaacgacatcgagatgagcagattcagaaagtaatgactcgaggtgaattcgcgccggcgtcgacgctcaacagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 127 - DNA, 2452 bp, Artificial Sequence
  anti-CD3 plasmid - 232
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggcgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgccaccatgggcgtgaaagtgctgttcgccctgatctgcatcgcagttgctgaagccgacatccagatgacccagtctcctagcagcctcagcgctagcgtgggcgatagagtgaccatcacatgtagcgccagcagcagcgtgtcctacatgaactggtaccagcaaacacctggaaaggcccctaaaaggtggatctatgacacatctaagctggcttctggagtgccatctagattttctggcagcggctccggcactgattatacattcaccatcagcagcctgcagcccgaggatatcgccacctactactgtcagcagtggtcctctaatcccttcaccttcggccagggcaccaagctgcagatcaccagaaccagcggcgggggaggaagcggcgggggaggatctggcggcggcggcagccaggtgcagctggtgcagagcggcggcggcgtggtgcaacctggcagaagcctgagactgagctgcaaggcctctggctacaccttcacccggtacaccatgcattgggtgcggcaggcccctggcaagggcctggaatggattggatacatcaaccccagcagaggctacaccaactacaaccagaaggtgaaggacagattcacaatttctcgggacaacagcaagaataccgccttcctgcaaatggactccctgcgcccagaagataccggcgtgtacttctgcgctagatattacgacgaccactactgcctggactactggggccagggcacccctgtgaccgtgtccagcgcctccggacacttcagcgagcctgagatcaccctgatcatcttcggcgtgatggccggagtgatcggcacaatcctgctgatcagctacggcatcagaagactgattaagaaatccccatctgatgtgaagcctctgccttctcctgacaccgacgtccccctgagcagcgtggaaatcgagaaccccgaaaccagcgaccagtgataagaattctagtcgacgctcaacagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 128 - DNA, 3871 bp, Artificial Sequence
  KanR_p57M-MND-(anti)medCD3-Cocal-wPREw-BGHpA-001
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaagcctgcagggccgccaccatggcactgcctgtgacagccctgctgctgccactggccctgctgctgcacgcagcacgcccagatatccagatgacccagtccccaagctccctgagcgcctccgtgggcgaccgggtgacaatcacctgcagcgcctctagctccgtgtcctacatgaactggtatcagcagacacctggcaaggccccaaagagatggatctacgataccagcaagctggcctccggcgtgccttctaggttttctggcagcggctccggcacagattatacattcaccatctctagcctgcagccagaggacatcgccacctactattgccagcagtggtcctctaatccctttacattcggccagggcaccaagctgcagatcacaagaacctctggaggaggaggaagcggaggaggaggatccggcggcggcggctctcaggtgcagctggtgcagagcggaggaggagtggtgcagccaggcagaagcctgaggctgtcctgtaaggcctctggctacacattcaccagatatacaatgcactgggtgaggcaggcaccaggcaagggactggagtggatcggctacatcaacccctccaggggctacaccaactataatcagaaggtgaaggatcggttcaccatcagcagggacaactccaagaataccgccttcctgcagatggacagcctgaggccagaggataccggcgtgtacttttgcgcccggtactatgacgatcactactgtctggattattggggccagggaacaccagtgaccgtgagctccgccgcagcaaagcctaccacaacccctgccccaaggccacctacacccgcccctaccatcgcctctcagccactgagcctgaggccagaggcatccaggcctgccgcagggggggccgtgcacacccggggcctggactttgcctctgatatggcactgatcgtgctgggaggagtggcaggactgctgctgttcatcggactgggcatcttcttttgcgtgcgctgtaggcaccggagaaggcagggatctggagagggaaggggaagcctgctgacatgcggcgacgtggaggagaacccaggaccaaattttctgctgctgaccttcatcgtgctgcctctgtgcagccacgccaagttttccatcgtgttcccacagtcccagaagggcaactggaagaatgtgccctctagctaccactattgcccttcctctagcgaccagaactggcacaatgatctgctgggcatcacaatgaaggtgaagatgcccaagacccacaaggccatccaggcagatggatggatgtgccacgcagccaagtggatcacaacctgtgactttcggtggtacggccccaagtatatcacacactccatccactctatccagcctacctccgagcagtgcaaggagtctatcaagcagacaaagcagggcacctggatgagccctggcttcccaccccagaactgtggctacgccacagtgaccgactccgtggcagtggtggtgcaggcaacacctcaccacgtgctggtggatgagtataccggcgagtggatcgacagccagtttccaaacggcaagtgcgagacagaggagtgtgagaccgtgcacaattctacagtgtggtacagcgattataaggtgacaggcctgtgcgacgccaccctggtggatacagagatcaccttcttttctgaggacggcaagaaggagagcatcggcaagcccaacaccggctacagatccaattacttcgcctatgagaagggcgataaggtgtgcaagatgaattattgtaagcacgccggggtgcggctgcctagcggcgtgtggtttgagttcgtggaccaggacgtgtacgcagcagcaaagctgcctgagtgcccagtgggagcaaccatctccgccccaacacagacctccgtggacgtgtctctgatcctggatgtggagcgcatcctggactacagcctgtgccaggagacctggagcaagatccggtccaagcagcccgtgtcccctgtggacctgtcttacctggcaccaaagaacccaggaaccggaccagcctttacaatcatcaatggcaccctgaagtacttcgagacccgctatatccggatcgacatcgataaccctatcatcagcaagatggtgggcaagatctctggcagccagacagagagagagctgtggaccgagtggttcccttacgagggcgtggagatcggcccaaatggcatcctgaagacaccaaccggctataagtttcccctgttcatgatcggccacggcatgctggacagcgatctgcacaagacctcccaggccgaggtgtttgagcacccacacctggcagaggcaccaaagcagctgcctgaggaggagacactgttctttggcgataccggcatctctaagaaccccgtggagctgatcgagggctggttttcctcttggaagagcacagtggtgaccttctttttcgccatcggcgtgttcatcctgctgtacgtggtggccagaatcgtgatcgccgtgagatacaggtatcagggctccaacaataagaggatctataatgacatcgagatgtctcgcttccggaagtgacgccggcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 129 - DNA, 4525 bp, Artificial Sequence
  p57M-CMV-BGi-(anti)-med-hCD3-Cocal-BG-polyA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattctgacactcctgcagggccgccaccatggcactgcctgtgacagccctgctgctgccactggccctgctgctgcacgcagcacgcccagatatccagatgacccagtccccaagctccctgagcgcctccgtgggcgaccgggtgacaatcacctgcagcgcctctagctccgtgtcctacatgaactggtatcagcagacacctggcaaggccccaaagagatggatctacgataccagcaagctggcctccggcgtgccttctaggttttctggcagcggctccggcacagattatacattcaccatctctagcctgcagccagaggacatcgccacctactattgccagcagtggtcctctaatccctttacattcggccagggcaccaagctgcagatcacaagaacctctggaggaggaggaagcggaggaggaggatccggcggcggcggctctcaggtgcagctggtgcagagcggaggaggagtggtgcagccaggcagaagcctgaggctgtcctgtaaggcctctggctacacattcaccagatatacaatgcactgggtgaggcaggcaccaggcaagggactggagtggatcggctacatcaacccctccaggggctacaccaactataatcagaaggtgaaggatcggttcaccatcagcagggacaactccaagaataccgccttcctgcagatggacagcctgaggccagaggataccggcgtgtacttttgcgcccggtactatgacgatcactactgtctggattattggggccagggaacaccagtgaccgtgagctccgccgcagcaaagcctaccacaacccctgccccaaggccacctacacccgcccctaccatcgcctctcagccactgagcctgaggccagaggcatccaggcctgccgcagggggggccgtgcacacccggggcctggactttgcctctgatatggcactgatcgtgctgggaggagtggcaggactgctgctgttcatcggactgggcatcttcttttgcgtgcgctgtaggcaccggagaaggcagggatctggagagggaaggggaagcctgctgacatgcggcgacgtggaggagaacccaggaccaaattttctgctgctgaccttcatcgtgctgcctctgtgcagccacgccaagttttccatcgtgttcccacagtcccagaagggcaactggaagaatgtgccctctagctaccactattgcccttcctctagcgaccagaactggcacaatgatctgctgggcatcacaatgaaggtgaagatgcccaagacccacaaggccatccaggcagatggatggatgtgccacgcagccaagtggatcacaacctgtgactttcggtggtacggccccaagtatatcacacactccatccactctatccagcctacctccgagcagtgcaaggagtctatcaagcagacaaagcagggcacctggatgagccctggcttcccaccccagaactgtggctacgccacagtgaccgactccgtggcagtggtggtgcaggcaacacctcaccacgtgctggtggatgagtataccggcgagtggatcgacagccagtttccaaacggcaagtgcgagacagaggagtgtgagaccgtgcacaattctacagtgtggtacagcgattataaggtgacaggcctgtgcgacgccaccctggtggatacagagatcaccttcttttctgaggacggcaagaaggagagcatcggcaagcccaacaccggctacagatccaattacttcgcctatgagaagggcgataaggtgtgcaagatgaattattgtaagcacgccggggtgcggctgcctagcggcgtgtggtttgagttcgtggaccaggacgtgtacgcagcagcaaagctgcctgagtgcccagtgggagcaaccatctccgccccaacacagacctccgtggacgtgtctctgatcctggatgtggagcgcatcctggactacagcctgtgccaggagacctggagcaagatccggtccaagcagcccgtgtcccctgtggacctgtcttacctggcaccaaagaacccaggaaccggaccagcctttacaatcatcaatggcaccctgaagtacttcgagacccgctatatccggatcgacatcgataaccctatcatcagcaagatggtgggcaagatctctggcagccagacagagagagagctgtggaccgagtggttcccttacgagggcgtggagatcggcccaaatggcatcctgaagacaccaaccggctataagtttcccctgttcatgatcggccacggcatgctggacagcgatctgcacaagacctcccaggccgaggtgtttgagcacccacacctggcagaggcaccaaagcagctgcctgaggaggagacactgttctttggcgataccggcatctctaagaaccccgtggagctgatcgagggctggttttcctcttggaagagcacagtggtgaccttctttttcgccatcggcgtgttcatcctgctgtacgtggtggccagaatcgtgatcgccgtgagatacaggtatcagggctccaacaataagaggatctataatgacatcgagatgtctcgcttccggaagtgacgccggcgctcaaatcctgcacaacagattcttcatgtttggaccaaatcaacttgtgataccatgctcaaagaggcctcaattatatttgagtttttaatttttatgaaaaaaaaaaaaaaaaacggaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctaatgctgtatttta

SEQ ID NO: 130 - DNA, 2773 bp, Artificial Sequence
  Cocal envelope plasmid - 143
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaagcctgcagggccgccaccatgaactttctgctgctgaccttcatcgtgctgcctctgtgcagccacgccaagttttccatcgtgttcccacagtcccagaagggcaactggaagaatgtgcccagctcctaccactattgtccttctagctccgaccagaactggcacaatgatctgctgggcatcaccatgaaggtgaagatgcctaagacacacaaggccatccaggcagatggatggatgtgccacgcagccaagtggatcaccacatgtgactttcggtggtacggccccaagtatatcacccacagcatccactccatccagcctacaagcgagcagtgcaaggagtccatcaagcagaccaagcagggcacatggatgtctcccggcttcccccctcagaactgtggctacgccaccgtgacagatagcgtggcagtggtggtgcaggcaaccccacaccacgtgctggtggatgagtatacaggcgagtggatcgacagccagtttcccaacggcaagtgcgagaccgaggagtgtgagacagtgcacaattctaccgtgtggtacagcgattataaggtgaccggcctgtgcgacgccacactggtggataccgagatcacattcttttccgaggacggcaagaaggagtctatcggcaagcccaacaccggctacaggtctaattacttcgcctatgagaagggcgataaggtgtgcaagatgaattattgtaagcacgccggggtgcggctgccaagcggcgtgtggtttgagttcgtggaccaggacgtgtacgcagcagcaaagctgccagagtgcccagtgggagcaaccatcagcgcccccacccagacatctgtggacgtgagcctgatcctggatgtggagagaatcctggactactccctgtgccaggagacatggtccaagatccgctctaagcagcccgtgagcccagtggacctgtcttacctggcaccaaagaaccctggaacaggacctgcctttaccatcatcaatggcacactgaagtacttcgagacccggtatatcagaatcgacatcgataacccaatcatctccaagatggtgggcaagatctccggctctcagaccgagagagagctgtggacagagtggttcccatacgagggcgtggagatcggccccaatggcatcctgaagacccctacaggctataagtttccactgttcatgatcggccacggcatgctggactctgatctgcacaagaccagccaggccgaggtgtttgagcacccacacctggcagaggcaccaaagcagctgcccgaggaggagaccctgttctttggcgatacaggcatctccaagaaccctgtggagctgatcgagggctggttttctagctggaagtctaccgtggtgacattctttttcgccatcggcgtgttcatcctgctgtacgtggtggcaaggatcgtgatcgccgtgcggtacagatatcagggcagcaacaataagagaatctataatgacatcgagatgtccaggttccgcaagtgacgccggcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 131 - DNA, 6468 bp, Artificial Sequence
  helper plasmid gag/pol - 89
ttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattcgagatctgccgccgccatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaattccggagcggccgcaggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttccgcggaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtatttta

SEQ ID NO: 132 - DNA, 972 bp, Artificial Sequence
  helper plasmid 84 - Rev
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagctcgagctcgtttagtgaaccgtcagatcgcctggagacgccatccacgctgttttgacctccatagaagacaccgggaccgatccagcctcccctcgaagctagtcgattaggcatctcctatggcaggaagaagcggagacagcgacgaagacctcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtgctgttagcttgctcaatgccacagctatagcagtagctgaggggacagatagggttatagaagtagtacaagaagcttggcactggccgtcgttttacaacgtcgtgatctgagcctgggagatctctggctaactagggaacccactgcttaagcctcaataaagcttgccttgagtgcttcaagtagtgtgtgcccgtctgttgtgtgactctggtaactagagatc

SEQ ID NO: 133 - protein, 10 aa, Artificial Sequence
  anti-CD3- CDR-L1
SASSSVSYMN

SEQ ID NO: 134 - protein, 8 aa, Artificial Sequence
  anti-CD3 - CDR-L2
DTSKLASG

SEQ ID NO: 135 - protein, 9 aa, Artificial Sequence
  anti-CD3 - CDR-L3
QQWSSNPFT

SEQ ID NO: 136 - protein, 17 aa, Artificial Sequence
  anti-CD3 - CDR-H2
YINPSRGYTNYNQKVKD

SEQ ID NO: 137 - protein, 10 aa, Artificial Sequence
  anti-CD3 - CDR-H3
YYDDHYCLDY

SEQ ID NO: 138 - protein, 11 aa, Artificial Sequence
  anti-CD19 - CDR-L1
RASQDISKYLN

SEQ ID NO: 139 - protein, 7 aa, Artificial Sequence
  anti-CD19 - CDR-L2
HTSRLHS

SEQ ID NO: 140 - protein, 9 aa, Artificial Sequence
  anti-CD19 - CDR-L3
QQGNTLPYT

SEQ ID NO: 141 - protein, 4 aa, Artificial Sequence
  anti-CD19 - CDR-H1
DYGV

SEQ ID NO: 142 - protein, 16 aa, Artificial Sequence
  anti-CD19 - CDR-H2
VIWGSETTYYNSALKS

SEQ ID NO: 143 - protein, 12 aa, Artificial Sequence
  anti-CD19 - CDR-H3
HYYYGGSYAMDY

SEQ ID NO: 144 - protein, 5 aa, Artificial Sequence
  anti-CD3 - CDR-H1
RYTMH

(19) United States
(12) Patent Application Publication
(10) Pub. No.: US 2024/0141375 A1
(43) Pub. Date: May 2, 2024

(54) LENTIVIRUS FOR GENERATING CELLS EXPRESSING ANTI-CD19 CHIMERIC ANTIGEN RECEPTOR

(71) Applicant: Umoja Biopharma, Inc., Seattle, WA (US)

(72) Inventors: Andrew Scharenberg, Seattle, WA (US); Christopher Nicolai, Seattle, WA (US); Ryan Crisman, Seattle, WA (US); Alessandra Sullivan, Seattle, WA (US); Kathryn Michels, Seattle, WA (US); Byoung Ryu, Seattle, WA (US); Shon Green, Seattle, WA (US); Laurie Beitz, Seattle, WA (US); Susana Hernandez Lopez, Seattle, WA (US)

(21) Appl. No.: 18/262,859

(22) PCT Filed: Jan. 26, 2022
(86) PCT No.: PCT/US22/13947

§ 371 Date: Jul. 25, 2023

Related U.S. Application Data
(60) Provisional application No. 63/142,347, filed on Jan. 27, 2021.
(60) Provisional application No. 63/185,765, filed on May 7, 2021.

(51) Int. Cl. C12N 15/86; A61K 35/76; C07K 14/005; C07K 16/28

(52) U.S. Cl. CPC C12N 15/86; A61K 35/76; C07K 14/005; C07K 16/2809; C07K 2317/622; C12N 2740/10043

(57) ABSTRACT

Provided are compositions and methods for transducing immune cells in vivo where a viral particle comprising a polynucleotide encoding a chimeric antigen receptor and a multipartite cell-surface receptor is administered to a subject.

### RELATED APPLICATIONS

This application claims priority to, and the benefit of, U.S. Provisional Application No. 63/142,347, filed Jan. 27, 2021, and U.S. Provisional Application No. 63/185,765, filed May 7, 2021. The contents of the aforementioned patent applications are incorporated herein by reference in their entirety.

### SEQUENCE LISTING

The instant application contains a Sequence Listing which has been submitted in ASCII format via EFS-Web and is hereby incorporated by reference in its entirety. Said ASCII copy, created on Jan. 26, 2022, is named “UMOJ-024-02WO_SeqList_ST25.txt” and is 246 KB in size.

### FIELD OF INVENTION

The invention relates generally to in vivo transduction of immune cells to treat cancer and/or B-cell malignancies.

### BACKGROUND

Cellular therapy generally employs the transduction of immune cells ex vivo to generate a population of therapeutic cells to be introduced into the patient. For example, T cells from an autologous or allogenic source can be transduced ex vivo with a vector encoding a chimeric antigen receptor. The resulting CAR T-cells are then infused into the patient.

It would be desirable to instead generate therapeutic cells in vivo by delivering a vector to the patient. Current methodologies for in vivo transduction of immune cells suffer from technical, logistical, consistency, cost, and efficacy challenges. The in vivo approach has not been widely pursued to date because of the technical challenges associated with it, the main hurdle being the need to activate T cells in the body in order to effectively engineer them as well as the need to “control” expansion of these engineered cells once transduced.

Currently, patients with aggressive B-cell malignancies that have failed standard therapies, including chemotherapy and often hematopoietic stem cell transplantation (HSCT), have the option to receive an autologous CAR-T cell product that redirects their T cells against the antigen CD19 by means of an ex-vivo manufacturing process. However, manufacture of these products requires a complex series of steps, starting with collection of the patient's peripheral blood mononuclear cells via a leukapheresis procedure, followed by genetic modification of the patient's T cells in a cGMP facility that introduces delays, risks, and complex logistics into patient care. This is followed by the administration of lymphodepleting chemotherapy prior to infusion of the final drug product. There is an unmet medical need for patients with relapsed/refractory B-cell malignancies, both in terms of their untreated disease as well as inability to manufacture or tolerate the timing of logistics of newer cellular products.

The present disclosure provides compositions and methods related to in vivo transduction of immune cells to treat cancer and/or B-cell malignancies.

### SUMMARY OF THE INVENTION

In one aspect, the present disclosure provides a viral particle comprising a vector genome comprising a polynucleotide sequence encoding an anti-CD19 chimeric antigen receptor, wherein the viral particle transduces immune cells in vivo.

In some embodiments, the viral particle is a lentiviral particle.

In some embodiments, the immune cells are T cells.

In some embodiments, the viral particle comprises a polynucleotide sequence encoding a multipartite cell-surface receptor.

In some embodiments, the multipartite cell-surface receptor is a chemically inducible cell-surface receptor.

In some embodiments, the viral particle comprises a polynucleotide sequence encoding a multipartite cell-surface receptor comprising a FKBP-rapamycin complex binding domain (FRB domain) or a functional variant thereof; and the polynucleotide comprises a polynucleotide sequence encoding a FK506 binding protein domain (FKBP) or a functional variant thereof.

In some embodiments, the multipartite cell-surface receptor is a rapamycin-activated cell-surface receptor.

In some embodiments, the viral particle comprises a sequence that confers resistance to an immunosuppressive agent.

In some embodiments, the viral particle comprises a sequence that confers resistance to an immunosuppressive agent encodes a polypeptide that binds rapamycin, wherein optionally, the polypeptide is an FRB.

In some embodiments, the viral particle comprises a sequence in 5′ to 3′ order on a polycistronic transcript: the polynucleotide sequence encoding the multipartite cell-surface receptor and the polynucleotide sequence encoding the anti-CD19 chimeric antigen receptor.

In some embodiments, the viral particle comprises a sequence in 5′ to 3′ order on a polycistronic transcript: the polynucleotide sequence encoding the anti-CD19 chimeric antigen receptor and the polynucleotide sequence encoding the multipartite cell-surface receptor, and/or wherein the anti-CD19 chimeric antigen receptor shares at least 80%, 90%, 95%, or 100% identity to SEQ ID NO: 51, 79, 89, 121, or 122.

In some embodiments, the polynucleotide encoding the anti-CD19 chimeric antigen receptor and/or the polynucleotide encoding the multipartite cell-surface receptor is operatively linked to one or more promoters.

In some embodiments, the promoter is an inducible promoter.

In some embodiments, the viral particle comprises a viral envelope comprising one or more immune cell-activating proteins exposed on the surface and/or conjugated to the surface of the viral envelope.

In some embodiments, the viral envelope comprises an anti-CD3 single-chain variable fragment exposed on the surface and/or conjugated to the surface of the viral envelope.

In some embodiments, the viral envelope comprises a Cocal glycoprotein exposed on the surface and/or conjugated to the surface of the viral envelope.

In some embodiments, the viral envelope comprises a Cocal glycoprotein exposed on the surface and/or conjugated to the surface of the viral envelope, optionally wherein the Cocal glycoprotein comprises the R354Q mutation compared to a reference sequence according to SEQ ID NO: 5.

In some embodiments, the viral envelope comprises an anti-CD3 single-chain variable fragment and a Cocal glycoprotein exposed on the surface and/or conjugated to the surface of the viral envelope.

In some embodiments, the viral envelope comprises an anti-CD3 single-chain variable fragment sequence that shares at least 80%, at least 85%, at least 90%, at least 95%, at least 99%, or 100% identity to SEQ ID NO: 2 or 12.

In some embodiments, the viral envelope comprises a Cocal glycoprotein sequence that shares at least 80%, at least 85%, at least 90%, at least 95%, at least 99%, or 100% identity to SEQ ID NO: 5, 13, 19, 123, 128, 129, or 130.

In some embodiments, the promoter is an MND promoter.

In some embodiments, the viral particle comprises a sequence that shares at least 80%, at least 85%, at least 90%, at least 95%, at least 99%, or 100% identity to SEQ ID NO: 49.

In some embodiments, the viral particle comprises a sequence that shares at least 80%, at least 85%, at least 90%, at least 95%, at least 99%, or 100% identity to SEQ ID NO: 75.

In some embodiments, the viral particle comprises a sequence that shares at least 80%, at least 85%, at least 90%, at least 95%, at least 99%, or 100% identity to SEQ ID NO: 87.

The present disclosure provides a method of treating a disease or disorder, transducing immune cells in vivo, and/or generating an immune cell expressing an anti-CD19 chimeric antigen receptor in a subject in need thereof, comprising administering the viral particle of the present disclosure to the subject.

In some embodiments, the viral particle is administered by intraperitoneal, subcutaneous, or intranodal injection.

In some embodiments, the transduced immune cells comprising the polynucleotides of the present disclosure are administered to the subject.

In another aspect, the present disclosure provides a method of treating a disease or disorder in a subject in need thereof, comprising administering a therapeutically effective amount of a viral particle to the subject by intraperitoneal, subcutaneous, or intranodal injection, wherein the viral particle transduces immune cells in vivo.

In some embodiments, the viral particle is administered by intra-nodal injection, optionally via inguinal lymph node.

In some embodiments, the viral particle is administered by intraperitoneal injection.

The present disclosure provides a viral particle for use in transducing immune cells in vivo, comprising a polynucleotide comprising a polynucleotide sequence encoding a chimeric antigen receptor.

In some embodiments, the viral particle further comprises a polynucleotide sequence encoding a dominant-negative TGFβ receptor.

In some embodiments, expression of the chimeric antigen receptor is modulated by a FRB-degron fusion polypeptide and wherein suppression of the FRB-degron fusion polypeptide is chemically inducible by a ligand.

In some embodiments, the ligand is rapamycin.

In some embodiments, expression of the chimeric antigen receptor is modulated by a degron fusion polypeptide and wherein suppression of the degron fusion polypeptide is chemically inducible by a ligand.

In some embodiments, the disease or disorder comprises B-cell malignancy, relapsed/refractory CD19-expressing malignancy, diffuse large B-cell lymphoma (DLBCL), Burkitt's type large B-cell lymphoma (B-LBL), follicular lymphoma (FL), chronic lymphocytic leukemia (CLL), acute lymphocytic leukemia (ALL), mantle cell lymphoma (MCL), hematological malignancy, colon cancer, lung cancer, liver cancer, breast cancer, renal cancer, prostate cancer, ovarian cancer, skin cancer, melanoma, bone cancer, brain cancer, squamous cell carcinoma, leukemia, myeloma, B cell lymphoma, kidney cancer, uterine cancer, adenocarcinoma, pancreatic cancer, chronic myelogenous leukemia, glioblastoma, neuroblastoma, medulloblastoma, sarcoma, and any combination thereof.

In some embodiments, the disease or disorder comprises diffuse large B-cell lymphoma (DLBCL).

In some embodiments, the disease or disorder comprises Burkitt's type large B-cell lymphoma (B-LBL).

In some embodiments, the disease or disorder comprises follicular lymphoma (FL).

In some embodiments, the disease or disorder comprises chronic lymphocytic leukemia (CLL).

In some embodiments, the disease or disorder comprises acute lymphocytic leukemia (ALL).

In some embodiments, the disease or disorder comprises mantle cell lymphoma (MCL).

The present disclosure provides a pharmaceutical composition comprising the viral particle of the present disclosure.

The present disclosure provides a kit comprising the pharmaceutical composition of the present disclosure and optionally a composition comprising a ligand, optionally rapamycin.

The present disclosure provides a viral particle for use in a method according to any viral particles of the present disclosure.

The present disclosure provides a use of a viral particle in a method according to any method of the present disclosure.

In some embodiments, a method of treating a disease or disorder associated with malignant CD19+ cells in a subject comprises administering the viral particle of the present disclosure to a subject and following administration of the viral particle, CD19+ B cells in a subject are depleted by at least 80%, at least 85%, at least 90%, or at least 95% as compared to a subject that did not receive viral particles.

In some embodiments, the CD19+ B cells are depleted in peripheral blood of the subject.

In some embodiments, the B cell depletion is sustained in the subject for at least 7, at least 10, at least 20, at least 30 days, at least 40 days, at least 50 days, at least 60 days, at least 70 days, or at least 80 days after administering the viral particle.

In some embodiments, at least 2 million, at least 4 million, at least 6 million, at least 8 million or at least 10 million transducing units of viral particle are administered to the subject.

In some embodiments, contacting immune cells with the ligand of the present disclosure increases the number of immune cells expressing an anti-CD19 chimeric antigen receptor in a subject by at least 10-fold, at least 50-fold, at least 100-fold, at least 200-fold, at least 500-fold, or at least 1000-fold.

The present disclosure provides a polypeptide comprising a single-chain variable fragment that specifically binds CD3 (anti-CD3 scFv) and a glycophorin A transmembrane fragment.

In some embodiments, the glycophorin A transmembrane fragment shares at least 80%, at least 85%, at least 90%, at least 95%, at least 99%, or 100% identity to

(SEQ ID NO: 105)
HFSEPEITLIIFGVMAGVIGTILLISYGIRRLIKKSPSDVKPLPSPDTD
VPLSSVEIENPETSDQ.

In some embodiments, the anti-CD3 scFv shares at least 80%, at least 85%, at least 90%, at least 95%, at least 99%, or 100% identity to SEQ ID NO: 2 or 12.

In some embodiments, the polypeptide shares at least 80%, at least 85%, at least 90%, at least 95%, at least 99%, or 100% identity to SEQ ID NO: 119.

In some embodiments, the transmembrane fragment shares at least 80%, at least 85%, at least 90%, at least 95%, at least 99%, or 100% identity to SEQ ID NO 13, 19, 25, 31, 37, 43, or 105.

The present disclosure provides a surface-engineered lentiviral particle comprising a polypeptide according to the present disclosure displayed on the surface of the lentiviral particle.

The present disclosure provides a method of transducing cells, comprising contacting a viral particle according to the present disclosure with an immune cell in vivo.

The present disclosure provides a polynucleotide comprising an anti-CD3 scFv and a glycophorin A transmembrane fragment.

In some embodiments, the glycophorin A transmembrane fragment shares at least 80%, at least 85%, at least 90%, at least 95%, at least 99%, or 100% identity to SEQ ID NO: 106.

In some embodiments, the anti-CD3 scFv shares at least 80%, at least 85%, at least 90%, at least 95%, at least 99%, or 100% identity to SEQ ID NO: 7 or 15.

In some embodiments, the polypeptide shares at least 80%, at least 85%, at least 90%, at least 95%, at least 99%, or 100% identity to SEQ ID NO: 120.

In some embodiments, the transmembrane fragment shares at least 80%, at least 85%, at least 90%, at least 95%, at least 99%, or 100% identity to SEQ ID NO 16, 22, 25, 28, 34, 40, 47, or 106.

The present disclosure provides a method of making a viral particle comprising:
a) providing a cell in a culture medium; and
b) transfecting the cell with a vector genome according to the present disclosure, a transfer plasmid, and a packaging plasmid, simultaneously or sequentially; whereby the cell expresses a surface-engineered viral particle.

The present disclosure provides a method of treating a disease or disorder associated with malignant CD19+ cells comprising transducing immune cells in vivo, and/or generating a viral particle expressing an anti-CD3 single-chain variable fragment exposed on the surface and/or conjugated to the surface of the viral envelope that shares at least 80%, at least 85%, at least 90%, at least 95%, at least 99%, or 100% identity to SEQ ID NO: 2 or 12 and administering the viral particle to a subject.

In some embodiments, the viral particle is administered by intraperitoneal, subcutaneous, or intranodal injection.

### BRIEF DESCRIPTION OF THE DRAWINGS

FIG. 1 is a graph of 293T titers of lentiviral vectors. Amicon-concentrated lentiviral vector preparations were tittered on 293T cells which were previously seeded in 12 well plates. Three days later, the transduced cells were assessed for mCherry expression. Calculated viral titers are shown.

FIG. 2A shows flow cytometry to measure CD25 expression and mCherry positively expressing T cells in PBMCs stimulated or not and transduced with no vector. 4 days after transduction PBMCs were harvested and stained.

FIG. 2B shows flow cytometry to measure CD25 expression and mCherry positively expressing T cells in PBMCs stimulated or not and transduced with 5 ul or 25 ul Cocal vector. 4 days after transduction PBMCs were harvested and stained.

FIG. 2C shows flow cytometry to measure CD25 expression and mCherry positively expressing T cells in PBMCs stimulated or not and transduced with 5 ul or 25 ul αCD3+Cocal vector. 4 days after transduction PBMCs were harvested and stained.

FIG. 2D shows flow cytometry to measure CD25 expression and mCherry positively expressing T cells in PBMCs stimulated or not and transduced with 5 ul or 25 ul αCD3-Blind-Cocal vector. 4 days after transduction PBMCs were harvested and stained.

FIG. 3 shows flow cytometry plots of αCD19 CAR and 2A peptide expression. Unstimulated PBMC cells were stained for RACR-αCD19 CAR and 2A following culture in Rapamycin. The unstimulated PBMCs were transduced with the indicated vectors (VT103, RACR-αCD19 CAR MND-Cocal, RACR-αCD19 CAR CMV-Cocal, RACR-αCD19 CAR CMV-αCD3-Cocal, RACR-αCD19 CAR CMV-αCD3-(B)Cocal) at 100 ul each. 4 days after transduction, rapamycin was added, and cells were cultured for 11 days. The PBMCs were then harvested and stained for αCD19 CAR and 2A peptide.

FIG. 4 shows flow cytometry plots of αCD19 CAR and 2A peptide expression. Blinatumomab-stimulated PBMC cells were stained for RACR-αCD19 CAR and 2A following culture in Rapamycin. The Blinatumomab-stimulated PBMCs were transduced with the indicated vectors (RACR-αCD19 CAR MND-Cocal, RACR-αCD19 CAR CMV-Cocal, RACR-αCD19 CAR CMV-αCD3-Cocal, RACR-αCD19 CAR CMV-αCD3-(B)Cocal) at 100 ul each. 4 days after transduction, rapamycin was added, and cells were cultured for 11 days. The PBMCs were then harvested and stained for αCD19 CAR and 2A peptide.

FIG. 5 shows flow cytometry plots of αCD19 CAR+ CD8 T cells. Unstimulated PBMCs were transduced with RACR-αCD19 CAR/αCD3-Cocal vector as described in Example 2. 4 days after transduction, Rapamycin was added to culture. 13 days later the cells were stimulated with Raji cells and intracellular cytokine production was measured as described in Example 2. Shown are Viable, CD3+, CD8+, 2A+ cells.

FIG. 6 shows flow cytometry plots of αCD19 CAR+ CD4 T cells. Unstimulated PBMCs were transduced with RACR-αCD19 CAR/αCD3-Cocal vector as described in Example 2. 4 days after transduction, Rapamycin was added to culture. 13 days later the cells were stimulated with Raji cells and intracellular cytokine production was measured as described in Example 2. Shown are Viable, CD3+, CD8+, 2A+ cells.

FIG. 7A shows flow cytometry plots of CD8+ T cells expressing CD25. Representative flow cytometry plots of PBMCs transduced with αCD19 CAR-TGFβDN and Cocal or αCD3-Cocal-pseudotyped viral envelope proteins analyzed for CD8+ T cells at MOI=2.

FIG. 7B shows a graph of % CD25 T cells transduced with αCD19 CAR-TGFβDN/αCD3-Cocal viral particles at 0.5 MOI, 1.0 MOI, or 2.0 MOI and added to unstimulated PBMCs for 3 days.

FIG. 8A shows flow cytometry plots of CD8+ T cells expressing 2A peptide. Representative flow cytometry plots of PBMCs transduced with αCD19 CAR-TGFβDN and Cocal or αCD3-Cocal-pseudotyped viral envelope proteins analyzed for CD8+ T cells at MOI=2.

FIG. 8B shows a graph of % αCD19 CAR+ T cells transduced with αCD19 CAR-TGFβDN/αCD3-Cocal viral particles at 0.5 MOI, 1.0 MOI, or 2.0 MOI and added to unstimulated PBMCs for 6 days.

FIG. 9A shows a graph of % αCD19 CAR+ CD4 T cells transduced with αCD19 CAR-TGFβDN/αCD3-Cocal viral particles at 0.5 MOI, 1.0 MOI, or 2.0 MOI and added to unstimulated PBMCs for 3, 6, or 12 days.

FIG. 9B shows a graph of % αCD19 CAR+ CD8 T cells transduced with αCD19 CAR-TGFβDN/αCD3-Cocal viral particles at 0.5 MOI, 1.0 MOI, or 2.0 MOI and added to unstimulated PBMCs for 3, 6, or 12 days.

FIG. 10A shows a graph of % αCD19 CAR+ 293T cells transduced with αCD19 CAR-TGFβDN, αCD19 CAR-RACR, or RACR-αCD19 CAR vectors with Cocal envelope protein. The 293T titers (TU/ml) of viral particle preparations are also shown.

FIG. 10B shows a graph of % αCD19 CAR+ 293T cells transduced with αCD19 CAR-TGFβDN, αCD19 CAR-RACR, or RACR-αCD19 CAR vectors with αCD3-Cocal envelope protein. The 293T titers (TU/ml) of viral particle preparations are also shown.

FIG. 11A shows flow cytometry to measure αCD19 CAR and 2A peptide expressing CD8 T cells in unstimulated PBMCs transduced with MOI=1.5 αCD19 CAR-TGFβDN vector and Cocal or αCD3-Cocal-pseudotyped viral envelope proteins 8 days after transduced PBMCs were harvested and stained. Gated on Viable, CD3+, CD8+ cells.

FIG. 11B shows flow cytometry to measure αCD19-CAR and 2A peptide expressing CD8 T cells in unstimulated PBMCs transduced with MOI=1.5 RACR-αCD19 CAR vector and Cocal or αCD3-Cocal-pseudotyped viral envelope proteins 8 days after transduced PBMCs were harvested and stained. Gated on Viable, CD3+, CD8+ cells.

FIG. 11C shows flow cytometry to measure αCD19-CAR and 2A peptide expressing CD8 T cells in unstimulated PBMCs transduced with MOI=1.5 αCD19-RACR vector and Cocal or αCD3-Cocal-pseudotyped viral envelope proteins 8 days after transduced PBMCs were harvested and stained. Gated on Viable, CD3+, CD8+ cells.

FIG. 12 shows a schematic of the study protocol described in Example 5

FIG. 13A shows flow cytometry plots for P2A (CAR) and cell activity (CD71) in single/live/CD3+/CD8+ cells from d8 (the start of rapamycin and/or Raji treatment), d15, d22 post-transduction of control, MOI=0 cells.

FIG. 13B shows flow cytometry plots for P2A (CAR) and cell activity (CD71) in single/live/CD3+/CD8+ cells from d8 (the start of rapamycin and/or Raji treatment), d15, d22 post-transduction of cells transduced with the RACR-αCD19 CAR oriented vector. Red arrows denote the RACR-αCD19 CAR 2A+ populations.

FIG. 13C shows flow cytometry plots for P2A (CAR) and cell activity (CD71) in single/live/CD3+/CD8+ cells from d8 (the start of rapamycin and/or Raji treatment), d15, d22 post-transduction of cells transduced with the αCD19 CAR-RACR oriented vector. Black arrows denote the αCD19 CAR-RACR 2A+ populations.

FIG. 14 shows flow cytometry plots for rapamycin enrichment of CD8+ CAR-T cells, and a “sneaky” RACR-αCD19 CAR-T population that is detectable only when cells are treated with rapamycin (red arrow).

FIG. 15 shows a graph of the total CD8+ CAR-T cells as they expand from day 8 to day 22 of the study. Cell expansion was increased in the presence of rapamycin by day 22 relative to the absence of rapamycin. The largest expansion occurred with both rapamycin and Raji cell addition.

FIG. 16A shows flow cytometry plots for CD3+ (T cells) and GFP (Raji cells) in the presence and absence of rapamycin. Raji cells were diminished in co-culture wells treated with rapamycin in control MOI=0 cells.

FIG. 16B shows flow cytometry plots for CD3+ (T cells) and GFP (Raji cells) in the presence and absence of rapamycin. Raji cells were diminished in co-culture wells transduced with the RACR-αCD19 CAR oriented vector.

FIG. 16C shows flow cytometry plots for CD3+ (T cells) and GFP (Raji cells) in the presence and absence of rapamycin. Raji cells were diminished in co-culture wells transduced with the αCD19 CAR-RACR oriented vector.

FIG. 17A shows a graph of the flow cytometry quantification of CAR+ T cells/ul blood in CD3+ cells.

FIG. 17B shows a graph of the flow cytometry quantification of CAR+ T cells/ul blood in Non-CD3+ cells.

FIG. 18A shows a graph of the flow cytometry quantification of the ratio of the % CD20+ to CD45+ T cells at the termination of the study.

FIG. 18B shows a graph of the flow cytometry quantification of the ratio of the % CD3+ to CD45+ T cells at the termination of the study.

FIG. 19A shows a graph of the flow cytometry quantification of B cells/ul whole blood throughout the study (Day 0-Day 30). Bars indicate +/−standard error of the mean.

FIG. 19B shows a graph of the flow cytometry quantification of B cells (frequency of total CD45+) in the spleen. Bars indicate median value of each group.

FIG. 19C shows a graph of the flow cytometry quantification of B cells (frequency of total CD45+) in the bone marrow. Bars indicate median value of each group.

FIG. 20A shows a graph of the flow cytometry quantification of αCD4 CART cells in the blood, spleen, and bone marrow upon study termination at day 29. Individual values represent the frequency of CAR+ cells within the indicated T cell population after background subtraction. Bars indicate median value of each group.

FIG. 20B shows a graph of the flow cytometry quantification of αCD8 CART cells in the blood, spleen, and bone marrow upon study termination at day 29. Individual values represent the frequency of CAR+ cells within the indicated T cell population after background subtraction. Bars indicate median value of each group.

FIG. 21A shows a graph of CAR payload integration in blood at days 3, 10, 14, and 21. Vector copy number was determined by digital droplet PCR (ddPCR) using human as the reference genome. Bar indicates median values for each group.

FIG. 21B shows a graph of CAR payload integration in bone marrow at days 10 and 29. Vector copy number was determined by digital droplet PCR using human as the reference genome. Bar indicates median values for each group.

FIG. 22A shows a diagram of an embodiment of the viral particle of the present disclosure.

FIG. 22B shows a diagram of an embodiment of the viral particle of the present disclosure.

FIGS. 23A and 23B show schematics of the drug product transgene. The transgene encodes an anti-CD19-CAR with a FMC63 scFv, a short IgG4 hinge, and a 4-1BB/CD3ζ signaling domain. The anti-CD19-CAR is followed by a P2A ribosomal skipping sequence, and a rapamycin activated cytokine receptor cassette (FRB-RACR).

FIG. 24 shows a schematic of the mechanism of action of the drug product. Lentivirus particles bind to T cells in vivo via an anti-CD3 scFv, which simultaneously activates T cells and facilitates lentivirus internalization. The αCD19 CAR-RACR construct-containing capsid is released into the cytosol, reverse-transcribed into DNA, and integrated into the genome. Transduced T cells express the anti-CD19 CAR and target CD19-expressing tumor cells.

FIG. 25 shows a schematic of the RACR system.

FIG. 26A shows representative flow plots of CD25 expression on CD8 T cells from a single donor. All samples were gated on viable, CD3+, and CD4+/CD8+ cells.

FIG. 26B shows show % CD25 expression from all three donors across multiple MOIs for both CD8+ T cells. All samples were gated on viable, CD3+, and CD4+/CD8+ cells.

FIG. 26C shows show % CD25 expression from all three donors across multiple MOIs for both CD4+ T cells. All samples were gated on viable, CD3+, and CD4+/CD8+ cells.

FIG. 27A shows representative flow plots of αCD19 CAR+ CD8+ T cells from a single donor, +/−Rapamycin, on Day 17. CAR+ cells are gated on viable, CD3+, and CD8+ cells.

FIG. 27B shows % CAR+ cells of total CD8+ T cells. CAR+ cells are gated on viable, CD3+, and CD8+ cells.

FIG. 27C shows % CAR+ cells of total counts per well of CAR+ CD8+ T cells. CAR+ cells are gated on viable, CD3+, and CD8+ cells. To calculate total CAR+ cells per well, total CAR counts were normalized to counting beads. Similar data was obtained in CAR+ CD4+ T cells (not shown).

FIG. 28 shows representative flow cytometry plots of GFP+ Raji cells co-cultured with non-transduced or drug product viral particle-transduced T cells at an approximate E:T ratio of 1:1 for one week. Circular gate represents the Raji cell population, which is absent in co-cultures with drug product viral particle-transduced T cells and also reduced in response to rapamycin treatment alone. UB-VV100 denotes the drug product.

FIG. 29A shows representative flow cytometry plots of drug product added to non-stimulated PBMCs from a dingle donor on Day=0. 3 days later (Day 3) the cells were washed to remove the viral particles and fresh media was added. The cells were then divided in half and one group received rapamycin (10 nM). On Day 20, functional responses were assessed by stimulating with Raji tumor cells for 5 hours in the presence of the golgi inhibitors Brefeldin A and monensin. Cells were collected and stained for Live/Dead™, surface markers, and intracellular IFN-γ. All sampled were gated on viable, Raji-, CD3+, and CAR+ cells.

FIG. 29B shows representative flow cytometry plots of drug product added to non-stimulated PBMCs from a dingle donor on Day=0. 3 days later (Day 3) the cells were washed to remove the viral particles and fresh media was added. The cells were then divided in half and one group received rapamycin (10 nM). On Day 20, functional responses were assessed by stimulating with Raji tumor cells for 5 hours in the presence of the golgi inhibitors Brefeldin A and monensin. Cells were collected and stained for Live/Dead™, surface markers, and TNFα. All sampled were gated on viable, Raji-, CD3+, and CAR+ cells.

FIG. 30A shows a graph of B cell populations assessed by flow cytometry (defined by live/singlets/human CD45+/CD3-CD20+) in the blood, spleen, and bone marrow. Error bars indicate +/−standard error of the mean. **** indicates p<0.0001, two-way ANOVA multiple comparisons. Bars indicate median values per group. * and *** indicate p<0.05 and <0.001 respectively, two-tailed T-test. UB-VV100 denotes the drug product.

FIG. 30B shows a graph of CAR T cells detected by peripheral leukocyte payload integration using ddPCR. UB-VV100 denotes the drug product.

FIG. 30C shows a graph of CAR T cells detected by peripheral leukocyte payload integration using flow cytometry. UB-VV100 denotes the drug product.

FIG. 31A shows the study timeline for an illustrative lentiviral vector dose exploration study.

FIG. 31B shows B Cells/ul of blood. A dose dependent antigen specific VV100 activity was observed, no B cell depletion present in the FITC RACR group. At day 25 B cell depletion plateaued, and on day 26 rapamycin treatment begun for all groups except vehicle group. Raji cells were implanted SC on day 40.

FIG. 31C shows CAR T cells/ul of blood. An increase in CAR T cells/ul was observed in the 10E6 VV100 treatment group and CAR T cells became detectable in the 2E06 VV100 treatment group after rapamycin treatment.

FIGS. 32A and 32B show tumor growth as measured by calipers using the formula (W\hat{ }2×L)/2 (A), and Tumor CAR T cells analyzed by flow cytometry (B). Inhibition of tumor growth appears to be dose dependent; no RAJI tumor growth inhibition was observed in FITC RACR treated mice. Higher CAR T cell frequency in tumors from 10E⁶ VV100 treated mice.

FIG. 33A shows cytometry analysis of CAR T cells in bone marrow (left) and spleen (right) at day 81. Higher dose of UB-VV100 sustains partial B cell depletion in bone marrow and spleen at 81 days post dosing.

FIG. 33B shows cytometry analysis of B cells in bone marrow (left) and spleen (right) at day 81. Higher dose of UB-VV100 sustains partial B cell depletion in bone marrow and spleen at 81 days post dosing.

FIG. 33C shows the analysis of transgene integration events in the blood, bone marrow, liver, ovary, and kidney normalized to DNA. Error bars indicate +/−1 SEM. Horizontal bars indicate median values. Dotted line indicates 50 vector copies/ug DNA as FDA guidance detection threshold.

FIG. 34A shows the study timeline for an illustrative lentiviral vector in vivo efficacy study using a Nalm-6 tumor model.

FIG. 34B shows the body weight changes in different groups of the Nalm-6 efficacy study.

FIG. 35A shows total flux (p/s) of different groups of the Nalm-6 efficacy study throughout the study as an indication of tumor burden. VV100 treatment significantly decreased tumor burden measured by Total Flux, all mice in the vehicle group succumbed to Nalm-6 tumor by study day 20, mice that received VV100 treatment extended their survival up to study day 41.

FIG. 35B shows survival curves of different groups of the Nalm-6 efficacy study throughout the study. VV100 treatment significantly increased survival, all mice in the vehicle group succumbed to Nalm-6 tumor by study day 20, mice that received VV100 treatment extended their survival up to study day 41

FIG. 36 shows Total flux data for each individual mouse in the Nalm-6 group throughout the study. Mice in Vehicle (upper left) and Vehicle+ rapamycin (upper right) groups had an elevated disease burden starting at study day 10. Mice in these groups had to be euthanized by day 17. Mice in VV100 group (lower left) had a decrease of disease burden starting at day 17, however the effects of VV100 in this group were temporary. All mice in the VV100+rapamycin group (lower right) had a significant decrease in disease burden starting at day 17, and low disease burden remained in two mice. Only one mouse from this group had tumor burden increase after the initial regression.

FIG. 37 shows Nalm-6 group bioluminescence imaging with total flux heatmap overlay. From left to right: Mice in the Vehicle group succumbed to Nalm-6 disease at day 17, mice in Vehicle+Rapamycin group succumbed to disease by day 20, most of the mice in the VV100 treatment group had a temporary decrease in disease burden, mice in the VV100+Rapamycin group had a significant decrease of disease burden that stayed low to undetectable in most of the mice, one mouse had a partial reduction in tumor burden that then increased

FIG. 38A shows the average CART cells/ul of blood. Mice treated with UB-VV100 alone have detectable circulating CAR T cells peaking at day 17 and overall low levels in the blood. In the UB-VV100+Rapamycin group, circulating CAR T cells increased over time and were significantly higher than in the UB-VV100 alone group. Mouse 10 had a significant expansion of CAR T cells in peripheral blood increasing from 228 CAR T cells/ul on D38 to 3397.69 on day 41.

FIG. 38B shows CAR T cells/ul of blood for each individual mouse. Mice treated with UB-VV100 alone have detectable circulating CAR T cells peaking at day 17 and overall low levels in the blood. In the UB-VV100+Rapamycin group, circulating CAR T cells increased over time and were significantly higher than in the UB-VV100 alone group. Mouse 10 had a significant expansion of CAR T cells in peripheral blood increasing from 228 CAR T cells/ul on D38 to 3397.69 on day 41.

FIG. 39A shows CAR T cell frequency of total immune cells in bone marrow and spleen. At day 41 CAR T cell frequency in the total immune cell population is higher in the VV100+Rapamycin treatment groups in both tissues and higher in the bone marrow than in the spleen.

FIG. 39B shows T cell frequency of human T cells in bone marrow and spleen. At day 41, the frequency of CAR T cells in the T cell population in bone marrow and spleen are significantly higher in the VV100+Rapamycin treatment group than in the VV100 treatment.

FIG. 40A shows activation of PBMCs by flow cytometry for CD25.

FIG. 40B shows transduction efficiency of PBMCs by flow cytometry for CAR expression.

FIG. 41A shows flow cytometry panels of CART cells in PBMCs transduced with UB-V100 and cultured with or without 10 nM rapamycin.

FIG. 41B shows charts of T cell yield and percentage in PBMCs transduced with UB-V100 and cultured with or without 10 nM rapamycin. Summarized plots combine data from 3 PBMC donors, error bars indicate +1 SEM. **, ***, and *** indicate p values of <0.01, 0.001, and 0.0001, 2-way ANOVA multiple comparisons for rapamycin treatment over time.

FIG. 42A shows flow cytometry and statistical analysis of intracellular staining of INFγ in CD8+ CAR T cells generated by UB-VV100 transduction.

FIG. 42B shows flow cytometry and statistical analysis of surface CD107a expression in CD8+ CAR T cells generated by UB-VV100 transduction.

FIG. 42C shows statistical analysis of Raji cell survival in the presence of PBMCs transduced with UB-VV100.

FIG. 43A shows flow cytometry panels of the PBMC sample from a B-ALL patient at the time of UB-VV100 transduction.

FIG. 43B shows flow cytometry panels of the PBMC sample from a B-ALL patient 7 days post UB-VV100 transduction.

FIG. 43C shows flow cytometry panels of the PBMC sample from a B-ALL patient 7 days post UB-VV100 transduction.

FIGS. 44A-44D show flow cytometry panels of the PBMC sample from a DLBCL patient at the time of UB-VV100 transduction.

FIGS. 45A and 45B show circulating B cell levels in CD34-NSG mice. Human B cell levels (hCD20+ population gated on single cells, live cells, and hCD45+ cells) were measured in the blood of CD34-NSG mice (Groups 7-10) using a flow cytometry human immune cell antibody panel. The data is shown as either (A) human B cell count (the quantified number of hCD20+ cells normalized to the volume of blood using counting beads) or (B) the human B cell frequency (% of hCD45+ cell population that was hCD20+). Data are mean±SEM. Number of animals in each group are according to outline in Study Design Table accounting for euthanasia and unscheduled deaths (n=2/vehicle group/timepoint; n=4/UB-VV100 group/timepoint). Statistics determined using two-way ANOVA main effects column test, with Tukey's adjustment; **=p<0.01, ***=p<0.001, ****=p<0.0001. CAR=chimeric antigen receptor; h=human.

FIGS. 46A and 46B show CAR-T cell levels in the blood of CD34-NSG mice. CAR-T cell levels (CAR+ population gated on single cells, live cells, hCD45+ cells, and hCD3+ cells) were measured in the blood of CD34-NSG mice (Group 7-10) using a flow cytometry human immune cell antibody panel containing an antibody targeting the FMC63 region of the αCD19 CAR construct. The data is shown as either (A) CAR-T cell frequency (% of hCD3+ cell population that was CAR+) or (B) the absolute number of CAR-T cells detected normalized to the volume of blood using counting beads. Data are mean±SEM. Number of animals in each group are according to outline in Study Design Table accounting for euthanasia and unscheduled deaths (n=2/vehicle group/timepoint; n=4/UB-VV100 group/timepoint). Statistics determined using two-way ANOVA main effects column test, with Tukey's adjustment; **=p<0.01, ***=p<0.001, ****=p<0.0001. CAR=chimeric antigen receptor; h=human.

FIG. 46C shows transduction of mouse immune cells in the spleen of CD34-NSG mice. CAR+ mouse immune cell levels (CAR+ population gated on single cells, live cells, and mCD45+ cells) were measured in the spleen after week 4 scheduled necropsies (Group 7, 8, 10). CAR+ cell detection was achieved using a flow cytometry human immune cell antibody panel containing an antibody targeting the FMC63 region of the αCD19 CAR construct. Bars are mean±SEM with dots representing individual mice. n=2 for the Vehicle+Rapamycin group and n=4 for Low Dose+Rapamycin and High Dose+Rapamycin groups. Statistics determined using one-way ANOVA, with Tukey's adjustment for multiple comparisons; **=p<0.01, ***=p<0.001, ****=p<0.0001. CAR=chimeric antigen receptor, m=mouse.

FIG. 46D shows UB-VV100 biodistribution in CD34-NSG mice. Copies of UB-VV100 integrated vector genomes were measured using ddPCR performed on genomic DNA extracted from bone marrow, spleen, liver, heart, lungs, kidneys, brain, and gonads of CD34-NSG mice at scheduled necropsies 1- or 4-weeks post treatment (Group 7-10). The data is shown as copies of amplified region (ssCD19) per ug of genomic DNA. Data bars are mean±SEM, with each dot representing an individual mouse. n=2 for Vehicle+Rapamycin (Group 7), n=4 for Low Dose+Rapamycin (Group 8) and High Dose+Rapamycin (Group 10), and n=3 for Low Dose—Rapamycin (Group 9). Data for gonads on Week 1 and Week 4 was not collected.

FIG. 47 shows multiplex RNA ISH staining in liver and spleen. Representative images of the liver and spleen from a CD34-NSG mouse treated with high dose UB-VV100 and rapamycin (TOX001_48). DAPI is shown in white, custom probe recognizing the RACR sequence of UB-VV100 is shown in yellow, human CD3 RNA ISH probe pool is shown in green, mouse CD68 RNA ISH probe is shown in red, and mouse Pecam RNA ISH probe is shown in blue. Human CAR-T cells are indicated with white arrows.

FIG. 48 shows CAR+ Nalm6 cells have reduced surface CD19 detection, but intracellular CD19 protein levels were the same as untransduced Nalm6 cells. Nalm6 cells were transduced with VV100 at MOIs 1, 10, and 20. On day 10, CAR+ Nalm6 cells were stained with (A) an anti-CD19 antibody (clone HIB19) to assess surface CD19 levels and (B) an anti-CD19 antibody that binds to an intracellular CD19 epitope (clone EPR5906) to determine the overall CD19 protein level. Orange curves represent data from CAR+ Nalm6 cells gated as CAR and P2A positive cells. Grey curves represent untransduced CD19+ Nalm6 parental cells or CD19 knockout Nalm6 cells.

FIG. 49 shows that anti-CD19 CAR-T cells can kill CAR+ Nalm6 cells in vitro. Transduced Nalm6 GFP cells (VV100, MOI 10) were co-cultured with transduced PBMCs (VV100, MOI 5) from 3 healthy donors at different CAR-T to Nalm6 ratios. To normalize for background non-specific killing, transduced Nalm6 cells were also cocultured with mock transduced PBMCs. Mock transduced PBMCs were treated with “empty” virus particles that displayed anti-CD3-scFv on the surface but did not carry a transgene payload. After 24 hours, transduced Nalm6 cells were gated as CAR+ Nalm6 cells based on intracellular transgene expression by flow cytometry. The percentage of lysis was calculated based on the frequency of dead CAR+ Nalm6 cells normalized to the mock transduced PBMC co-culture well.

FIG. 50 shows a schematic of VV100 transduction in a high tumor burden model. In this high tumor burden model, 5e5 Nalm6 GFP and 5e5 PBMCs were mixed and transduced with VV100 at a MOI of 5.

FIG. 51 shows that transducing a mixed population of Nalm6 cells and PBMCs with VV100 generates CAR+ Nalm6 cells, which were eliminated by anti-CD19 CAR-T cells. On day 0, 5e5 Nalm6 GFP cells and 5e5 PBMCs from healthy donors were mixed and transduced with either VV100 or anti-FITC CAR at a MOI of 5 or left untransduced. A total of eight healthy donors were evaluated. After transduction, (A) the frequency of Nalm6 cells that expressed the CAR transgene, (B) the total number of CAR+ Nalm6 cells, (C) the frequency of Nalm6 GFP cells in culture and (D) the total number of live Nalm6 GFP cells were determined by flow cytometry. Each line represents data from an individual healthy donor.

FIG. 52 shows that transducing a mixed population of Nalm6 cells and PBMCs with VV100 generates anti-CD19 CAR-T cells that can eliminate CAR+ Nalm6 cells. On day 0, 5e5 Nalm6 GFP cells and 5e5 PBMCs from healthy donors were mixed and transduced with either VV100 or anti-FITC CAR at a MOI of 5 or left untransduced. A total of eight healthy donors were evaluated. After transduction, (A) the frequency of CD3+ T cells that expressed the CAR transgene and (B) total number of αCD3 CAR-T cells were determined by flow cytometry. Each line represents data from an individual healthy donor.

FIG. 53 shows the study timeline for an illustrative lentiviral vector in vivo efficacy study.

FIG. 54 shows the study the survival of animals treated with UB-VV100. Vehicle (N=5), 142 (SEQ ID NO: 121) adherent 20E6 (N=5), 201 (SEQ ID NO: 122) adherent 2E6 (N=6), VPN38 20E6 (N=6), VPN38 100E6 (N=6), VPN68 20E6 (N=6), VPN68 100E6 (N=6).

FIG. 55 shows T cell phenotype 3 days after UB-VV100 administration. Peripheral blood was collected from mice and analyzed by flow cytometry to determine CD71 expression. Bars indicate median value. *, *, **, and **** indicate p value of <0.05, <0.01, and <0.0001, one way ANOVA Tukey's post comparisons test.

FIGS. 56A and 56B show circulating CAR T cells in mice treated with UB-VV100. Blood was collected from animals during serial weekly bleeds. CAR T cells were enumerated by flow cytometry. Error bars equal +/−1 SD. Vehicle (N=5), 142 (SEQ ID NO: 121) adherent 20E6 (N=5), 201 (SEQ ID NO: 122) adherent 2E6 (N=6), VPN38 20E6 (N=6), VPN38 100E6 (N=6), VPN68 20E6 (N=6), VPN68 100E6 (N=6).

FIG. 57 shows the average tumor burden throughout the study. All mice were monitored biweekly via bioluminescence imaging to evaluate NALM-6 progression. Error bars equal +/−1 SD. Vehicle (N=5), 142 (SEQ ID NO: 121) adherent 20E6 (N=5), 201 (SEQ ID NO: 122) adherent 2E6 (N=6), VPN38 20E6 (N=6), VPN38 100E6 (N=6), VPN68 20E6 (N=6), VPN68 100E6 (N=6). These mice received NALM=6 cells via retroorbital injection causing eye tumor.

FIG. 58A shows the percentage of NALM-6 GFP ffLUC tumor cells present in bone marrow at sacrifice. When humane endpoint was reached mice were euthanized, their bone marrow was collected and processed for flowcytometry analysis looking at the frequency of GFP+/CD45−/live cells.

FIG. 58B shows the percentage of NALM-6 GFP ffLUC tumor cells present in spleen at sacrifice. When humane endpoint was reached mice were euthanized, their spleen was collected and processed for flowcytometry analysis looking at the frequency of GFP+/CD45−/live cells.

FIG. 59 shows illustrative αCD3 scFv constructs for lentiviral surface expression plasmids of the present disclosure.

FIG. 60 shows the percentage of αCD3 scFv expression in lentiviral particles with the various illustrative αCD3 scFv surface constructs. 293T cells were transfected with the 5 plasmid system with variation in the surface plasmid construct. Producer 293T cells were analyzed for αCD3 scFv expression using the anti-teplizumab antibody with flow cytometry. Virus was harvested and used to transduce Supt1 cells which were analyzed for titer by flow cytometry.

FIG. 61 shows the titer of Supt1 cells transduced with lentivirus comprising various illustrative αCD3 scFv surface constructs.

FIG. 62 is a graph depicting relative light units (RLU) as a function of MOI in a T Cell Activation Bioassay (NEAT) bioluminescent cell-based assay showing T cell activation.

FIG. 63 is a graph depicting the correlation of αCD3 scFv expression and T cell activation by way of a NEAT reporter assay (RLU (MOI2)).

FIG. 64 shows representative flow cytometry plots for cells expressing various illustrative αCD3 scFv surface constructs and stained to visualize αCD3 scFv.

FIG. 65 shows the biodistribution of αCD3-Cocal-GFP in canine blood samples collected 24 Hours post-dose and prior to necropsy. Prior necropsy: Day 8 for Groups 1, 2 and 3 and Day 29 for Group 4. Each symbol represents a single individual. Results were extrapolated in order to be represent as copy number per μg of total canine DNA for tissue samples. The dotted line represents 2 parameters of the qPCR assay that were part of the validation: the lower limit of quantification (LLOQ) at 50 copies/μg of DNA after extrapolation (10 copies/200 ng of DNA) and the limit of detection (LOD) at 25 copies/μg of DNA after extrapolation (5 copies/200 ng of DNA). In order to visualize negative samples on the logarithmic scale, samples that gave an undetectable result were given a value of 1 for graphical representation but remain undetectable in this study.

FIG. 66 shows the biodistribution of αCD3-Cocal-GFP in canine tissues. Each symbol represents a single individual. Results were extrapolated in order to be represent as copy number per μg of total canine DNA for tissue samples. The dotted line represents 2 parameters of the qPCR assay that were part of the validation: the lower limit of quantification (LLOQ) at 50 copies/μg of DNA after extrapolation (10 copies/200 ng of DNA) and the limit of detection (LOD) at 25 copies/μg of DNA after extrapolation (5 copies/200 ng of DNA). Samples that were below the limit of quantification (BLQ) were given a value of 37, which is between the LLOQ and LOD values after extrapolation for graphical representation. In order to visualize negative samples on the logarithmic scale, samples that gave an undetectable result were given a value of 1 for graphical representation but remain undetectable in this study.

### DETAILED DESCRIPTION OF THE INVENTION

The disclosure relates generally to a viral particle comprising a vector genome comprising a polynucleotide sequence encoding an anti-CD19 chimeric antigen receptor, wherein the viral particle transduces immune cells in vivo.

The methods and compositions described herein may facilitate administering the viral particles directly into the subjects in need of treatment.

In vitro and in vivo studies demonstrate the ability of the methods and compositions described herein to activate and transduce non-stimulated T cells, which then express the αCD19 CAR, respond to rapamycin treatment with enhanced proliferation, and kill B-cell malignant models that express CD19.

The present disclosure provides a method of treating a disease or disorder, transducing immune cells in vivo, and/or generating an immune cell expressing an anti-CD19 chimeric antigen receptor in a subject in need thereof, comprising administering the viral particle of the present disclosure to the subject. In some embodiments, the method further comprises administering rapamycin to the subject. In some embodiments, the method of the disclosure eliminates the need for pre-activation of the immune cells prior to administration of the viral particle. In some embodiments, the method comprises no pre-activation of the immune cells in the subject prior to administration of the viral particle (e.g., no pre-activation within about 1, 2, 3, 4, 5, 6, or 7 days, or within about 1, 2, 3, 4, 5, 6, 7, 8, 9, 10, 11, or 12 weeks prior to administration of the viral particle). In some embodiments, pre-activation of the immune cells comprises activating the CD3 and/or CD28 signaling in the immune cells (e.g., T cells), optionally by administering anti-CD3 and/or anti-CD28 antibodies, respectively. Accordingly, in some embodiments, the method of the disclosure does not comprise administering separate CD3 and/or CD28 activating agents prior to administration of the viral particle.

In Vivo Delivery of Polynucleotides

In some embodiments, a polynucleotide encoding a chimeric antigen receptor (CAR) is administered to the subject which allows the production of the CAR in vivo. In some embodiments, the administration of such polynucleotide generates similar effect in vivo as direct administration of the CAR. In some embodiments, the administration of such polynucleotide improves the in vivo transduction efficiency of a particle. In some embodiments, the polynucleotide is an mRNA.

In some embodiments, in vivo delivery of such polynucleotides generates CAR expression over time (e.g., starting within hours and lasting several days). In some embodiments, in vivo delivery of such polypeptides results in desirable pharmacokinetics, pharmacodynamics and/or safety profile of the encoded CAR. In some embodiments, the polynucleotide may be optimized by one or more means to prevent immune activation, increase stability, reduce any tendency to aggregate, such as over time, and/or to avoid impurities. Such optimization may include the use of modified nucleosides, modified, and/or particular 5′ UTRs, 3′UTRs, and/or poly(A) tail modifications for improved intracellular stability and translational efficiency (see, e.g., Stadler et al., 2017, Nat. Med.). Such modifications are known in the art.

Strategies for in vivo delivery of polynucleotides (e.g., mRNA) are known in the art. For a summary of strategies, see Mol. Ther. 2019 Apr. 10; 27(4): 710-728, which is incorporated herein by reference in its entirety.

In some embodiments, the viral particle of the present disclosure can transduce T cells in vivo to express an anti-CD19 CAR and target CD19-expressing tumor cells.

In some embodiments, the viral particle has a multi-step mechanism of action:
(a) the viral particle binds to T cells in vivo via an anti-CD3 scFv, activates the T cells and facilitates viral particle internalization through interaction with the Cocal glycoprotein
(b) the vector RNA genome, αCD19 CAR-FRB-RACR, is reverse-transcribed into DNA, shuttled to the nucleus, and integrated into the genome
(c) the transduced T cells express the anti-CD19 CAR and target CD19-expressing cells, while also expressing the FRB and RACR system for rapamycin-controlled cytokine signaling.

Administration Route

In some embodiments, the viral particle is administered via a route selected from the group consisting of parenteral, intravenous, intramuscular, subcutaneous, intratumoral, intraperitoneal, and intralymphatic. In some embodiments, the viral particle is administered multiple times. In some embodiments, the viral particle is administered by intralymphatic injection of the viral particle. In some embodiments, the viral particle is administered by intraperitoneal injection of the viral particle. In some embodiments, the viral particle is administered by intra-nodal injection—that is, the viral particle may be administered via injection into a lymph node, such as an inguinal lymph node. In some embodiments, the viral particle is administered by injection of the viral particle into tumor sites (i.e. intratumoral). In some embodiments, the viral particle is administered subcutaneously. In some embodiments, the viral particle is administered systemically. In some embodiments, the viral particle is administered intravenously. In some embodiments, the viral particle is administered intra-arterially. In some embodiments, the viral particle is a lentiviral particle.

In some embodiments, the viral particle is administered by intraperitoneal, subcutaneous, or intranodal injection. In some embodiments, the viral particle is administered by intraperitoneal injection. In some embodiments, the viral particle is administered by subcutaneous injection. In some embodiments, the viral particle is administered by intranodal injection.

In some embodiments, the transduced immune cells comprising the polynucleotide of the present disclosure is administered to the subject.

In some embodiments, the viral particle is administered as a single injection. In some embodiments, the viral particle is administered as at least 1, at least 2, at least 3, at least 4, at least 5, at least 6, at least 7, at least 8, at least 9, or at least 10 injections.

Viral Particle

In some embodiments, the viral particle comprises a polynucleotide. In some embodiments, the polynucleotide encodes at least one therapeutic polypeptide. The term “therapeutic polypeptide” refers to a polypeptide which is being developed for therapeutic use, or which has been developed for therapeutic use. In some embodiments, the therapeutic polypeptide is expressed in target cells (e.g., host T cells) for therapeutic use. In some embodiments, the therapeutic polypeptide comprises a T cell receptor, a chimeric antigen receptor, or a cytokine receptor.

In some embodiments, the viral particle as described herein is a retroviral particle. In some embodiments, the viral particle is a lentiviral particle. In some embodiments, the viral particle is an adeno-associated virus particle.

The term viral particle refers to a macromolecular complex capable of transferring a nucleic acid into a cell. Viral vectors contain structural and/or functional genetic elements that are primarily derived from a virus. The term “retroviral vector” refers to a viral vector containing structural and functional genetic elements, or portions thereof, that are primarily derived from a retrovirus. The term “lentiviral vector” refers to a viral vector containing structural and functional genetic elements, or portions thereof, including LTRs that are primarily derived from a lentivirus. The term “hybrid” refers to a vector, LTR or other nucleic acid containing both retroviral, e.g., lentiviral, sequences and non-lentiviral viral sequences. In some embodiments, a hybrid vector refers to a vector or transfer plasmid comprising retroviral, e.g., lentiviral, sequences for reverse transcription, replication, integration and/or packaging.

In some embodiments, the lentiviral particle of the present disclosure is a replication incompetent, self-inactivating (SIN) lentiviral vector (LVV) particle comprising:
A surface-engineered viral envelope that includes expression of a membrane-bound anti-CD3 single-chain variable fragment (scFv) and the Cocal glycoprotein.
A transgene encoding:

A 2^{nd} generation anti-CD19 chimeric antigen receptor (CAR) comprising the binding domain FMC63 and the 4-1BB and CD3zeta signaling domains;
An inducible T-cell proliferative signaling system (rapamycin-activated cytokine receptor, RACR); and
A human protein domain (FRB) derived from the mammalian target of rapamycin (mTOR) complex that binds intracellular rapamycin to confer rapamycin-resistance to transduced cells.

Retroviral Particle

Retroviruses include lentiviruses, gamma-retroviruses, and alpha-retroviruses, each of which may be used to deliver polynucleotides to cells using methods known in the art. Lentiviruses are complex retroviruses, which, in addition to the common retroviral genes gag, pol, and env, contain other genes with regulatory or structural function. The higher complexity enables the virus to modulate its life cycle, as in the course of latent infection. Illustrative lentiviruses include but are not limited to: HIV (human immunodeficiency virus; including HIV type 1, and HIV type 2; visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, the backbones are HIV-based vector backbones (i.e., HIV cis-acting sequence elements). Retroviral particles have been generated by multiply attenuating the HIV virulence genes, for example, the genes env, vif, vpr, vpu and nef are deleted, making the vector biologically safe.

Illustrative lentiviral particles include those described in Naldini et al. (1996) Science 272:263-7; Zufferey et al. (1998) J. Virol. 72:9873-9880; Dull et al. (1998) J. Virol. 72:8463-8471; U.S. Pat. Nos. 6,013,516; and 5,994,136, which are each incorporated herein by reference in their entireties. In general, these particles are configured to carry the essential sequences for selection of cells containing the particle, for incorporating foreign nucleic acid into a lentiviral particle, and for transfer of the nucleic acid into a target cell.

A commonly used lentiviral particles system is the so-called third-generation system. Third-generation lentiviral particles systems include four plasmids. The “transfer plasmid” encodes the polynucleotide sequence that is delivered by the lentiviral vector system to the target cell. The transfer plasmid generally has one or more transgene sequences of interest flanked by long terminal repeat (LTR) sequences, which facilitate integration of the transfer plasmid sequences into the host genome. For safety reasons, transfer plasmids are generally designed to make the resulting particles replication incompetent. For example, the transfer plasmid lacks gene elements necessary for generation of infective particles in the host cell. In addition, the transfer plasmid may be designed with a deletion of the 3′ LTR, rendering the virus “self-inactivating” (SIN). See Dull et al. (1998) J. Virol. 72:8463-71; Miyoshi et al. (1998) J. Virol. 72:8150-57. The viral particle may also comprise a 3′ untranslated region (UTR) and a 5′ UTR. The UTRs comprise retroviral regulatory elements that support packaging, reverse transcription and integration of a proviral genome into a cell following contact of the cell by the retroviral particle.

Third-generation systems also generally include two “packaging plasmids” and an “envelope plasmid.” The “envelope plasmid” generally encodes an Env gene operatively linked to a promoter. In an exemplary third-generation system, the Env gene is VSV-G and the promoter is the CMV promoter. In an exemplary third-generation system, the Env gene is Cocal G protein (Cocal glycoprotein) and the promoter is the MND (myeloproliferative sarcoma virus enhancer, negative control region deleted, d1587rev primer-binding site substituted) promoter. In an exemplary third-generation system, the Env gene is Cocal G protein (Cocal glycoprotein) and the promoter is the CMV promoter. The third-generation system uses two packaging plasmids, one encoding gag and pol and the other encoding rev as a further safety feature—an improvement over the single packaging plasmid of so-called second-generation systems. Although safer, the third-generation system can be more cumbersome to use and result in lower viral titers due to the addition of an additional plasmid. Exemplary packing plasmids include, without limitation, pMD2.G, pRSV-rev, pMDLG-pRRE, and pRRL-GOI.

Many retroviral particle systems rely on the use of a “packaging cell line.” In general, the packaging cell line is a cell line whose cells are capable of producing infectious retroviral particles when the transfer plasmid, packaging plasmid(s), and envelope plasmid are introduced into the cells. Various methods of introducing the plasmids into the cells may be used, including transfection or electroporation. In some cases, a packaging cell line is adapted for high-efficiency packaging of a retroviral particle system into retroviral particles.

As used herein, the terms “retroviral particle” or “lentiviral particle” refers to a viral particle that includes a polynucleotide encoding a heterologous protein (e.g. a chimeric antigen receptor), one or more capsid proteins, and other proteins necessary for transduction of the polynucleotide into a target cell. Retroviral particles and lentiviral particles generally include an RNA genome (derived from the transfer plasmid), a lipid-bilayer envelope in which the Env protein is embedded, and other accessory proteins including integrase, protease, and matrix protein.

The ex vivo efficiency of a retroviral or lentiviral particle system may be assessed in various ways known in the art, including measurement of vector copy number (VCN) or vector genomes (vg) such as by quantitative polymerase chain reaction (qPCR), digital droplet PCR (ddPCR) or titer of the virus in infectious units per milliliter (IU/mL) For example, the titer may be assessed using a functional assay performed on the cultured tumor cell line HT1080 as described in Humbert et al. Development of Third-generation Cocal Envelope Producer Cell Lines for Robust Retroviral Gene Transfer into Hematopoietic Stem Cells and T-cells. Molecular Therapy 24:1237-1246 (2016). When titer is assessed on a cultured cell line that is continually dividing, no stimulation is required and hence the measured titer is not influenced by surface engineering of the retroviral particle. Other methods for assessing the efficiency of retroviral vector systems are provided in Gaererts et al. Comparison of retroviral vector titration methods. BMC Biotechnol 6:34 (2006).

In some embodiments, the retroviral particles and/or lentiviral particles of the disclosure comprise a polynucleotide comprising a sequence encoding a receptor that specifically binds to a hapten. In some embodiments, a sequence encoding a receptor that specifically binds to the hapten is operatively linked to a promoter. Illustrative promoters include, without limitation, a cytomegalovirus (CMV) promoter, a CAG promoter, an SV40 promoter, an SV40/CD43 promoter, an EF-1α promoter, and a MND promoter.

In some embodiments, the polynucleotide encoding the chimeric antigen receptor is operatively linked to one or more promoters. In some embodiments, the promoter is an inducible promoter. In some embodiments, the promoter is CMV. In some embodiments, the promoter is MND.

In some embodiments, the polynucleotide encoding the RACR is operatively linked to one or more promoters. In some embodiments, the promoter is an inducible promoter. In some embodiments, the promoter is CMV. In some embodiments, the promoter is MND.

In some embodiments, the retroviral particles comprise transduction enhancers. In some embodiments, the retroviral particles comprise a polynucleotide comprising a sequence encoding a T cell activator protein. In some embodiments, the retroviral particles comprise a polynucleotide comprising a sequence encoding a hapten-binding receptor. In some embodiments, the retroviral particles comprise tagging proteins.

In some embodiments, each of the retroviral particles comprises a polynucleotide comprising, in 5′ to 3′ order: (i) a 5′ long terminal repeat (LTR) or untranslated region (UTR), (ii) a promoter, (iii) a sequence encoding a receptor that specifically binds to the hapten, and (iv) a 3′ LTR or UTR.

Viral Envelope

In some embodiments, the retroviral particles comprise a cell surface receptor that binds to a ligand on a target host cell, allowing host cell transduction. The viral particle may comprise a heterologous viral envelope glycoprotein yielding a pseudotyped viral particle. For example, the viral envelope glycoprotein may be derived from RD114 or one of its variants, VSV-G, Gibbon-ape leukemia virus (GALV), or is the Amphotropic envelope, Measles envelope or baboon retroviral envelope glycoprotein. In some embodiments, the viral envelope glycoprotein is a VSV G protein from the Cocal strain (Cocal glycoprotein) or a functional variant thereof.

In some embodiments, the viral envelope glycoprotein is a VSV G protein from the Cocal strain (Cocal glycoprotein) is a Cocal envelope variant containing the R354Q mutation, this variant may be referred to as “blinded” Cocal envelope. Illustrative Cocal envelope variants are provided in, e.g., US 2020/0216502 A1, which is incorporated herein by reference in its entirety.

In some embodiments, the viral particle comprises a polypeptide comprising a Cocal glycoprotein that shares at least 75%, at least 80%, at least 85%, at least 90%, at least 95%, at least 99%, or 100% identity to SEQ ID NO: 5.

Cocal Env:
(SEQ ID NO: 5)
NFLLLTFIVLPLCSHAKFSIVFPQSQKGNWKNVPSSYHYCPSSSDQNWH
NDLLGITMKVKMPKTHKAIQADGWMCHAAKWITTCDFRWYGPKYITHSI
HSIQPTSEQCKESIKQTKQGTWMSPGFPPQNCGYATVTDSVAVVVQATP
HHVLVDEYTGEWIDSQFPNGKCETEECETVHNSTVWYSDYKVTGLCDAT
LVDTEITFFSEDGKKESIGKPNTGYRSNYFAYEKGDKVCKMNYCKHAGV
RLPSGVWFEFVDQDVYAAAKLPECPVGATISAPTQTSVDVSLILDVERI
LDYSLCQETWSKIRSKQPVSPVDLSYLAPKNPGTGPAFTIINGTLKYFE
TRYIRIDIDNPIISKMVGKISGSQTERELWTEWFPYEGVEIGPNGILKT
PTGYKFPLFMIGHGMLDSDLHKTSQAEVFEHPHLAEAPKQLPEEETLFF
GDTGISKNPVELIEGWFSSWKSTVVTFFFAIGVFILLYVVARIVIAVRY
RYQGSNNKRIYNDIEMSRFRK.

In some embodiments, the viral particle comprises a nucleic acid sequence encoding a Cocal glycoprotein that shares at least 75%, at least 80%, at least 85%, at least 90%, at least 95%, at least 99%, or 100% identity to SEQ ID NO: 10.

Cocal Env:
(SEQ ID NO: 10)
AATTTTCTGCTGCTGACCTTCATCGTGCTGCCTCTGTGCAGCCACGCCA
AGTTTTCCATCGTGTTCCCACAGTCCCAGAAGGGCAACTGGAAGAATGT
GCCCTCTAGCTACCACTATTGCCCTTCCTCTAGCGACCAGAACTGGCAC
AATGATCTGCTGGGCATCACAATGAAGGTGAAGATGCCCAAGACCCACA
AGGCCATCCAGGCAGATGGATGGATGTGCCACGCAGCCAAGTGGATCAC
AACCTGTGACTTTCGGTGGTACGGCCCCAAGTATATCACACACTCCATC
CACTCTATCCAGCCTACCTCCGAGCAGTGCAAGGAGTCTATCAAGCAGA
CAAAGCAGGGCACCTGGATGAGCCCTGGCTTCCCACCCCAGAACTGTGG
CTACGCCACAGTGACCGACTCCGTGGCAGTGGTGGTGCAGGCAACACCT
CACCACGTGCTGGTGGATGAGTATACCGGCGAGTGGATCGACAGCCAGT
TTCCAAACGGCAAGTGCGAGACAGAGGAGTGTGAGACCGTGCACAATTC
TACAGTGTGGTACAGCGATTATAAGGTGACAGGCCTGTGCGACGCCACC
CTGGTGGATACAGAGATCACCTTCTTTTCTGAGGACGGCAAGAAGGAGA
GCATCGGCAAGCCCAACACCGGCTACAGATCCAATTACTTCGCCTATGA
GAAGGGCGATAAGGTGTGCAAGATGAATTATTGTAAGCACGCCGGGGTG
CGGCTGCCTAGCGGCGTGTGGTTTGAGTTCGTGGACCAGGACGTGTACG
CAGCAGCAAAGCTGCCTGAGTGCCCAGTGGGAGCAACCATCTCCGCCCC
AACACAGACCTCCGTGGACGTGTCTCTGATCCTGGATGTGGAGCGCATC
CTGGACTACAGCCTGTGCCAGGAGACCTGGAGCAAGATCCGGTCCAAGC
AGCCCGTGTCCCCTGTGGACCTGTCTTACCTGGCACCAAAGAACCCAGG
AACCGGACCAGCCTTTACAATCATCAATGGCACCCTGAAGTACTTCGAG
ACCCGCTATATCCGGATCGACATCGATAACCCTATCATCAGCAAGATGG
TGGGCAAGATCTCTGGCAGCCAGACAGAGAGAGAGCTGTGGACCGAGTG
GTTCCCTTACGAGGGCGTGGAGATCGGCCCAAATGGCATCCTGAAGACA
CCAACCGGCTATAAGTTTCCCCTGTTCATGATCGGCCACGGCATGCTGG
ACAGCGATCTGCACAAGACCTCCCAGGCCGAGGTGTTTGAGCACCCACA
CCTGGCAGAGGCACCAAAGCAGCTGCCTGAGGAGGAGACACTGTTCTTT
GGCGATACCGGCATCTCTAAGAACCCCGTGGAGCTGATCGAGGGCTGGT
TTTCCTCTTGGAAGAGCACAGTGGTGACCTTCTTTTTCGCCATCGGCGT
GTTCATCCTGCTGTACGTGGTGGCCAGAATCGTGATCGCCGTGAGATAC
AGGTATCAGGGCTCCAACAATAAGAGGATCTATAATGACATCGAGATGT
CTCGCTTCCGGAAG.

In some embodiments, the viral particle comprises a nucleic acid sequence encoding a Cocal glycoprotein that shares at least 75%, at least 80%, at least 85%, at least 90%, at least 95%, at least 99%, or 100% identity to SEQ ID NO: 104.

Cocal Env:
(SEQ ID NO: 104)
ATGAACTTTCTGCTGCTGACCTTCATCGTGCTGCCTCTGTGCAGCCACG
CCAAGTTTTCCATCGTGTTCCCACAGTCCCAGAAGGGCAACTGGAAGAA
TGTGCCCAGCTCCTACCACTATTGTCCTTCTAGCTCCGACCAGAACTGG
CACAATGATCTGCTGGGCATCACCATGAAGGTGAAGATGCCTAAGACAC
ACAAGGCCATCCAGGCAGATGGATGGATGTGCCACGCAGCCAAGTGGAT
CACCACATGTGACTTTCGGTGGTACGGCCCCAAGTATATCACCCACAGC
ATCCACTCCATCCAGCCTACAAGCGAGCAGTGCAAGGAGTCCATCAAGC
AGACCAAGCAGGGCACATGGATGTCTCCCGGCTTCCCCCCTCAGAACTG
TGGCTACGCCACCGTGACAGATAGCGTGGCAGTGGTGGTGCAGGCAACC
CCACACCACGTGCTGGTGGATGAGTATACAGGCGAGTGGATCGACAGCC
AGTTTCCCAACGGCAAGTGCGAGACCGAGGAGTGTGAGACAGTGCACAA
TTCTACCGTGTGGTACAGCGATTATAAGGTGACCGGCCTGTGCGACGCC
ACACTGGTGGATACCGAGATCACATTCTTTTCCGAGGACGGCAAGAAGG
AGTCTATCGGCAAGCCCAACACCGGCTACAGGTCTAATTACTTCGCCTA
TGAGAAGGGCGATAAGGTGTGCAAGATGAATTATTGTAAGCACGCCGGG
GTGCGGCTGCCAAGCGGCGTGTGGTTTGAGTTCGTGGACCAGGACGTGT
ACGCAGCAGCAAAGCTGCCAGAGTGCCCAGTGGGAGCAACCATCAGCGC
CCCCACCCAGACATCTGTGGACGTGAGCCTGATCCTGGATGTGGAGAGA
ATCCTGGACTACTCCCTGTGCCAGGAGACATGGTCCAAGATCCGCTCTA
AGCAGCCCGTGAGCCCAGTGGACCTGTCTTACCTGGCACCAAAGAACCC
TGGAACAGGACCTGCCTTTACCATCATCAATGGCACACTGAAGTACTTC
GAGACCCGGTATATCAGAATCGACATCGATAACCCAATCATCTCCAAGA
TGGTGGGCAAGATCTCCGGCTCTCAGACCGAGAGAGAGCTGTGGACAGA
GTGGTTCCCATACGAGGGCGTGGAGATCGGCCCCAATGGCATCCTGAAG
ACCCCTACAGGCTATAAGTTTCCACTGTTCATGATCGGCCACGGCATGC
TGGACTCTGATCTGCACAAGACCAGCCAGGCCGAGGTGTTTGAGCACCC
ACACCTGGCAGAGGCACCAAAGCAGCTGCCCGAGGAGGAGACCCTGTTC
TTTGGCGATACAGGCATCTCCAAGAACCCTGTGGAGCTGATCGAGGGCT
GGTTTTCTAGCTGGAAGTCTACCGTGGTGACATTCTTTTTCGCCATCGG
CGTGTTCATCCTGCTGTACGTGGTGGCAAGGATCGTGATCGCCGTGCGG
TACAGATATCAGGGCAGCAACAATAAGAGAATCTATAATGACATCGAGA
TGTCCAGGTTCCGCAAGTGA

In some embodiments, the viral particle comprises a polynucleotide comprising CD8 derived signal peptide sequence that shares at least 75%, at least 80%, at least 85%, at least 90%, at least 95%, at least 99%, or 100% identity to SEQ ID NO: 1.

CD8 signal peptide:
(SEQ ID NO: 1)
MALPVTALLLPLALLLHAARP.

In some embodiments, the viral particle comprises a nucleic acid sequence encoding a CD8 derived signal peptide that shares at least 75%, at least 80%, at least 85%, at least 90%, at least 95%, at least 99%, or 100% identity to SEQ ID NO: 6.

CD8 signal peptide:
(SEQ ID NO: 6)
ATGGCACTGCCTGTGACAGCCCTGCTGCTGCCACTGGCCCTGCTGCTGC
ACGCAGCACGCCCA.

In some embodiments, the cell surface receptor is anti-CD3 single-chain variable fragment or a functional variant thereof.

Various fusion glycoproteins can be used to pseudotype lentiviral particles. While the most commonly used example is the envelope glycoprotein from vesicular stomatitis virus (VSV-G), many other viral proteins have also been used for pseudotyping of lentiviral particles. See Joglekar et al. Human Gene Therapy Methods 28:291-301 (2017). The present disclosure contemplates substitution of various fusion glycoproteins. Notably, some fusion glycoproteins result in higher viral particle efficiency.

In some embodiments, pseudotyping a fusion glycoprotein or functional variant thereof facilitates targeted transduction of specific cell types, including, but not limited to, T cells or NK-cells. In some embodiments, the fusion glycoprotein or functional variant thereof is/are full-length polypeptide(s), functional fragment(s), homolog(s), or functional variant(s) of Human immunodeficiency virus (HIV) gp160, Murine leukemia virus (MLV) gp70, Gibbon ape leukemia virus (GALV) gp70, Feline leukemia virus (RD114) gp70, Amphotropic retrovirus (Ampho) gp70, 10A1 MLV (10A1) gp70, Ecotropic retrovirus (Eco) gp70, Baboon ape leukemia virus (BaEV) gp70, Measles virus (MV) H and F, Nipah virus (NiV) H and F, Rabies virus (RabV) G, Mokola virus (MOKV) G, Ebola Zaire virus (EboZ) G, Lymphocytic choriomeningitis virus (LCMV) GP1 and GP2, Baculovirus GP64, Chikungunya virus (CHIKV) E1 and E2, Ross River virus (RRV) E1 and E2, Semliki Forest virus (SFV) E1 and E2, Sindbis virus (SV) E1 and E2, Venezualan equine encephalitis virus (VEEV) E1 and E2, Western equine encephalitis virus (WEEV) E1 and E2, Influenza A, B, C, or D HA, Fowl Plague Virus (FPV) HA, anti-CD3 scFv, (CD3), Vesicular stomatitis virus VSV-G, or Chandipura virus and Piry virus CNV-G and PRV-G.

In some embodiments, the fusion glycoprotein or functional variant thereof is a full-length polypeptide, functional fragment, homolog, or functional variant of the G protein of Vesicular Stomatitis Alagoas Virus (VSAV), Carajas Vesiculovirus (CJSV), Chandipura Vesiculovirus (CHPV), Cocal Vesiculovirus (COCV), Vesicular Stomatitis Indiana Virus (VSIV), Isfahan Vesiculovirus (ISFV), Maraba Vesiculovirus (MARAV), Vesicular Stomatitis New Jersey virus (VSNJV), Bas-Congo Virus (BASV). In some embodiments, the fusion glycoprotein or functional variant thereof is the Cocal virus G protein.

In some embodiments, the viral particle is a Nipah virus (NiV) envelope pseudotyped lentivirus particle (“Nipah envelope pseudotyped vector”). In some embodiments, the Nipah envelope pseudotyped vector is pseudotyped using Nipah virus envelope glycoproteins NiV-F and NiV-G. In some embodiments, the NiV-F and/or NiV-G glycoproteins on such Nipah envelope pseudotyped vector are modified variants. In some embodiments, the NiV-F and/or NiV-G glycoproteins on such Nipah envelope pseudotyped vector are modified to include an antigen binding domain. In some embodiments, the antigen is EpCAM, CD4, or CD8. In some embodiments, the Nipah envelope pseudotyped vector can efficiently transduce cells expressing EpCAM, CD4, or CD8. See U.S. Pat. No. 9,486,539 and Bender et al. PLoS Pathog. (2016) June; 12(6): e1005641.

Viral Particle Envelope Antigen Binding Domain

In some embodiments, the glycoprotein on an envelope pseudotyped viral particle is modified to include an antigen binding domain. In some embodiments, the antigen is CD3. In some embodiments, the envelope pseudotyped viral particle can efficiently transduce cells expressing CD3. In some embodiments, the antigen binding domain is an anti-CD3 single-chain variable fragment (scFv). In some embodiments, the antigen binding domain is an anti-CD3 humanized murine scFv.

In some embodiments, the envelope pseudotyped viral particle is modified to include a fusion glycoprotein or functional variant thereof and an antigen binding domain or functional variant thereof. In some embodiments, the envelope pseudotyped viral particle is modified to include the Cocal virus G protein or functional variant thereof and an anti-CD3 scFv or functional variant thereof.

In some embodiments, the retroviral vector particle is surface-engineered. Illustrative methods of surface-engineering a retroviral vector particle are provided in, e.g., WO 2019/200056, PCT/US2019/062675, and U.S. 62/916,110, each of which is incorporated herein by reference in its entirety.

In some embodiments, the retroviral particle is surface-engineered to include a fusion glycoprotein or functional variant thereof and an antigen binding domain or functional variant thereof. In some embodiments, the retroviral particle is surface-engineered to include the Cocal virus G protein or functional variant thereof and an anti-CD3 scFv or functional variant thereof.

Various non-viral proteins capable of viral surface display are provided by the present disclosure. In some embodiments, the non-viral proteins are co-stimulatory molecules. Conventionally, lentiviral transduction in vitro requires additional of an exogenous activating agent, such as a “stimbead,” for example Dynabeads™ Human T-Activator αCD3/αCD28. In some embodiments, the retroviral (e.g. lentiviral) vectors of the present disclosure incorporate one or more copies of non-viral proteins such as T-cell activation or co-stimulation molecule(s). The incorporation of T-cell activation or co-stimulation molecule(s) in the particle may render the particle capable of activating and efficiently transducing T cells in the absence of, or in the presence of lower amounts of, an exogenous activating agent, i.e. without a stimbead or equivalent agent.

In some embodiments, the T-cell activation or co-stimulation molecule may be selected from the group consisting of an anti-CD3 antibody, CD28 ligand (CD28L), and 41bb ligand (41BBL or CD137L). Various T-cell activation or co-stimulation molecules are known in the art and include, without limitation, agents that specifically bind any of the T-cell expressed proteins CD3, CD28, CD134 also known as OX40, or 41bb also known as 4-1BB or CD137 or TNFRSF9. For example, an agent that specifically binds CD3 may be an anti-CD3 antibody (e.g., OKT3, CRIS-7 or I2C) or an antigen-binding fragment of an anti-CD3 antibody.

In some embodiments, an agent that specifically binds CD3 is a single chain Fv fragment (scFv) of an anti-CD3 antibody.

In some embodiments, the viral particle comprises a polypeptide comprising an anti-CD3 scFv (CD3 VL—linked to a CD3 VH by 3×G4S linkers) that shares at least 75%, at least 80%, at least 85%, at least 90%, at least 95%, at least 99%, or 100% identity to SEQ ID NO: 2.

Anti-CD3 scFv (VL-G4S x 3 linker-VH):
(SEQ ID NO: 2)
DIQMTQSPSSLSASVGDRVTITCSASSSVSYMNWYQQTPGKAPKRWIYD
TSKLASGVPSRFSGSGSGTDYTFTISSLQPEDIATYYCQQWSSNPFTFG
QGTKLQITRTSGGGGSGGGGSGGGGSQVQLVQSGGGVVQPGRSLRLSCK
ASGYTFTRYTMHWVRQAPGKGLEWIGYINPSRGYTNYNQKVKDRFTISR
DNSKNTAFLQMDSLRPEDTGVYFCARYYDDHYCLDYWGQGTPVTVSSAA
AKP.

The complementary determining regions (CDR) of this scFv are SASSSVSYMN (CDR-L1; SEQ ID NO: 133), DTSKLASG (CDR-L2; SEQ ID NO: 134), QQWSSNPFT (CDR-L3; SEQ ID NO: 135), RYTMH (CDR-H1; SEQ ID NO: 144), YINPSRGYTNYNQKVKD (CDR-H2; SEQ ID NO: 136), and YYDDHYCLDY (CDR-H3; SEQ ID NO: 137). In some embodiments, the viral particle comprises a polypeptide comprising an anti-CD3 scFv having these CDRs, wherein optionally the anti-CD3 scFv shares at least 75%, at least 80%, at least 85%, at least 90%, at least 95%, at least 99%, or 100% identity to SEQ ID NO: 2.

In some embodiments, the viral particle comprises a nucleic acid sequence encoding an anti-CD3 scFv (CD3 VL—linked to a CD3 VH by 3×G4S linkers) that shares at least 75%, at least 80%, at least 85%, at least 90%, at least 95%, at least 99%, or 100% identity to SEQ ID NO: 7.

Anti-CD3 scFv (VL-G4S x 3 linker-VH):
(SEQ ID NO: 7)
GATATCCAGATGACCCAGTCCCCAAGCTCCCTGAGCGCCTCCGTGGGCG
ACCGGGTGACAATCACCTGCAGCGCCTCTAGCTCCGTGTCCTACATGAA
CTGGTATCAGCAGACACCTGGCAAGGCCCCAAAGAGATGGATCTACGAT
ACCAGCAAGCTGGCCTCCGGCGTGCCTTCTAGGTTTTCTGGCAGCGGCT
CCGGCACAGATTATACATTCACCATCTCTAGCCTGCAGCCAGAGGACAT
CGCCACCTACTATTGCCAGCAGTGGTCCTCTAATCCCTTTACATTCGGC
CAGGGCACCAAGCTGCAGATCACAAGAACCTCTGGAGGAGGAGGAAGCG
GAGGAGGAGGATCCGGCGGCGGCGGCTCTCAGGTGCAGCTGGTGCAGAG
CGGAGGAGGAGTGGTGCAGCCAGGCAGAAGCCTGAGGCTGTCCTGTAAG
GCCTCTGGCTACACATTCACCAGATATACAATGCACTGGGTGAGGCAGG
CACCAGGCAAGGGACTGGAGTGGATCGGCTACATCAACCCCTCCAGGGG
CTACACCAACTATAATCAGAAGGTGAAGGATCGGTTCACCATCAGCAGG
GACAACTCCAAGAATACCGCCTTCCTGCAGATGGACAGCCTGAGGCCAG
AGGATACCGGCGTGTACTTTTGCGCCCGGTACTATGACGATCACTACTG
TCTGGATTATTGGGGCCAGGGAACACCAGTGACCGTGAGCTCCGCCGCA
GCAAAGCCT.

In some embodiments, the viral particle comprises a polypeptide comprising an anti-CD3 scFv (CD3 VL—linked to a CD3 VH by 3×G4S linkers) that shares at least 75%, at least 80%, at least 85%, at least 90%, at least 95%, at least 99%, or 100% identity to SEQ ID NO: 12.

Anti-CD3 scFv (VL-G4S x 3 linker-VH):
(SEQ ID NO: 12)
DIQMTQSPSSLSASVGDRVTITCSASSSVSYMNWYQQTPGKAPKRWIYD
TSKLASGVPSRFSGSGSGTDYTFTISSLQPEDIATYYCQQWSSNPFTFG
QGTKLQITRTSGGGGSGGGGSGGGGSQVQLVQSGGGVVQPGRSLRLSCK
ASGYTFTRYTMHWVRQAPGKGLEWIGYINPSRGYTNYNQKVKDRFTISR
DNSKNTAFLQMDSLRPEDTGVYFCARYYDDHYCLDYWGQGTPVTVSSA
S.

The complementary determining regions (CDR) of this scFv are SASSSVSYMN (CDR-L1; SEQ ID NO: 133), DTSKLASG (CDR-L2; SEQ ID NO: 134), QQWSSNPFT (CDR-L3; SEQ ID NO: 135), RYTMH (CDR-H1; SEQ ID NO: 144), YINPSRGYTNYNQKVKD (CDR-H2; SEQ ID NO: 136), and YYDDHYCLDY (CDR-H3; SEQ ID NO: 137). In some embodiments, the viral particle comprises a polypeptide comprising an anti-CD3 scFv having these CDRs, wherein optionally the anti-CD3 scFv shares at least 75%, at least 80%, at least 85%, at least 90%, at least 95%, at least 99%, or 100% identity to SEQ ID NO: 12.

In some embodiments, the viral particle comprises a nucleic acid sequence encoding an anti-CD3 scFv (CD3 VL—linked to a CD3 VH by 3×G4S linkers) that shares at least 75%, at least 80%, at least 85%, at least 90%, at least 95%, at least 99%, or 100% identity to SEQ ID NO: 15.

Anti-CD3 scFv (VL-G4S x 3 linker-VH):
(SEQ ID NO: 15)
GACATCCAGATGACCCAGTCTCCTAGCAGCCTCAGCGCTAGCGTGGGCG
ATAGAGTGACCATCACATGTAGCGCCAGCAGCAGCGTGTCCTACATGAA
CTGGTACCAGCAAACACCTGGAAAGGCCCCTAAAAGGTGGATCTATGAC
ACATCTAAGCTGGCTTCTGGAGTGCCATCTAGATTTTCTGGCAGCGGCT
CCGGCACTGATTATACATTCACCATCAGCAGCCTGCAGCCCGAGGATAT
CGCCACCTACTACTGTCAGCAGTGGTCCTCTAATCCCTTCACCTTCGGC
CAGGGCACCAAGCTGCAGATCACCAGAACCAGCGGCGGGGGAGGAAGCG
GCGGGGGAGGATCTGGCGGCGGCGGCAGCCAGGTGCAGCTGGTGCAGAG
CGGCGGCGGCGTGGTGCAACCTGGCAGAAGCCTGAGACTGAGCTGCAAG
GCCTCTGGCTACACCTTCACCCGGTACACCATGCATTGGGTGCGGCAGG
CCCCTGGCAAGGGCCTGGAATGGATTGGATACATCAACCCCAGCAGAGG
CTACACCAACTACAACCAGAAGGTGAAGGACAGATTCACAATTTCTCGG
GACAACAGCAAGAATACCGCCTTCCTGCAAATGGACTCCCTGCGCCCAG
AAGATACCGGCGTGTACTTCTGCGCTAGATATTACGACGACCACTACTG
CCTGGACTACTGGGGCCAGGGCACCCCTGTGACCGTGTCCAGCGCCTC
C.

In some embodiments, the viral particle comprises a polypeptide comprising an anti-CD3 scFv comprising a CD3 VL linked to a CD3 VH by 3×G4S linkers.

In some embodiments, the viral particle comprises a nucleic acid sequence encoding an anti-CD3 scFv comprising a CD3 VL linked to a CD3 VH by 3×G4S linkers.

In some embodiments, the viral particle comprises a polypeptide comprising a Gaussia luciferase signal peptide, operably linked to an anti-CD3 scFv, operably linked to a hinge domain, operably linked to a Cocal envelope derived transmembrane domain and cytoplasmic tail that shares at least 75%, at least 80%, at least 85%, at least 90%, at least 95%, at least 99%, or 100% identity to SEQ ID NO: 107.

αCD3scFv_short hinge-TM-CT (pUMJ_224)
(SEQ ID NO: 107)
MGVKVLFALICIAVAEADIQMTQSPSSLSASVGDRVTITCSASSSVSYM
NWYQQTPGKAPKRWIYDTSKLASGVPSRFSGSGSGTDYTFTISSLQPED
IATYYCQQWSSNPFTFGQGTKLQITRTSGGGGSGGGGSGGGGSQVQLVQ
SGGGVVQPGRSLRLSCKASGYTFTRYTMHWVRQAPGKGLEWIGYINPSR
GYTNYNQKVKDRFTISRDNSKNTAFLQMDSLRPEDTGVYFCARYYDDHY
CLDYWGQGTPVTVSSASGVELIEGWFSSWKSTVVTFFFAIGVFILLYVV
ARIVIAVRYRYQGSNNKRIYNDIEMSRFRK

In some embodiments, the viral particle comprises a nucleic acid encoding a Gaussia luciferase signal peptide, operably linked to an anti-CD3 scFv, operably linked to a hinge domain, operably linked to a Cocal envelope derived transmembrane domain and cytoplasmic tail that shares at least 75%, at least 80%, at least 85%, at least 90%, at least 95%, at least 99%, or 100% identity to SEQ ID NO: 108.

αCD3scFv_short hinge-TM-CT (pUMJ_224)
(SEQ ID NO: 108)
ATGGGCGTGAAAGTGCTGTTCGCCCTGATCTGCATCGCAGTTGCTGAAG
CCGACATCCAGATGACCCAGTCTCCTAGCAGCCTCAGCGCTAGCGTGGG
CGATAGAGTGACCATCACATGTAGCGCCAGCAGCAGCGTGTCCTACATG
AACTGGTACCAGCAAACACCTGGAAAGGCCCCTAAAAGGTGGATCTATG
ACACATCTAAGCTGGCTTCTGGAGTGCCATCTAGATTTTCTGGCAGCGG
CTCCGGCACTGATTATACATTCACCATCAGCAGCCTGCAGCCCGAGGAT
ATCGCCACCTACTACTGTCAGCAGTGGTCCTCTAATCCCTTCACCTTCG
GCCAGGGCACCAAGCTGCAGATCACCAGAACCAGCGGCGGGGGAGGAAG
CGGCGGGGGAGGATCTGGCGGCGGCGGCAGCCAGGTGCAGCTGGTGCAG
AGCGGCGGCGGCGTGGTGCAACCTGGCAGAAGCCTGAGACTGAGCTGCA
AGGCCTCTGGCTACACCTTCACCCGGTACACCATGCATTGGGTGCGGCA
GGCCCCTGGCAAGGGCCTGGAATGGATTGGATACATCAACCCCAGCAGA
GGCTACACCAACTACAACCAGAAGGTGAAGGACAGATTCACAATTTCTC
GGGACAACAGCAAGAATACCGCCTTCCTGCAAATGGACTCCCTGCGCCC
AGAAGATACCGGCGTGTACTTCTGCGCTAGATATTACGACGACCACTAC
TGCCTGGACTACTGGGGCCAGGGCACCCCTGTGACCGTGTCCAGCGCCT
CCGGAGTGGAACTGATCGAGGGCTGGTTCAGCAGCTGGAAAAGCACCGT
GGTTACATTCTTTTTCGCCATCGGCGTGTTCATCCTGCTGTACGTGGTC
GCCAGAATTGTGATCGCCGTGCGGTATAGATACCAGGGCAGCAACAACA
AGCGGATCTACAACGACATCGAGATGAGCAGATTCAGAAAG

In some embodiments, the viral particle comprises a polypeptide comprising a Gaussia luciferase signal peptide, operably linked to an anti-CD3 scFv, operably linked to a hinge domain, operably linked to a Cocal envelope derived transmembrane domain and cytoplasmic tail that shares at least 75%, at least 80%, at least 85%, at least 90%, at least 95%, at least 99%, or 100% identity to SEQ ID NO: 109.

αCD3scFv_long hinge_TM_CT (pUMJ_163)
(SEQ ID NO: 109)
MGVKVLFALICIAVAEADIQMTQSPSSLSASVGDRVTITCSASSSVSYM
NWYQQTPGKAPKRWIYDTSKLASGVPSRFSGSGSGTDYTFTISSLQPED
IATYYCQQWSSNPFTFGQGTKLQITRTSGGGGSGGGGSGGGGSQVQLVQ
SGGGVVQPGRSLRLSCKASGYTFTRYTMHWVRQAPGKGLEWIGYINPSR
GYTNYNQKVKDRFTISRDNSKNTAFLQMDSLRPEDTGVYFCARYYDDHY
CLDYWGQGTPVTVSSASSGFEHPHLAEAPKQLPEEETLFFGDTGISKNP
VELIEGWFSSWKSTVVTFFFAIGVFILLYVVARIVIAVRYRYQGSNNKR
IYNDIEMSRFRK

In some embodiments, the viral particle comprises a nucleic acid encoding a Gaussia luciferase signal peptide, operably linked to an anti-CD3 scFv, operably linked to a hinge domain, operably linked to a Cocal envelope derived transmembrane domain and cytoplasmic tail that shares at least 75%, at least 80%, at least 85%, at least 90%, at least 95%, at least 99%, or 100% identity to SEQ ID NO: 110.

αCD3scFv_long hinge_TM_CT (pUMJ_163)
(SEQ ID NO: 110)
ATGGGCGTGAAAGTGCTGTTCGCCCTGATCTGCATCGCAGTTGCTGAAG
CCGACATCCAGATGACCCAGTCTCCTAGCAGCCTCAGCGCTAGCGTGGG
CGATAGAGTGACCATCACATGTAGCGCCAGCAGCAGCGTGTCCTACATG
AACTGGTACCAGCAAACACCTGGAAAGGCCCCTAAAAGGTGGATCTATG
ACACATCTAAGCTGGCTTCTGGAGTGCCATCTAGATTTTCTGGCAGCGG
CTCCGGCACTGATTATACATTCACCATCAGCAGCCTGCAGCCCGAGGAT
ATCGCCACCTACTACTGTCAGCAGTGGTCCTCTAATCCCTTCACCTTCG
GCCAGGGCACCAAGCTGCAGATCACCAGAACCAGCGGCGGGGGAGGAAG
CGGCGGGGGAGGATCTGGCGGCGGCGGCAGCCAGGTGCAGCTGGTGCAG
AGCGGCGGCGGCGTGGTGCAACCTGGCAGAAGCCTGAGACTGAGCTGCA
AGGCCTCTGGCTACACCTTCACCCGGTACACCATGCATTGGGTGCGGCA
GGCCCCTGGCAAGGGCCTGGAATGGATTGGATACATCAACCCCAGCAGA
GGCTACACCAACTACAACCAGAAGGTGAAGGACAGATTCACAATTTCTC
GGGACAACAGCAAGAATACCGCCTTCCTGCAAATGGACTCCCTGCGCCC
AGAAGATACCGGCGTGTACTTCTGCGCTAGATATTACGACGACCACTAC
TGCCTGGACTACTGGGGCCAGGGCACCCCTGTGACCGTGTCCAGCGCCT
CCGGATTCGAGCACCCCCACCTGGCCGAGGCCCCTAAGCAGCTGCCTGA
AGAAGAGACACTGTTTTTCGGAGATACCGGCATCAGCAAAAACCCCGTG
GAGCTGATCGAGGGCTGGTTCAGCTCTTGGAAGAGCACCGTGGTCACAT
TCTTTTTCGCCATCGGCGTCTTTATCCTGCTGTACGTGGTAGCCAGAAT
CGTGATCGCCGTGCGGTACAGATACCAGGGCAGCAACAACAAGCGGATC
TACAACGACATCGAGATGAGCCGGTTCAGAAAG

In some embodiments, the viral particle comprises a polypeptide comprising a Gaussia luciferase signal peptide, operably linked to an anti-CD3 scFv, operably linked to a linker, operably linked to a Glycophorin A derived transmembrane domain and HIV envelope derived cytoplasmic tail that shares at least 75%, at least 80%, at least 85%, at least 90%, at least 95%, at least 99%, or 100% identity to SEQ ID NO: 111.

αCD3scFv_hGlycophorinA_TM_HIV Env CT (pUMJ_194)
(SEQ ID NO: 111)
MGVKVLFALICIAVAEADIQMTQSPSSLSASVGDRVTITCSASSSVSYM
NWYQQTPGKAPKRWIYDTSKLASGVPSRFSGSGSGTDYTFTISSLQPED
IATYYCQQWSSNPFTFGQGTKLQITRTSGGGGSGGGGSGGGGSQVQLVQ
SGGGVVQPGRSLRLSCKASGYTFTRYTMHWVRQAPGKGLEWIGYINPSR
GYTNYNQKVKDRFTISRDNSKNTAFLQMDSLRPEDTGVYFCARYYDDHY
CLDYWGQGTPVTVSSASGGSTSGSGKPGSGEGSTKGPEITLIIFGVMAG
VIGTILLISYGIRRLALKYWWNLLQYWSQELKNSAVSLLNATAIAVAEG
TDRVIEVVQGACRAIRHIPRRIRQGLERILL

In some embodiments, the viral particle comprises a nucleic acid encoding a Gaussia luciferase signal peptide, operably linked to an anti-CD3 scFv, operably linked to a linker, operably linked to a Glycophorin A derived transmembrane domain and HIV envelope derived cytoplasmic tail that shares at least 75%, at least 80%, at least 85%, at least 90%, at least 95%, at least 99%, or 100% identity to SEQ ID NO: 112.

αCD3scFv_hGlycophorinA_TM_HIV Env CT (pUMJ_194)
(SEQ ID NO: 112)
ATGGGCGTGAAAGTGCTGTTCGCCCTGATCTGCATCGCAGTTGCTGAAG
CCGACATCCAGATGACCCAGTCTCCTAGCAGCCTCAGCGCTAGCGTGGG
CGATAGAGTGACCATCACATGTAGCGCCAGCAGCAGCGTGTCCTACATG
AACTGGTACCAGCAAACACCTGGAAAGGCCCCTAAAAGGTGGATCTATG
ACACATCTAAGCTGGCTTCTGGAGTGCCATCTAGATTTTCTGGCAGCGG
CTCCGGCACTGATTATACATTCACCATCAGCAGCCTGCAGCCCGAGGAT
ATCGCCACCTACTACTGTCAGCAGTGGTCCTCTAATCCCTTCACCTTCG
GCCAGGGCACCAAGCTGCAGATCACCAGAACCAGCGGCGGGGGAGGAAG
CGGCGGGGGAGGATCTGGCGGCGGCGGCAGCCAGGTGCAGCTGGTGCAG
AGCGGCGGCGGCGTGGTGCAACCTGGCAGAAGCCTGAGACTGAGCTGCA
AGGCCTCTGGCTACACCTTCACCCGGTACACCATGCATTGGGTGCGGCA
GGCCCCTGGCAAGGGCCTGGAATGGATTGGATACATCAACCCCAGCAGA
GGCTACACCAACTACAACCAGAAGGTGAAGGACAGATTCACAATTTCTC
GGGACAACAGCAAGAATACCGCCTTCCTGCAAATGGACTCCCTGCGCCC
AGAAGATACCGGCGTGTACTTCTGCGCTAGATATTACGACGACCACTAC
TGCCTGGACTACTGGGGCCAGGGCACCCCTGTGACCGTGTCCAGCGCCT
CCGGAGGATCTACAAGCGGCTCTGGCAAGCCTGGCAGCGGAGAAGGCAG
CACCAAGGGCCCTGAGATCACACTGATCATCTTCGGCGTGATGGCCGGC
GTCATCGGCACCATCCTGCTGATCAGCTACGGCATCAGAAGACTGGCTC
TGAAGTACTGGTGGAATCTGCTGCAATACTGGAGCCAGGAGCTGAAAAA
CAGCGCCGTGTCCCTGCTCAACGCCACCGCCATCGCCGTGGCCGAGGGC
ACCGACAGAGTGATCGAGGTGGTGCAGGGAGCCTGCAGAGCTATTCGGC
ACATCCCCAGACGGATCAGGCAGGGCCTGGAAAGAATCCTGCTG

In some embodiments, the viral particle comprises a polypeptide comprising a Gaussia luciferase signal peptide, operably linked to an anti-CD3 scFv, operably linked to a linker, operably linked to a HIV envelope derived transmembrane domain and cytoplasmic tail that shares at least 75%, at least 80%, at least 85%, at least 90%, at least 95%, at least 99%, or 100% identity to SEQ ID NO: 113.

αCD3scFv_218 linker_HIV Env ecto-TM-CT (pUMJ_195)
(SEQ ID NO: 113)
MGVKVLFALICIAVAEADIQMTQSPSSLSASVGDRVTITCSASSSVSYM
NWYQQTPGKAPKRWIYDTSKLASGVPSRFSGSGSGTDYTFTISSLQPED
IATYYCQQWSSNPFTFGQGTKLQITRTSGGGGSGGGGSGGGGSQVQLVQ
SGGGVVQPGRSLRLSCKASGYTFTRYTMHWVRQAPGKGLEWIGYINPSR
GYTNYNQKVKDRFTISRDNSKNTAFLQMDSLRPEDTGVYFCARYYDDHY
CLDYWGQGTPVTVSSASGGSTSGSGKPGSGEGSTKGNWLWYIRIFIIIV
GSLIGLRIVFAVLSLVNRGWEALKYWWNLLQYWSQELKNSAVSLLNATA
IAVAEGTDRVIEVVQGACRAIRHIPRRIRQGLERILL

In some embodiments, the viral particle comprises a nucleic acid encoding a Gaussia luciferase signal peptide, operably linked to an anti-CD3 scFv, operably linked to a linker, operably linked to a HIV envelope derived transmembrane domain and cytoplasmic tail that shares at least 75%, at least 80%, at least 85%, at least 90%, at least 95%, at least 99%, or 100% identity to SEQ ID NO: 114.

αCD3scFv_218 linker_HIV Env ecto-TM-CT (pUMJ_195)
(SEQ ID NO: 114)
ATGGGCGTGAAAGTGCTGTTCGCCCTGATCTGCATCGCAGTTGCTGAAG
CCGACATCCAGATGACCCAGTCTCCTAGCAGCCTCAGCGCTAGCGTGGG
CGATAGAGTGACCATCACATGTAGCGCCAGCAGCAGCGTGTCCTACATG
AACTGGTACCAGCAAACACCTGGAAAGGCCCCTAAAAGGTGGATCTATG
ACACATCTAAGCTGGCTTCTGGAGTGCCATCTAGATTTTCTGGCAGCGG
CTCCGGCACTGATTATACATTCACCATCAGCAGCCTGCAGCCCGAGGAT
ATCGCCACCTACTACTGTCAGCAGTGGTCCTCTAATCCCTTCACCTTCG
GCCAGGGCACCAAGCTGCAGATCACCAGAACCAGCGGCGGGGGAGGAAG
CGGCGGGGGAGGATCTGGCGGCGGCGGCAGCCAGGTGCAGCTGGTGCAG
AGCGGCGGCGGCGTGGTGCAACCTGGCAGAAGCCTGAGACTGAGCTGCA
AGGCCTCTGGCTACACCTTCACCCGGTACACCATGCATTGGGTGCGGCA
GGCCCCTGGCAAGGGCCTGGAATGGATTGGATACATCAACCCCAGCAGA
GGCTACACCAACTACAACCAGAAGGTGAAGGACAGATTCACAATTTCTC
GGGACAACAGCAAGAATACCGCCTTCCTGCAAATGGACTCCCTGCGCCC
AGAAGATACCGGCGTGTACTTCTGCGCTAGATATTACGACGACCACTAC
TGCCTGGACTACTGGGGCCAGGGCACCCCTGTGACCGTGTCCAGCGCCT
CCGGAGGAAGCACCAGCGGCTCTGGCAAGCCTGGCAGCGGCGAGGGCTC
TACCAAGGGCAATTGGCTGTGGTACATCAGAATCTTCATCATCATCGTG
GGCAGCCTGATCGGCCTGAGAATCGTGTTCGCCGTGCTGAGCCTGGTGA
ACCGGGGCTGGGAAGCTCTGAAGTACTGGTGGAACCTGCTGCAATACTG
GTCCCAGGAGCTGAAAAACAGCGCTGTGTCCCTGCTCAACGCCACCGCC
ATCGCCGTCGCCGAGGGAACAGACAGAGTGATCGAGGTGGTGCAGGGAG
CCTGCAGAGCCATTCGGCACATCCCCAGACGCATCAGACAGGGCCTGGA
AAGAATCCTGCTG

In some embodiments, the viral particle comprises a polypeptide comprising a Gaussia luciferase signal peptide, operably linked to an anti-CD3 scFv, operably linked to a triple G4Slinker, operably linked to a HIV envelope derived transmembrane domain and cytoplasmic tail that shares at least 75%, at least 80%, at least 85%, at least 90%, at least 95%, at least 99%, or 100% identity to SEQ ID NO: 115.

αCD3scFv_G4S linker_HIV Env ecto-TM-CT (pUMJ_196)
(SEQ ID NO: 115)
MGVKVLFALICIAVAEADIQMTQSPSSLSASVGDRVTITCSASSSVSYM
NWYQQTPGKAPKRWIYDTSKLASGVPSRFSGSGSGTDYTFTISSLQPED
IATYYCQQWSSNPFTFGQGTKLQITRTSGGGGSGGGGSGGGGSQVQLVQ
SGGGVVQPGRSLRLSCKASGYTFTRYTMHWVRQAPGKGLEWIGYINPSR
GYTNYNQKVKDRFTISRDNSKNTAFLQMDSLRPEDTGVYFCARYYDDHY
CLDYWGQGTPVTVSSASGGGGGSGGGGSGGGGSYIRIFIIIVGSLIGLR
IVFAVLSLVNRGWEALKYWWNLLQYWSQELKNSAVSLLNATAIAVAEGT
DRVIEVVQGACRAIRHIPRRIRQGLERILL

In some embodiments, the viral particle comprises a nucleic acid encoding a Gaussia luciferase signal peptide, operably linked to an anti-CD3 scFv, operably linked to a triple G4Slinker, operably linked to a HIV envelope derived transmembrane domain and cytoplasmic tail that shares at least 75%, at least 80%, at least 85%, at least 90%, at least 95%, at least 99%, or 100% identity to SEQ ID NO: 116.

αCD3scFv_G4S linker_IV Env ecto-TM-CT (pUMJ_196)
(SEQ ID NO: 116)
ATGGGCGTGAAAGTGCTGTTCGCCCTGATCTGCATCGCAGTTGCTGAAG
CCGACATCCAGATGACCCAGTCTCCTAGCAGCCTCAGCGCTAGCGTGGG
CGATAGAGTGACCATCACATGTAGCGCCAGCAGCAGCGTGTCCTACATG
AACTGGTACCAGCAAACACCTGGAAAGGCCCCTAAAAGGTGGATCTATG
ACACATCTAAGCTGGCTTCTGGAGTGCCATCTAGATTTTCTGGCAGCGG
CTCCGGCACTGATTATACATTCACCATCAGCAGCCTGCAGCCCGAGGAT
ATCGCCACCTACTACTGTCAGCAGTGGTCCTCTAATCCCTTCACCTTCG
GCCAGGGCACCAAGCTGCAGATCACCAGAACCAGCGGCGGGGGAGGAAG
CGGCGGGGGAGGATCTGGCGGCGGCGGCAGCCAGGTGCAGCTGGTGCAG
AGCGGCGGCGGCGTGGTGCAACCTGGCAGAAGCCTGAGACTGAGCTGCA
AGGCCTCTGGCTACACCTTCACCCGGTACACCATGCATTGGGTGCGGCA
GGCCCCTGGCAAGGGCCTGGAATGGATTGGATACATCAACCCCAGCAGA
GGCTACACCAACTACAACCAGAAGGTGAAGGACAGATTCACAATTTCTC
GGGACAACAGCAAGAATACCGCCTTCCTGCAAATGGACTCCCTGCGCCC
AGAAGATACCGGCGTGTACTTCTGCGCTAGATATTACGACGACCACTAC
TGCCTGGACTACTGGGGCCAGGGCACCCCTGTGACCGTGTCCAGCGCCT
CCGGAGGCGGTGGAGGCTCTGGTGGCGGAGGGAGCGGTGGCGGAGGCAG
CTACATCAGAATCTTCATCATCATCGTGGGCAGCCTGATCGGCCTGAGA
ATCGTGTTCGCCGTTCTGAGCCTGGTGAACCGGGGCTGGGAAGCCCTGA
AGTACTGGTGGAATCTGCTCCAGTACTGGTCTCAGGAGCTGAAGAACAG
CGCCGTGTCCCTGCTGAACGCTACAGCTATCGCCGTCGCCGAGGGCACC
GACAGAGTGATCGAGGTGGTGCAGGGCGCCTGCAGAGCCATCCGGCACA
TCCCTAGAAGGATTCGGCAAGGCCTGGAAAGAATCCTGCTG

In some embodiments, the viral particle comprises a polypeptide comprising a Gaussia luciferase signal peptide, operably linked to an anti-CD3 scFv, operably linked to a hinge domain, operably linked to a Cocal envelope derived transmembrane domain, cytoplasmic tail, and T2A self-cleaving peptide, operably linked to a Cocal envelope that shares at least 75%, at least 80%, at least 85%, at least 90%, at least 95%, at least 99%, or 100% identity to SEQ ID NO: 117.

anti-CD3scFv_short hinge_TM_CT_T2A_Cocal envelope:
(SEQ ID NO: 117)
MGVKVLFALICIAVAEADIQMTQSPSSLSASVGDRVTITCSASSSVSYM
NWYQQTPGKAPKRWIYDTSKLASGVPSRFSGSGSGTDYTFTISSLQPED
IATYYCQQWSSNPFTFGQGTKLQITRTSGGGGSGGGGSGGGGSQVQLVQ
SGGGVVQPGRSLRLSCKASGYTFTRYTMHWVRQAPGKGLEWIGYINPSR
GYTNYNQKVKDRFTISRDNSKNTAFLQMDSLRPEDTGVYFCARYYDDHY
CLDYWGQGTPVTVSSASGVELIEGWFSSWKSTVVTFFFAIGVFILLYVV
ARIVIAVRYRYQGSNNKRIYNDIEMSRFRKGSGEGRGSLLTCGDVEENP
GPNFLLLTFIVLPLCSHAKFSIVFPQSQKGNWKNVPSSYHYCPSSSDQN
WHNDLLGITMKVKMPKTHKAIQADGWMCHAAKWITTCDFRWYGPKYITH
SIHSIQPTSEQCKESIKQTKQGTWMSPGFPPQNCGYATVTDSVAVVVQA
TPHHVLVDEYTGEWIDSQFPNGKCETEECETVHNSTVWYSDYKVTGLCD
ATLVDTEITFFSEDGKKESIGKPNTGYRSNYFAYEKGDKVCKMNYCKHA
GVRLPSGVWFEFVDQDVYAAAKLPECPVGATISAPTQTSVDVSLILDVE
RILDYSLCQETWSKIRSKQPVSPVDLSYLAPKNPGTGPAFTIINGTLKY
FETRYIRIDIDNPIISKMVGKISGSQTERELWTEWFPYEGVEIGPNGIL
KTPTGYKFPLFMIGHGMLDSDLHKTSQAEVFEHPHLAEAPKQLPEEETL
FFGDTGISKNPVELIEGWFSSWKSTVVTFFFAIGVFILLYVVARIVIAV
RYRYQGSNNKRIYNDIEMSRFRK

In some embodiments, the viral particle comprises a nucleic acid encoding a Gaussia luciferase signal peptide, operably linked to an anti-CD3 scFv, operably linked to a hinge domain, operably linked to a Cocal envelope derived transmembrane domain, cytoplasmic tail, and T2A self-cleaving peptide, operably linked to a Cocal envelope that shares at least 75%, at least 80%, at least 85%, at least 90%, at least 95%, at least 99%, or 100% identity to SEQ ID NO: 118.

anti-CD3scFv_short hinge_TM_CT_T2A_Cocal envelope:
(SEQ ID NO: 118)
ATGGGCGTGAAAGTGCTGTTCGCCCTGATCTGCATCGCAGTTGCTGAAG
CCGACATCCAGATGACCCAGTCTCCTAGCAGCCTCAGCGCTAGCGTGGG
CGATAGAGTGACCATCACATGTAGCGCCAGCAGCAGCGTGTCCTACATG
AACTGGTACCAGCAAACACCTGGAAAGGCCCCTAAAAGGTGGATCTATG
ACACATCTAAGCTGGCTTCTGGAGTGCCATCTAGATTTTCTGGCAGCGG
CTCCGGCACTGATTATACATTCACCATCAGCAGCCTGCAGCCCGAGGAT
ATCGCCACCTACTACTGTCAGCAGTGGTCCTCTAATCCCTTCACCTTCG
GCCAGGGCACCAAGCTGCAGATCACCAGAACCAGCGGCGGGGGAGGAAG
CGGCGGGGGAGGATCTGGCGGCGGCGGCAGCCAGGTGCAGCTGGTGCAG
AGCGGCGGCGGCGTGGTGCAACCTGGCAGAAGCCTGAGACTGAGCTGCA
AGGCCTCTGGCTACACCTTCACCCGGTACACCATGCATTGGGTGCGGCA
GGCCCCTGGCAAGGGCCTGGAATGGATTGGATACATCAACCCCAGCAGA
GGCTACACCAACTACAACCAGAAGGTGAAGGACAGATTCACAATTTCTC
GGGACAACAGCAAGAATACCGCCTTCCTGCAAATGGACTCCCTGCGCCC
AGAAGATACCGGCGTGTACTTCTGCGCTAGATATTACGACGACCACTAC
TGCCTGGACTACTGGGGCCAGGGCACCCCTGTGACCGTGTCCAGCGCCT
CCGGAGTGGAACTGATCGAGGGCTGGTTCAGCAGCTGGAAAAGCACCGT
GGTTACATTCTTTTTCGCCATCGGCGTGTTCATCCTGCTGTACGTGGTC
GCCAGAATTGTGATCGCCGTGCGGTATAGATACCAGGGCAGCAACAACA
AGCGGATCTACAACGACATCGAGATGAGCAGATTCAGAAAGGGATCTGG
AGAGGGAAGGGGAAGCCTGCTGACATGCGGCGACGTGGAGGAGAACCCA
GGACCAAATTTTCTGCTGCTGACCTTCATCGTGCTGCCTCTGTGCAGCC
ACGCCAAGTTTTCCATCGTGTTCCCACAGTCCCAGAAGGGCAACTGGAA
GAATGTGCCCTCTAGCTACCACTATTGCCCTTCCTCTAGCGACCAGAAC
TGGCACAATGATCTGCTGGGCATCACAATGAAGGTGAAGATGCCCAAGA
CCCACAAGGCCATCCAGGCAGATGGATGGATGTGCCACGCAGCCAAGTG
GATCACAACCTGTGACTTTCGGTGGTACGGCCCCAAGTATATCACACAC
TCCATCCACTCTATCCAGCCTACCTCCGAGCAGTGCAAGGAGTCTATCA
AGCAGACAAAGCAGGGCACCTGGATGAGCCCTGGCTTCCCACCCCAGAA
CTGTGGCTACGCCACAGTGACCGACTCCGTGGCAGTGGTGGTGCAGGCA
ACACCTCACCACGTGCTGGTGGATGAGTATACCGGCGAGTGGATCGACA
GCCAGTTTCCAAACGGCAAGTGCGAGACAGAGGAGTGTGAGACCGTGCA
CAATTCTACAGTGTGGTACAGCGATTATAAGGTGACAGGCCTGTGCGAC
GCCACCCTGGTGGATACAGAGATCACCTTCTTTTCTGAGGACGGCAAGA
AGGAGAGCATCGGCAAGCCCAACACCGGCTACAGATCCAATTACTTCGC
CTATGAGAAGGGCGATAAGGTGTGCAAGATGAATTATTGTAAGCACGCC
GGGGTGCGGCTGCCTAGCGGCGTGTGGTTTGAGTTCGTGGACCAGGACG
TGTACGCAGCAGCAAAGCTGCCTGAGTGCCCAGTGGGAGCAACCATCTC
CGCCCCAACACAGACCTCCGTGGACGTGTCTCTGATCCTGGATGTGGAG
CGCATCCTGGACTACAGCCTGTGCCAGGAGACCTGGAGCAAGATCCGGT
CCAAGCAGCCCGTGTCCCCTGTGGACCTGTCTTACCTGGCACCAAAGAA
CCCAGGAACCGGACCAGCCTTTACAATCATCAATGGCACCCTGAAGTAC
TTCGAGACCCGCTATATCCGGATCGACATCGATAACCCTATCATCAGCA
AGATGGTGGGCAAGATCTCTGGCAGCCAGACAGAGAGAGAGCTGTGGAC
CGAGTGGTTCCCTTACGAGGGCGTGGAGATCGGCCCAAATGGCATCCTG
AAGACACCAACCGGCTATAAGTTTCCCCTGTTCATGATCGGCCACGGCA
TGCTGGACAGCGATCTGCACAAGACCTCCCAGGCCGAGGTGTTTGAGCA
CCCACACCTGGCAGAGGCACCAAAGCAGCTGCCTGAGGAGGAGACACTG
TTCTTTGGCGATACCGGCATCTCTAAGAACCCCGTGGAGCTGATCGAGG
GCTGGTTTTCCTCTTGGAAGAGCACAGTGGTGACCTTCTTTTTCGCCAT
CGGCGTGTTCATCCTGCTGTACGTGGTGGCCAGAATCGTGATCGCCGTG
AGATACAGGTATCAGGGCTCCAACAATAAGAGGATCTATAATGACATCG
AGATGTCTCGCTTCCGGAAG

In some embodiments, the viral particle comprises a polypeptide comprising a Gaussia luciferase signal peptide, operably linked to an anti-CD3 scFv, operably linked to a linker, operably linked to a Glycophorin A derived hinge, transmembrane domain, and cytoplasmic tail that shares at least 75%, at least 80%, at least 85%, at least 90%, at least 95%, at least 99%, or 100% identity to SEQ ID NO: 119.

αCD3scFv_human Glycophorin A hinge-TM-CT (pUMJ_232, used in VV100)
(SEQ ID NO: 119)
MGVKVLFALICIAVAEADIQMTQSPSSLSASVGDRVTITCSASSSVSYMNWYQ
QTPGKAPKRWIYDTSKLASGVPSRFSGSGSGTDYTFTISSLQPEDIATYYCQQWSSNP
FTFGQGTKLQITRTSGGGGSGGGGSGGGGSQVQLVQSGGGVVQPGRSLRLSCKASG
YTFTRYTMHWVRQAPGKGLEWIGYINPSRGYTNYNQKVKDRFTISRDNSKNTAFLQ
MDSLRPEDTGVYFCARYYDDHYCLDYWGQGTPVTVSSASHFSEPEITLIIFGVMAGV
IGTILLISYGIRRLIKKSPSDVKPLPSPDTDVPLSSVEIENPETSDQ

In some embodiments, the viral particle comprises a nucleic acid encoding a Gaussia luciferase signal peptide, operably linked to an anti-CD3 scFv, operably linked to a linker, operably linked to a Glycophorin A derived hinge, transmembrane domain, and cytoplasmic tail that shares at least 75%, at least 80%, at least 85%, at least 90%, at least 95%, at least 99%, or 100% identity to SEQ ID NO: 120.

αCD3scFv_human Glycophorin A hinge-TM-CT (pUMJ_232, used in VV100)
(SEQ ID NO: 120)
ATGGGCGTGAAAGTGCTGTTCGCCCTGATCTGCATCGCAGTTGCTGAAGC
CGACATCCAGATGACCCAGTCTCCTAGCAGCCTCAGCGCTAGCGTGGGCGATAG
AGTGACCATCACATGTAGCGCCAGCAGCAGCGTGTCCTACATGAACTGGTACCA
GCAAACACCTGGAAAGGCCCCTAAAAGGTGGATCTATGACACATCTAAGCTGGC
TTCTGGAGTGCCATCTAGATTTTCTGGCAGCGGCTCCGGCACTGATTATACATTC
ACCATCAGCAGCCTGCAGCCCGAGGATATCGCCACCTACTACTGTCAGCAGTGGT
CCTCTAATCCCTTCACCTTCGGCCAGGGCACCAAGCTGCAGATCACCAGAACCAG
CGGCGGGGGAGGAAGCGGCGGGGGAGGATCTGGCGGCGGCGGCAGCCAGGTGC
AGCTGGTGCAGAGCGGCGGCGGCGTGGTGCAACCTGGCAGAAGCCTGAGACTGA
GCTGCAAGGCCTCTGGCTACACCTTCACCCGGTACACCATGCATTGGGTGCGGCA
GGCCCCTGGCAAGGGCCTGGAATGGATTGGATACATCAACCCCAGCAGAGGCTA
CACCAACTACAACCAGAAGGTGAAGGACAGATTCACAATTTCTCGGGACAACAG
CAAGAATACCGCCTTCCTGCAAATGGACTCCCTGCGCCCAGAAGATACCGGCGT
GTACTTCTGCGCTAGATATTACGACGACCACTACTGCCTGGACTACTGGGGCCAG
GGCACCCCTGTGACCGTGTCCAGCGCCTCCCACTTCAGCGAGCCTGAGATCACCC
TGATCATCTTCGGCGTGATGGCCGGAGTGATCGGCACAATCCTGCTGATCAGCTA
CGGCATCAGAAGACTGATTAAGAAATCCCCATCTGATGTGAAGCCTCTGCCTTCT
CCTGACACCGACGTCCCCCTGAGCAGCGTGGAAATCGAGAACCCCGAAACCAGC
GACCAG

In some embodiments, the T-cell activation or co-stimulation molecule is selected from the group consisting of an anti-CD3 antibody, a ligand for CD28 (e.g., CD28L), and 41bb ligand (41BBL or CD137L). CD86, also known as B7-2, is a ligand for both CD28 and CTLA-4. In some embodiments, the ligand for CD28 is CD86. CD80 is an additional ligand for CD28. In some embodiments, the ligand for CD28 is CD80. In some embodiments, the ligand for CD28 is an anti-CD28 antibody or an anti-CD28 scFv coupled to a transmembrane domain for display on the surface of the vector. In some embodiments, the co-stimulation molecule is CD80. Viral particles comprising one or more T-cell activation or co-stimulation molecule(s) may be made by engineering the packaging cell line by methods provided by WO 2016/139463; or by expression of the T-cell activation or co-stimulation molecule(s) from a polycistronic helper vector as described in Int'l Pat. Pub. No. WO 2020/106992 A1.

In some embodiments, the viral particle comprises CD19, or a functional fragment thereof, coupled to its native transmembrane domain or a heterologous transmembrane domain. In some embodiments, CD19 acts as a ligand for blinatumomab, thus providing an adapter for coupling the particle to T-cells via the anti-CD3 moiety of blinatumomab. In some embodiments, another type of particle surface ligand can serve to couple an appropriately surface engineered lentiviral particle to a T-cell using a multispecific antibody comprising a binding moiety for the particle surface ligand. In some embodiments, the multispecific antibody is a bispecific antibody, for example, a Bispecific T-cell engager (BiTE).

The non-viral protein may be a cytokine. In some embodiments, the cytokine may be selected from the group consisting of IL-15, IL-7, and IL-2. Where the non-viral protein used is a soluble protein (such as an scFv or a cytokine) it may be tethered to the surface of the lentiviral particle by fusion to a transmembrane domain, such as the transmembrane domain of CD8. Alternatively, it may be indirectly tethered to the lentiviral particle by use of a transmembrane protein engineered to bind the soluble protein. Further inclusion of one or more cytoplasmic residues may increase the stability of the fusion protein.

In some embodiments, the surface-engineered vector comprises a transmembrane protein comprising a mitogenic domain and/or cytokine-based domain. In particular embodiments, the mitogenic domain binds a T cell surface antigen, such as CD3, CD28, CD134 and CD137. In some embodiments, the mitogenic domain binds to a CD3ε chain.

CD28 is one of the proteins expressed on T cells that provide co-stimulatory signals required for T cell activation and survival. T cell stimulation through CD28 in addition to the T-cell receptor (TCR) can provide a potent signal for the production of various interleukins (IL-6 in particular).

CD134, also known as OX40, is a member of the TNFR-superfamily of receptors which is not constitutively expressed on resting naive T cells, unlike CD28. Expression of OX40 is dependent on full activation of the T cell; without CD28, expression of OX40 is delayed and of fourfold lower levels.

CD137, also known as 4-1BB, is a member of the tumor necrosis factor (TNF) receptor family. CD137 can be expressed by activated T cells, but to a larger extent on CD8 than on CD4 T cells. In addition, CD137 expression is found on dendritic cells, follicular dendritic cells, natural killer cells, granulocytes and cells of blood vessel walls at sites of inflammation. The best characterized activity of CD137 is its costimulatory activity for activated T cells. Crosslinking of CD137 enhances T cell proliferation, IL-2 secretion survival and cytolytic activity.

The mitogenic domain may comprise all or part of an antibody or other molecule which specifically binds a T-cell surface antigen. The antibody may activate the TCR or CD28. The antibody may bind the TCR, CD3 or CD28. Examples of such antibodies include: OKT3, 15E8 and TGN1412. Other suitable antibodies include: Anti-CD28: CD28.2, 10F3; Anti-CD3/TCR: UCHT1, YTH12.5, TR66. The mitogenic domain may comprise the binding domain from OKT3, 15E8, TGN1412, CD28.2, 10F3, UCHT1, YTH12.5 or TR66. The mitogenic domain may comprise all or part of a co-stimulatory molecule such as OX40L and 41 BBL. For example, the mitogenic domain may comprise the binding domain from OX40L or 41 BBL.

In some embodiments, the vector comprises an anti-CD3ε antibody, or antigen-binding fragment thereof, coupled to a transmembrane domain. An illustrative anti-CD3ε antibody is OKT3. OKT3, also known as Muromonab-CD3, is a monoclonal antibody targeted at the CD3ε chain.

In some embodiments, the vector comprises a ligand for 4-1BB, or functional fragment thereof, coupled to its native transmembrane domain or a heterologous transmembrane domain. 4-1BBL is a cytokine that belongs to the tumor necrosis factor (TNF) ligand family. This transmembrane cytokine is a bidirectional signal transducer that acts as a ligand for 4-1BB, which is a costimulatory receptor molecule in T lymphocytes. 4-1BBL has been shown to reactivate anergic T lymphocytes in addition to promoting T lymphocyte proliferation.

Transduction Enhancer Spacer Domains

The mitogenic transduction enhancer and/or cytokine-based transduction enhancer may comprise a “spacer sequence” to connect the antigen-binding domain with the transmembrane domain. A flexible spacer allows the antigen-binding domain to orient in different directions to facilitate binding. As used herein, the term “coupled to” refers to a chemical linkage, a direct C-terminal to N-terminal fusion of two protein; chemical linkage to a non-peptide space; chemical linkage to a polypeptide space; and C-terminal to N-terminal fusion of two protein via peptide bonds to a polypeptide spacer, e.g., a spacer sequence.

The spacer sequence may, for example, comprise an lgG1 Fc region, an lgG1 hinge or a human CD8 stalk or the mouse CD8 stalk. The spacer may alternatively comprise an alternative linker sequence which has similar length and/or domain spacing properties as an IgG1 Fc region, an lgG1 hinge or a CD8 stalk. A human lgG1 spacer may be altered to remove Fc binding motifs. In some embodiments, the spacer sequence may be derived from a human protein.

In some embodiments, the spacer sequence comprises a CD8 derived hinge.

In some embodiments, the spacer sequence comprises a ‘short’ hinge. The short hinge is described as hinge region comprising fewer nucleotides relative to CAR hinge regions known in the art.

In some embodiments, the viral particle comprises a polypeptide comprising a CD8 hinge that shares at least 75%, at least 80%, at least 85%, at least 90%, at least 95%, at least 99%, or 100% identity to SEQ ID NO: 3.

CD8 hinge:
(SEQ ID NO: 3)
TTTPAPRPPTPAPTIASQPLSLRPEASRPAAGGAVHTRGLDFASD.

In some embodiments, the viral particle comprises a nucleic acid sequence encoding a CD8 hinge that shares at least 75%, at least 80%, at least 85%, at least 90%, at least 95%, at least 99%, or 100% identity to SEQ ID NO: 8.

CD8 hinge:
(SEQ ID NO: 8)
ACCACAACCCCTGCCCCAAGGCCACCTACACCCGCCCCTACCATCGCCT
CTCAGCCACTGAGCCTGAGGCCAGAGGCATCCAGGCCTGCCGCAGGGGG
GGCCGTGCACACCCGGGGCCTGGACTTTGCCTCTGAT.

In some embodiments, the viral particle comprises a polypeptide comprising a short hinge operably linked to a transmembrane domain operably linked to a cytoplasmic tail derived from the Cocal glycoprotein that shares at least 75%, at least 80%, at least 85%, at least 90%, at least 95%, at least 99%, or 100% identity to SEQ ID NO: 13.

Short hinge-TM-CT from Cocal Env:
(SEQ ID NO: 13)
GVELIEGWFSSWKSTVVTFFFAIGVFILLYVVARIVIAVRYRYQGSNNK
RIYNDIEMSRFRK.

In some embodiments, the viral particle comprises a nucleic acid sequence encoding a short hinge operably linked to a transmembrane domain operably linked to a cytoplasmic tail derived from the Cocal glycoprotein that shares at least 75%, at least 80%, at least 85%, at least 90%, at least 95%, at least 99%, or 100% identity to SEQ ID NO: 16.

Short hinge-TM-CT from Cocal Env:
(SEQ ID NO: 16)
GGAGTGGAACTGATCGAGGGCTGGTTCAGCAGCTGGAAAAGCACCGTGG
TTACATTCTTTTTCGCCATCGGCGTGTTCATCCTGCTGTACGTGGTCGC
CAGAATTGTGATCGCCGTGCGGTATAGATACCAGGGCAGCAACAACAAG
CGGATCTACAACGACATCGAGATGAGCAGATTCAGAAAG.

In some embodiments, the viral particle comprises a polypeptide comprising a long hinge operably linked to a transmembrane domain operably linked to a cytoplasmic tail derived from the Cocal glycoprotein that shares at least 75%, at least 80%, at least 85%, at least 90%, at least 95%, at least 99%, or 100% identity to SEQ ID NO: 19.

Long hinge-TM-CT from Cocal Env:
(SEQ ID NO: 19)
SGFEHPHLAEAPKQLPEEETLFFGDTGISKNPVELIEGWFSSWKSTVVT
FFFAIGVFILLYVVARIVIAVRYRYQGSNNKRIYNDIEMSRFRK.

In some embodiments, the viral particle comprises a nucleic acid sequence encoding a long hinge operably linked to a transmembrane domain operably linked to a cytoplasmic tail derived from the Cocal glycoprotein that shares at least 75%, at least 80%, at least 85%, at least 90%, at least 95%, at least 99%, or 100% identity to SEQ ID NO: 22.

Long hinge-TM-CT from Cocal Env:
(SEQ ID NO: 22)
TCCGGATTCGAGCACCCCCACCTGGCCGAGGCCCCTAAGCAGCTGCCTG
AAGAAGAGACACTGTTTTTCGGAGATACCGGCATCAGCAAAAACCCCGT
GGAGCTGATCGAGGGCTGGTTCAGCTCTTGGAAGAGCACCGTGGTCACA
TTCTTTTTCGCCATCGGCGTCTTTATCCTGCTGTACGTGGTAGCCAGAA
TCGTGATCGCCGTGCGGTACAGATACCAGGGCAGCAACAACAAGCGGAT
CTACAACGACATCGAGATGAGCCGGTTCAGAAAG.

In some embodiments, the viral particle comprises a polypeptide comprising a 218 linker operably linked to a human Glycophorin A ectodomain transmembrane domain operably linked to a cytoplasmic tail derived from a HIV viral envelope that shares at least 75%, at least 80%, at least 85%, at least 90%, at least 95%, at least 99%, or 100% identity to SEQ ID NO: 25.

218 linker human Glycophorin A ecto-TM HIV Env CT:
(SEQ ID NO: 25)
SGGSTSGSGKPGSGEGSTKGPEITLIIFGVMAGVIGTILLISYGIRRLA
LKYWWNLLQYWSQELKNSAVSLLNATAIAVAEGTDRVIEVVQGACRAIR
HIPRRIRQGLERILL.

In some embodiments, the viral particle comprises a nucleic acid sequence encoding a 218 linker operably linked to a human Glycophorin A ectodomain transmembrane domain operably linked to a cytoplasmic tail derived from a HIV viral envelope that shares at least 75%, at least 80%, at least 85%, at least 90%, at least 95%, at least 99%, or 100% identity to SEQ ID NO: 28.

218 linker_human Glycophorin A ecto-TM_HIV Env CT:
(SEQ ID NO: 28)
TCCGGAGGATCTACAAGCGGCTCTGGCAAGCCTGGCAGCGGAGAAGGCA
GCACCAAGGGCCCTGAGATCACACTGATCATCTTCGGCGTGATGGCCGG
CGTCATCGGCACCATCCTGCTGATCAGCTACGGCATCAGAAGACTGGCT
CTGAAGTACTGGTGGAATCTGCTGCAATACTGGAGCCAGGAGCTGAAAA
ACAGCGCCGTGTCCCTGCTCAACGCCACCGCCATCGCCGTGGCCGAGGG
CACCGACAGAGTGATCGAGGTGGTGCAGGGAGCCTGCAGAGCTATTCGG
CACATCCCCAGACGGATCAGGCAGGGCCTGGAAAGAATCCTGCTG.

In some embodiments, the viral particle comprises a polypeptide comprising a 218 linker operably linked to a HIV viral envelope transmembrane domain and cytoplasmic tail that shares at least 75%, at least 80%, at least 85%, at least 90%, at least 95%, at least 99%, or 100% identity to SEQ ID NO: 31.

218 linker_HIV Env-TM-CT:
(SEQ ID NO: 31)
SGGSTSGSGKPGSGEGSTKGNWLWYIRIFIIIVGSLIGLRIVFAVLSLV
NRGWEALKYWWNLLQYWSQELKNSAVSLLNATAIAVAEGTDRVIEVVQG
ACRAIRHIPRRIRQGLERILL.

In some embodiments, the viral particle comprises a nucleic acid sequence encoding a 218 linker operably linked to a HIV viral envelope transmembrane domain and cytoplasmic tail that shares at least 75%, at least 80%, at least 85%, at least 90%, at least 95%, at least 99%, or 100% identity to SEQ ID NO: 34.

218 linker_HIV Env-TM-CT:
(SEQ ID NO: 34)
TCCGGAGGAAGCACCAGCGGCTCTGGCAAGCCTGGCAGCGGCGAGGGCT
CTACCAAGGGCAATTGGCTGTGGTACATCAGAATCTTCATCATCATCGT
GGGCAGCCTGATCGGCCTGAGAATCGTGTTCGCCGTGCTGAGCCTGGTG
AACCGGGGCTGGGAAGCTCTGAAGTACTGGTGGAACCTGCTGCAATACT
GGTCCCAGGAGCTGAAAAACAGCGCTGTGTCCCTGCTCAACGCCACCGC
CATCGCCGTCGCCGAGGGAACAGACAGAGTGATCGAGGTGGTGCAGGGA
GCCTGCAGAGCCATTCGGCACATCCCCAGACGCATCAGACAGGGCCTGG
AAAGAATCCTGCTG.

In some embodiments, the viral particle comprises a polypeptide comprising a triple G4S linker operably linked to a HIV viral envelope transmembrane domain and cytoplasmic tail that shares at least 75%, at least 80%, at least 85%, at least 90%, at least 95%, at least 99%, or 100% identity to SEQ ID NO: 37.

G4Sx3 linker_HIV Env-TM-CT:
(SEQ ID NO: 37)
SGGGGGSGGGGSGGGGSYIRIFIIIVGSLIGLRIVFAVLSLVNRGWEAL
KYWWNLLQYWSQELKNSAVSLLNATAIAVAEGTDRVIEVVQGACRAIRH
IPRRIRQGLERILL

In some embodiments, the viral particle comprises a nucleic acid sequence encoding a triple G4S linker operably linked to a HIV viral envelope transmembrane domain and cytoplasmic tail that shares at least 75%, at least 80%, at least 85%, at least 90%, at least 95%, at least 99%, or 100% identity to SEQ ID NO: 40.

G4Sx3 linker_HIV Env-TM-CT:
(SEQ ID NO: 40)
TCCGGAGGCGGTGGAGGCTCTGGTGGCGGAGGGAGCGGTGGCGGAGGCA
GCTACATCAGAATCTTCATCATCATCGTGGGCAGCCTGATCGGCCTGAG
AATCGTGTTCGCCGTTCTGAGCCTGGTGAACCGGGGCTGGGAAGCCCTG
AAGTACTGGTGGAATCTGCTCCAGTACTGGTCTCAGGAGCTGAAGAACA
GCGCCGTGTCCCTGCTGAACGCTACAGCTATCGCCGTCGCCGAGGGCAC
CGACAGAGTGATCGAGGTGGTGCAGGGCGCCTGCAGAGCCATCCGGCAC
ATCCCTAGAAGGATTCGGCAAGGCCTGGAAAGAATCCTGCTG.

In some embodiments, the viral particle comprises a polypeptide comprising a Ser-Gly peptide operably linked to small ectodomain, transmembrane and cytoplasmic tail sequences derived from human Glycophorin A that shares at least 75%, at least 80%, at least 85%, at least 90%, at least 95%, at least 99%, or 100% identity to SEQ ID NO: 97. The underlined sequence denotes the transmembrane domain fragment.

human Glycophorin A TM_CT:
(SEQ ID NO: 97)
SGHFSEPEITLIIFGVMAGVIGTILLISYGIRRLIKKSPSDVKPLPSPD
TDVPLSSVEIENPETSDQ

In some embodiments, the viral particle comprises a nucleic acid sequence encoding a Ser-Gly peptide operably linked to small ectodomain, transmembrane and cytoplasmic tail sequences derived from human Glycophorin A that shares that shares at least 75%, at least 80%, at least 85%, at least 90%, at least 95%, at least 99%, or 100% identity to SEQ ID NO: 98.

218 linker_human Glycophorin A ecto-TM_HIV Env CT:
(SEQ ID NO: 98)
TCCGGACACTTCAGCGAGCCTGAGATCACCCTGATCATCTTCGGCGTGA
TGGCCGGAGTGATCGGCACAATCCTGCTGATCAGCTACGGCATCAGAAG
ACTGATTAAGAAATCCCCATCTGATGTGAAGCCTCTGCCTTCTCCTGAC
ACCGACGTCCCCCTGAGCAGCGTGGAAATCGAGAACCCCGAAACCAGCG
ACCAG.

In some embodiments, the viral particle comprises a polypeptide comprising transmembrane domain and cytoplasmic tail sequences derived from human Glycophorin A that shares at least 75%, at least 80%, at least 85%, at least 90%, at least 95%, at least 99%, or 100% identity to SEQ ID NO: 105.

human Glycophorin A hinge-TM-CT:
(SEQ ID NO: 105)
HFSEPEITLIIFGVMAGVIGTILLISYGIRRLIKKSPSDVKPLPSPDTD
VPLSSVEIENPETSDQ

In some embodiments, the viral particle comprises a nucleic acid sequence encoding a hinge operably linked to a Glycophorin A transmembrane domain and cytoplasmic tail that shares at least 75%, at least 80%, at least 85%, at least 90%, at least 95%, at least 99%, or 100% identity to SEQ ID NO: 106.

human Glycophorin A hinge-TM-CT
(SEQ ID NO: 106)
CACTTCAGCGAGCCTGAGATCACCCTGATCATCTTCGGCGTGATGGCCG
GAGTGATCGGCACAATCCTGCTGATCAGCTACGGCATCAGAAGACTGAT
TAAGAAATCCCCATCTGATGTGAAGCCTCTGCCTTCTCCTGACACCGAC
GTCCCCCTGAGCAGCGTGGAAATCGAGAACCCCGAAACCAGCGACCAG

In some embodiments, the viral particle comprises a polypeptide comprising a short hinge operably linked to a Cocal glycoprotein transmembrane domain and cytoplasmic tail that shares at least 75%, at least 80%, at least 85%, at least 90%, at least 95%, at least 99%, or 100% identity to SEQ ID NO: 43.

Short hinge-TM-CT from Cocal Env T2A:
(SEQ ID NO: 43)
GVELIEGWFSSWKSTVVTFFFAIGVFILLYVVARIVIAVRYRYQGSNNK
RIYNDIEMSRFRKGSGEGRGSLLTCGDVEENPGP

In some embodiments, the viral particle comprises a nucleic acid sequence encoding a short hinge operably linked to a Cocal glycoprotein transmembrane domain and cytoplasmic tail operably linked to a T2A linker that shares at least 75%, at least 80%, at least 85%, at least 90%, at least 95%, at least 99%, or 100% identity to SEQ ID NO: 47.

Short hinge-TM-CT from Cocal Env T2A:
(SEQ ID NO: 47)
GGAGTGGAACTGATCGAGGGCTGGTTCAGCAGCTGGAAAAGCACCGTGG
TTACATTCTTTTTCGCCATCGGCGTGTTCATCCTGCTGTACGTGGTCGC
CAGAATTGTGATCGCCGTGCGGTATAGATACCAGGGCAGCAACAACAAG
CGGATCTACAACGACATCGAGATGAGCAGATTCAGAAAGGGATCTGGAG
AGGGAAGGGGAAGCCTGCTGACATGCGGCGACGTGGAGGAGAACCCAGG
ACCA.

In some embodiments, the viral particle comprises a polypeptide comprising a CD4 derived transmembrane domain and cytoplasmic tail operably linked to a T2A linker that shares at least 75%, at least 80%, at least 85%, at least 90%, at least 95%, at least 99%, or 100% identity to SEQ ID NO: 4.

CD4 TM and cytoplasmic tail_T2A:
(SEQ ID NO: 4)
MALIVLGGVAGLLLFIGLGIFFCVRCRHRRRQGSGEGRGSLLTCGDVEE
NPGP .

In some embodiments, the viral particle comprises a nucleic acid sequence encoding a CD4 derived transmembrane domain and cytoplasmic tail operably linked to a T2A linker that shares at least 75%, at least 80%, at least 85%, at least 90%, at least 95%, at least 99%, or 100% identity to SEQ ID NO: 9.

CD4 TM and cytoplasmic tail_T2A:
(SEQ ID NO: 9)
ATGGCACTGATCGTGCTGGGAGGAGTGGCAGGACTGCTGCTGTTCATCG
GACTGGGCATCTTCTTTTGCGTGCGCTGTAGGCACCGGAGAAGGCAGGG
ATCTGGAGAGGGAAGGGGAAGCCTGCTGACATGCGGCGACGTGGAGGAG
AACCCAGGACCA.

In some embodiments, the viral particle comprises a polypeptide comprising a Gaussia luciferase derived signal peptide sequence that shares at least 75%, at least 80%, at least 85%, at least 90%, at least 95%, at least 99%, or 100% identity to SEQ ID NO: 11.

Gaussia luciferase SP: MGVKVLFALICIAVAEA (SEQ ID NO: 11).

In some embodiments, the viral particle comprises a nucleic acid sequence encoding a Gaussia luciferase derived signal peptide that shares at least 75%, at least 80%, at least 85%, at least 90%, at least 95%, at least 99%, or 100% identity to SEQ ID NO: 14.

Gaussia luciferase SP:
(SEQ ID NO: 14)
ATGGGCGTGAAAGTGCTGTTCGCCCTGATCTGCATCGCAGTTGCTGAAG
CC.

The transmembrane domain is the sequence of the mitogenic transduction enhancer and/or cytokine-based transduction enhancer that spans the membrane. The transmembrane domain may comprise a hydrophobic alpha helix. The transmembrane domain may be derived from CD28. In some embodiments, the transmembrane domain is derived from a human protein.

The viral particle of the present invention may comprise a cytokine-based transduction enhancer in the viral envelope. In some embodiments, the cytokine-based transduction enhancer is derived from the host cell during viral particle production. In some embodiments, the cytokine-based transduction enhancer is made by the host cell and expressed at the cell surface. When the nascent viral particle buds from the host cell membrane, the cytokine-based transduction enhancer may be incorporated in the viral envelope as part of the packaging cell-derived lipid bilayer.

The cytokine-based transduction enhancer may comprise a cytokine domain and a transmembrane domain. It may have the structure C-S-TM, where C is the cytokine domain, S is an optional spacer domain (e.g., a spacer sequence) and TM is the transmembrane domain. The spacer domain and transmembrane domains are as defined above.

The cytokine domain may comprise a T-cell activating cytokine, such as from IL2, IL7 and IL15, or a functional fragment thereof. As used herein, a “functional fragment” of a cytokine is a fragment of a polypeptide that retains the capacity to bind its particular receptor and activate T-cells.

IL2 is one of the factors secreted by T cells to regulate the growth and differentiation of T cells and certain B cells. IL2 is a lymphokine that induces the proliferation of responsive T cells. It is secreted as a single glycosylated polypeptide, and cleavage of a signal sequence is required for its activity. Solution NMR suggests that the structure of IL2 comprises a bundle of 4 helices (termed A-D), flanked by 2 shorter helices and several poorly defined loops. Residues in helix A, and in the loop region between helices A and B, are important for receptor binding.

Viral Particle Envelope Expression Cassettes

In some embodiments, the viral particles of the present disclosure comprise a viral envelope expression cassette encoding, in 5′ to 3′ order:
(a) a CD8 derived signal peptide
(b) an anti-CD3 scFV
(c) a CD8 derived hinge domain
(d) a CD4 transmembrane domain and cytoplasmic tail
(e) a T2A linker
(f) a Cocal glycoprotein

In some embodiments, the viral particles of the present disclosure comprise a viral envelope expression cassette encoding, in 5′ to 3′ order:
(a) a Gaussia luciferase derived signal peptide
(b) an anti-CD3 scFV
(c) a short hinge domain
(d) a Cocal glycoprotein derived transmembrane domain and cytoplasmic tail

In some embodiments, the viral particles of the present disclosure comprise a viral envelope expression cassette encoding, in 5′ to 3′ order:
(a) a Gaussia luciferase derived signal peptide
(b) an anti-CD3 scFV
(c) a long hinge domain
(d) a Cocal glycoprotein derived transmembrane domain and cytoplasmic tail

In some embodiments, the viral particles of the present disclosure comprise a viral envelope expression cassette encoding, in 5′ to 3′ order:
(a) a Gaussia luciferase derived signal peptide
(b) an anti-CD3 scFV
(c) a 218 linker
(d) a human Glycophorin A ectodomain derived transmembrane domain
(e) a HIV viral envelope derived cytoplasmic tail

In some embodiments, the viral particles of the present disclosure comprise a viral envelope expression cassette encoding, in 5′ to 3′ order:
(a) a Gaussia luciferase derived signal peptide
(b) an anti-CD3 scFV
(c) a 218 linker
(d) a HIV viral envelope derived transmembrane domain and cytoplasmic tail

In some embodiments, the viral particles of the present disclosure comprise a viral envelope expression cassette encoding, in 5′ to 3′ order:
(a) a Gaussia luciferase derived signal peptide
(b) an anti-CD3 scFV
(c) a triple G4S linker
(d) a HIV viral envelope derived transmembrane domain and cytoplasmic tail

In some embodiments, the viral particles of the present disclosure comprise a viral envelope expression cassette encoding, in 5′ to 3′ order:
(a) a Gaussia luciferase derived signal peptide
(b) an anti-CD3 scFV
(c) a short hinge domain
(d) a Cocal glycoprotein derived transmembrane domain and cytoplasmic tail
(e) a T2A linker
(f) a Cocal glycoprotein

Adeno Associated Virus

In some embodiments, the viral particle is an adeno-associated virus (AAV) particle. AAV is a 4.7 kb, single stranded DNA virus. Recombinant particles based on AAV are associated with excellent clinical safety, since wild-type AAV is nonpathogenic and has no etiologic association with any known diseases. In addition, AAV offers the capability for highly efficient gene delivery and sustained transgene expression in numerous tissues. By an “AAV particle” is meant a particle derived from an adeno-associated virus serotype, including without limitation, AAV1, AAV2, AAV3, AAV4, AAV5, AAV6, AAV7, AAV8, AAV9, AAV10, AAVrh.10, AAVrh.74, etc. AAV vectors can have one or more of the AAV wild-type genes deleted in whole or part, e.g., the rep and/or cap genes, but retain functional flanking inverted terminal repeat (ITR) sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g. by the insertion, deletion or substitution of nucleotides, as long as the sequences provide for functional rescue, replication and packaging. AAV particles may comprise other modifications, including but not limited to one or more modified capsid protein (e.g., VP1, VP2 and/or VP3). For example, a capsid protein may be modified to alter tropism and/or reduce immunogenicity.

The serotype of a recombinant AAV particle is determined by its capsid. International Patent Publication No. WO2003042397A2 discloses various capsid sequences including those of AAV1, AAV2, AAV3, AAV8, AAV9, and rh10. International Patent Publication No. WO2013078316A1 discloses the polypeptide sequence of the VP1 from AAVrh74. Numerous diverse naturally occurring or genetically modified AAV capsid sequences are known in the art.

Gene delivery viral particles useful in the practice of the present disclosure can be constructed utilizing methodologies known in the art of molecular biology. Typically, viral vectors carrying transgenes are assembled from polynucleotides encoding the transgene, suitable regulatory elements and elements necessary for production of viral proteins, which mediate cell transduction. Such recombinant viruses may be produced by techniques known in the art, e.g., by transfecting packaging cells or by transient transfection with helper plasmids or viruses. Examples of virus packaging cells include but are not limited to HeLa cells, SF9 cells (optionally with a baculovirus helper vector), 293 cells, etc. Detailed protocols for producing such replication-defective recombinant viruses may be found for instance in WO95/14785, WO96/22378, U.S. Pat. Nos. 5,882,877, 6,013,516, 4,861,719, 5,278,056 and WO94/19478, the complete contents of each of which is hereby incorporated by reference.

Illustrative examples of viral vectors usable in the compositions and methods of the present disclosure are disclosed in WO2016/139463, WO2017/165245, WO2018111834, each of which is incorporated herein in its entirety.

Chimeric Antigen Receptors

In some embodiments, the viral particles described herein are used to transduce a nucleic acid sequence (polynucleotide) encoding one or more chimeric antigen receptor (CARs) into a cell (e.g., a T lymphocyte). In some embodiments, the transduction of the viral particle results in expression of one or more CARs in the transduced cells.

CARs are artificial membrane-bound proteins that direct a T lymphocyte to an antigen and stimulate the T lymphocyte to kill cells displaying the antigen. See, e.g., Eshhar, U.S. Pat. No. 7,741,465. Generally, CARs are genetically engineered receptors comprising an extracellular domain that binds to an antigen, e.g., an antigen on a cell, an optional linker, a transmembrane domain, and an intracellular (cytoplasmic) domain comprising a costimulatory domain and/or a signaling domain that transmits an activation signal to an immune cell. With a CAR, a single receptor can be programmed to both recognize a specific antigen and, when bound to that antigen, activate the immune cell to attack and destroy the cell bearing that antigen. When these antigens exist on tumor cells, an immune cell that expresses the CAR can target and kill the tumor cell. All other conditions being satisfied, when a CAR is expressed on the surface of, e.g., a T lymphocyte, and the extracellular domain of the CAR binds to an antigen, the intracellular signaling domain transmits a signal to the T lymphocyte to activate and/or proliferate, and, if the antigen is present on a cell surface, to kill the cell expressing the antigen. Because T lymphocytes require two signals, a primary activation signal and a costimulatory signal, in order to maximally activate, CARs can comprise a stimulatory and a costimulatory domain such that binding of the antigen to the extracellular domain results in transmission of both a primary activation signal and a costimulatory signal.

In some embodiments, expression of the polycistronic transgene payload is driven by the MND promoter. The MND promoter (myeloproliferative sarcoma virus enhancer, negative control region deleted, dl587rev primer-binding site substituted) is a viral-derived synthetic promoter that contains the U3 region of a modified Moloney murine leukemia virus (MoMuLV) LTR with myeloproliferative sarcoma virus enhancer13 and has high expression in human CD34+ stem cells, lymphocytes, and other tissues. In some embodiments, four separate proteins are expressed, separated by 2A peptide sequences that induce ribosomal skipping and cleavage during translation. In some embodiments, the CAR is a second-generation CAR comprised of the FMC63 mouse anti-human CD19 scFv linked to the 4-1BB costimulatory domain and the CD3zeta intracellular signaling domain.

CAR Intracellular Domain

In some embodiments, the intracellular domain of the CAR is or comprises an intracellular domain or motif of a protein that is expressed on the surface of T lymphocytes and triggers activation and/or proliferation of said T lymphocytes. Such a domain or motif is able to transmit a primary antigen-binding signal that is necessary for the activation of a T lymphocyte in response to the antigen's binding to the CAR's extracellular portion. Typically, this domain or motif comprises, or is, an ITAM (immunoreceptor tyrosine-based activation motif). ITAM-containing polypeptides suitable for CARs include, for example, the zeta CD3 chain (CD3ζ) or ITAM-containing portions thereof. In some embodiments, the intracellular domain is a CD3ζ intracellular signaling domain. In some embodiments, the intracellular domain is from a lymphocyte receptor chain, a TCR/CD3 complex protein, an Fc receptor subunit or an IL-2 receptor subunit. In some embodiments, the intracellular signaling domain of CAR may be derived from the signaling domains of for example OO3ζ, CD3ε, CD22, CD79a, CD66d or CD39. “Intracellular signaling domain,” refers to the part of a CAR polypeptide that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited following antigen binding to the extracellular CAR domain.

In some embodiments, the intracellular domain of the CAR is the zeta CD3 chain (CD3 zeta).

In some embodiments, the viral particle comprises a polypeptide comprising a CAR whose intracellular domain comprises a CD3zeta domain that shares at least 75%, at least 80%, at least 85%, at least 90%, at least 95%, at least 99%, or 100% identity to SEQ ID NO: 54.

CD3zeta:
(SEQ ID NO: 54)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR.

In some embodiments, the viral particle comprises a nucleic acid encoding the intracellular domain of a CAR comprising a CD3zeta domain that shares at least 75%, at least 80%, at least 85%, at least 90%, at least 95%, at least 99%, or 100% identity to SEQ ID NO: 66.

CD3zeta:
(SEQ ID NO: 66)
CGCGTGAAGTTCAGCCGGTCCGCCGATGCCCCTGCCTACCAGCAGGGCC
AGAACCAGCTGTATAACGAGCTGAATCTGGGCCGGAGAGAGGAGTACGA
CGTGCTGGATAAGAGGAGGGGAAGGGACCCAGAGATGGGAGGCAAGCCT
CGGAGAAAGAACCCACAGGAGGGCCTGTACAATGAGCTGCAGAAGGACA
AGATGGCCGAGGCCTATTCTGAGATCGGCATGAAGGGAGAGAGGCGCCG
GGGCAAGGGACACGATGGCCTGTACCAGGGCCTGAGCACCGCCACAAAG
GACACATATGATGCCCTGCACATGCAGGCCCTGCCACCTAGG.

In some embodiments, the CAR additionally comprises one or more co-stimulatory domains or motifs, e.g., as part of the intracellular domain of the polypeptide. Co-stimulatory molecules are well-known cell surface molecules other than antigen receptors or Fc receptors that provide a second signal required for efficient activation and function of T lymphocytes upon binding to antigen. The one or more co-stimulatory domains or motifs can, for example, be, or comprise, one or more of a co-stimulatory CD27 polypeptide sequence, a co-stimulatory CD28 polypeptide sequence, a co-stimulatory OX40 (CD134) polypeptide sequence, a co-stimulatory 4-1BB (CD137) polypeptide sequence, or a co-stimulatory inducible T-cell costimulatory (ICOS) polypeptide sequence, or other costimulatory domain or motif, or any combination thereof. In some embodiments, the one or more co-stimulatory domains are selected from the group consisting of intracellular domains of 4-1BB, CD2, CD7, CD27, CD28, CD30, CD40, CD54 (ICAM), CD83, CD134 (OX40), CD150 (SLAMF1), CD152 (CTLA4), CD223 (LAG3), CD270 (HVEM), CD278 (ICOS), DAP10, LAT, NKD2C SLP76, TRIM, and ZAP70.

In some embodiments, the co-stimulatory domain is the intracellular domains of 4-1BB.

In some embodiments, the viral particle comprises a polypeptide comprising a CAR whose intracellular domain comprises a co-stimulatory 4-1BB polypeptide sequence that shares at least 75%, at least 80%, at least 85%, at least 90%, at least 95%, at least 99%, or 100% identity to SEQ ID NO: 53.

4-1BB signal domain:
(SEQ ID NO: 53)
KRGRKKLLYIFKQPFMRPVQTTQEEDGCSCRFPEEEEGGCEL.

In some embodiments, the viral particle comprises a nucleic acid encoding the intracellular domain of a CAR comprising a co-stimulatory 4-1BB sequence that shares at least 75%, at least 80%, at least 85%, at least 90%, at least 95%, at least 99%, or 100% identity to SEQ ID NO: 65.

4-1BB signal domain:
(SEQ ID NO: 65)
AAGAGAGGCAGGAAGAAGCTGCTGTATATCTTTAAGCAGCCCTTCATGC
GCCCTGTGCAGACCACACAGGAGGAGGACGGCTGCAGCTGTCGGTTTCC
AGAGGAGGAGGAGGGAGGATGCGAGCTG.

In some embodiments, the viral particle comprises a polypeptide comprising a CAR whose intracellular domain comprises an IgG4 linker operatively linked to a CD28 derived transmembrane domain operatively linked to a co-stimulatory 4-1BB polypeptide operatively linked to a CD3zeta domain that shares at least 75%, at least 80%, at least 85%, at least 90%, at least 95%, at least 99%, or 100% identity to SEQ ID NO: 80.

IgG4 linker-CD28 TM 4-1BB-CD3zeta:
(SEQ ID NO: 80)
ESKYGPPCPPCPMFWVLVVVGGVLACYSLLVTVAFIIFWVKRGRKKLLY
IFKQPFMRPVQTTQEEDGCSCRFPEEEEGGCELRVKFSRSADAPAYQQG
QNQLYNELNLGRREEYDVLDKRRGRDPEMGGKPRRKNPQEGLYNELQKD
KMAEAYSEIGMKGERRRGKGHDGLYQGLSTATKDTYDALHMQALPPR.

In some embodiments, the viral particle comprises a nucleic acid encoding the intracellular domain of a CAR comprising an IgG4 linker operatively linked to a CD28 derived transmembrane domain operatively linked to a co-stimulatory 4-1BB polypeptide operatively linked to a CD3zeta domain that shares at least 75%, at least 80%, at least 85%, at least 90%, at least 95%, at least 99%, or 100% identity to SEQ ID NO: 86.

IgG4 linker-CD28 TM_4-1BB-CD3zeta:
(SEQ ID NO: 86)
GAGTCTAAGTATGGCCCTCCATGCCCCCCTTGTCCTATGTTCTGGGTGC
TGGTGGTGGTGGGAGGCGTGCTGGCCTGTTACTCCCTGCTGGTGACCGT
GGCCTTTATCATCTTCTGGGTGAAGCGCGGCCGGAAGAAGCTGCTGTAT
ATCTTTAAGCAGCCCTTCATGAGACCTGTGCAGACCACACAGGAGGAGG
ACGGCTGCAGCTGTAGGTTTCCAGAGGAGGAGGAGGGAGGATGCGAGCT
GCGCGTGAAGTTCTCTCGGAGCGCCGATGCCCCTGCCTACCAGCAGGGA
CAGAACCAGCTGTATAACGAGCTGAATCTGGGCCGGAGAGAGGAGTACG
ACGTGCTGGATAAGAGGAGGGGAAGAGACCCAGAGATGGGAGGCAAGCC
TCGGAGAAAGAACCCACAGGAGGGCCTGTACAATGAGCTGCAGAAGGAC
AAGATGGCCGAGGCCTATTCCGAGATCGGCATGAAGGGAGAGAGGCGCC
GGGGCAAGGGACACGATGGCCTGTACCAGGGCCTGAGCACCGCCACAAA
GGACACCTATGATGCCCTGCACATGCAGGCCCTGCCACCCAGG.

In some embodiments, the viral particle comprises a polypeptide comprising a CAR whose intracellular domain comprises an IgG4 linker operatively linked to a CD28 derived transmembrane domain operatively linked to a co-stimulatory 4-1BB polypeptide operatively linked to a CD3zeta domain that shares at least 75%, at least 80%, at least 85%, at least 90%, at least 95%, at least 99%, or 100% identity to SEQ ID NO: 90.

IgG4 linker-CD28 TM_4-1BB-CD3zeta P2A:
(SEQ ID NO: 90)
ESKYGPPCPPCPMFWVLVVVGGVLACYSLLVTVAFIIFWVKRGRKKLLY
IFKQPFMRPVQTTQEEDGCSCRFPEEEEGGCELRVKFSRSADAPAYQQG
QNQLYNELNLGRREEYDVLDKRRGRDPEMGGKPRRKNPQEGLYNELQKD
KMAEAYSEIGMKGERRRGKGHDGLYQGLSTATKDTYDALHMQALPPRGS
GATNFSLLKQAGDVEENPGP.

In some embodiments, the viral particle comprises a nucleic acid encoding the intracellular domain of a CAR comprising an IgG4 linker operatively linked to a CD28 derived transmembrane domain operatively linked to a co-stimulatory 4-1BB polypeptide operatively linked to a CD3zeta domain that shares at least 75%, at least 80%, at least 85%, at least 90%, at least 95%, at least 99%, or 100% identity to SEQ ID NO: 95.

IgG4 linker-CD28 TM_4-1BB-CD3zeta P2A:
(SEQ ID NO: 95)
GAGTCCAAGTACGGCCCACCCTGCCCTCCATGTCCCATGTTTTGGGTGC
TGGTGGTGGTGGGAGGCGTGCTGGCCTGTTATTCCCTGCTGGTGACCGT
GGCCTTCATCATCTTTTGGGTGAAGCGCGGCCGGAAGAAGCTGCTGTAC
ATCTTCAAGCAGCCCTTCATGAGACCCGTGCAGACCACACAGGAGGAGG
ACGGCTGCAGCTGTAGGTTCCCAGAGGAGGAGGAGGGAGGATGCGAGCT
GAGGGTGAAGTTTTCCCGGTCTGCCGATGCCCCTGCCTATCAGCAGGGC
CAGAATCAGCTGTACAACGAGCTGAATCTGGGCAGGCGCGAGGAGTACG
ACGTGCTGGATAAGAGGAGAGGAAGGGACCCTGAGATGGGAGGCAAGCC
AAGGCGCAAGAACCCTCAGGAGGGCCTGTATAATGAGCTGCAGAAGGAC
AAGATGGCCGAGGCCTACTCCGAGATCGGCATGAAGGGAGAGCGGAGAA
GGGGCAAGGGACACGATGGCCTGTATCAGGGCCTGAGCACCGCCACAAA
GGACACCTACGATGCACTGCACATGCAGGCCCTGCCACCTAGAGGATCT
GGAGCCACAAACTTCAGCCTGCTGAAGCAGGCCGGCGATGTGGAGGAGA
ATCCTGGACCA.

In some embodiments, the intracellular domain can be further modified to encode a detectable, for example, a fluorescent, protein (e.g., green fluorescent protein) or any variants known thereof.

CAR Transmembrane Region

The transmembrane region can be any transmembrane region that can be incorporated into a functional CAR, e.g., a transmembrane region from a CD4 or a CD8 molecule.

In some embodiments, the transmembrane domain of CAR may be derived from the transmembrane domain of CD8, an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1 BB (CD137), 4-1 BBL, GITR, CD40, BAFFR, HVEM (LIGHTR), SLAMF7, NKp80 (KLRFI), CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C. In some embodiments, the transmembrane domain of CAR may be derived from the transmembrane domain of CD28.

CAR Linker Region

The optional linker of CAR positioned between the extracellular domain and the transmembrane domain may be a polypeptide of about 2 to 100 amino acids in length. The linker can include or be composed of flexible residues such as glycine and serine so that the adjacent protein domains are free to move relative to one another. Longer linkers may be used, e.g., when it is desirable to ensure that two adjacent domains do not sterically interfere with one another. Linkers may be cleavable or non-cleavable. Examples of cleavable linkers include 2A linkers (for example T2A), 2A-like linkers or functional equivalents thereof and combinations thereof.

In some embodiments, the linker is P2A self-cleaving peptide. In some embodiments, the viral particle comprises a polypeptide comprising a P2A linker that shares at least 75%, at least 80%, at least 85%, at least 90%, at least 95%, at least 99%, or 100% identity to SEQ ID NO: 55.

P2A self-cleaving peptide:
(SEQ ID NO: 55)
GSGATNFSLLKQAGDVEENPGP.

In some embodiments, the viral particle comprises a nucleic acid encoding a P2A linker that shares at least 75%, at least 80%, at least 85%, at least 90%, at least 95%, at least 99%, or 100% identity to SEQ ID NO: 67.

P2A self-cleaving peptide:
(SEQ ID NO: 67)
GGATCTGGAGCCACCAACTTTAGCCTGCTGAAGCAGGCAGGCGATGTGG
AGGAGAATCCAGGACCT.

In some embodiments, the viral particle comprises a nucleic acid encoding a P2A linker that shares at least 75%, at least 80%, at least 85%, at least 90%, at least 95%, at least 99%, or 100% identity to SEQ ID NO: 69.

P2A self-cleaving peptide:
(SEQ ID NO: 69)
GGCTCCGGCGCCACCAACTTCTCCCTGCTGAAGCAGGCCGGCGATGTGG
AAGAAAATCCAGGACCA.

In some embodiments, the linker is derived from a hinge region or portion of the hinge region of any immunoglobulin. In some embodiments, the linker is derived from IgG4.

In some embodiments, the linker is an IgG4 linker operably linked to a CD28 derived transmembrane domain that shares at least 75%, at least 80%, at least 85%, at least 90%, at least 95%, at least 99%, or 100% identity to SEQ ID NO: 52.

IgG4 linker-CD28 TM:
(SEQ ID NO: 52)
ESKYGPPCPPCPMFWVLVVVGGVLACYSLLVTVAFIIFWV.

In some embodiments, the linker is an IgG4 linker operably linked to a CD28 derived transmembrane domain that shares at least 75%, at least 80%, at least 85%, at least 90%, at least 95%, at least 99%, or 100% identity to SEQ ID NO: 64.

IgG4 linker-CD28 TM:
(SEQ ID NO: 64)
GAGTCTAAGTATGGCCCACCCTGCCCTCCATGTCCAATGTTCTGGGTGC
TGGTGGTGGTGGGAGGCGTGCTGGCCTGTTACTCCCTGCTGGTGACCGT
GGCCTTTATCATCTTCTGGGTG.

CAR Extracellular Domain

In some embodiments, the nucleic acid transduced into cells using the methods described herein comprises a sequence that encodes a polypeptide, wherein the extracellular domain of the polypeptide binds to an antigen of interest. In some embodiments, the extracellular domain comprises a receptor, or a portion of a receptor, that binds to said antigen. In some embodiments, the extracellular domain comprises, or is, an antibody or an antigen-binding portion thereof. In some embodiments, the extracellular domain comprises, or is, a single-chain Fv domain. The single-chain Fv domain can comprise, for example, a VL linked to VH by a flexible linker, wherein said VL and VH are from an antibody that binds said antigen.

In some embodiments, the extracellular domain of CAR may contain any polypeptide that binds the desired antigen (e.g. prostate neoantigen). The extracellular domain may comprise a scFv, a portion of an antibody or an alternative scaffold. CARS may also be engineered to bind two or more desired antigens that may be arranged in tandem and separated by linker sequences. For example, one or more domain antibodies, scFvs, llama VHH antibodies or other VH only antibody fragments may be organized in tandem via a linker to provide bispecificity or multispecificity to the CAR.

The antigen to which the extracellular domain of the polypeptide binds can be any antigen of interest, e.g., can be an antigen on a tumor cell. The tumor cell may be, e.g., a cell in a solid tumor, or a cell of a blood cancer. The antigen can be any antigen that is expressed on a cell of any tumor or cancer type, e.g., cells of a lymphoma, a lung cancer, a breast cancer, a prostate cancer, an adrenocortical carcinoma, a thyroid carcinoma, a nasopharyngeal carcinoma, a melanoma, e.g., a malignant melanoma, a skin carcinoma, a colorectal carcinoma, a desmoid tumor, a desmoplastic small round cell tumor, an endocrine tumor, an Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipoma, or the like. In some embodiments, said lymphoma can be chronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, or a non-Hodgkin lymphoma. In some embodiments, in which the cancer is chronic lymphocytic leukemia (CLL), the B cells of the CLL have a normal karyotype. In some embodiments, in which the cancer is chronic lymphocytic leukemia (CLL), the B cells of the CLL carry a 17p deletion, an 11q deletion, a 12q trisomy, a 13q deletion or a p53 deletion.

In some embodiments, the antigen is expressed on a B-cell malignancy cell, relapsed/refractory CD19-expressing malignancy cell, diffuse large B-cell lymphoma (DLBCL) cell, Burkitt's type large B-cell lymphoma (B-LBL) cell, follicular lymphoma (FL) cell, chronic lymphocytic leukemia (CLL) cell, acute lymphocytic leukemia (ALL) cell, mantle cell lymphoma (MCL) cell, hematological malignancy cell, colon cancer cell, lung cancer cell, liver cancer cell, breast cancer cell, renal cancer cell, prostate cancer cell, ovarian cancer cell, skin cancer cell, melanoma cell, bone cancer cell, brain cancer cell, squamous cell carcinoma cell, leukemia cell, myeloma cell, B cell lymphoma cell, kidney cancer cell, uterine cancer cell, adenocarcinoma cell, pancreatic cancer cell, chronic myelogenous leukemia cell, glioblastoma cell, neuroblastoma cell, medulloblastoma cell, or a sarcoma cell.

In some embodiments, the antigen is a tumor-associated antigen (TAA) or a tumor-specific antigen (TSA). In some embodiments, without limitation, the tumor-associated antigen or tumor-specific antigen is B cell maturation antigen (BCMA), B cell Activating Factor (BAFF), Her2, prostate stem cell antigen (PSCA), prostate-specific membrane antigen (PSMA) alpha-fetoprotein (AFP), carcinoembryonic antigen (CEA), EGFRvIII, cancer antigen-125 (CA-125), CA19-9, calretinin, MUC-1, epithelial membrane protein (EMA), epithelial tumor antigen (ETA), tyrosinase, melanoma-associated antigen (MAGE), CD19, CD20, CD34, CD45, CD99, CD117, chromogranin, cytokeratin, desmin, glial fibrillary acidic protein (GFAP), gross cystic disease fluid protein (GCDFP-15), HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysis, thyroglobulin, thyroid transcription factor-1, vascular endothelial growth factor receptor (VEGFR), the dimeric form of the pyruvate kinase isoenzyme type M2 (tumor M2-PK), an abnormal ras protein, or an abnormal p53 protein.

In some embodiments, the antigen is CD19.

In some embodiments, a CAR comprises an extracellular domain comprising a FMC63 scFv binding domain for CD19 binding.

In some embodiments, the viral particle comprises a polypeptide comprising a CAR whose extracellular domain comprises a signal peptide that shares at least 75%, at least 80%, at least 85%, at least 90%, at least 95%, at least 99%, or 100% identity to SEQ ID NO: 50.

Signal peptide (Human CSF2R): MLLLVTSLLLCELPHPAFLLIP (SEQ ID NO: 50).

In some embodiments, the viral particle comprises a polynucleotide encoding a CAR whose extracellular domain comprises a αCD19 scFv (CD19 VL linked to a CD19 VH) that shares at least 75%, at least 80%, at least 85%, at least 90%, at least 95%, at least 99%, or 100% identity to SEQ ID NO: 51.

CD19 VL_linker VH:
(SEQ ID NO: 51)
DIQMTQTTSSLSASLGDRVTISCRASQDISKYLNWYQQKPDGTVKLLIY
HTSRLHSGVPSRFSGSGSGTDYSLTISNLEQEDIATYFCQQGNTLPYTF
GGGTKLEITGSTSGSGKPGSGEGSTKGEVKLQESGPGLVAPSQSLSVTC
TVSGVSLPDYGVSWIRQPPRKGLEWLGVIWGSETTYYNSALKSRLTIIK
DNSKSQVFLKMNSLQTDDTAIYYCAKHYYYGGSYAMDYWGQGTSVTVS
S.

The complementary determining regions (CDR) of this scFv are RASQDISKYLN, (CDR-L1; SEQ ID NO: 138), HTSRLHS (CDR-L2; SEQ ID NO: 139), QQGNTLPYT (CDR-L3; SEQ ID NO: 140), DYGV (CDR-H1; SEQ ID NO: 141), VIWGSETTYYNSALKS (CDR-H2; SEQ ID NO: 142), HYYYGGSYAMDY (CDR-H3; SEQ ID NO: 143). In some embodiments, the viral particle comprises a polynucleotide encoding a CAR whose extracellular domain comprises a αCD19 scFv having these CDRs, wherein optionally the αCD19 scFv shares at least 75%, at least 80%, at least 85%, at least 90%, at least 95%, at least 99%, or 100% identity to SEQ ID NO: 51.

In some embodiments, the viral particle comprises a polynucleotide encoding a CAR whose extracellular domain comprises a αCD19 scFv having these CDRs, wherein optionally the αCD19 scFv shares at least 75%, at least 80%, at least 85%, at least 90%, at least 95%, at least 99%, or 100% identity to SEQ ID NO: 51 or 89.

In some embodiments, the viral particle comprises a nucleic acid encoding a signal peptide for the extracellular domain of CAR that shares at least 75%, at least 80%, at least 85%, at least 90%, at least 95%, at least 99%, or 100% identity to SEQ ID NO: 62.

Signal peptide (Human CSF2R):
(SEQ ID NO: 62)
ATGCTGCTGCTGGTGACCTCCCTGCTGCTGTGCGAGCTGCCTCACCCAG
CCTTTCTGCTGATCCCC.

In some embodiments, the viral particle comprises a nucleic acid encoding the extracellular domain of a CAR comprising a αCD19 scFv that shares at least 75%, at least 80%, at least 85%, at least 90%, at least 95%, at least 99%, or 100% identity to SEQ ID NO:

63.

αCD19 scFv (VL_linker_VH):
(SEQ ID NO: 63)
GACATCCAGATGACACAGACCACAAGCTCCCTGTCTGCCAGCCTGGGCG
ACAGAGTGACCATCTCCTGTAGGGCCTCTCAGGATATCAGCAAGTACCT
GAACTGGTATCAGCAGAAGCCAGATGGCACAGTGAAGCTGCTGATCTAC
CACACCTCCAGGCTGCACTCTGGAGTGCCAAGCCGGTTCTCCGGATCTG
GAAGCGGCACCGACTATTCCCTGACAATCTCTAACCTGGAGCAGGAGGA
TATCGCCACATACTTTTGCCAGCAGGGCAATACCCTGCCATATACATTC
GGCGGAGGAACCAAGCTGGAGATCACCGGATCCACATCTGGAAGCGGCA
AGCCAGGAAGCGGAGAGGGATCCACAAAGGGAGAGGTGAAGCTGCAGGA
GAGCGGACCAGGACTGGTGGCACCATCCCAGTCTCTGAGCGTGACCTGT
ACAGTGTCCGGCGTGTCTCTGCCTGACTACGGCGTGTCCTGGATCAGGC
AGCCACCTAGGAAGGGACTGGAGTGGCTGGGCGTGATCTGGGGCTCTGA
GACCACATACTATAATTCTGCCCTGAAGAGCCGCCTGACCATCATCAAG
GACAACTCCAAGTCTCAGGTGTTTCTGAAGATGAATAGCCTGCAGACCG
ACGATACAGCCATCTACTATTGCGCCAAGCACTACTATTACGGCGGCTC
CTACGCCATGGATTATTGGGGCCAGGGCACCTCCGTGACAGTGTCTAG
C.

In some embodiments, the viral particle comprises a polypeptide comprising a CAR whose extracellular domain comprises a αCD19 scFv that shares at least 75%, at least 80%, at least 85%, at least 90%, at least 95%, at least 99%, or 100% identity to SEQ ID NO: 79.

αCD19 scFv:
(SEQ ID NO: 79)
MLLLVTSLLLCELPHPAFLLIPDIQMTQTTSSLSASLGDRVTISCRASQ
DISKYLNWYQQKPDGTVKLLIYHTSRLHSGVPSRFSGSGSGTDYSLTIS
NLEQEDIATYFCQQGNTLPYTFGGGTKLEITGSTSGSGKPGSGEGSTKG
EVKLQESGPGLVAPSQSLSVTCTVSGVSLPDYGVSWIRQPPRKGLEWLG
VIWGSETTYYNSALKSRLTIIKDNSKSQVFLKMNSLQTDDTAIYYCAKH
YYYGGSYAMDYWGQGTSVTVSS.

In some embodiments, the viral particle comprises a nucleic acid encoding the extracellular domain of a CAR comprising a αCD19 scFv that shares at least 75%, at least 80%, at least 85%, at least 90%, at least 95%, at least 99%, or 100% identity to SEQ ID NO:

85.

αCD19 scFv:
(SEQ ID NO: 85)
ATGCTGCTGCTGGTGACCAGCCTGCTGCTGTGCGAGCTGCCACACCCTG
CCTTCCTGCTGATCCCAGATATCCAGATGACACAGACCACATCCTCTCT
GTCCGCCTCTCTGGGCGACAGAGTGACCATCTCTTGTAGGGCCAGCCAG
GATATCTCCAAGTACCTGAACTGGTATCAGCAGAAGCCTGACGGCACAG
TGAAGCTGCTGATCTACCACACCTCTAGGCTGCACAGCGGAGTGCCATC
CCGGTTCAGCGGATCCGGATCTGGAACAGACTATTCTCTGACCATCAGC
AACCTGGAGCAGGAGGATATCGCCACATACTTTTGCCAGCAGGGCAATA
CCCTGCCATATACATTCGGCGGAGGAACCAAGCTGGAGATCACCGGAAG
CACATCCGGATCTGGCAAGCCAGGATCCGGAGAGGGATCTACAAAGGGA
GAGGTGAAGCTGCAGGAGAGCGGACCAGGACTGGTGGCACCCAGCCAGT
CCCTGTCTGTGACCTGTACAGTGTCTGGCGTGAGCCTGCCCGATTACGG
CGTGTCCTGGATCAGACAGCCACCAAGGAAGGGACTGGAGTGGCTGGGC
GTGATCTGGGGCTCTGAGACCACATACTATAATAGCGCCCTGAAGTCCC
GGCTGACCATCATCAAGGACAACAGCAAGTCCCAGGTGTTTCTGAAGAT
GAATAGCCTGCAGACCGACGATACAGCCATCTACTATTGCGCCAAGCAC
TACTATTACGGCGGCTCCTACGCCATGGATTATTGGGGCCAGGGCACCT
CCGTGACAGTGAGCTCC.

In some embodiments, the viral particle comprises a polynucleotide encoding a CAR whose extracellular domain comprises a αCD19 scFv that shares at least 75%, at least 80%, at least 85%, at least 90%, at least 95%, at least 99%, or 100% identity to SEQ ID NO: 89.

αCD19 scFv:
(SEQ ID NO: 89)
MLLLVTSLLLCELPHPAFLLIPDIQMTQTTSSLSASLGDRVTISCRASQ
DISKYLNWYQQKPDGTVKLLIYHTSRLHSGVPSRFSGSGSGTDYSLTIS
NLEQEDIATYFCQQGNTLPYTFGGGTKLEITGSTSGSGKPGSGEGSTKG
EVKLQESGPGLVAPSQSLSVTCTVSGVSLPDYGVSWIRQPPRKGLEWLG
VIWGSETTYYNSALKSRLTIIKDNSKSQVFLKMNSLQTDDTAIYYCAKH
YYYGGSYAMDYWGQGTSVTVSS.

The complementary determining regions (CDR) of this scFv are RASQDISKYLN, (CDR-L1; SEQ ID NO: 138), HTSRLHS (CDR-L2; SEQ ID NO: 139), QQGNTLPYT (CDR-L3; SEQ ID NO: 140), DYGV (CDR-H1; SEQ ID NO: 141), VIWGSETTYYNSALKS (CDR-H2; SEQ ID NO: 142), HYYYGGSYAMDY (CDR-H3; SEQ ID NO: 143). In some embodiments, the viral particle comprises a polynucleotide encoding a CAR whose extracellular domain comprises a αCD19 scFv having these CDRs, wherein optionally the αCD19 scFv shares at least 75%, at least 80%, at least 85%, at least 90%, at least 95%, at least 99%, or 100% identity to SEQ ID NO: 89.

In some embodiments, the viral particle comprises a nucleic acid encoding the extracellular domain of a CAR comprising a αCD19 scFv that shares at least 75%, at least 80%, at least 85%, at least 90%, at least 95%, at least 99%, or 100% identity to SEQ ID NO: 94.

αCD19 scFv:
(SEQ ID NO: 94)
ATGCTGCTGCTGGTGACATCCCTGCTGCTGTGCGAGCTGCCACACCCAG
CCTTCCTGCTGATCCCCGATATCCAGATGACCCAGACCACAAGCTCCCT
GAGCGCCTCCCTGGGCGACAGGGTGACAATCTCTTGTCGGGCCAGCCAG
GATATCTCCAAGTATCTGAATTGGTACCAGCAGAAGCCCGACGGCACCG
TGAAGCTGCTGATCTATCACACATCTAGACTGCACAGCGGCGTGCCTTC
CAGGTTTTCTGGCAGCGGCTCCGGCACCGACTACTCTCTGACAATCAGC
AACCTGGAGCAGGAGGATATCGCCACCTATTTCTGCCAGCAGGGCAATA
CCCTGCCTTACACATTTGGCGGCGGCACAAAGCTGGAGATCACCGGCTC
TACAAGCGGATCCGGCAAGCCAGGATCCGGAGAGGGATCTACCAAGGGA
GAGGTGAAGCTGCAGGAGAGCGGACCTGGACTGGTGGCACCATCTCAGA
GCCTGTCCGTGACCTGTACAGTGTCTGGCGTGAGCCTGCCAGATTATGG
CGTGAGCTGGATCAGGCAGCCACCTAGGAAGGGACTGGAGTGGCTGGGC
GTGATCTGGGGCTCCGAGACCACATACTATAACAGCGCCCTGAAGTCCC
GCCTGACCATCATCAAGGACAACTCTAAGAGCCAGGTGTTCCTGAAGAT
GAATTCCCTGCAGACCGACGATACAGCCATCTACTATTGCGCCAAGCAC
TACTATTACGGCGGCTCTTATGCCATGGATTACTGGGGCCAGGGCACCA
GCGTGACAGTGTCTAGC.

In some embodiments, the TAA or TSA is a cancer/testis (CT) antigen, e.g., BAGE, CAGE, CTAGE, FATE, GAGE, HCA661, HOM-TES-85, MAGEA, MAGEB, MAGEC, NA88, NY-ESO-1, NY-SAR-35, OY-TES-1, SPANXB1, SPA17, SSX, SYCP1, or TPTE.

In some embodiments, the TAA or TSA is a carbohydrate or ganglioside, e.g., fuc-GM1, GM2 (oncofetal antigen-immunogenic-1; OFA-I-1); GD2 (OFA-I-2), GM3, GD3, and the like.

In some embodiments, the TAA or TSA is alpha-actinin-4, Bage-1, BCR-ABL, Bcr-Abl fusion protein, beta-catenin, CA 125, CA 15-3 (CA 27.29\BCAA), CA 195, CA 242, CA-50, CAM43, Casp-8, cdc27, cdk4, cdkn2a, CEA, coa-1, dek-can fusion protein, EBNA, EF2, Epstein Barr virus antigens, ETV6-AML1 fusion protein, HLA-A2, HLA-All, hsp70-2, KIAAO205, Mart2, Mum-1, 2, and 3, neo-PAP, myosin class I, OS-9, pml-RARα fusion protein, PTPRK, K-ras, N-ras, triosephosphate isomerase, Gage 3,4,5,6,7, GnTV, Herv-K-mel, Lage-1, NA-88, NY-Eso-1/Lage-2, SP17, SSX-2, TRP2-Int2, gp100 (Pmel 17), tyrosinase, TRP-1, TRP-2, MAGE-1, MAGE-3, RAGE, GAGE-1, GAGE-2, p15(58), RAGE, SCP-1, Hom/Mel-40, PRAME, p53, H-Ras, HER-2/neu, E2A-PRL, H4-RET, IGH-IGK, MYL-RAR, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, 13-Catenin, Mum-1, p16, TAGE, PSMA, CT7, telomerase, 43-9F, 5T4, 791Tgp72, 13HCG, BCA225, BTAA, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90, TAAL6, TAG72, TLP, TPS, CD19, CD20, CD22, CD27, CD30, CD70, CD123, CD133, B-cell maturation antigen, CSI, GPCR5, GD2 (ganglioside G2), EGFRvIII (epidermal growth factor variant III), sperm protein 17 (Sp17), mesothelin, PAP (prostatic acid phosphatase), prostein, TARP (T cell receptor gamma alternate reading frame protein), Trp-p8, STEAP1 (six-transmembrane epithelial antigen of the prostate 1), an abnormal ras protein, or an abnormal p53 protein. In some embodiments, said tumor-associated antigen or tumor-specific antigen is integrin αvβ3 (CD61), galactin, K-Ras (V-Ki-ras2 Kirsten rat sarcoma viral oncogene), or Ral-B. Other tumor-associated and tumor-specific antigens are known to those in the art.

Antibodies, and scFvs, that bind to TSAs and TAAs include antibodies and scFVs that are known in the art, as are nucleotide sequences that encode them.

In some embodiments, the antigen is an antigen not considered to be a TSA or a TAA, but which is nevertheless associated with tumor cells, or damage caused by a tumor. In some embodiments, for example, the antigen is, e.g., a growth factor, cytokine or interleukin, e.g., a growth factor, cytokine, or interleukin associated with angiogenesis or vasculogenesis. Such growth factors, cytokines, or interleukins can include, e.g., vascular endothelial growth factor (VEGF), basic fibroblast growth factor (bFGF), platelet-derived growth factor (PDGF), hepatocyte growth factor (HGF), insulin-like growth factor (IGF), or interleukin-8 (IL-8). Tumors can also create a hypoxic environment local to the tumor. As such, in some embodiments, the antigen is a hypoxia-associated factor, e.g., HIF-1α, HIF-1β, HIF-2a, HIF-2β, HIF-3α, or HIF-3β. Tumors can also cause localized damage to normal tissue, causing the release of molecules known as damage associated molecular pattern molecules (DAMPs; also known as alarmins). In some embodiments, therefore, the antigen is a DAMP, e.g., a heat shock protein, chromatin-associated protein high mobility group box 1 (HMGB1), S100A8 (MRP8, calgranulin A), S100A9 (MRP14, calgranulin B), serum amyloid A (SAA), or can be a deoxyribonucleic acid, adenosine triphosphate, uric acid, or heparin sulfate.

In some embodiments of the polypeptides described herein, the extracellular domain is joined to said transmembrane domain directly or by a linker, spacer or hinge polypeptide sequence, e.g., a sequence from CD28 or a sequence from CTLA4.

In some embodiments, the extracellular domain that binds the desired antigen may be derived from antibodies or their antigen binding fragments generated using the technologies described herein.

Rapamycin-Activated Cell-Surface Receptor (RACR)

In some embodiments, the viral particle comprises a polynucleotide sequence encoding a multipartite cell-surface receptor. In some embodiments, the multipartite cell-surface receptor is a proliferatory receptor.

In some embodiments, the multipartite cell-surface receptor is a rapamycin-activated cell-surface receptor (RACR).

In some embodiments, the multipartite cell-surface receptor is a chemically inducible cell-surface receptor.

In some embodiments, the multipartite cell-surface receptor comprises a polynucleotide sequence encoding FKBP-rapamycin complex binding domain (FRB domain) or a functional variant thereof. In some embodiments, the multipartite cell-surface receptor further comprises a polynucleotide sequence encoding a FK506 binding protein domain (FKBP) or a functional variant thereof. In some embodiments, the FKBP is FKBP12.

In some embodiments, the viral particle comprises a RACR polypeptide comprising a signal peptide operably linked to FKBP12 that shares at least 75%, at least 80%, at least 85%, at least 90%, at least 95%, at least 99%, or 100% identity to SEQ ID NO: 57.

Signal peptide-FKBP12:
(SEQ ID NO: 57)
MPLGLLWLGLALLGALHAQAGVQVETISPGDGRTFPKRGQTCVVHYTGM
LEDGKKFDSSRDRNKPFKFMLGKQEVIRGWEEGVAQMSVGQRAKLTISP
DYAYGATGHPGIIPPHATLVFDVELLKL

In some embodiments, the viral particle comprises a RACR polypeptide comprising an IL-2R gamma transmembrane domain operably linked to a cytoplasmic domain that shares at least 75%, at least 80%, at least 85%, at least 90%, at least 95%, at least 99%, or 100% identity to SEQ ID NO: 58.

IL-2R gamma TM-Cytoplasmic domain:
(SEQ ID NO: 58)
GEGSNTSKENPFLFALEAVVISVGSMGLIISLLCVYFWLERTMPRIPTL
KNLEDLVTEYHGNFSAWSGVSKGLAESLQPDYSERLCLVSEIPPKGGAL
GEGPGASPCNQHSPYWAPPCYTLKPET.

In some embodiments, the viral particle comprises a RACR polypeptide comprising a P2A self-cleaving peptide that shares at least 75%, at least 80%, at least 85%, at least 90%, at least 95%, at least 99%, or 100% identity to SEQ ID NO: 55.

P2A:
(SEQ ID NO: 55)
GSGATNFSLLKQAGDVEENPGP.

In some embodiments, the viral particle comprises a RACR polypeptide comprising a signal peptide operably linked to FRB that shares at least 75%, at least 80%, at least 85%, at least 90%, at least 95%, at least 99%, or 100% identity to SEQ ID NO: 59.

Signal peptide-FRB:
(SEQ ID NO: 59)
MALPVTALLLPLALLLHAARPILWHEMWHEGLEEASRLYFGERNVKGMF
EVLEPLHAMMERGPQTLKETSFNQAYGRDLMEAQEWCRKYMKSGNVKDL
LQAWDLYYHVFRRISK.

In some embodiments, the viral particle comprises a RACR polypeptide comprising an IL-2R beta transmembrane domain operably linked to a cytoplasmic domain that shares at least 75%, at least 80%, at least 85%, at least 90%, at least 95%, at least 99%, or 100% identity to SEQ ID NO: 60.

IL-2R beta TM-Cytoplasmic domain:
(SEQ ID NO: 60)
GKDTIPWLGHLLVGLSGAFGFIILVYLLINCRNTGPWLKK
VLKCNTPDPSKFFSQLSSEHGGDVQKWLSSPFPSSSFSPG
GLAPEISPLEVLERDKVTQLLLQQDKVPEPASLSSNHSLT
SCFTNQGYFFFHLPDALEIEACQVYFTYDPYSEEDPDEGV
AGAPTGSSPQPLQPLSGEDDAYCTFPSRDDLLLFSPSLLG
GPSPPSTAPGGSGAGEERMPPSLQERVPRDWDPQPLGPPT
PGVPDLVDFQPPPELVLREAGEEVPDAGPREGVSFPWSRP
PGQGEFRALNARLPLNTDAYLSLQELQGQDPTHLV.

In some embodiments, the viral particle comprises a nucleic acid encoding a signal peptide operably linked to FKBP12 that shares at least 75%, at least 80%, at least 85%, at least 90%, at least 95%, at least 99%, or 100% identity to SEQ ID NO: 70.

Signal peptide-FKBP12:
(SEQ ID NO: 70)
ATGCCTCTGGGACTGCTGTGGCTGGGACTGGCCCTGCTGG
GCGCCCTGCACGCCCAGGCCGGCGTGCAGGTGGAGACAAT
CAGCCCTGGCGACGGCAGAACCTTTCCAAAGAGGGGCCAG
ACATGCGTGGTGCACTACACCGGCATGCTGGAGGATGGCA
AGAAGTTCGACTCCTCTCGCGATCGGAACAAGCCCTTTAA
GTTCATGCTGGGCAAGCAGGAAGTGATCAGAGGCTGGGAG
GAGGGCGTGGCCCAGATGTCTGTGGGCCAGAGGGCCAAGC
TGACAATCAGCCCAGACTATGCATACGGAGCAACCGGACA
CCCTGGAATCATCCCACCACACGCCACACTGGTGTTCGAT
GTGGAGCTGCTGAAGCTG.

In some embodiments, the viral particle comprises a nucleic acid encoding an IL-2R gamma transmembrane domain operably linked to a cytoplasmic domain that shares at least 75%, at least 80%, at least 85%, at least 90%, at least 95%, at least 99%, or 100% identity to SEQ ID NO: 71.

IL-2R gamma TM-Cytoplasmic domain:
(SEQ ID NO: 71)
GGCGAGGGCTCTAACACCAGCAAGGAGAATCCATTTCTGT
TCGCACTGGAGGCAGTGGTCATCTCCGTGGGCTCTATGGG
CCTGATCATCTCCCTGCTGTGCGTGTACTTTTGGCTGGAG
AGAACAATGCCAAGGATCCCCACCCTGAAGAACCTGGAGG
ACCTGGTGACCGAGTACCACGGCAATTTCAGCGCCTGGTC
CGGCGTGTCTAAGGGACTGGCAGAGTCCCTGCAGCCAGAT
TATTCTGAGCGGCTGTGCCTGGTGAGCGAGATCCCTCCAA
AGGGAGGCGCCCTGGGAGAGGGACCAGGAGCCAGCCCCTG
CAACCAGCACTCCCCTTACTGGGCCCCCCCTTGTTATACC
CTGAAGCCAGAGACA.

In some embodiments, the viral particle comprises a nucleic acid encoding a P2A self-cleaving peptide that shares at least 75%, at least 80%, at least 85%, at least 90%, at least 95%, at least 99%, or 100% identity to SEQ ID NO: 72.

P2A:
(SEQ ID NO: 72)
GGCTCTGGCGCCACCAACTTCAGCCTGCTGAAGCAAGCCG
GCGACGTGGAAGAAAACCCAGGACCA.

In some embodiments, the viral particle comprises a nucleic acid encoding a signal peptide operably linked to FRB that shares at least 75%, at least 80%, at least 85%, at least 90%, at least 95%, at least 99%, or 100% identity to SEQ ID NO: 73.

Signal peptide-FRB:
(SEQ ID NO: 73)
ATGGCACTGCCAGTGACCGCCCTGCTGCTGCCTCTGGCCC
TGCTGCTGCACGCAGCCAGACCCATCCTGTGGCACGAAAT
GTGGCATGAAGGCCTGGAGGAGGCAAGCAGACTGTACTTT
GGCGAGAGAAATGTGAAAGGAATGTTTGAGGTGCTGGAGC
CTCTGCACGCCATGATGGAGAGGGGCCCTCAGACCCTGAA
GGAGACATCCTTTAACCAGGCCTACGGCAGAGACCTGATG
GAGGCCCAGGAGTGGTGCAGGAAGTATATGAAGAGCGGAA
ATGTGAAAGACCTGCTGCAGGCCTGGGATCTGTACTACCA
CGTGTTCCGCCGGATCTCTAAG.

In some embodiments, the viral particle comprises a nucleic acid encoding an IL-2R beta transmembrane domain operably linked to a cytoplasmic domain that shares at least 75%, at least 80%, at least 85%, at least 90%, at least 95%, at least 99%, or 100% identity to SEQ ID NO: 74.

IL-2R beta TM-Cytoplasmic domain:
(SEQ ID NO: 74)
GGCAAGGATACAATCCCTTGGCTGGGACACCTGCTGGTGG
GACTGAGCGGAGCCTTTGGCTTCATCATCCTGGTGTATCT
GCTGATCAACTGCAGAAATACAGGCCCATGGCTGAAGAAG
GTGCTGAAGTGTAACACCCCTGACCCATCCAAGTTCTTTT
CTCAGCTGAGCTCCGAGCACGGCGGCGATGTGCAGAAGTG
GCTGTCTAGCCCCTTTCCTTCCTCTAGCTTCAGCCCTGGA
GGACTGGCACCTGAGATCTCCCCACTGGAGGTGCTGGAGA
GGGACAAGGTGACCCAGCTGCTGCTGCAGCAGGATAAGGT
GCCAGAGCCCGCCTCCCTGTCCTCTAACCACAGCCTGACC
TCCTGCTTTACAAATCAGGGCTACTTCTTTTTCCACCTGC
CAGACGCACTGGAGATCGAGGCATGTCAGGTGTATTTCAC
ATACGATCCCTATAGCGAGGAGGACCCTGATGAGGGAGTG
GCCGGCGCCCCAACCGGAAGCTCCCCTCAGCCACTGCAGC
CACTGAGCGGAGAGGACGATGCATATTGTACATTTCCTTC
CCGCGACGATCTGCTGCTGTTCTCTCCAAGCCTGCTGGGA
GGACCATCTCCACCCAGCACCGCACCTGGAGGATCCGGGG
CAGGGGAGGAGCGGATGCCTCCATCTCTGCAGGAGAGAGT
GCCAAGGGACTGGGATCCACAGCCTCTGGGACCACCTACC
CCTGGAGTGCCAGACCTGGTGGATTTCCAGCCACCCCCTG
AGCTGGTGCTGCGGGAGGCAGGAGAGGAGGTGCCAGACGC
AGGACCTAGAGAGGGCGTGAGCTTTCCCTGGTCCAGGCCA
CCAGGACAGGGAGAGTTCCGCGCCCTGAACGCCCGGCTGC
CCCTGAATACAGACGCCTACCTGTCTCTGCAGGAGCTGCA
GGGCCAGGATCCTACCCACCTGGTG.

In some embodiments, the viral particle comprises a RACR polypeptide comprising a FKBP12 operably linked to an IL-2R gamma domain operably linked to a P2A peptide that shares at least 75%, at least 80%, at least 85%, at least 90%, at least 95%, at least 99%, or 100% identity to SEQ ID NO: 77.

RACRg (FKBP12_IL-2R gamma)_P2A:
(SEQ ID NO: 77)
MPLGLLWLGLALLGALHAQAGVQVETISPGDGRTFPKRGQ
TCVVHYTGMLEDGKKFDSSRDRNKPFKFMLGKQEVIRGWE
EGVAQMSVGQRAKLTISPDYAYGATGHPGIIPPHATLVFD
VELLKLGEGSNTSKENPFLFALEAVVISVGSMGLIISLLC
VYFWLERTMPRIPTLKNLEDLVTEYHGNFSAWSGVSKGLA
ESLQPDYSERLCLVSEIPPKGGALGEGPGASPCNQHSPYW
APPCYTLKPETGSGATNFSLLKQAGDVEENPGP.

In some embodiments, the viral particle comprises a RACR polypeptide comprising a FRB operably linked to an IL-2R beta domain operably linked to a P2A peptide that shares at least 75%, at least 80%, at least 85%, at least 90%, at least 95%, at least 99%, or 100% identity to SEQ ID NO: 78.

RACRb (FRB_IL-2R beta)_P2A:
(SEQ ID NO: 78)
MALPVTALLLPLALLLHAARPILWHEMWHEGLEEASRLYF
GERNVKGMFEVLEPLHAMMERGPQTLKETSFNQAYGRDLM
EAQEWCRKYMKSGNVKDLLQAWDLYYHVFRRISKGKDTIP
WLGHLLVGLSGAFGFIILVYLLINCRNTGPWLKKVLKCNT
PDPSKFFSQLSSEHGGDVQKWLSSPFPSSSFSPGGLAPEI
SPLEVLERDKVTQLLLQQDKVPEPASLSSNHSLTSCFTNQ
GYFFFHLPDALEIEACQVYFTYDPYSEEDPDEGVAGAPTG
SSPQPLQPLSGEDDAYCTFPSRDDLLLFSPSLLGGPSPPS
TAPGGSGAGEERMPPSLQERVPRDWDPQPLGPPTPGVPDL
VDFQPPPELVLREAGEEVPDAGPREGVSFPWSRPPGQGEF
RALNARLPLNTDAYLSLQELQGQDPTHLVGSGATNFSLLK
QAGDVEENPGP.

In some embodiments, the viral particle comprises a nucleic acid encoding a FKBP12 operably linked to an IL-2R gamma domain operably linked to a P2A that shares at least 75%, at least 80%, at least 85%, at least 90%, at least 95%, at least 99%, or 100% identity to SEQ ID NO: 83.

RACRg (FKBP12_IL-2R gamma)_P2A:
(SEQ ID NO: 83)
ATGCCACTGGGACTGCTGTGGCTGGGACTGGCCCTGCTGG
GCGCCCTGCACGCCCAGGCCGGCGTGCAGGTGGAGACAAT
CAGCCCTGGCGACGGACGCACCTTTCCAAAGAGGGGACAG
ACATGCGTGGTGCACTACACCGGCATGCTGGAGGATGGCA
AGAAGTTCGACAGCTCCAGAGATAGGAATAAGCCCTTTAA
GTTCATGCTGGGCAAGCAGGAAGTGATCAGGGGATGGGAG
GAGGGAGTGGCACAGATGTCTGTGGGACAGCGGGCCAAGC
TGACAATCAGCCCAGACTATGCATACGGAGCAACCGGACA
CCCTGGAATCATCCCACCTCACGCCACACTGGTGTTTGAT
GTGGAGCTGCTGAAGCTGGGCGAGGGCAGCAACACCTCCA
AGGAGAATCCATTTCTGTTCGCCCTGGAGGCCGTGGTCAT
CTCTGTGGGCAGCATGGGCCTGATCATCTCCCTGCTGTGC
GTGTACTTTTGGCTGGAGCGCACAATGCCACGGATCCCCA
CCCTGAAGAACCTGGAGGACCTGGTGACCGAGTACCACGG
CAATTTCTCCGCCTGGTCTGGCGTGAGCAAGGGACTGGCA
GAGTCTCTGCAGCCAGATTATAGCGAGCGGCTGTGCCTGG
TGAGCGAGATCCCACCCAAGGGAGGCGCCCTGGGAGAGGG
ACCAGGAGCCTCCCCTTGCAACCAGCACTCTCCTTACTGG
GCCCCTCCATGTTATACCCTGAAGCCAGAGACAGGCAGCG
GAGCTACTAACTTCTCCCTGCTGAAGCAAGCAGGCGACGT
GGAAGAAAATCCTGGACCA.

In some embodiments, the viral particle comprises a nucleic acid encoding a FRB operably linked to an IL-2R beta domain operably linked to a P2A that shares at least 75%, at least 80%, at least 85%, at least 90%, at least 95%, at least 99%, or 100% identity to SEQ ID NO: 84.

RACRb (FRB_IL-2R beta)_P2A:
(SEQ ID NO: 84)
ATGGCACTGCCAGTGACCGCCCTGCTGCTGCCTCTGGCCC
TGCTGCTGCACGCAGCCAGACCCATCCTGTGGCACGAAAT
GTGGCATGAAGGCCTGGAGGAGGCAAGCAGGCTGTACTTT
GGCGAGCGGAATGTGAAAGGAATGTTTGAAGTGCTGGAGC
CTCTGCACGCCATGATGGAGAGGGGCCCTCAGACCCTGAA
GGAGACATCCTTTAACCAGGCCTACGGCAGAGACCTGATG
GAGGCCCAGGAGTGGTGCAGGAAGTATATGAAGTCTGGAA
ATGTGAAAGACCTGCTGCAGGCCTGGGATCTGTATTATCA
CGTGTTCAGGCGCATCTCTAAGGGCAAGGATACAATCCCT
TGGCTGGGACACCTGCTGGTGGGACTGAGCGGAGCCTTTG
GCTTCATCATCCTGGTGTATCTGCTGATCAACTGCCGCAA
TACAGGCCCATGGCTGAAGAAGGTGCTGAAGTGTAACACC
CCCGACCCTTCCAAGTTCTTTTCTCAGCTGTCTAGCGAGC
ACGGCGGCGATGTGCAGAAGTGGCTGTCCTCTCCATTTCC
CAGCTCCTCTTTCAGCCCAGGAGGACTGGCACCAGAGATC
TCCCCACTGGAGGTGCTGGAGAGGGACAAGGTGACCCAGC
TGCTGCTGCAGCAGGATAAGGTGCCTGAGCCAGCCTCCCT
GAGCTCCAACCACTCCCTGACCTCTTGCTTTACAAATCAG
GGCTACTTCTTTTTCCACCTGCCAGACGCACTGGAGATCG
AGGCATGTCAGGTGTATTTCACATACGATCCCTATAGCGA
GGAGGACCCTGATGAGGGAGTGGCCGGCGCCCCAACCGGA
TCTAGCCCACAGCCTCTGCAGCCACTGAGCGGAGAGGACG
ATGCATATTGTACATTTCCTTCCCGCGACGATCTGCTGCT
GTTCTCTCCAAGCCTGCTGGGAGGACCAAGCCCACCTTCC
ACCGCACCAGGCGGCTCCGGGGCAGGGGAGGAGCGGATGC
CACCCTCTCTGCAGGAGAGAGTGCCAAGGGACTGGGATCC
ACAGCCACTGGGACCTCCAACCCCTGGAGTGCCAGACCTG
GTGGATTTCCAGCCCCCTCCAGAGCTGGTGCTGAGAGAGG
CAGGAGAGGAGGTGCCTGACGCAGGACCAAGAGAGGGCGT
GAGCTTTCCTTGGTCCAGGCCACCTGGACAGGGAGAGTTC
AGAGCCCTGAACGCCAGGCTGCCCCTGAATACAGACGCCT
ACCTGTCTCTGCAGGAGCTGCAGGGCCAGGATCCTACACA
CCTGGTCGGATCTGGCGCCACCAACTTTAGCCTGCTGAAG
CAGGCAGGCGACGTGGAAGAGAACCCTGGACCA.

In some embodiments, the FKBP domain and FRB domain form a T cell activator protein complex. The complex formed by the FKBP and FRB domains promote growth and/or survival of a cell. In some embodiments, the complex formed by the FKBP and FRB domains is controlled by a ligand.

In some embodiments, the ligand is rapamycin.

In some embodiments, the FRB domain and FKBP form a tripartite complex with rapamycin that sequesters rapamycin in the transduced cell.

In some embodiments, the ligand is a protein, an antibody, a small molecule, or a drug. In some embodiments, the ligand is rapamycin or a rapamycin analog (rapalogs). In some embodiments, the rapalog comprises variants of rapamycin having one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and alternative substitution on the cyclohexyl ring or replacement of the cyclohexyl ring with a substituted cyclopentyl ring. Thus, in some embodiments, the rapalog is everolimus, novolimus, pimecrolimus, ridaforolimus, tacrolimus, temsirolimus, umirolimus, zotarolimus, CCI-779, C20-methallylrapamycin, C16-(S)-3-methylindolerapamycin, C16-iRap, AP21967, sodium mycophernolic acid, benidipine hydrochloride, rapamine, AP23573, or AP1903, or metabolites, derivatives, and/or combinations thereof. In some embodiments, the ligand is an MID-class drug (e.g., thalidomide, pomalidimide, lenalidomide or related analogues).

In some embodiments, the molecule is selected from FK1012, tacrolimus (FK506), FKCsA, rapamycin, coumermycin, gibberellin, HaXS, TMP-HTag, and ABT-737 or functional derivatives thereof.

In some embodiments, the FKBP domain is operably linked to an IL2R gamma domain. In some embodiments, the FRB domain is operably linked to an IL2R beta domain. In some embodiments, the IL2R gamma domain and IL2R beta domain heterodimerize. In some embodiments, the IL2R gamma domain and IL2R beta domain heterodimerize in the presence of a ligand to promote growth and/or survival of a cell. In some embodiments, the IL2R gamma domain and IL2R beta domain heterodimerize in the presence of rapamycin to promote growth and/or survival of a cell. In some embodiments, the IL2R gamma domain and IL2R beta domain heterodimerize in the presence of rapamycin to promote T cell activation.

Cytosolic FRB

In some embodiments, vector genome comprises a polynucleotide sequence that confers resistance to an immunosuppressive agent.

In some embodiments, the polynucleotide that confers resistance to an immunosuppressive agent binds rapamycin. In some embodiments, the polynucleotide that confers resistance to an immunosuppressive agent encodes a cytosolic (“naked”) FRB domain. The naked FRB domain is an approximately 100 amino acid domain extracted from the mTOR protein kinase. It is expressed in the cytosol as a freely diffusible soluble protein. The purpose of the FRB domain is to reduce the inhibitory effects of rapamycin on mTOR in the transduced cells, which should allow for consistent activation of transduced T cells and give them a proliferative advantage over native T cells.

In some embodiments, the viral particle comprises a polypeptide comprising a cytosolic FRB domain that shares at least 75%, at least 80%, at least 85%, at least 90%, at least 95%, at least 99%, or 100% identity to SEQ ID NO: 56.

Naked FRB:
(SEQ ID NO: 56)
EMWHEGLEEASRLYFGERNVKGMFEVLEPLHAMMERGPQT
LKETSFNQAYGRDLMEAQEWCRKYMKSGNVKDLLQAWDLY
YHVFRRISK.

In some embodiments, the viral particle comprises a nucleic acid encoding a cytosolic FRB domain that shares at least 75%, at least 80%, at least 85%, at least 90%, at least 95%, at least 99%, or 100% identity to SEQ ID NO: 68.

Naked FRB:
(SEQ ID NO: 68)
GAGATGTGGCACGAGGGACTGGAGGAGGCAAGCAGGCTGT
ACTTTGGCGAGCGGAATGTGAAGGGCATGTTCGAGGTGCT
GGAGCCACTGCACGCAATGATGGAGAGGGGACCACAGACC
CTGAAGGAGACATCCTTCAACCAGGCATACGGAAGGGACC
TGATGGAGGCACAGGAGTGGTGCCGGAAGTATATGAAGTC
TGGCAATGTGAAGGACCTGCTGCAGGCCTGGGATCTGTAT
TACCACGTGTTTAGAAGGATCAGCAAG.

In some embodiments, the viral particle comprises a polypeptide comprising a cytosolic FRB domain operably linked to a P2A peptide that shares at least 75%, at least 80%, at least 85%, at least 90%, at least 95%, at least 99%, or 100% identity to SEQ ID NO: 76.

Naked FRB P2A:
(SEQ ID NO: 76)
MEMWHEGLEEASRLYFGERNVKGMFEVLEPLHAMMERGPQ
TLKETSFNQAYGRDLMEAQEWCRKYMKSGNVKDLLQAWDL
YYHVFRRISKGSGATNFSLLKQAGDVEENPGP.

In some embodiments, the viral particle comprises a nucleic acid encoding a cytosolic FRB domain operable linked to a P2A that shares at least 75%, at least 80%, at least 85%, at least 90%, at least 95%, at least 99%, or 100% identity to SEQ ID NO: 82.

Naked FRB P2A:
(SEQ ID NO: 82)
ATGGAGATGTGGCACGAGGGACTGGAGGAGGCAAGCAGAC
TGTACTTTGGCGAGAGGAACGTGAAGGGCATGTTCGAGGT
GCTGGAGCCACTGCACGCAATGATGGAGAGGGGACCACAG
ACCCTGAAGGAGACATCTTTCAACCAGGCATACGGAAGGG
ACCTGATGGAGGCACAGGAGTGGTGCCGGAAGTATATGAA
GAGCGGCAATGTGAAGGACCTGCTGCAGGCCTGGGATCTG
TACTATCACGTGTTTCGGAGAATCTCCAAGGGCTCTGGCG
CCACCAACTTCTCCCTGCTGAAGCAGGCCGGCGATGTGGA
GGAGAATCCTGGACCA.

In some embodiments, the viral particle comprises a polypeptide comprising a cytosolic FRB domain operably linked to a P2A peptide that shares at least 75%, at least 80%, at least 85%, at least 90%, at least 95%, at least 99%, or 100% identity to SEQ ID NO: 88.

Naked FRB P2A:
(SEQ ID NO: 88)
MEMWHEGLEEASRLYFGERNVKGMFEVLEPLHAMMERGPQ
TLKETSFNQAYGRDLMEAQEWCRKYMKSGNVKDLLQAWDL
YYHVFRRISKGSGATNFSLLKQAGDVEENPGP.

In some embodiments, the viral particle comprises a nucleic acid encoding a cytosolic FRB domain operable linked to a P2A that shares at least 75%, at least 80%, at least 85%, at least 90%, at least 95%, at least 99%, or 100% identity to SEQ ID NO: 93.

Naked FRB_P2A:
(SEQ ID NO: 93)
ATGGAGATGTGGCACGAGGGACTGGAGGAGGCATCCAGACTGTAC
TTCGGCGAGAGGAACGTGAAGGGCATGTTTGAGGTGCTGGAGCCA
CTGCACGCCATGATGGAGAGAGGCCCCCAGACCCTGAAGGAGACA
TCTTTCAACCAGGCCTATGGAAGGGACCTGATGGAGGCACAGGAG
TGGTGCCGGAAGTACATGAAGAGCGGCAATGTGAAGGACCTGCTG
CAGGCCTGGGATCTGTACTATCACGTGTTCCGGAGAATCAGCAAG
GGCTCCGGCGCCACCAACTTTAGCCTGCTGAAGCAGGCAGGCGAC
GTGGAGGAGAATCCAGGACCT.

In some embodiments, expression of the chimeric antigen receptor is modulated by a degron fusion polypeptide and wherein suppression of the degron fusion polypeptide is chemically inducible by a ligand.

In some embodiments, expression of the chimeric antigen receptor is modulated by a FRB-degron fusion polypeptide and wherein suppression of the FRB-degron fusion polypeptide is chemically inducible by a ligand.

In some embodiments, the ligand is rapamycin or a rapalog as described herein.

TGF-β Double Negative (TGF-β DN)

Tumor cells secrete transforming growth factor β (TGF-β) as a means to inhibit immunity while allowing for cancer progression. Blocking TGF-β signaling in T cells increases their ability to infiltrate, proliferate, and mediate antitumor responses (Kloss et al., Mol. Therapy 26(7):1855-1866 (2018)). The dominant-negative TGF-β (TGF-β DN) is truncated and lacks the intracellular domain necessary for downstream signaling

In some embodiments, the viral particle of the present disclosure comprises a polynucleotide sequence of a dominant-negative TGF-β. In some embodiments, the viral particle comprises a polypeptide comprising a dominant-negative TGF-β that shares at least 75%, at least 80%, at least 85%, at least 90%, at least 95%, at least 99%, or 100% identity to SEQ ID NO: 91.

TGF beta DN:
(SEQ ID NO: 91)
MGRGLLRGLWPLHIVLWTRIASTIPPHVQKSVNNDMIVTDNNGAV
KFPQLCKFCDVRFSTCDNQKSCMSNCSITSICEKPQEVCVAVWRK
NDENITLETVCHDPKLPYHDFILEDAASPKCIMKEKKKPGETFFM
CSCSSDECNDNIIFSEEYNTSNPDLLLVIFQVTGISLLPPLGVAI
SVIIIFYCYRVNRQQKRRR.

In some embodiments, the viral particle comprises a nucleic acid encoding a dominant-negative TGF-β that shares at least 75%, at least 80%, at least 85%, at least 90%, at least 95%, at least 99%, or 100% identity to SEQ ID NO: 96.

TGF beta DN:
(SEQ ID NO: 96)
ATGGGAAGAGGACTGCTGAGGGGACTGTGGCCACTGCACATCGTG
CTGTGGACCAGGATCGCCTCTACAATCCCACCCCACGTGCAGAAG
AGCGTGAACAATGACATGATCGTGACCGATAACAATGGCGCCGTG
AAGTTTCCCCAGCTGTGCAAGTTCTGTGACGTGCGCTTTTCCACC
TGTGATAACCAGAAGTCCTGCATGTCTAATTGTAGCATCACATCC
ATCTGCGAGAAGCCTCAGGAGGTGTGCGTGGCCGTGTGGCGGAAG
AACGACGAGAATATCACCCTGGAGACAGTGTGCCACGATCCCAAG
CTGCCTTATCACGACTTCATCCTGGAGGATGCCGCCTCTCCTAAG
TGTATCATGAAGGAGAAGAAGAAGCCAGGCGAGACCTTCTTTATG
TGCAGCTGTTCCTCTGACGAGTGCAACGATAATATCATCTTCTCC
GAGGAGTACAACACCTCTAATCCTGACCTGCTGCTGGTCATCTTT
CAGGTGACAGGCATCTCCCTGCTGCCTCCACTGGGCGTGGCCATC
TCTGTGATCATCATCTTTTATTGTTACAGAGTGAACAGGCAGCAG
AAGCGCCGGCGCTAG.

Vector Genome

Payload Plasmids

In some embodiments, the viral particles of the present disclosure comprise a polynucleotide sequence encoding, in 5′ to 3′ order on a polycistronic transcript:
(a) a MND promoter;
(b) a CAR comprising a polypeptide that binds CD19;
(c) a cytosolic FRB domain or a portion thereof;
(d) a RACR cell-surface receptor; and
(e) a WPRE sequence

In some embodiments, the viral particles of the present disclosure comprise a polynucleotide sequence encoding, in 5′ to 3′ order:
(a) a CAR comprising a polypeptide that binds CD19;
(b) a cytosolic FRB domain or a portion thereof; and
(c) a RACR cell-surface receptor.

In some embodiments, the viral particle comprises a nucleic acid sequence that shares at least 75%, at least 80%, at least 85%, at least 90%, at least 95%, at least 99%, or 100% identity to SEQ ID NO: 35.

MND Promoter:
(SEQ ID NO: 35)
GAACAGAGAAACAGGAGAATATGGGCCAAACAGGATATCTGTGGT
AAGCAGTTCCTGCCCCGGCTCAGGGCCAAGAACAGTTGGAACAGC
AGAATATGGGCCAAACAGGATATCTGTGGTAAGCAGTTCCTGCCC
CGGCTCAGGGCCAAGAACAGATGGTCCCCAGATGCGGTCCCGCCC
TCAGCAGTTTCTAGAGAACCATCAGATGTTTCCAGGGTGCCCCAA
GGACCTGAAATGACCCTGTGCCTTATTTGAACTAACCAATCAGTT
CGCTTCTCGCTTCTGTTCGCGCGCTTCTGCTCCCCGAGCTCTATA
TAAGCAGAGCTCGTTTAGTGAACCGTCAGATCGCTAGC.

In some embodiments, the viral particle comprises a polypeptide sequence that shares at least 75%, at least 80%, at least 85%, at least 90%, at least 95%, at least 99%, or 100% identity to SEQ ID NO: 49.

αCD19 CAR_FRB_RACR_lentiviral vector:
(SEQ ID NO: 49)
MLLLVTSLLLCELPHPAFLLIPDIQMTQTTSSLSASLGDRVTISC
RASQDISKYLNWYQQKPDGTVKLLIYHTSRLHSGVPSRFSGSGSG
TDYSLTISNLEQEDIATYFCQQGNTLPYTFGGGTKLEITGSTSGS
GKPGSGEGSTKGEVKLQESGPGLVAPSQSLSVTCTVSGVSLPDYG
VSWIRQPPRKGLEWLGVIWGSETTYYNSALKSRLTIIKDNSKSQV
FLKMNSLQTDDTAIYYCAKHYYYGGSYAMDYWGQGTSVTVSSESK
YGPPCPPCPMFWVLVVVGGVLACYSLLVTVAFIIFWVKRGRKKLL
YIFKQPFMRPVQTTQEEDGCSCRFPEEEEGGCELRVKFSRSADAP
AYQQGQNQLYNELNLGRREEYDVLDKRRGRDPEMGGKPRRKNPQE
GLYNELQKDKMAEAYSEIGMKGERRRGKGHDGLYQGLSTATKDTY
DALHMQALPPRGSGATNFSLLKQAGDVEENPGPEMWHEGLEEASR
LYFGERNVKGMFEVLEPLHAMMERGPQTLKETSFNQAYGRDLMEA
QEWCRKYMKSGNVKDLLQAWDLYYHVFRRISKGSGATNFSLLKQA
GDVEENPGPMPLGLLWLGLALLGALHAQAGVQVETISPGDGRTFP
KRGQTCVVHYTGMLEDGKKFDSSRDRNKPFKFMLGKQEVIRGWEE
GVAQMSVGQRAKLTISPDYAYGATGHPGIIPPHATLVFDVELLKL
GEGSNTSKENPFLFALEAVVISVGSMGLIISLLCVYFWLERTMPR
IPTLKNLEDLVTEYHGNFSAWSGVSKGLAESLQPDYSERLCLVSE
IPPKGGALGEGPGASPCNQHSPYWAPPCYTLKPETGSGATNFSLL
KQAGDVEENPGPMALPVTALLLPLALLLHAARPILWHEMWHEGLE
EASRLYFGERNVKGMFEVLEPLHAMMERGPQTLKETSFNQAYGRD
LMEAQEWCRKYMKSGNVKDLLQAWDLYYHVFRRISKGKDTIPWLG
HLLVGLSGAFGFIILVYLLINCRNTGPWLKKVLKCNTPDPSKFFS
QLSSEHGGDVQKWLSSPFPSSSFSPGGLAPEISPLEVLERDKVTQ
LLLQQDKVPEPASLSSNHSLTSCFTNQGYFFFHLPDALEIEACQV
YFTYDPYSEEDPDEGVAGAPTGSSPQPLQPLSGEDDAYCTFPSRD
DLLLFSPSLLGGPSPPSTAPGGSGAGEERMPPSLQERVPRDWDPQ
PLGPPTPGVPDLVDFQPPPELVLREAGEEVPDAGPREGVSFPWSR
PPGQGEFRALNARLPLNTDAYLSLQELQGQDPTHLV.

In some embodiments, the viral particle comprises a nucleic acid sequence that shares at least 75%, at least 80%, at least 85%, at least 90%, at least 95%, at least 99%, or 100% identity to SEQ ID NO: 61.

αCD19 CAR_FRB_RACR_lentiviral vector:
(SEQ ID NO: 61)
ATGCTGCTGCTGGTGACCTCCCTGCTGCTGTGCGAGCTGCCTCAC
CCAGCCTTTCTGCTGATCCCCGACATCCAGATGACACAGACCACA
AGCTCCCTGTCTGCCAGCCTGGGCGACAGAGTGACCATCTCCTGT
AGGGCCTCTCAGGATATCAGCAAGTACCTGAACTGGTATCAGCAG
AAGCCAGATGGCACAGTGAAGCTGCTGATCTACCACACCTCCAGG
CTGCACTCTGGAGTGCCAAGCCGGTTCTCCGGATCTGGAAGCGGC
ACCGACTATTCCCTGACAATCTCTAACCTGGAGCAGGAGGATATC
GCCACATACTTTTGCCAGCAGGGCAATACCCTGCCATATACATTC
GGCGGAGGAACCAAGCTGGAGATCACCGGATCCACATCTGGAAGC
GGCAAGCCAGGAAGCGGAGAGGGATCCACAAAGGGAGAGGTGAAG
CTGCAGGAGAGCGGACCAGGACTGGTGGCACCATCCCAGTCTCTG
AGCGTGACCTGTACAGTGTCCGGCGTGTCTCTGCCTGACTACGGC
GTGTCCTGGATCAGGCAGCCACCTAGGAAGGGACTGGAGTGGCTG
GGCGTGATCTGGGGCTCTGAGACCACATACTATAATTCTGCCCTG
AAGAGCCGCCTGACCATCATCAAGGACAACTCCAAGTCTCAGGTG
TTTCTGAAGATGAATAGCCTGCAGACCGACGATACAGCCATCTAC
TATTGCGCCAAGCACTACTATTACGGCGGCTCCTACGCCATGGAT
TATTGGGGCCAGGGCACCTCCGTGACAGTGTCTAGCGAGTCTAAG
TATGGCCCACCCTGCCCTCCATGTCCAATGTTCTGGGTGCTGGTG
GTGGTGGGAGGCGTGCTGGCCTGTTACTCCCTGCTGGTGACCGTG
GCCTTTATCATCTTCTGGGTGAAGAGAGGCAGGAAGAAGCTGCTG
TATATCTTTAAGCAGCCCTTCATGCGCCCTGTGCAGACCACACAG
GAGGAGGACGGCTGCAGCTGTCGGTTTCCAGAGGAGGAGGAGGGA
GGATGCGAGCTGCGCGTGAAGTTCAGCCGGTCCGCCGATGCCCCT
GCCTACCAGCAGGGCCAGAACCAGCTGTATAACGAGCTGAATCTG
GGCCGGAGAGAGGAGTACGACGTGCTGGATAAGAGGAGGGGAAGG
GACCCAGAGATGGGAGGCAAGCCTCGGAGAAAGAACCCACAGGAG
GGCCTGTACAATGAGCTGCAGAAGGACAAGATGGCCGAGGCCTAT
TCTGAGATCGGCATGAAGGGAGAGAGGCGCCGGGGCAAGGGACAC
GATGGCCTGTACCAGGGCCTGAGCACCGCCACAAAGGACACATAT
GATGCCCTGCACATGCAGGCCCTGCCACCTAGGGGATCTGGAGCC
ACCAACTTTAGCCTGCTGAAGCAGGCAGGCGATGTGGAGGAGAAT
CCAGGACCTGAGATGTGGCACGAGGGACTGGAGGAGGCAAGCAGG
CTGTACTTTGGCGAGCGGAATGTGAAGGGCATGTTCGAGGTGCTG
GAGCCACTGCACGCAATGATGGAGAGGGGACCACAGACCCTGAAG
GAGACATCCTTCAACCAGGCATACGGAAGGGACCTGATGGAGGCA
CAGGAGTGGTGCCGGAAGTATATGAAGTCTGGCAATGTGAAGGAC
CTGCTGCAGGCCTGGGATCTGTATTACCACGTGTTTAGAAGGATC
AGCAAGGGCTCCGGCGCCACCAACTTCTCCCTGCTGAAGCAGGCC
GGCGATGTGGAAGAAAATCCAGGACCAATGCCTCTGGGACTGCTG
TGGCTGGGACTGGCCCTGCTGGGCGCCCTGCACGCCCAGGCCGGC
GTGCAGGTGGAGACAATCAGCCCTGGCGACGGCAGAACCTTTCCA
AAGAGGGGCCAGACATGCGTGGTGCACTACACCGGCATGCTGGAG
GATGGCAAGAAGTTCGACTCCTCTCGCGATCGGAACAAGCCCTTT
AAGTTCATGCTGGGCAAGCAGGAAGTGATCAGAGGCTGGGAGGAG
GGCGTGGCCCAGATGTCTGTGGGCCAGAGGGCCAAGCTGACAATC
AGCCCAGACTATGCATACGGAGCAACCGGACACCCTGGAATCATC
CCACCACACGCCACACTGGTGTTCGATGTGGAGCTGCTGAAGCTG
GGCGAGGGCTCTAACACCAGCAAGGAGAATCCATTTCTGTTCGCA
CTGGAGGCAGTGGTCATCTCCGTGGGCTCTATGGGCCTGATCATC
TCCCTGCTGTGCGTGTACTTTTGGCTGGAGAGAACAATGCCAAGG
ATCCCCACCCTGAAGAACCTGGAGGACCTGGTGACCGAGTACCAC
GGCAATTTCAGCGCCTGGTCCGGCGTGTCTAAGGGACTGGCAGAG
TCCCTGCAGCCAGATTATTCTGAGCGGCTGTGCCTGGTGAGCGAG
ATCCCTCCAAAGGGAGGCGCCCTGGGAGAGGGACCAGGAGCCAGC
CCCTGCAACCAGCACTCCCCTTACTGGGCCCCCCCTTGTTATACC
CTGAAGCCAGAGACAGGCTCTGGCGCCACCAACTTCAGCCTGCTG
AAGCAAGCCGGCGACGTGGAAGAAAACCCAGGACCAATGGCACTG
CCAGTGACCGCCCTGCTGCTGCCTCTGGCCCTGCTGCTGCACGCA
GCCAGACCCATCCTGTGGCACGAAATGTGGCATGAAGGCCTGGAG
GAGGCAAGCAGACTGTACTTTGGCGAGAGAAATGTGAAAGGAATG
TTTGAGGTGCTGGAGCCTCTGCACGCCATGATGGAGAGGGGCCCT
CAGACCCTGAAGGAGACATCCTTTAACCAGGCCTACGGCAGAGAC
CTGATGGAGGCCCAGGAGTGGTGCAGGAAGTATATGAAGAGCGGA
AATGTGAAAGACCTGCTGCAGGCCTGGGATCTGTACTACCACGTG
TTCCGCCGGATCTCTAAGGGCAAGGATACAATCCCTTGGCTGGGA
CACCTGCTGGTGGGACTGAGCGGAGCCTTTGGCTTCATCATCCTG
GTGTATCTGCTGATCAACTGCAGAAATACAGGCCCATGGCTGAAG
AAGGTGCTGAAGTGTAACACCCCTGACCCATCCAAGTTCTTTTCT
CAGCTGAGCTCCGAGCACGGCGGCGATGTGCAGAAGTGGCTGTCT
AGCCCCTTTCCTTCCTCTAGCTTCAGCCCTGGAGGACTGGCACCT
GAGATCTCCCCACTGGAGGTGCTGGAGAGGGACAAGGTGACCCAG
CTGCTGCTGCAGCAGGATAAGGTGCCAGAGCCCGCCTCCCTGTCC
TCTAACCACAGCCTGACCTCCTGCTTTACAAATCAGGGCTACTTC
TTTTTCCACCTGCCAGACGCACTGGAGATCGAGGCATGTCAGGTG
TATTTCACATACGATCCCTATAGCGAGGAGGACCCTGATGAGGGA
GTGGCCGGCGCCCCAACCGGAAGCTCCCCTCAGCCACTGCAGCCA
CTGAGCGGAGAGGACGATGCATATTGTACATTTCCTTCCCGCGAC
GATCTGCTGCTGTTCTCTCCAAGCCTGCTGGGAGGACCATCTCCA
CCCAGCACCGCACCTGGAGGATCCGGGGCAGGGGAGGAGCGGATG
CCTCCATCTCTGCAGGAGAGAGTGCCAAGGGACTGGGATCCACAG
CCTCTGGGACCACCTACCCCTGGAGTGCCAGACCTGGTGGATTTC
CAGCCACCCCCTGAGCTGGTGCTGCGGGAGGCAGGAGAGGAGGTG
CCAGACGCAGGACCTAGAGAGGGCGTGAGCTTTCCCTGGTCCAGG
CCACCAGGACAGGGAGAGTTCCGCGCCCTGAACGCCCGGCTGCCC
CTGAATACAGACGCCTACCTGTCTCTGCAGGAGCTGCAGGGCCAG
GATCCTACCCACCTGGTGTGA.

In some embodiments, the viral particles of the present disclosure comprises a polynucleotide sequence encoding, in 5′ to 3′ order on a polycistronic transcript:
(a) a MND promoter;
(b) a cytosolic FRB domain or a portion thereof;
(c) a RACR cell-surface receptor;
(d) a CAR comprising a polypeptide that binds CD19; and
(e) a WPRE sequence.

In some embodiments, the viral particles of the present disclosure comprises a polynucleotide sequence encoding, in 5′ to 3′ order:
(a) a cytosolic FRB domain or a portion thereof;
(b) a RACR cell-surface receptor; and
(c) a CAR comprising a polypeptide that binds CD19.

In some embodiments, the viral particle comprises a polypeptide sequence that shares at least 75%, at least 80%, at least 85%, at least 90%, at least 95%, at least 99%, or 100% identity to SEQ ID NO: 75.

FRB_RACR_αCD19 CAR lentiviral vector
(SEQ ID NO: 75)
MEMWHEGLEEASRLYFGERNVKGMFEVLEPLHAMMERGPQTLKET
SFNQAYGRDLMEAQEWCRKYMKSGNVKDLLQAWDLYYHVFRRIS
KGSGATNFSLLKQAGDVEENPGPMPLGLLWLGLALLGALHAQAGV
QVETISPGDGRTFPKRGQTCVVHYTGMLEDGKKFDSSRDRNKPFK
FMLGKQEVIRGWEEGVAQMSVGQRAKLTISPDYAYGATGHPGIIP
PHATLVFDVELLKLGEGSNTSKENPFLFALEAVVISVGSMGLIIS
LLCVYFWLERTMPRIPTLKNLEDLVTEYHGNFSAWSGVSKGLAES
LQPDYSERLCLVSEIPPKGGALGEGPGASPCNQHSPYWAPPCYTL
KPETGSGATNFSLLKQAGDVEENPGPMALPVTALLLPLALLLHAA
RPILWHEMWHEGLEEASRLYFGERNVKGMFEVLEPLHAMMERGPQ
TLKETSFNQAYGRDLMEAQEWCRKYMKSGNVKDLLQAWDLYYHVF
RRISKGKDTIPWLGHLLVGLSGAFGFIILVYLLINCRNTGPWLKK
VLKCNTPDPSKFFSQLSSEHGGDVQKWLSSPFPSSSFSPGGLAPE
ISPLEVLERDKVTQLLLQQDKVPEPASLSSNHSLTSCFTNQGYFF
FHLPDALEIEACQVYFTYDPYSEEDPDEGVAGAPTGSSPQPLQPL
SGEDDAYCTFPSRDDLLLFSPSLLGGPSPPSTAPGGSGAGEERMP
PSLQERVPRDWDPQPLGPPTPGVPDLVDFQPPPELVLREAGEEVP
DAGPREGVSFPWSRPPGQGEFRALNARLPLNTDAYLSLQELQGQD
PTHLVGSGATNFSLLKQAGDVEENPGPMLLLVTSLLLCELPHPAF
LLIPDIQMTQTTSSLSASLGDRVTISCRASQDISKYLNWYQQKPD
GTVKLLIYHTSRLHSGVPSRFSGSGSGTDYSLTISNLEQEDIATY
FCQQGNTLPYTFGGGTKLEITGSTSGSGKPGSGEGSTKGEVKLQE
SGPGLVAPSQSLSVTCTVSGVSLPDYGVSWIRQPPRKGLEWLGVI
WGSETTYYNSALKSRLTIIKDNSKSQVFLKMNSLQTDDTAIYYCA
KHYYYGGSYAMDYWGQGTSVTVSSESKYGPPCPPCPMFWVLVVVG
GVLACYSLLVTVAFIIFWVKRGRKKLLYIFKQPFMRPVQTTQEED
GCSCRFPEEEEGGCELRVKFSRSADAPAYQQGQNQLYNELNLGRR
EEYDVLDKRRGRDPEMGGKPRRKNPQEGLYNELQKDKMAEAYSEI
GMKGERRRGKGHDGLYQGLSTATKDTYDALHMQALPPR

In some embodiments, the viral particle comprises a nucleic acid sequence that shares at least 75%, at least 80%, at least 85%, at least 90%, at least 95%, at least 99%, or 100% identity to SEQ ID NO: 81.

FRB_RACR_αCD19 CAR lentiviral vector
(SEQ ID NO: 81)
ATGGAGATGTGGCACGAGGGACTGGAGGAGGCAAGCAGACTGTAC
TTTGGCGAGAGGAACGTGAAGGGCATGTTCGAGGTGCTGGAGCCA
CTGCACGCAATGATGGAGAGGGGACCACAGACCCTGAAGGAGACA
TCTTTCAACCAGGCATACGGAAGGGACCTGATGGAGGCACAGGAG
TGGTGCCGGAAGTATATGAAGAGCGGCAATGTGAAGGACCTGCTG
CAGGCCTGGGATCTGTACTATCACGTGTTTCGGAGAATCTCCAAG
GGCTCTGGCGCCACCAACTTCTCCCTGCTGAAGCAGGCCGGCGAT
GTGGAGGAGAATCCTGGACCAATGCCACTGGGACTGCTGTGGCTG
GGACTGGCCCTGCTGGGCGCCCTGCACGCCCAGGCCGGCGTGCAG
GTGGAGACAATCAGCCCTGGCGACGGACGCACCTTTCCAAAGAGG
GGACAGACATGCGTGGTGCACTACACCGGCATGCTGGAGGATGGC
AAGAAGTTCGACAGCTCCAGAGATAGGAATAAGCCCTTTAAGTTC
ATGCTGGGCAAGCAGGAAGTGATCAGGGGATGGGAGGAGGGAGTG
GCACAGATGTCTGTGGGACAGCGGGCCAAGCTGACAATCAGCCCA
GACTATGCATACGGAGCAACCGGACACCCTGGAATCATCCCACCT
CACGCCACACTGGTGTTTGATGTGGAGCTGCTGAAGCTGGGCGAG
GGCAGCAACACCTCCAAGGAGAATCCATTTCTGTTCGCCCTGGAG
GCCGTGGTCATCTCTGTGGGCAGCATGGGCCTGATCATCTCCCTG
CTGTGCGTGTACTTTTGGCTGGAGCGCACAATGCCACGGATCCCC
ACCCTGAAGAACCTGGAGGACCTGGTGACCGAGTACCACGGCAAT
TTCTCCGCCTGGTCTGGCGTGAGCAAGGGACTGGCAGAGTCTCTG
CAGCCAGATTATAGCGAGCGGCTGTGCCTGGTGAGCGAGATCCCA
CCCAAGGGAGGCGCCCTGGGAGAGGGACCAGGAGCCTCCCCTTGC
AACCAGCACTCTCCTTACTGGGCCCCTCCATGTTATACCCTGAAG
CCAGAGACAGGCAGCGGAGCTACTAACTTCTCCCTGCTGAAGCAA
GCAGGCGACGTGGAAGAAAATCCTGGACCAATGGCACTGCCAGTG
ACCGCCCTGCTGCTGCCTCTGGCCCTGCTGCTGCACGCAGCCAGA
CCCATCCTGTGGCACGAAATGTGGCATGAAGGCCTGGAGGAGGCA
AGCAGGCTGTACTTTGGCGAGCGGAATGTGAAAGGAATGTTTGAA
GTGCTGGAGCCTCTGCACGCCATGATGGAGAGGGGCCCTCAGACC
CTGAAGGAGACATCCTTTAACCAGGCCTACGGCAGAGACCTGATG
GAGGCCCAGGAGTGGTGCAGGAAGTATATGAAGTCTGGAAATGTG
AAAGACCTGCTGCAGGCCTGGGATCTGTATTATCACGTGTTCAGG
CGCATCTCTAAGGGCAAGGATACAATCCCTTGGCTGGGACACCTG
CTGGTGGGACTGAGCGGAGCCTTTGGCTTCATCATCCTGGTGTAT
CTGCTGATCAACTGCCGCAATACAGGCCCATGGCTGAAGAAGGTG
CTGAAGTGTAACACCCCCGACCCTTCCAAGTTCTTTTCTCAGCTG
TCTAGCGAGCACGGCGGCGATGTGCAGAAGTGGCTGTCCTCTCCA
TTTCCCAGCTCCTCTTTCAGCCCAGGAGGACTGGCACCAGAGATC
TCCCCACTGGAGGTGCTGGAGAGGGACAAGGTGACCCAGCTGCTG
CTGCAGCAGGATAAGGTGCCTGAGCCAGCCTCCCTGAGCTCCAAC
CACTCCCTGACCTCTTGCTTTACAAATCAGGGCTACTTCTTTTTC
CACCTGCCAGACGCACTGGAGATCGAGGCATGTCAGGTGTATTTC
ACATACGATCCCTATAGCGAGGAGGACCCTGATGAGGGAGTGGCC
GGCGCCCCAACCGGATCTAGCCCACAGCCTCTGCAGCCACTGAGC
GGAGAGGACGATGCATATTGTACATTTCCTTCCCGCGACGATCTG
CTGCTGTTCTCTCCAAGCCTGCTGGGAGGACCAAGCCCACCTTCC
ACCGCACCAGGCGGCTCCGGGGCAGGGGAGGAGCGGATGCCACCC
TCTCTGCAGGAGAGAGTGCCAAGGGACTGGGATCCACAGCCACTG
GGACCTCCAACCCCTGGAGTGCCAGACCTGGTGGATTTCCAGCCC
CCTCCAGAGCTGGTGCTGAGAGAGGCAGGAGAGGAGGTGCCTGAC
GCAGGACCAAGAGAGGGCGTGAGCTTTCCTTGGTCCAGGCCACCT
GGACAGGGAGAGTTCAGAGCCCTGAACGCCAGGCTGCCCCTGAAT
ACAGACGCCTACCTGTCTCTGCAGGAGCTGCAGGGCCAGGATCCT
ACACACCTGGTCGGATCTGGCGCCACCAACTTTAGCCTGCTGAAG
CAGGCAGGCGACGTGGAAGAGAACCCTGGACCAATGCTGCTGCTG
GTGACCAGCCTGCTGCTGTGCGAGCTGCCACACCCTGCCTTCCTG
CTGATCCCAGATATCCAGATGACACAGACCACATCCTCTCTGTCC
GCCTCTCTGGGCGACAGAGTGACCATCTCTTGTAGGGCCAGCCAG
GATATCTCCAAGTACCTGAACTGGTATCAGCAGAAGCCTGACGGC
ACAGTGAAGCTGCTGATCTACCACACCTCTAGGCTGCACAGCGGA
GTGCCATCCCGGTTCAGCGGATCCGGATCTGGAACAGACTATTCT
CTGACCATCAGCAACCTGGAGCAGGAGGATATCGCCACATACTTT
TGCCAGCAGGGCAATACCCTGCCATATACATTCGGCGGAGGAACC
AAGCTGGAGATCACCGGAAGCACATCCGGATCTGGCAAGCCAGGA
TCCGGAGAGGGATCTACAAAGGGAGAGGTGAAGCTGCAGGAGAGC
GGACCAGGACTGGTGGCACCCAGCCAGTCCCTGTCTGTGACCTGT
ACAGTGTCTGGCGTGAGCCTGCCCGATTACGGCGTGTCCTGGATC
AGACAGCCACCAAGGAAGGGACTGGAGTGGCTGGGCGTGATCTGG
GGCTCTGAGACCACATACTATAATAGCGCCCTGAAGTCCCGGCTG
ACCATCATCAAGGACAACAGCAAGTCCCAGGTGTTTCTGAAGATG
AATAGCCTGCAGACCGACGATACAGCCATCTACTATTGCGCCAAG
CACTACTATTACGGCGGCTCCTACGCCATGGATTATTGGGGCCAG
GGCACCTCCGTGACAGTGAGCTCCGAGTCTAAGTATGGCCCTCCA
TGCCCCCCTTGTCCTATGTTCTGGGTGCTGGTGGTGGTGGGAGGC
GTGCTGGCCTGTTACTCCCTGCTGGTGACCGTGGCCTTTATCATC
TTCTGGGTGAAGCGCGGCCGGAAGAAGCTGCTGTATATCTTTAAG
CAGCCCTTCATGAGACCTGTGCAGACCACACAGGAGGAGGACGGC
TGCAGCTGTAGGTTTCCAGAGGAGGAGGAGGGAGGATGCGAGCTG
CGCGTGAAGTTCTCTCGGAGCGCCGATGCCCCTGCCTACCAGCAG
GGACAGAACCAGCTGTATAACGAGCTGAATCTGGGCCGGAGAGAG
GAGTACGACGTGCTGGATAAGAGGAGGGGAAGAGACCCAGAGATG
GGAGGCAAGCCTCGGAGAAAGAACCCACAGGAGGGCCTGTACAAT
GAGCTGCAGAAGGACAAGATGGCCGAGGCCTATTCCGAGATCGGC
ATGAAGGGAGAGAGGCGCCGGGGCAAGGGACACGATGGCCTGTAC
CAGGGCCTGAGCACCGCCACAAAGGACACCTATGATGCCCTGCAC
ATGCAGGCCCTGCCACCCAGGTGA.

In some embodiments, the viral particles of the present disclosure comprise a polynucleotide sequence encoding, in 5′ to 3′ order on a polycistronic transcript:
(a) a MND promoter;
(b) a cytosolic FRB domain or a portion thereof;
(c) a CAR comprising a polypeptide that binds CD19,
(d) TGF-β DN domain or portion thereof and
(e) a WPRE sequence.

In some embodiments, the viral particles of the present disclosure comprise a polynucleotide sequence encoding, in 5′ to 3′ order:
(a) a cytosolic FRB domain or a portion thereof;
(b) a CAR comprising a polypeptide that binds CD19, and
(c) a TGF-β DN domain or portion thereof.

In some embodiments, the viral particle comprises a polypeptide sequence that shares at least 75%, at least 80%, at least 85%, at least 90%, at least 95%, at least 99%, or 100% identity to SEQ ID NO: 87.

FRB_αCD19 CAR_TGFbDN lentiviral vector
(SEQ ID NO: 87)
MEMWHEGLEEASRLYFGERNVKGMFEVLEPLHAMMERGPQTLKET
SFNQAYGRDLMEAQEWCRKYMKSGNVKDLLQAWDLYYHVFRRISK
GSGATNFSLLKQAGDVEENPGPMLLLVTSLLLCELPHPAFLLIPD
IQMTQTTSSLSASLGDRVTISCRASQDISKYLNWYQQKPDGTVKL
LIYHTSRLHSGVPSRFSGSGSGTDYSLTISNLEQEDIATYFCQQG
NTLPYTFGGGTKLEITGSTSGSGKPGSGEGSTKGEVKLQESGPGL
VAPSQSLSVTCTVSGVSLPDYGVSWIRQPPRKGLEWLGVIWGSET
TYYNSALKSRLTIIKDNSKSQVFLKMNSLQTDDTAIYYCAKHYYY
GGSYAMDYWGQGTSVTVSSESKYGPPCPPCPMFWVLVVVGGVLAC
YSLLVTVAFIIFWVKRGRKKLLYIFKQPFMRPVQTTQEEDGCSCR
FPEEEEGGCELRVKFSRSADAPAYQQGQNQLYNELNLGRREEYDV
LDKRRGRDPEMGGKPRRKNPQEGLYNELQKDKMAEAYSEIGMKGE
RRRGKGHDGLYQGLSTATKDTYDALHMQALPPRGSGATNFSLLKQ
AGDVEENPGPMGRGLLRGLWPLHIVLWTRIASTIPPHVQKSVNND
MIVTDNNGAVKFPQLCKFCDVRFSTCDNQKSCMSNCSITSICEKP
QEVCVAVWRKNDENITLETVCHDPKLPYHDFILEDAASPKCIMKE
KKKPGETFFMCSCSSDECNDNIIFSEEYNTSNPDLLLVIFQVTGI
SLLPPLGVAISVIIIFYCYRVNRQQKRRR.

In some embodiments, the viral particle comprises a nucleic acid sequence that shares at least 75%, at least 80%, at least 85%, at least 90%, at least 95%, at least 99%, or 100% identity to SEQ ID NO: 92.

FRB_αCD19 CAR TGFbDN lentiviral vector
(SEQ ID NO: 92)
ATGGAGATGTGGCACGAGGGACTGGAGGAGGCATCCAGACTGTAC
TTCGGCGAGAGGAACGTGAAGGGCATGTTTGAGGTGCTGGAGCCA
CTGCACGCCATGATGGAGAGAGGCCCCCAGACCCTGAAGGAGACA
TCTTTCAACCAGGCCTATGGAAGGGACCTGATGGAGGCACAGGAG
TGGTGCCGGAAGTACATGAAGAGCGGCAATGTGAAGGACCTGCTG
CAGGCCTGGGATCTGTACTATCACGTGTTCCGGAGAATCAGCAAG
GGCTCCGGCGCCACCAACTTTAGCCTGCTGAAGCAGGCAGGCGAC
GTGGAGGAGAATCCAGGACCTATGCTGCTGCTGGTGACATCCCTG
CTGCTGTGCGAGCTGCCACACCCAGCCTTCCTGCTGATCCCCGAT
ATCCAGATGACCCAGACCACAAGCTCCCTGAGCGCCTCCCTGGGC
GACAGGGTGACAATCTCTTGTCGGGCCAGCCAGGATATCTCCAAG
TATCTGAATTGGTACCAGCAGAAGCCCGACGGCACCGTGAAGCTG
CTGATCTATCACACATCTAGACTGCACAGCGGCGTGCCTTCCAGG
TTTTCTGGCAGCGGCTCCGGCACCGACTACTCTCTGACAATCAGC
AACCTGGAGCAGGAGGATATCGCCACCTATTTCTGCCAGCAGGGC
AATACCCTGCCTTACACATTTGGCGGCGGCACAAAGCTGGAGATC
ACCGGCTCTACAAGCGGATCCGGCAAGCCAGGATCCGGAGAGGGA
TCTACCAAGGGAGAGGTGAAGCTGCAGGAGAGCGGACCTGGACTG
GTGGCACCATCTCAGAGCCTGTCCGTGACCTGTACAGTGTCTGGC
GTGAGCCTGCCAGATTATGGCGTGAGCTGGATCAGGCAGCCACCT
AGGAAGGGACTGGAGTGGCTGGGCGTGATCTGGGGCTCCGAGACC
ACATACTATAACAGCGCCCTGAAGTCCCGCCTGACCATCATCAAG
GACAACTCTAAGAGCCAGGTGTTCCTGAAGATGAATTCCCTGCAG
ACCGACGATACAGCCATCTACTATTGCGCCAAGCACTACTATTAC
GGCGGCTCTTATGCCATGGATTACTGGGGCCAGGGCACCAGCGTG
ACAGTGTCTAGCGAGTCCAAGTACGGCCCACCCTGCCCTCCATGT
CCCATGTTTTGGGTGCTGGTGGTGGTGGGAGGCGTGCTGGCCTGT
TATTCCCTGCTGGTGACCGTGGCCTTCATCATCTTTTGGGTGAAG
CGCGGCCGGAAGAAGCTGCTGTACATCTTCAAGCAGCCCTTCATG
AGACCCGTGCAGACCACACAGGAGGAGGACGGCTGCAGCTGTAGG
TTCCCAGAGGAGGAGGAGGGAGGATGCGAGCTGAGGGTGAAGTTT
TCCCGGTCTGCCGATGCCCCTGCCTATCAGCAGGGCCAGAATCAG
CTGTACAACGAGCTGAATCTGGGCAGGCGCGAGGAGTACGACGTG
CTGGATAAGAGGAGAGGAAGGGACCCTGAGATGGGAGGCAAGCCA
AGGCGCAAGAACCCTCAGGAGGGCCTGTATAATGAGCTGCAGAAG
GACAAGATGGCCGAGGCCTACTCCGAGATCGGCATGAAGGGAGAG
CGGAGAAGGGGCAAGGGACACGATGGCCTGTATCAGGGCCTGAGC
ACCGCCACAAAGGACACCTACGATGCACTGCACATGCAGGCCCTG
CCACCTAGAGGATCTGGAGCCACAAACTTCAGCCTGCTGAAGCAG
GCCGGCGATGTGGAGGAGAATCCTGGACCAATGGGAAGAGGACTG
CTGAGGGGACTGTGGCCACTGCACATCGTGCTGTGGACCAGGATC
GCCTCTACAATCCCACCCCACGTGCAGAAGAGCGTGAACAATGAC
ATGATCGTGACCGATAACAATGGCGCCGTGAAGTTTCCCCAGCTG
TGCAAGTTCTGTGACGTGCGCTTTTCCACCTGTGATAACCAGAAG
TCCTGCATGTCTAATTGTAGCATCACATCCATCTGCGAGAAGCCT
CAGGAGGTGTGCGTGGCCGTGTGGCGGAAGAACGACGAGAATATC
ACCCTGGAGACAGTGTGCCACGATCCCAAGCTGCCTTATCACGAC
TTCATCCTGGAGGATGCCGCCTCTCCTAAGTGTATCATGAAGGAG
AAGAAGAAGCCAGGCGAGACCTTCTTTATGTGCAGCTGTTCCTCT
GACGAGTGCAACGATAATATCATCTTCTCCGAGGAGTACAACACC
TCTAATCCTGACCTGCTGCTGGTCATCTTTCAGGTGACAGGCATC
TCCCTGCTGCCTCCACTGGGCGTGGCCATCTCTGTGATCATCATC
TTTTATTGTTACAGAGTGAACAGGCAGCAGAAGCGCCGGCGCTAG.

In some embodiments, the viral particles of the present disclosure comprise a polynucleotide sequence encoding, in 5′ to 3′ order:
(a) RSV promoter, (b) 5′ LTR, (c) HIV-1 packaging signal (Psi), (d) Rev response element (RRE) of HIV-1, (e) gp41 peptide, (f) cPPT/CTS, (g) MND promoter, (h) CMV2 extension, (i) Human CSF2R signal peptide, (j) anti-CD19 scFv, (k) IgG4 hinge domain, (l) human CD28 transmembrane domain, (m) 41BB, (n) CD3, (o) P2A, (p) cytosolic FRB domain, (q) P2A, (r) neutrophil gelatinase-associated lipocalin, ER signaling domain, (s) FKBP12, (t) IL2RG, (u) transmembrane domain, (v) cytoplasmic domain, (w) P2A, (x) CD8a signal peptide, (y) Frb (DmrC) [T2098L mutation], (z) IL2RB, (aa) transmembrane domain, (bb) cytoplasmic domain, (cc) WPRE, and (dd) 3′ LTR, and the polynucleotide sequence shares at least 75%, at least 80%, at least 85%, at least 90%, at least 95%, at least 99%, or 100% identity to SEQ ID NO: 121.

In some embodiments, the viral particle comprises a nucleic acid sequence that shares at least 75%, at least 80%, at least 85%, at least 90%, at least 95%, at least 99%, or 100% identity to SEQ ID NO: 121.

In some embodiments, the viral particles of the present disclosure comprise a polynucleotide sequence encoding, in 5′ to 3′ order:
(a) human cytomegalovirus (CMV) immediate early enhancer and CMV promoter, (b) 5′ LTR from HIV-1, (c) HIV-1 packaging signal (Psi), (d) Rev response element (RRE) of HIV-1, (e) central polypurine tract and central termination (cPPT/CTS) sequence of HIV-1, (f) MND promoter (g) Human CSF2R signal peptide, (h) anti-CD19 scFv, (i) IgG4 hinge domain, (j) human CD28 transmembrane domain, (k) human CD28 transmembrane domain, (l) 41BB domain, (m) CD3c, (n) P2A, (o) cytosolic FRB domain, (p) P2A, (q) neutrophil gelatinase-associated lipocalin, ER signaling domain, (r) FKBP12, (s) IL2RG, (t) transmembrane domain, (u) cytoplasmic domain, (v) P2A, (w) CD8a signal peptide, (x) Frb (DmrC) [T2098L mutation], (y) IL2RB, (z) transmembrane domain, (aa) cytoplasmic domain, (bb) 3′ LTR, and (cc) synthetic polyA signal, and the polynucleotide sequence shares at least 75%, at least 80%, at least 85%, at least 90%, at least 95%, at least 99%, or 100% identity to SEQ ID NO: 122.

In some embodiments, the viral particle comprises a nucleic acid sequence that shares at least 75%, at least 80%, at least 85%, at least 90%, at least 95%, at least 99%, or 100% identity to SEQ ID NO: 122.

### Helper Plasmid

In some embodiments, the viral particles of the present disclosure comprise a polynucleotide sequence encoding, in 5′ to 3′ order on a polycistronic transcript:
(a) a gag protein; and
(b) a Pol protein.

In some embodiments, the viral particle comprises a gag protein amino acid sequence that shares at least 75%, at least 80%, at least 85%, at least 90%, at least 95%, at least 99%, or 100% identity to SEQ ID NO: 99.

Gag:
(SEQ ID NO: 99)
MGARASVLSGGELDRWEKIRLRPGGKKKYKLKHIVWASRELERFA
VNPGLLETSEGCRQILGQLQPSLQTGSEELRSLYNTVATLYCVHQ
RIEIKDTKEALDKIEEEQNKSKKKAQQAAADTGHSNQVSQNYPIV
QNIQGQMVHQAISPRTLNAWVKVVEEKAFSPEVIPMFSALSEGAT
PQDLNTMLNTVGGHQAAMQMLKETINEEAAEWDRVHPVHAGPIAP
GQMREPRGSDIAGTTSTLQEQIGWMTHNPPIPVGEIYKRWIILGL
NKIVRMYSPTSILDIRQGPKEPFRDYVDRFYKTLRAEQASQEVKN
WMTETLLVQNANPDCKTILKALGPGATLEEMMTACQGVGGPGHKA
RVLAEAMSQVTNPATIMIQKGNFRNQRKTVKCFNCGKEGHIAKNC
RAPRKKGCWKCGKEGHQMKDCTERQANFLGKIWPSHKGRPGNFLQ
SRPEPTAPPEESFRFGEETTTPSQKQEPIDKELYPLASLRSLFGS
DPSSQ

In some embodiments, the viral particle comprises a Pol protein amino acid sequence that shares at least 75%, at least 80%, at least 85%, at least 90%, at least 95%, at least 99%, or 100% identity to SEQ ID NO: 100.

Pol:
(SEQ ID NO: 100)
FFREDLAFPQGKAREFSSEQTRANSPTRRELQVWGRDNNSLSEAG
ADRQGTVSFSFPQITLWQRPLVTIKIGGQLKEALLDTGADDTVLE
EMNLPGRWKPKMIGGIGGFIKVRQYDQILIEICGHKAIGTVLVGP
TPVNIIGRNLLTQIGCTLNFPISPIETVPVKLKPGMDGPKVKQWP
LTEEKIKALVEICTEMEKEGKISKIGPENPYNTPVFAIKKKDSTK
WRKLVDFRELNKRTQDFWEVQLGIPHPAGLKQKKSVTVLDVGDAY
FSVPLDKDFRKYTAFTIPSINNETPGIRYQYNVLPQGWKGSPAIF
QCSMTKILEPFRKQNPDIVIYQYMDDLYVGSDLEIGQHRTKIEEL
RQHLLRWGFTTPDKKHQKEPPFLWMGYELHPDKWTVQPIVLPEKD
SWTVNDIQKLVGKLNWASQIYAGIKVRQLCKLLRGTKALTEVVPL
TEEAELELAENREILKEPVHGVYYDPSKDLIAEIQKQGQGQWTYQ
IYQEPFKNLKTGKYARMKGAHTNDVKQLTEAVQKIATESIVIWGK
TPKFKLPIQKETWEAWWTEYWQATWIPEWEFVNTPPLVKLWYQLE
KEPIIGAETFYVDGAANRETKLGKAGYVTDRGRQKVVPLTDTTNQ
KTELQAIHLALQDSGLEVNIVTDSQYALGIIQAQPDKSESELVSQ
IIEQLIKKEKVYLAWVPAHKGIGGNEQVDKLVSAGIRKVLFLDGI
DKAQEEHEKYHSNWRAMASDFNLPPVVAKEIVASCDKCQLKGEAM
HGQVDCSPGIWQLDCTHLEGKVILVAVHVASGYIEAEVIPAETGQ
ETAYFLLKLAGRWPVKTVHTDNGSNFTSTTVKAACWWAGIKQEFG
IPYNPQSQGVIESMNKELKKIIGQVRDQAEHLKTAVQMAVFIHNF
KRKGGIGGYSAGERIVDIIATDIQTKELQKQITKIQNFRVYYRDS
RDPVWKGPAKLLWKGEGAVVIQDNSDIKVVPRRKAKIIRDYGKQM
AGDDCVASRQDED

In some embodiments, the viral particle comprises a gag-pol nucleic acid sequence that shares at least 75%, at least 80%, at least 85%, at least 90%, at least 95%, at least 99%, or 100% identity to SEQ ID NO: 101.

In some embodiments, the viral particle comprises a gag-pol nucleic acid sequence that shares at least 75%, at least 80%, at least 85%, at least 90%, at least 95%, at least 99%, or 100% identity to SEQ ID NO: 124.

In some embodiments, the viral particles of the present disclosure comprise a polynucleotide sequence encoding, in 5′ to 3′ order:
(a) human CMV enhancer and CMV promoter, (b) human beta-globin intron, (c) HIV-1 gag, (d) HIV-1 pol, (d) cPPT/CTS, (e) RRE, (f) beta-globin polyA signal, and the polynucleotide sequence shares at least 75%, at least 80%, at least 85%, at least 90%, at least 95%, at least 99%, or 100% identity to SEQ ID NO: 131.

In some embodiments, the viral particle comprises a gag-pol nucleic acid sequence that shares at least 75%, at least 80%, at least 85%, at least 90%, at least 95%, at least 99%, or 100% identity to SEQ ID NO: 131.

In some embodiments, the viral particle comprises a Rev protein amino acid sequence that shares at least 75%, at least 80%, at least 85%, at least 90%, at least 95%, at least 99%, or 100% identity to SEQ ID NO: 102.

Rev protein:
(SEQ ID NO: 102)
MAGRSGDSDEDLLKAVRLIKFLYQSNPPPNPEGTRQARRNRRRRW
RERQRQIHSISERILSTYLGRSAEPVPLQLPPLERLTLDCNEDCG
TSGTQGVGSPQILVESPTILESGAKE*

In some embodiments, the viral particle comprises a Rev nucleic acid sequence that shares at least 75%, at least 80%, at least 85%, at least 90%, at least 95%, at least 99%, or 100% identity to SEQ ID NO: 103.

Rev nucleic acid sequence:
(SEQ ID NO: 103)
ATGGCCGGCAGAAGCGGCGACAGCGACGAGGATCTGCTGAAAGCC
GTGCGGCTGATCAAGTTCCTGTACCAGAGCAACCCTCCTCCTAAC
CCCGAGGGCACCAGACAGGCTAGACGGAACCGCAGAAGAAGGTGG
CGGGAACGGCAAAGACAGATCCACTCTATCAGCGAGAGAATCCTG
AGCACCTACCTGGGAAGATCCGCCGAGCCTGTCCCCCTGCAGCTG
CCTCCACTGGAAAGACTGACCCTGGATTGTAATGAGGACTGCGGC
ACAAGCGGAACCCAGGGCGTGGGCAGCCCCCAGATTCTGGTGGAA
TCCCCTACAATCCTCGAGTCTGGCGCCAAGGAATGA

In some embodiments, the viral particle comprises a Rev nucleic acid sequence that shares at least 75%, at least 80%, at least 85%, at least 90%, at least 95%, at least 99%, or 100% identity to SEQ ID NO: 125.

In some embodiments, the viral particles of the present disclosure comprise a polynucleotide sequence encoding, in 5′ to 3′ order:
(a) RSV promoter, (b) HXB3 Rev, (c) HIV-1 polyA LTR, and the polynucleotide sequence shares at least 75%, at least 80%, at least 85%, at least 90%, at least 95%, at least 99%, or 100% identity to SEQ ID NO: 132.

In some embodiments, the viral particle comprises a gag-pol nucleic acid sequence that shares at least 75%, at least 80%, at least 85%, at least 90%, at least 95%, at least 99%, or 100% identity to SEQ ID NO: 132.

Cocal Envelope Plasmids

In some embodiments, the viral particle comprises a nucleic acid encoding a Cocal envelope, anti-CD3 scFv

In some embodiments, the viral particle comprises a Cocal envelope and anti-CD3 scFv nucleic acid sequence that shares at least 75%, at least 80%, at least 85%, at least 90%, at least 95%, at least 99%, or 100% identity to SEQ ID NO: 128.

In some embodiments, the viral particles of the present disclosure comprise a polynucleotide sequence encoding, in 5′ to 3′ order:
(a) MND promoter;
(b) CD8 derived signal peptide;
(c) anti-CD3 scFv;
(d) CD8 derived hinge;
(e) CD4 derived transmembrane domain and cytoplasmic tail;
(f) T2A;
(g) Cocal envelope;
(h) WPRE; and
(i) polyA signal, and the polynucleotide sequence shares at least 75%, at least 80%, at least 85%, at least 90%, at least 95%, at least 99%, or 100% identity to SEQ ID NO: 128.

In some embodiments, the viral particles of the present disclosure comprise a polynucleotide sequence encoding, in 5′ to 3′ order:
(a) human CMV enhancer and CMV promoter, (b) human beta-globin intron, (c) anti-CD3 scFv, (d) Cocal envelope, (d) transmembrane domain, (e) cytoplasmic tail domain, (f) T2A peptide, (g) BGH polyA signal, and the polynucleotide sequence shares at least 75%, at least 80%, at least 85%, at least 90%, at least 95%, at least 99%, or 100% identity to SEQ ID NO: 129.

In some embodiments, the viral particle comprises a nucleic acid sequence that shares at least 75%, at least 80%, at least 85%, at least 90%, at least 95%, at least 99%, or 100% identity to SEQ ID NO: 129.

In some embodiments, the viral particles of the present disclosure comprise a polynucleotide sequence encoding, in 5′ to 3′ order:
(a) human CMV enhancer and CMV promoter, (b) human beta-globin intron, (c) Cocal envelope, (d) transmembrane domain, (e) cytoplasmic tail domain, (f) bovine growth hormone polyadenylation (BGH polyA) signal, and the polynucleotide sequence shares at least 75%, at least 80%, at least 85%, at least 90%, at least 95%, at least 99%, or 100% identity to SEQ ID NO: 123.

In some embodiments, the viral particle comprises a nucleic acid sequence that shares at least 75%, at least 80%, at least 85%, at least 90%, at least 95%, at least 99%, or 100% identity to SEQ ID NO: 123.

In some embodiments, the viral particles of the present disclosure comprise a polynucleotide sequence encoding, in 5′ to 3′ order:
(a) MND promoter, (b) Cocal envelope, (c) transmembrane domain, (d) cytoplasmic tail domain, (e) WPRE, (f) BGH polyA signal, and the polynucleotide sequence shares at least 75%, at least 80%, at least 85%, at least 90%, at least 95%, at least 99%, or 100% identity to SEQ ID NO: 130.

In some embodiments, the viral particle comprises a nucleic acid sequence that shares at least 75%, at least 80%, at least 85%, at least 90%, at least 95%, at least 99%, or 100% identity to SEQ ID NO: 130.

Anti-CD3 Plasmids

In some embodiments, the viral particle comprises an anti-CD3 nucleic acid sequence that shares at least 75%, at least 80%, at least 85%, at least 90%, at least 95%, at least 99%, or 100% identity to SEQ ID NO: 126.

In some embodiments, the viral particles of the present disclosure comprise a polynucleotide sequence encoding, in 5′ to 3′ order:
(a) human CMV enhancer and CMV promoter, (b) human beta-globin intron, (c) Gaussia luc signal peptide, (d) anti-CD3 VL chain, (e) G4S linker, (f) anti-CD3 VH chain, (g) hinge domain, (h) transmembrane domain, (i) cytoplasmic tail domain, (j) BGH polyA signal, and the polynucleotide sequence shares at least 75%, at least 80%, at least 85%, at least 90%, at least 95%, at least 99%, or 100% identity to SEQ ID NO: 126.

In some embodiments, the viral particle comprises an anti-CD3 nucleic acid sequence that shares at least 75%, at least 80%, at least 85%, at least 90%, at least 95%, at least 99%, or 100% identity to SEQ ID NO: 127.

In some embodiments, the viral particles of the present disclosure comprise a polynucleotide sequence encoding, in 5′ to 3′ order:
(a) human CMV enhancer and CMV promoter, (b) human beta-globin intron, (c) Gaussia luc signal peptide, (d) anti-CD3 VL chain, (e) G4S linker, (f) anti-CD3 VH chain, (g) Glycophorin A transmembrane domain, (h) Glycophorin A cytoplasmic tail domain, (i) BGH polyA signal, and the polynucleotide sequence shares at least 75%, at least 80%, at least 85%, at least 90%, at least 95%, at least 99%, or 100% identity to SEQ ID NO: 127.

Gene Editing

Numerous gene-editing methods are known in the art and additional methods are continuously being created. The methods and compositions of the present disclosure are capable of delivering a variety of genetic payloads, including polynucleotides intended for insertion into the genome of the target cell and/or gene editing systems (CRISPR-Cas, meganucleases, homing endonucleases, zinc finger enzymes and the like). In embodiments, a polynucleotide (e.g. transgene), enzyme, and/or guide RNA are delivered in one, two, three or more vectors of the same type (e.g. lentivirus, AAV, etc.) or different types (including e.g. combinations of non-viral and virus vectors or different types of viral vectors). The methods and systems of the disclosure can be used for generating point mutation(s), insertions, deletions, etc. Random mutagenesis and multi-locus gene editing are also within the scope of the disclosure.

### Target Immune Cells

Non-limiting examples of cells that can be the target of the viral particle described herein include T lymphocytes, dendritic cells (DC), Treg cells, B cells, Natural Killer cells, and macrophages.

T Cells

T cells (“T lymphocytes”) are a type of lymphocyte (itself a type of white blood cell) that play a central role in cell-mediated immunity. There are several subsets of T cells, each with a distinct function. T cells can be distinguished from other lymphocytes, such as B cells and NK cells, by the presence of a T cell receptor (TCR) on the cell surface. The TCR is responsible for recognizing antigens bound to major histocompatibility complex (MHC) molecules and is composed of two different protein chains. In 95% of the T cells, the TCR consists of an alpha (α) and beta (β) chain. When the TCR engages with antigenic peptide and MHC (peptide/MHC complex), the T lymphocyte is activated through a series of biochemical events mediated by associated enzymes, co-receptors, specialized adaptor molecules, and activated or released transcription factors.

In some embodiments, the cells used in the methods provided herein are primary T lymphocytes (e.g., primary human T lymphocytes). The primary T lymphocytes used in the methods provided herein may be naive T lymphocytes or MHC-restricted T lymphocytes. In some embodiments, the T lymphocytes are CD4+. In other embodiments, the T lymphocytes are CD8+. In some embodiments, the primary T lymphocytes are tumor infiltrating lymphocytes (TILs). In some embodiments, the primary T lymphocytes have been isolated from a tumor biopsy or have been expanded from T lymphocytes isolated from a tumor biopsy. In some embodiments, the primary T lymphocytes have been isolated from, or are expanded from T lymphocytes isolated from, peripheral blood, cord blood, or lymph. In some embodiments, the T lymphocytes are allogeneic with respect to a particular individual, e.g., a recipient of said T lymphocytes. In certain other embodiments, the T lymphocytes are not allogeneic with respect to a certain individual, e.g., a recipient of said T lymphocytes. In some embodiments, the T lymphocytes are autologous with respect to a particular individual, e.g., a recipient of said T lymphocytes.

In some embodiments, primary T lymphocytes used in the methods described herein are isolated from a tumor, e.g., are tumor-infiltrating lymphocytes. In some embodiments, such T lymphocytes are specific for a tumor specific antigen (TSA) or tumor associated antigen (TAA). In some embodiments, primary T lymphocytes are obtained from an individual, optionally expanded, and then transduced, using the methods described herein, with a nucleic acid encoding one or more chimeric antigen receptors (CARs), and optionally then expanded. T lymphocytes can be expanded, for example, by contacting the T lymphocytes in culture with antibodies to CD3 and/or CD28, e.g., antibodies attached to beads, or to the surface of a cell culture plate; see, e.g., U.S. Pat. Nos. 5,948,893; 6,534,055; 6,352,694; 6,692,964; 6,887,466; and 6,905,681. In some embodiments, the antibodies are anti-CD3 and/or anti-CD28, and the antibodies are not bound to a solid surface (e.g., the antibodies contact the T lymphocytes in solution). In some embodiments, either of the anti-CD3 antibody or anti-CD28 antibody is bound to a solid surface (e.g. bead, tissue culture dish plastic), and the other antibody is not bound to a solid surface (e.g., is present in solution).

NK Cells

Natural killer (NK) cells are cytotoxic lymphocytes that constitute a major component of the innate immune system. NK cells typically comprise approximately 10 to 15% of the mononuclear cell fraction in normal peripheral blood. NK cells do not express T-cell antigen receptors (TCR), CD3 or surface immunoglobulins (Ig) B cell receptor, but usually express the surface markers CD16 (FcγRIII) and CD56 in humans. NK cells are cytotoxic; small granules in their cytoplasm contain special proteins such as perforin and proteases known as granzymes. Upon release in close proximity to a cell slated for killing, perforin forms pores in the cell membrane of the target cell through which the granzymes and associated molecules can enter, inducing apoptosis. One granzyme, granzyme B (also known as granzyme 2 and cytotoxic T-lymphocyte-associated serine esterase 1), is a serine protease crucial for rapid induction of target cell apoptosis in the cell-mediated immune response.

NK cells are activated in response to interferons or macrophage-derived cytokines Activated NK cells are referred to as lymphokine activated killer (LAK) cells. NK cells possess two types of surface receptors, labeled “activating receptors” and “inhibitory receptors,” that control the cells' cytotoxic activity.

Among other activities, NK cells play a role in the host rejection of tumors. Because many cancer cells have reduced or no class I MHC expression, they can become targets of NK cells. Natural killer cells can become activated by cells lacking, or displaying reduced levels of, major histocompatibility complex (MHC) proteins. In addition to being involved in direct cytotoxic killing, NK cells also serve a role in cytokine production, which can be important to control cancer and infection. Activated and expanded NK cells and LAK cells have been used in both ex vivo therapy and in vivo treatment of patients having advanced cancer, with some success against bone marrow related diseases, such as leukemia; breast cancer; and certain types of lymphoma.

Immune Cell Activation

In some embodiments, administration of the particle to a subject results in the activation of immune cells. In some embodiments, the activation of immune cells is mediated by the CAR's binding to both immune cells and cells expressing specific antigens.

In some embodiments, activation of immune cells is measured by the level of one or more cell markers. In some embodiments, activation of immune cells is measured by the percentage of the immune cells that are positive for one or more cell markers. In some embodiments, the immune cells are T cells (T lymphocytes) or NK cells. In some embodiments, the immune cells are CD4+ T cells or CD8+ T cells. In some embodiments, the one or more cell markers are selected from the groups consisting of CD71, CD25, and any combination thereof.

In some embodiments, activation of immune cells is measured by the percentage of the immune cells that are CD71 positive. In some embodiments, administration of the viral particle increases the percentage of the CD71+ immune cells by at least 10%, at least 20%, at least 30%, at least 40%, at least 50%, at least 60%, at least 70%, at least 80%, or at least 90%. In some embodiments, activation of immune cells is measured by the level of CD71 expressed on the surface of the immune cells. In some embodiments, administration of the viral particle increases the level of CD71 expressed on the surface of the immune cells by at least 10%, at least 20%, at least 30%, at least 40%, at least 50%, at least 60%, at least 70%, at least 80%, at least 90%, at least 1-fold, at least 2-fold, at least 3-fold, at least 5-fold, at least 7-fold, or at least 10-fold.

In some embodiments, activation of immune cells is measured by the percentage of the immune cells that are CD25 positive. In some embodiments, administration of the viral particle increases the percentage of the CD25+ immune cells by at least 10%, at least 20%, at least 30%, at least 40%, at least 50%, at least 60%, at least 70%, at least 80%, or at least 90%. In some embodiments, activation of immune cells is measured by the level of CD25 expressed on the surface of the immune cells. In some embodiments, administration of the viral particle increases the level of CD25 expressed on the surface of the immune cells by at least 10%, at least 20%, at least 30%, at least 40%, at least 50%, at least 60%, at least 70%, at least 80%, at least 90%, at least 1-fold, at least 2-fold, at least 3-fold, at least 5-fold, at least 7-fold, or at least 10-fold.

In some embodiments, administration of the viral particle in a subject results in active proliferation of immune cells. In some embodiments, the proliferation of immune cells increase the number and/or susceptibility to transduction by vector.

In some embodiments, administration of the viral particle in a subject results in a decrease of numbers of immune cells (e.g., T cells) in the GO phase and/or an increase of numbers of immune cells (e.g., T cells) in the non-GO phase.

In some embodiments, administration of the viral particle in a subject increases the number and/or percentage of immune cells that are in a state of metabolic fitness for transduction of vector.

In some embodiments, administration of the viral particle in a subject results in the accumulation of immune cells in lymph nodes. In some embodiments, administration of the viral particle in a subject results in the accumulation of immune cells in tumor sites.

In some embodiments, the viral particle is a lentiviral particle. In some embodiments, the immune cells are T cells. In some embodiments, the immune cells here are a subset of immune cells in vivo that can be recognized by at least one antigen-specific binding domain of the CAR. In some embodiments, the immune cells reside in the lymph nodes.

Dosage Form and Dosing Regimen

Viral Particle

A viral particle may be used to infect cells in vivo at an any effective dosage. In some embodiments, the viral particle is administered to a subject in vivo, by direct injection to the cell, tissue, organ or subject in need of therapy.

Viral particles may also be delivered according to viral titer (TU/mL). The amount of lentivirus directly injected is determined by total TU and can vary based on both the volume that could be feasibly injected to the site and the type of tissue to be injected. In some embodiments, the viral titer delivered is about 1×10⁵ to 1×10⁶, about 1×10⁵ to 1×10⁷, 1×10⁵ to 1×10⁷, about 1×10⁶ to 1×10⁹, about 1×10⁷ to 1×10¹⁰, about 1×10⁷ to 1×10¹¹, or about 1×10⁹ to 1×10¹¹ TU or more per injection could be used. In some embodiments, the viral titer delivered is about 1×10⁶ to 1×10⁷, about 1×10⁶ to 1×10⁸, 1×10⁶ to 1×10⁹, about 1×10⁷ to 1×10¹⁰, about 1×10⁸ to 1×10¹¹, about 1×10⁸ to 1×10¹², or about 1×10¹⁰ to 1×10¹² or more per injection could be used. For example, a brain injection site may only allow for a very small volume of virus to be injected, so a high titer prep would be preferred, a TU of about 1×10⁶ to 1×10⁷, about 1×10⁶ to 1×10⁸, 1×10⁶ to 1×10⁹ about 1×10⁷ to 1×10¹⁰, about 1×10⁸ to 1×10¹¹, about 1×10⁸ to 1×10¹², or about 1×10¹⁰ to 1×10¹² or more per injection could be used. However, a systemic delivery could accommodate a much larger TU, a load of about 1×10⁸, about 1×10⁹, about 1×10¹⁰, about 1×10¹¹, about 1×10¹² about 1×10¹³, about 1×10¹⁴, or about 1×10¹⁵, could be delivered.

In some embodiments, the vector is administered at a dose of between about 1×10¹² and 5×10¹⁴ vector genomes (vg) of the vector per kilogram (vg) of total body mass of the subject (vg/kg). In some embodiments, the vector is administered at a dose of between about 1×10¹³ and 5×10¹⁴ vg/kg. In some embodiments, the vector is administered at a dose of between about 5×10¹³ and 3×10¹⁴ vg/kg. In some embodiments, the vector is administered at a dose of between about 5×10¹³ and 1×10¹⁴ vg/kg. In some embodiments, the vector is administered at a dose of less than about 1×10¹² vg/kg, less than about 3×10¹² vg/kg, less than about 5×10¹² vg/kg, less vg/kg, than about 7×10¹² vg/kg, less than about 1×10¹³ vg/kg, less than about 3×10¹³ vg/kg, less than about 5×10¹³ vg/kg, less than about 7×10¹³ vg/kg, less than about 1×10¹⁴ vg/kg, less than about 3×10¹⁴ vg/kg, 5×10¹⁴ vg/kg, less than about 5×10¹⁴ vg/kg, less than about 7×10¹⁴ vg/kg, less than about 1×10¹⁵ vg/kg, less than about 3×10¹⁵ vg/kg, less than about 5×10¹⁵ vg/kg, or less than about 7×10¹⁵ vg/kg.

In some embodiments, the vector is administered at a dose of between about 1×10¹² and 5×10¹⁴ vector particles (vp) of the vector per kilogram (vp) of total body mass of the subject (vp/kg). In some embodiments, the vector is administered at a dose of between about 1×10¹³ 5×10¹³ and 5×10¹⁴ vp/kg. In some embodiments, the vector is administered at a dose of between about 5×10¹³ and 3×10¹⁴ vp/kg. In some embodiments, the vector is administered at a dose of between about 5×10¹³ and 1×10¹⁴ vp/kg. In some embodiments, the vector is administered at a dose of less than about 1×10¹² vp/kg, less than about 3×10¹² vp/kg, less than about 5×10¹² vp/kg, less vp/kg, than about 7×10¹² vp/kg, less than about 1×10¹³ vp/kg, less than about 3×10¹³ vp/kg, less than about 5×10¹³ vp/kg, less than about 7×10¹³ vp/kg, less than about 1×10¹⁴ vp/kg, less than about 3×10¹⁴ vp/kg less than about 5×10¹⁴ vp/kg, less than about 7×10¹⁴ vp/kg, less than about 1×10¹⁵ vp/kg, less than about 3×10¹⁵ vp/kg, less than about 5×10¹⁵ vp/kg, or less than about 7×10¹⁵ vp/kg.

In some embodiments, administration of the viral particles of the present disclosure decreases the number of B cells in the subject by at least 1%, at least 2%, at least 3%, at least 5%, at least 7%, at least 10%, at least 20%, at least 30%, at least 40%, at least 50%, at least 60%, at least 70%, at least 80%, or at least 90%. In some embodiments, the decrease is evaluated by the number of B cells 1, 2, 3, 4, 5, 6, 7, 8, 9, 10, 11, or 12 weeks after the viral particle is administered, wherein the reference number is the number of B cells in a subject that was administered a vehicle control. In some embodiments, administration of the viral particles of the present disclosure decreases the number of B cells in the subject by at least 95%.

In some embodiments, the B cells are in the peripheral blood of the subject. In some embodiments, the B cells are in the bone marrow of the subject. In some embodiments, the B cells are in the spleen of the subject

In some embodiments, the B cells are depleted in the subject for at least 7 days, at least 10 days, at least 20 days, at least 30 days, at least 40 days, at least 50 days, at least 60 days, at least 70 days, or at least 80 days after administering the viral particle.

In some embodiments, the B cells are depleted in the subject for at least 80 days after administering the viral particle.

Rapamycin

Rapamune® (sirolimus, rapamycin) is available as an oral solution or tablet and is FDA approved for the following indications:
Prophylaxis of organ rejection on renal transplantation
Limitations of use in renal transplantation
Treatment of patients with lymphangioleiomyomatosis

Per the US Prescribing Information (USPI), rapamycin is available in 1 mg/mL oral solution or 0.5, 1, or 2 mg tablets and is to be administered once daily. Rapamycin may also be delivered in other dosage forms and/or by other administration routes.

In some embodiments, rapamycin is administered at a dose of between about 0.1 mg/m² and 100 mg/m² of surface area of the subject. In some embodiments, the subject is a human. In some embodiments, rapamycin is administered at a dose of between about 0.5 mg/m² and 50 mg/m². In some embodiments, rapamycin is administered at a dose of between about 0.5 mg/m² and 10 mg/m². In some embodiments, rapamycin is administered at a dose of between about 0.5 mg/m² and 3 mg/m². In some embodiments, rapamycin is administered at a dose of between about 0.5 mg/m² and 5 mg/m². In some embodiments, rapamycin is administered at a dose of between about 1 mg/m² and 5 mg/m². In some embodiments, rapamycin is administered at a dose of between about 2 mg/m² and 6 mg/m². In some embodiments, rapamycin is administered at a dose of about 1 mg/m². In some embodiments, rapamycin is administered at a dose of about 2 mg/m². In some embodiments, rapamycin is administered at a dose of about 3 mg/m². In some embodiments, rapamycin is administered at a dose of between about 2 mg/m² and 6 mg/m². In some embodiments, rapamycin is administered at a dose of between about 3 mg/m² and 9 mg/m². In some embodiments, rapamycin is administered at a dose of between about 4 mg/m² and 12 mg/m². In some embodiments, rapamycin is administered at a dose of between about 5 mg/m² and 15 mg/m². In some embodiments, rapamycin is administered at a dose of between about 6 mg/m² and 20 mg/m². In some embodiments, rapamycin is administered at a dose of between about 10 mg/m² and 50 mg/m². In some embodiments, the dose of rapamycin is the total dose within a 24-hour time period.

In some embodiments, rapamycin is administered at a dose of between about 0.001 mg/m² and 100 mg/m² of surface area of the subject. In some embodiments, the subject is a human. In some embodiments, rapamycin is administered at a dose of between about 0.001 mg/m² and 0.1 mg/m², between about 0.01 mg/m² and 1 mg/m², between about 0.1 mg/m² and 10 mg/m², between about 1 mg/m² and 100 mg/m², between about 0.001 mg/m² and 0.05 mg/m², between about 0.005 mg/m² and 0.25 mg/m², between about 0.01 mg/m² and 0.5 mg/m², between about 0.05 mg/m² and 2.5 mg/m², between about 0.1 mg/m² and 5 mg/m², between about 0.5 mg/m² and 25 mg/m², between about 1 mg/m² and 50 mg/m², between about 2 mg/m² and 100 mg/m², between about 0.001 mg/m² and 0.01 mg/m², between about 0.005 mg/m² and 0.05 mg/m², between about 0.01 mg/m² and 0.1 mg/m², between about 0.05 mg/m² and 0.5 mg/m², between about 0.1 mg/m² and 1 mg/m², between about 0.5 mg/m² and 5 mg/m², between about 1 mg/m² and 10 mg/m², between about 5 mg/m² and 50 mg/m², or between about 10 mg/m² and 100 mg/m², including all ranges and subranges in between. In some embodiments, rapamycin is administered at a dose of between about 0.001 mg/m² and 0.005 mg/m². In some embodiments, rapamycin is administered at a dose of between about 0.002 mg/m² and 0.01 mg/m². In some embodiments, rapamycin is administered at a dose of between about 0.003 mg/m² and 0.015 mg/m². In some embodiments, rapamycin is administered at a dose of between about 0.004 mg/m² and 0.02 mg/m². In some embodiments, rapamycin is administered at a dose of between about 0.005 mg/m² and 0.025 mg/m². In some embodiments, rapamycin is administered at a dose of between about 0.006 mg/m² and 0.03 mg/m². In some embodiments, rapamycin is administered at a dose of between about 0.007 mg/m² and 0.035 mg/m². In some embodiments, rapamycin is administered at a dose of between about 0.008 mg/m² and 0.04 mg/m². In some embodiments, rapamycin is administered at a dose of between about 0.009 mg/m² and 0.045 mg/m². In some embodiments, rapamycin is administered at a dose of between about 0.01 mg/m² and 0.05 mg/m². In some embodiments, rapamycin is administered at a dose of between about 0.02 mg/m² and 0.1 mg/m². In some embodiments, rapamycin is administered at a dose of between about 0.03 mg/m² and 0.15 mg/m². In some embodiments, rapamycin is administered at a dose of between about 0.04 mg/m² and 0.2 mg/m². In some embodiments, rapamycin is administered at a dose of between about 0.05 mg/m² and 0.25 mg/m². In some embodiments, rapamycin is administered at a dose of between about 0.06 mg/m² and 0.3 mg/m². In some embodiments, rapamycin is administered at a dose of between about 0.07 mg/m² and 0.35 mg/m². In some embodiments, rapamycin is administered at a dose of between about 0.08 mg/m² and 0.4 mg/m². In some embodiments, rapamycin is administered at a dose of between about 0.09 mg/m² and 0.45 mg/m². In some embodiments, rapamycin is administered at a dose of between about 0.1 mg/m² and 0.5 mg/m². In some embodiments, rapamycin is administered at a dose of between about 0.2 mg/m² and 1 mg/m². In some embodiments, rapamycin is administered at a dose of between about 0.3 mg/m² and 1.5 mg/m². In some embodiments, rapamycin is administered at a dose of between about 0.4 mg/m² and 2 mg/m². In some embodiments, rapamycin is administered at a dose of between about 0.5 mg/m² and 2.5 mg/m². In some embodiments, rapamycin is administered at a dose of between about 0.6 mg/m² and 3 mg/m². In some embodiments, rapamycin is administered at a dose of between about 0.7 mg/m² and 3.5 mg/m². In some embodiments, rapamycin is administered at a dose of between about 0.8 mg/m² and 4 mg/m². In some embodiments, rapamycin is administered at a dose of between about 0.9 mg/m² and 4.5 mg/m². In some embodiments, rapamycin is administered at a dose of between about 1 mg/m² and 5 mg/m². In some embodiments, rapamycin is administered at a dose of between about 2 mg/m² and 10 mg/m². In some embodiments, rapamycin is administered at a dose of between about 3 mg/m² and 15 mg/m². In some embodiments, rapamycin is administered at a dose of between about 4 mg/m² and 20 mg/m². In some embodiments, rapamycin is administered at a dose of between about 5 mg/m² and 25 mg/m². In some embodiments, rapamycin is administered at a dose of between about 6 mg/m² and 30 mg/m². In some embodiments, rapamycin is administered at a dose of between about 7 mg/m² and 35 mg/m². In some embodiments, rapamycin is administered at a dose of between about 8 mg/m² and 40 mg/m². In some embodiments, rapamycin is administered at a dose of between about 9 mg/m² and 45 mg/m². In some embodiments, rapamycin is administered at a dose of between about 10 mg/m² and 50 mg/m². In some embodiments, rapamycin is administered at a dose of between about 20 mg/m² and 100 mg/m². In some embodiments, rapamycin is administered at a dose of between about 0.001 mg/m² and 0.02 mg/m². In some embodiments, rapamycin is administered at a dose of between about 0.002 mg/m² and 0.04 mg/m². In some embodiments, rapamycin is administered at a dose of between about 0.003 mg/m² and 0.06 mg/m². In some embodiments, rapamycin is administered at a dose of between about 0.004 mg/m² and 0.08 mg/m². In some embodiments, rapamycin is administered at a dose of between about 0.005 mg/m² and 0.1 mg/m². In some embodiments, rapamycin is administered at a dose of between about 0.006 mg/m² and 0.12 mg/m². In some embodiments, rapamycin is administered at a dose of between about 0.007 mg/m² and 0.14 mg/m². In some embodiments, rapamycin is administered at a dose of between about 0.008 mg/m² and 0.16 mg/m². In some embodiments, rapamycin is administered at a dose of between about 0.009 mg/m² and 0.18 mg/m². In some embodiments, rapamycin is administered at a dose of between about 0.01 mg/m² and 0.2 mg/m². In some embodiments, rapamycin is administered at a dose of between about 0.02 mg/m² and 0.4 mg/m². In some embodiments, rapamycin is administered at a dose of between about 0.03 mg/m² and 0.6 mg/m². In some embodiments, rapamycin is administered at a dose of between about 0.04 mg/m² and 0.8 mg/m². In some embodiments, rapamycin is administered at a dose of between about 0.05 mg/m² and 1 mg/m². In some embodiments, rapamycin is administered at a dose of between about 0.06 mg/m² and 1.2 mg/m². In some embodiments, rapamycin is administered at a dose of between about 0.07 mg/m² and 1.4 mg/m². In some embodiments, rapamycin is administered at a dose of between about 0.08 mg/m² and 1.6 mg/m². In some embodiments, rapamycin is administered at a dose of between about 0.09 mg/m² and 1.8 mg/m². In some embodiments, rapamycin is administered at a dose of between about 0.1 mg/m² and 2 mg/m². In some embodiments, rapamycin is administered at a dose of between about 0.2 mg/m² and 4 mg/m². In some embodiments, rapamycin is administered at a dose of between about 0.3 mg/m² and 6 mg/m². In some embodiments, rapamycin is administered at a dose of between about 0.4 mg/m² and 8 mg/m². In some embodiments, rapamycin is administered at a dose of between about 0.5 mg/m² and 10 mg/m². In some embodiments, rapamycin is administered at a dose of between about 0.6 mg/m² and 12 mg/m². In some embodiments, rapamycin is administered at a dose of between about 0.7 mg/m² and 14 mg/m². In some embodiments, rapamycin is administered at a dose of between about 0.8 mg/m² and 16 mg/m². In some embodiments, rapamycin is administered at a dose of between about 0.9 mg/m² and 18 mg/m². In some embodiments, rapamycin is administered at a dose of between about 1 mg/m² and 20 mg/m². In some embodiments, rapamycin is administered at a dose of between about 2 mg/m² and 40 mg/m². In some embodiments, rapamycin is administered at a dose of between about 3 mg/m² and 60 mg/m². In some embodiments, rapamycin is administered at a dose of between about 4 mg/m² and 80 mg/m². In some embodiments, rapamycin is administered at a dose of between about 5 mg/m² and 100 mg/m². In some embodiments, the dose of rapamycin is the total dose within a 24-hour time period.

In some embodiments, a dose of rapamycin is administered every day. In some embodiments, a dose of rapamycin is administered about every 1, 2, 3, 4, 5, 6, 7, 8, 9, 10, 11, 12, 13, or 14 days. In some embodiments, a dose of rapamycin is administered about every 1, 2, 3, 4, 5, 6, 7, 8, 9, or 10 weeks. In some embodiments, a dose of rapamycin is administered about every 1, 2, 3, 4, 5, 6, 7, 8, 9, or 10 months.

In some embodiments, after the first administration of the viral particle, the first dose of rapamycin is administered about 1, 2, 3, 4, 5, 6, 7, 8, 9, 10, 11, 12, 13, or 14 days post first administration of the viral particle. In some embodiments, after the first administration of the viral particle, the first dose of rapamycin is administered about 1, 2, 3, 4, 5, 6, 7, 8, 9, 10, 11, 12, 13, or 14 weeks post first administration of the viral particle. In some embodiments, after the first administration of the viral particle, the first dose of rapamycin is administered about 1, 2, 3, 4, 5, 6, 7, 8, 9, 10, 11, 12, 13, or 14 months post first administration of the viral particle. In some embodiments, after the first administration of the viral particle, the first dose of rapamycin is administered between about 1-3 days, between about 2-6 days, between about 3-9 days, between about 4-12 days, between about 5-15 days, between about 1-3 weeks, between about 2-4 weeks, between about 3-6 weeks, or between about 4-8 weeks post first administration of the viral particle.

In some embodiments, administration of rapamycin increases the number of viral particle transduced immune cells (e.g., CAR T cells) in the subject, or in a particular organ/region of the subject. In some embodiments, the organ/region of the subject is blood. In some embodiments, the organ/region of the subject is spleen. In some embodiments, the organ/region of the subject is bone marrow. In some embodiments, administration of rapamycin increases the number of viral particle transduced immune cells (e.g., CAR T cells) by at least 10%, at least 20%, at least 30%, at least 40%, at least 50%, at least 60%, at least 70%, at least 80%, at least 90%, at least 1-fold, at least 2-fold, at least 3-fold, at least 5-fold, at least 7-fold, or at least 10-fold, in the subject. In some embodiments, the increase is evaluated by the number of viral particle transduced immune cells 1, 2, 3, 4, 5, 6, 7, 8, 9, 10, 11, or 12 weeks after the first dose of the rapamycin (once the viral particle is administered), wherein the reference number is the number of viral particle transduced immune cells on the day of the first dose of rapamycin. In some embodiments, the increase is evaluated by the number of viral particle transduced immune cells 1, 2, 3, 4, 5, 6, 7, 8, 9, 10, 11, or 12 months after the first dose of the rapamycin (once the viral particle is administered), wherein the reference number is the number of viral particle transduced immune cells on the day of the first dose of rapamycin.

In some embodiments, administration of rapamycin increases the percentage of viral particle transduced immune cells (e.g., CAR T cells) in the subject, or in a particular organ/region of the subject. In some embodiments, the organ/region of the subject is blood. In some embodiments, the organ/region of the subject is spleen. In some embodiments, the organ/region of the subject is bone marrow. In some embodiments, administration of rapamycin increases the percentage of viral particle transduced immune cells (e.g., CAR T cells) by at least 1%, at least 2%, at least 3%, at least 5%, at least 7%, at least 10%, at least 20%, at least 30%, at least 40%, at least 50%, at least 60%, at least 70%, at least 80%, or at least 90% in the subject. In some embodiments, the increase is evaluated by the percentage of viral particle transduced immune cells 1, 2, 3, 4, 5, 6, 7, 8, 9, 10, 11, or 12 weeks after the first dose of the rapamycin (once the viral particle is administered), wherein the reference percentage is the percentage of viral particle transduced immune cells on the day of the first dose of rapamycin. In some embodiments, the increase is evaluated by the percentage of viral particle transduced immune cells 1, 2, 3, 4, 5, 6, 7, 8, 9, 10, 11, or 12 months after the first dose of the rapamycin (once the viral particle is administered), wherein the reference percentage is the percentage of viral particle transduced immune cells on the day of the first dose of rapamycin. In some embodiments, the percentage is the percentage of viral particle transduced immune cells in total immune cells in the subject or in the particular organ/region of the subject. In some embodiments, the percentage is the percentage of viral particle transduced immune cells in immune cells of the same type (e.g., T cells) in the subject or in the particular organ/region of the subject.

### Pharmaceutical Compositions and Formulations

The formulations and compositions of the present disclosure may comprise a combination of any number of viral particles, and optionally one or more additional pharmaceutical agents (polypeptides, polynucleotides, compounds etc.) formulated in pharmaceutically acceptable or physiologically-acceptable compositions for administration to a cell, tissue, organ, or an animal, either alone, or in combination with one or more other modalities of therapy. In some embodiments, the one or more additional pharmaceutical agent further increases transduction efficiency of vectors.

In some embodiments, the present disclosure provides compositions comprising a therapeutically-effective amount of a viral particle, as described herein, formulated together with one or more pharmaceutically acceptable carriers (additives) and/or diluents. In some embodiments, the composition further comprises other agents, such as, e.g., cytokines, growth factors, hormones, small molecules or various pharmaceutically active agents.

In some embodiments, compositions and formulations of the viral particles used in accordance with the present disclosure may be prepared for storage by mixing a viral particle having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed. In some embodiments, one or more pharmaceutically acceptable surface-active agents (surfactant), buffers, isotonicity agents, salts, amino acids, sugars, stabilizers and/or antioxidant are used in the formulation.

Suitable pharmaceutically acceptable surfactants comprise but are not limited to polyethylene-sorbitan-fatty acid esters, polyethylene-polypropylene glycols, polyoxyethylene-stearates and sodium dodecyl sulphates. Suitable buffers comprise but are not limited to histidine-buffers, citrate-buffers, succinate-buffers, acetate-buffers and phosphate-buffers.

Isotonicity agents are used to provide an isotonic formulation. An isotonic formulation is liquid, or liquid reconstituted from a solid form, e.g. a lyophilized form and denotes a solution having the same tonicity as some other solution with which it is compared, such as physiologic salt solution and the blood serum. Suitable isotonicity agents comprise but are not limited to salts, including but not limited to sodium chloride (NaCl) or potassium chloride, sugars including but not limited to glucose, sucrose, trehalose or and any component from the group of amino acids, sugars, salts and combinations thereof. In some embodiments, isotonicity agents are generally used in a total amount of about 5 mM to about 350 mM.

Non-limiting examples of salts include salts of any combinations of the cations sodium potassium, calcium or magnesium with anions chloride, phosphate, citrate, succinate, sulphate or mixtures thereof. Non-limiting examples of amino acids comprise arginine, glycine, ornithine, lysine, histidine, glutamic acid, asparagic acid, isoleucine, leucine, alanine, phenylalanine, tyrosine, tryptophane, methionine, serine, proline. Non-limiting examples of sugars according to the invention include trehalose, sucrose, mannitol, sorbitol, lactose, glucose, mannose, maltose, galactose, fructose, sorbose, raffinose, glucosamine, N-methylglucosamine (also referred to as “meglumine”), galactosamine and neuraminic acid and combinations thereof. Non-limiting examples of stabilizer includes amino acids and sugars as described above as well as commercially available cyclodextrins and dextrans of any kind and molecular weight as known in the art. Non-limiting examples of antioxidants include excipients such as methionine, benzylalcohol or any other excipient used to minimize oxidation.

The phrase “pharmaceutically acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

As used herein “pharmaceutically acceptable carrier” includes any and all solvents, dispersion media, coatings, antibacterial and antifungal agents, isotonic and absorption delaying agents, and the like that are physiologically compatible, including pharmaceutically acceptable cell culture media. In some embodiments, a composition comprising a carrier is suitable for parenteral administration, e.g., intravascular (intravenous or intraarterial), intraperitoneal or intramuscular administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the transduced cells, use thereof in the pharmaceutical compositions of the present disclosure is contemplated.

The compositions may further comprise one or more polypeptides, polynucleotides, vectors comprising same, compounds that increase the transduction efficiency of vectors, formulated in pharmaceutically acceptable or physiologically-acceptable solutions for administration to a cell or an animal, either alone, or in combination with one or more other modalities of therapy. It will also be understood that, if desired, the compositions of the present disclosure may be administered in combination with other agents as well, such as, e.g., cytokines, growth factors, hormones, small molecules or various pharmaceutically active agents. There is virtually no limit to other components that may also be included in the compositions, provided that the additional agents do not adversely affect the ability of the composition to deliver the intended therapy.

The present disclosure also provides pharmaceutical compositions comprising an expression cassette or vector (e.g., therapeutic vector) disclosed herein and one or more pharmaceutically acceptable carriers, diluents or excipients. In some embodiments, the pharmaceutical composition comprises a lentiviral vector comprising an expression cassette disclosed herein, e.g., wherein the expression cassette comprises one or more polynucleotide sequences encoding one or more chimeric antigen receptor (CARs) and variants thereof.

The pharmaceutical compositions that contain the expression cassette or vector may be in any form that is suitable for the selected mode of administration, for example, for intraventricular, intramyocardial, intracoronary, intravenous, intra-arterial, intra-renal, intraurethral, epidural, intrathecal, intraperitoneal, or intramuscular administration. The vector can be administered, as sole active agent, or in combination with other active agents, in a unit administration form, as a mixture with conventional pharmaceutical supports, to animals and human beings. In some embodiments, the pharmaceutical composition comprises cells transduced ex vivo with any of the vectors according to the present disclosure.

In some embodiments, the viral particle (e.g., lentiviral particle), or a pharmaceutical composition comprising that viral particle, is effective when administered systemically. For example, the viral vectors of the disclosure, in some cases, demonstrate efficacy when administered intravenously to subject (e.g., a primate, such as a non-human primate or a human). In some embodiments, the viral vectors of the disclosure are capable of inducing expression of CAR in various immune cells when administered systemically (e.g., in T-cells, dendritic cells, NK cells).

In various embodiments, the pharmaceutical compositions contain vehicles (e.g., carriers, diluents and excipients) that are pharmaceutically acceptable for a formulation capable of being injected. Exemplary excipients include a poloxamer. Formulation buffers for viral vectors general contains salts to prevent aggregation and other excipients (e.g., poloxamer) to reduce stickiness of the viral particle. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. In some embodiments, the formulation is stable for storage and use when frozen (e.g., at less than 0° C., about −60° C., or about −72° C.). In some embodiments, the formulation is a cryopreserved solution.

The pharmaceutical compositions of the present disclosure, formulation of pharmaceutically acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, intraperitoneal, and intramuscular administration and formulation.

In certain circumstances, it will be desirable to deliver the compositions disclosed herein parenterally, intravenously, intramuscularly, or intraperitoneally, for example, in U.S. Pat. Nos. 5,543,158; 5,641,515 and 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In some embodiments, isotonic agents, for example, sugars or sodium chloride, are added. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington: The Science and Practice of Pharmacy, 20th Edition. Baltimore, Md.: Lippincott Williams & Wilkins, 2005). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

In some embodiments, the present disclosure provides formulations or compositions suitable for the delivery of viral vector systems (i.e., viral-mediated transduction) including, but not limited to, retroviral (e.g., lentiviral) vectors.

Combinatorial Therapy

The present disclosure further contemplates that one or more additional agents that improve the transduction efficiency of viral particle may be used.

In some embodiments, the method further comprises administering to the subject one or more anti-cancer therapies.

In some embodiments, the one or more anti-cancer therapies is selected from the group consisting of an autologous stem cell transplant (ASCT), radiation, surgery, a chemotherapeutic agent, an immunomodulatory agent and a targeted cancer therapy.

In some embodiments, the one or more anti-cancer therapies is selected from the group consisting of lenalidomide, thalidomide, pomalidomide, bortezomib, carfilzomib, elotuzumab, ixazomib, melphalan, dexamethasone, vincristine, cyclophosphamide, hydroxy daunorubicin, prednisone, rituximab, imatinib, dasatinib, nilotinib, bosutinib, ponatinib, bafetinib, saracatinib, tozasertib or danusertib, cytarabine, daunorubicin, idarubicin, mitoxantrone, hydroxyurea, decitabine, cladribine, fludarabine, topotecan, etoposide 6-thioguanine, corticosteroid, methotrexate, 6-mercaptopurine, azacitidine, arsenic trioxide and all-trans retinoic acid, or any combination thereof.

### Diseases

The disclosure also provides a viral particle that can be used for treatment of diseases, disorders or conditions. In some embodiments, the disease or disorder is cancer. In some embodiments, the cancer is a hematological malignancy or a solid tumor. In some embodiments, the subject is relapsed or refractory to treatment with a prior anti-cancer therapeutic.

In some embodiments, a therapeutic application of the viral particles disclosed herein is to treat CD19-expressing B-cell malignancies that have failed other non-CAR T-cell treatment options.

Hematological Malignancy

In some embodiments, the cancer is a hematological malignancy.

In some embodiments, the hematological malignancy is lymphoma, a B cell malignancy, Hodgkin's lymphoma, non-Hodgkin's lymphoma, a DLBLC, a FL, a MCL, a marginal zone B-cell lymphoma (MZL), a mucosa-associated lymphatic tissue lymphoma (MALT), a CLL, an ALL, an AML, Waldenstrom's Macroglobulinemia or a T-cell lymphoma.

In some embodiments, the solid tumor is a lung cancer, a liver cancer, a cervical cancer, a colon cancer, a breast cancer, an ovarian cancer, a pancreatic cancer, a melanoma, a glioblastoma, a prostate cancer, an esophageal cancer or a gastric cancer. WO2019057124A1 discloses cancers that are amenable to treatment with T cell redirecting therapeutics that bind CD19.

In some embodiments, the hematological malignancy is a multiple myeloma, a smoldering multiple myeloma, a monoclonal gammopathy of undetermined significance (MGUS), an acute lymphoblastic leukemia (ALL), a diffuse large B-cell lymphoma (DLBCL), a Burkitt's lymphoma (BL), a follicular lymphoma (FL), a mantle-cell lymphoma (MCL), Waldenstrom's macroglobulinemia, a plasma cell leukemia, a light chain amyloidosis (AL), a precursor B-cell lymphoblastic leukemia, a precursor B-cell lymphoblastic leukemia, an acute myeloid leukemia (AML), a myelodysplastic syndrome (MDS), a chronic lymphocytic leukemia (CLL), a B cell malignancy, a chronic myeloid leukemia (CML), a hairy cell leukemia (HCL), a blastic plasmacytoid dendritic cell neoplasm, Hodgkin's lymphoma, non-Hodgkin's lymphoma, a marginal zone B-cell lymphoma (MZL), a mucosa-associated lymphatic tissue lymphoma (MALT), plasma cell leukemia, anaplastic large-cell lymphoma (ALCL), leukemia or lymphoma.

In some embodiments, the at least one genetic abnormality is a translocation between chromosomes 8 and 21, a translocation or an inversion in chromosome 16, a translocation between chromosomes 15 and 17, changes in chromosome 11, or mutation in fins-related tyrosine kinase 3 (FLT3), nucleophosmin (NPM1), isocitrate dehydrogenase 1 (IDH1), isocitrate dehydrogenase 2 (IDH2), DNA (cytosine-5)-methyltransferase 3 (DNMT3A), CCAAT/enhancer binding protein alpha (CEBPA), U2 small nuclear RNA auxiliary factor 1 (U2AF1), enhancer of zeste 2 polycomb repressive complex 2 subunit (EZH2), structural maintenance of chromosomes 1A (SMC1A) or structural maintenance of chromosomes 3 (SMC3).

In some embodiments, the hematological malignancy is the ALL.

In some embodiments, the ALL is B-cell lineage ALL, T-cell lineage ALL, adult ALL or pediatric ALL.

In some embodiments, the subject with ALL has a Philadelphia chromosome or is resistant or has acquired resistance to treatment with a BCR-ABL kinase inhibitor.

The Ph chromosome is present in about 20% of adults with ALL and a small percentage of children with ALL and is associated with poor prognosis. At a time of relapse, patients with Ph+ positive ALL may be on tyrosine kinase inhibitor (TKI) regimen and may have therefore become resistant to the TKI. The method as described herein may thus be administered to a subject who has become resistant to selective or partially selective BCR-ABL inhibitors. Exemplary BCR-ABL inhibitors are for example imatinib, dasatinib, nilotinib, bosutinib, ponatinib, bafetinib, saracatinib, tozasertib or danusertib.

In some embodiments, the subject has ALL with t(v;11q23) (MLL rearranged), t(1;19)(q23;p13.3); TCF3-PBX1 (E2A-PBX1), t(12;21)(p13;q22); ETV6-RUNX1 (TEL-AML1) or t(5;14)(q31;q32); IL3-IGH chromosomal rearrangement.

Chromosomal rearrangements can be identified using well known methods, for example fluorescent in situ hybridization, karyotyping, pulsed field gel electrophoresis, or sequencing.

In some embodiments, the hematological malignancy is the smoldering multiple myeloma, MGUS, ALL, DLBLC, BL, FL, MCL, Waldenstrom's macroglobulinemia, plasma cell leukemia, AL, precursor B-cell lymphoblastic leukemia, precursor B-cell lymphoblastic leukemia, myelodysplastic syndrome (MDS), CLL, B cell malignancy, CML, HCL, blastic plasmacytoid dendritic cell neoplasm, Hodgkin's lymphoma, non-Hodgkin's lymphoma, MZL, MALT, plasma cell leukemia, ALCL, leukemia, or lymphoma.

In some embodiments, the cancer is diffuse large B-cell lymphoma (DLBCL). In some embodiments, the cancer is Burkitt's type large B-cell lymphoma (B-LBL). In some embodiments, the cancer is follicular lymphoma (FL). In some embodiments, the cancer is chronic lymphocytic leukemia (CLL). In some embodiments, the cancer is acute lymphocytic leukemia (ALL). In some embodiments, the cancer is mantle cell lymphoma (MCL).

Solid Tumor

In some embodiments, the cancer is a solid tumor.

In some embodiments, the solid tumor is a prostate cancer, a lung cancer, a non-small cell lung cancer (NSCLC), a liver cancer, a cervical cancer, a colon cancer, a breast cancer, an ovarian cancer, an endometrial cancer, a pancreatic cancer, a melanoma, an esophageal cancer, a gastric cancer, a stomach cancer, a renal carcinoma, a bladder cancer, a hepatocellular carcinoma, a renal cell carcinoma, an urothelial carcinoma, a head and neck cancer, a glioma, a glioblastoma, a colorectal cancer, a thyroid cancer, epithelial cancers, or adenocarcinomas.

In some embodiments, the prostate cancer is a relapsed prostate cancer. In some embodiments, the prostate cancer is a refractory prostate cancer. In some embodiments, the prostate cancer is a malignant prostate cancer. In some embodiments, the prostate cancer is a castration resistant prostate cancer.

### Definitions

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description is for the purpose of describing particular embodiments only and is not intended to be limiting. All publications, patent applications, patents and other references mentioned herein are incorporated by reference in their entirety. In case of a conflict in terminology, the present specification is controlling.

The terms “identical” or percent “identity,” in the context of two or more nucleic acids or polypeptide sequences, refer to two or more sequences or subsequences that are the same or have a specified percentage of amino acid residues or nucleotides that are the same (i.e., share at least about 80% identity, for example, at least about 85%, 90%, 91%, 92%, 93%, 94%, 95%, 96%, 97%, 98%, 99% identity over a specified region to a reference sequence, when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. In some embodiments, the identity exists over a region that is at least about 25 amino acids or nucleotides in length, for example, over a region that is 50, 100, 200, 300, 400 amino acids or nucleotides in length, or over the full-length of a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. In some embodiments, BLAST and BLAST 2.0 algorithms and the default parameters are used.

A “comparison window,” as used herein, includes reference to a segment of any one of the number of contiguous positions selected from the group consisting of 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., eds., Current Protocols in Molecular Biology (1995 supplement)). Examples of algorithms that are suitable for determining percent sequence identity and sequence similarity are the BLAST and BLAST 2.0 algorithms, which are described in Altschul et al., J. Mol. Biol. 215:403-410 (1990) and Altschul et al., Nucleic Acids Res. 25:3389-3402 (1977), respectively. Software for performing BLAST analyses is publicly available through the National Center for Biotechnology Information (on the worldwide web at ncbi.nlm.nih.gov/).

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions. Yet another indication that two nucleic acid sequences are substantially identical is that the same primers can be used to amplify the sequence.

As used in the description of the invention and the appended claims, the singular forms “a”, “an”, and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (or).

As used herein, “administering” refers to local and systemic administration, e.g., including enteral, parenteral, pulmonary, and topical/transdermal administration. Routes of administration for pharmaceutical ingredients (e.g., vectors) that find use in the methods described herein include, e.g., oral (per os (P.O.)) administration, nasal or inhalation administration, administration as a suppository, topical contact, transdermal delivery (e.g., via a transdermal patch), intrathecal (IT) administration, intravenous (“iv”) administration, intraperitoneal (“ip”) administration, intramuscular (“im”) administration, intralesional administration, or subcutaneous (“sc”) administration, or the implantation of a slow-release device e.g., a mini-osmotic pump, a depot formulation, etc., to a subject. Administration can be by any route including parenteral and transmucosal (e.g., oral, nasal, vaginal, rectal, or transdermal). Parenteral administration includes, e.g., intravenous, intramuscular, intraarterial, intrarenal, intraurethral, intracardiac, intracoronary, intramyocardial, intradermal, epidural, subcutaneous, intraperitoneal, intraventricular, iontophoretic and intracranial. Other modes of delivery include, but are not limited to, the use of liposomal formulations, intravenous infusion, transdermal patches, etc.

The terms “systemic administration” and “systemically administered” refer to a method of administering a pharmaceutical ingredient or composition to a mammal so that the pharmaceutical ingredient or composition is delivered to sites in the body, including the targeted site of pharmaceutical action, via the circulatory system. Systemic administration includes, but is not limited to, oral, intranasal, rectal and parenteral (e.g., other than through the alimentary tract, such as intramuscular, intravenous, intra-arterial, transdermal and subcutaneous) administration.

The term “co-administering” or “concurrent administration”, when used, for example with respect to the pharmaceutical ingredient (e.g., vector) and/or analogs thereof and another active agent (e.g., multispecific antibody), refers to administration of the pharmaceutical ingredient and/or analogs and the active agent such that both can simultaneously achieve a physiological effect. The two agents, however, need not be administered together. In some embodiments, administration of one agent can precede administration of the other. Simultaneous physiological effect need not necessarily require presence of both agents in the circulation at the same time. However, in some embodiments, co-administering typically results in both agents being simultaneously present in the body (e.g., in the plasma) at a significant fraction (e.g., 20% or greater, e.g., 30% or 40% or greater, e.g., 50% or 60% or greater, e.g., 70% or 80% or 90% or greater) of their maximum serum concentration for any given dose.

The term “effective amount” or “pharmaceutically effective amount” refer to the amount and/or dosage, and/or dosage regime of one or more pharmaceutical ingredients (e.g., vectors) necessary to bring about the desired result.

The phrase “cause to be administered” refers to the actions taken by a medical professional (e.g., a physician), or a person controlling medical care of a subject, that control and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject. Such prescribing can include, for example, drafting a prescription form, annotating a medical record, and the like.

As used herein, the terms “treating” and “treatment” refer to delaying the onset of, retarding or reversing the progress of, reducing the severity of, or alleviating or preventing either the disease or condition to which the term applies, or one or more symptoms of such disease or condition. The terms “treating” and “treatment” also include preventing, mitigating, ameliorating, reducing, inhibiting, eliminating and/or reversing one or more symptoms of the disease or condition.

The term “mitigating” refers to reduction or elimination of one or more symptoms of that pathology or disease, and/or a reduction in the rate or delay of onset or severity of one or more symptoms of that pathology or disease, and/or the prevention of that pathology or disease. In some embodiments, the reduction or elimination of one or more symptoms of pathology or disease can include, e.g., measurable and sustained decrease of tumor volume.

As used herein, the phrase “consisting essentially of” refers to the genera or species of active pharmaceutical agents recited in a method or composition, and further can include other agents that, on their own do not have substantial activity for the recited indication or purpose.

The terms “subject,” “individual,” and “patient” interchangeably refer to a mammal, preferably a human or a non-human primate, but also domesticated mammals (e.g., canine or feline), laboratory mammals, and agricultural mammals. In various embodiments, the subject can be a human (e.g., adult male, adult female, adolescent male, adolescent female, male child, female child).

The term “viral particle” as used herein refers a macromolecular complex capable of delivering a foreign nucleic acid molecule into a cell independent of another agent. A particle can be a viral particle or non-viral particle. Viral particle includes retroviral particle and lentiviral particle. Non-viral particles are limited to liposomes, nanoparticles, and other encapsulation systems for delivery of polynucleotides into cells.

The abbreviations “a” or “anti-” before the name of a gene refers to an antibody or antigen binding fragment of an antibody (such as an scFv) that specifically binds to a target. For example, αCD19 refers to an anti-CD19 antibody or antigen binding fragment thereof and αCD3 refers to an anti-CD3 antibody or antigen binding fragment thereof.

As used herein, the terms “expression cassette” or “vector genome” refer to a DNA segment that is capable in an appropriate setting of driving the expression of a polynucleotide (a “transgene” or “payload”) encoding a polypeptide (e.g., chimeric antigen receptor) that is incorporated in said expression cassette. When introduced into a host cell, an expression cassette inter alia is capable of directing the cell's machinery to transcribe the transgene into RNA, which is then usually further processed and finally translated into the polypeptide. The expression cassette can be comprised in a particle (e.g., viral particle). Generally, the term expression cassette excludes polynucleotide sequences 5′ to the 5′ ITR and 3′ to the 3′ ITR.

The terms “transgene” or “payload” refer to the transferred nucleic acid itself. The transgene may be a naked nucleic acid molecule (such as a plasmid) or RNA. The transgene may include a polynucleotide encoding one or more polypeptides (e.g., chimeric antigen receptor). The transgene may include a polynucleotide encoding one or more heterologous protein (e.g., a chimeric antigen receptor), one or more capsid proteins, and other proteins necessary for transduction of the polynucleotide into a target cell.

The term “derived” is used to indicate that the cells have been obtained from their biological source and grown or otherwise manipulated in vitro (e.g., cultured in a growth medium to expand the population and/or to produce a cell line).

The term “transduce” refers to introduction of a nucleic acid into a cell or host organism by way of a particle (e.g., a lentiviral particle). Introduction of a transgene into a cell by a viral particle can therefore be referred to as “transduction” of the cell. The transgene may or may not be integrated into the genomic nucleic acid of a transduced cell. If an introduced transgene becomes integrated into the nucleic acid (genomic DNA) of the recipient cell or organism it can be stably maintained in that cell. Alternatively, the introduced transgene may exist in the recipient cell or host organism extra-chromosomally, or only transiently. A “transduced cell” is therefore a cell into which the transgene has been introduced by way of transduction. Thus, a “transduced” cell is a cell into which, a polynucleotide has been introduced.

The term “transduction efficiency” is an expression of the proportion of cells that express or transduce a transgene when a cell culture is contacted with particles. In some embodiments, the efficiency can be expressed as the number of cells expressing a transgene when a given number of cells are contacted with a given number of particles. In some embodiments, “Relative transduction efficiency” is the proportion of cells transduced by a given number of viral particles in one condition relative to the proportion of cells transduced by that same number of particles in another condition comprising a similar number of cells of the same cell type. Relative transduction efficiency is most often used to compare the effects of a modulator of transduction efficiency on cells and/or animals treated or not treated with that modulator.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

The section headings used herein are for organizational purposes only and are not to be construed as limiting the subject matter described.

While illustrative embodiments have been illustrated and described, it will be appreciated that various changes can be made therein without departing from the spirit and scope of the invention.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what is regarding as the invention.

### Example 1: Lentiviral Particle Production and In Vitro Transduction

To generate lentiviral particles, a VT103 transgene plasmid which expresses mCherry (Red Fluorescence Protein derived from Discosoma) in place of a chimeric antigen receptor (CAR) and contains the rapamycin activated cytokine receptor (RACR) was co-transfected into 293 cells with an envelope plasmid encoding the Cocal G protein and a membrane tethered anti-CD3 antibody.

### Virus Production

1.2e6 293T cells were seeded into TC-treated 6 well plates in a total volume of 2.5 ml Complete DMEM media. 24 hours later, cells were transfected. (protocol written for 1 well of 6 well plate; all reagents should be room temperature)

The following DNA was added to 500 ul serum free OptiMEM™ media: 2 ug transfer plasmid, 1 ug Gag/pol plasmid, 1 ug REV plasmid, 1 ug Cocal envelope plasmid. 15 ul (15 ug) PEI was then added to the media/DNA mix. Mixture was mixed well and incubated at room temperature for 20 minutes. The media/DNA/PEI mix was then added to 2.5 ml fresh Complete DMEM media. The seeding media in 293T-containing well was removed and replaced with fresh media containing the transfection reagents and placed in 37° C. humidified incubator. 48 hours later, the supernatant was collected and filtered through a 0.45 um PVDF filter. The supernatant was concentrated using Amicon-Ultra 15 100K column and centrifuged at 3000×g for 30 minutes at 4° C. The virus was then stored at 4° C. until use.

### 293T Transduction Titers

1e5 293T cells were seeded into TC-treated 12 well plates in 1 ml Complete DMEM media. 24 hours later, empty wells were counted 3× to calculate titer. Then add virus to wells in the amount: 2 ul, 1 ul, 0.5 ul, 0.2 ul, 0.1 ul, 0.05 ul virus per well. Virus was diluted 1:100 before adding to 293T cells. 3 days later, 293T cells were harvested for analysis by flow cytometry. Media was removed, cells were washed in PBS, cells were then washed in Trypsin and incubate for ˜3-5 minutes in 37° C. incubator. Cells were resuspended in 1 ml FACS buffer and ˜100-200 ul were added to a 96 well V bottom plate. Flow cytometry analysis was performed for mCherry expression.

293T Titer calculation:

TU/ml=(#of cells at time of transduction x % mCherry+x 100)/(vector volume in ul×1000)

### PBMC Transduction and Staining for Flow Cytometry

PBMCs were thawed, resuspended into 10 ml RPMI complete media, and the PBMCs were diluted to 1e6 cells/ml in complete RPMI media. IL-2 was added to a final concentration of 50U/ml. PBMCs were separated into 3× groups of 15 ml each for various stimulations:
Group #1—Blinatumomab added to a final concentration of 1 ng/ml
Group #2-15e6 αCD3/αCD28 Dynabeads added
Group #3—IL-2 only

For each group, 5 ml of stimulation solution was added to 3× wells of Non-TC treated 6 well plate and incubated at 37° C. for 48 hours. Beads were removed from applicable wells, cells were counted and pelleted. Cells were then resuspended in fresh complete RPMI media at 1e6 cells/ml with IL-2 at a final concentration of 50U/ml. 1 ml (1e6 cells) was added to the wells of a Non TC-treated 12 well plate. 5 ul and 25 ul of Amicon-concentrated Preps were added to the plate and then placed in 37° C. incubator.
5 ul=multiplicity of infection (MOI)˜0.25
25 ul=MOI˜1

After 4 days, cells were resuspended with 200 ul into wells in a 96 well V-bottom plate. Cells were then washed with 200 ul FACS buffer. The cell pellets were resuspended in 100 ul PBS containing Live/Dead™ Stain (1:1000) and incubate at 4° C. for 20 min followed by another wash in 200 ul FACS buffer. Cells were resuspended in 50 ul of surface antibody cocktail (Anti-CD3-PerCP, Anti-CD19-FITC, Anti-CD56-APC, and Anti-CD25-BV421), incubated for 20 min at 4° C., washed in 200 ul FACS buffer, and resuspended in 100 ul FACS buffer and analyzed by flow cytometry.

### Results and Conclusions

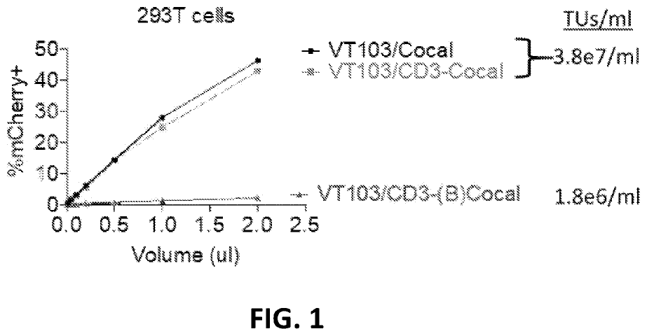

As shown in FIG. 1, lentiviral particles packaged with the αCD3-Cocal envelope plasmid (SEQ ID NO: 129) were successfully packaged and exhibit similar 293T titers compared to vectors produced with the regular Cocal envelope. Minimal transduction was observed with vector pseudotyped with the “blinded” Cocal envelope containing the R354Q mutation. This was expected as the R354Q mutation has been shown to limit binding of the VSV-G (˜70% homologous to Cocal) envelope protein to the low-density lipoprotein receptor.

In order to assess vector-induced activation and transduction, concentrated vector preparations were added to human PBMCs stimulated with either IL-2 alone, Blinatumomab+IL-2, or anti-CD3/anti-CD28 beads+IL-2 as described above. 4 days later the PBMCs were harvested and stained for flow cytometry analysis as described above. αCD3-Cocal-pseudotyped vectors activated and transduced non-stimulated human PBMCs.

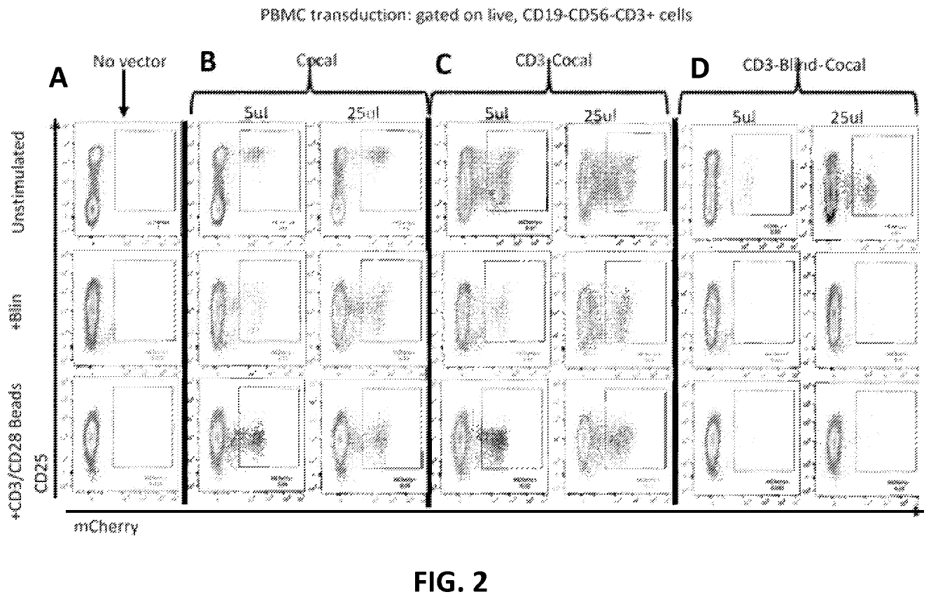

T cells were analyzed for CD25 expression (FIG. 2). Blinatumomab and αCD3/αCD28 beads potently activated the T cells as evidenced by greatly increased CD25 expression. αCD3-Cocal, but not regular Cocal-pseudotyped vectors (FIG. 2B), were also able to activate unstimulated PBMCs to a similar degree as Blinatumomab alone (FIG. 2C). The increased level of activation induced by the αCD3-Cocal envelope corresponded to an increased level of mCherry transgene expression in the unstimulated PBMCs. Although similar levels of activation on T cells stimulated with the Blinded Cocal envelope were detected, high levels of mCherry transgene expression were not detected (FIG. 2D). αCD3-Cocal-pseudotyped lentiviral vectors activate unstimulated PBMCs and lead to successfully transduction and transgene expression.

Off target transduction in the cultures was examined via mCherry expression on NK cells (Live, CD3−, CD19−, CD56+) and B cells (Live, CD3−, CD56−, CD19+) (data not shown). Very minimal levels of mCherry expression were seen in both NK cells and B cells signifying off target transduction is a very rare event in these cultures.

This data shows that αCD3-Cocal vectors can be packaged successfully with similar 293T titers to vectors expressing the regular Cocal envelope. When added to unstimulated human PBMCs, the vectors induced T cell activation, as shown by increased CD25 expression, and transduction. The level of transduction was greatest for the unstimulated PBMCs transduced with αCD3-Cocal vector. The data show that αCD3-Cocal expressing viral vector particles potently activate and transduce unstimulated PBMCs.

αCD3-Cocal-pseudotyped vectors containing a “blinding” mutation in the Cocal coding sequence (R345Q) were analyzed for their ability to transduce unstimulated PBMCs. Although activation was induced by these particles, there was a very small amount of mCherry expression, indicating that this vector transduced T cells at a low level.

Other cells in the PBMC culture were analyzed for expression of mCherry as an indication of off-target transduction from the vector. Transduction of NK cells or B cells was not observed.

The data show that particles containing αCD3-Cocal envelopes activate and transduce unstimulated PBMCs in vitro, and as such, these particles are suitable candidates for in vivo CAR T cell manufacturing.

### Example 2: Comparing Cocal Vs αCD3-Cocal Vs αCD3-Cocal (Blinded) Viral Particle Envelopes and In Vitro Transduction of T Cells

This study assessed in vitro transduction of T cells by a lentiviral particle surface engineered with the Cocal glycoprotein and packaged transgenes containing an αCD19 CAR in addition to the RACR component. The experimental readout was transduction of non-stimulated PBMCs as measured by CAR+ T cells by flow cytometry. This study also incorporated staining with an anti-2A flow reagent which is specific to the 2A cleavage peptides in the vector transgene.

Another aim of this study was to examine vector particle production using two envelope plasmid backbones. One used the CMV promoter and the other used the MND promoter. The MND promoter-containing plasmid also had a Woodchuck Hepatitis Virus Posttranscriptional regulatory element (WPRE) sequence. A WPRE sequence is included to enhance transcription of the viral particle payload.

In addition to examining % CAR+ T cells, viral particles were analyzed by their ability to secrete cytokines after stimulation with CD19+ Raji cells.

### PBMC Transduction and Staining for CAR+ Cells Flow Cytometry

PBMCs were thawed, resuspended into 10 ml RPMI complete media, and the PBMCs were diluted to 1e6 cells/ml in complete RPMI media. IL-2 was added to a final concentration of 50U/ml. PBMCs were separated into 2× groups for various stimulations:
Group #1—Blinatumomab added to a final concentration of 1 ng/ml
Group #2—IL-2 only

For each group, 5 ml of stimulation solution was added to Non-TC treated 6 well plate and incubated at 37° C. for 3 days. Cells were counted and pelleted. Cells were then resuspended in fresh complete RPMI media at 1e6 cells/ml with IL-2 at a final concentration of 50U/ml. 1 ml (1e6 cells) was added to the wells of a Non-TC-treated 24 well plate. 100 ul of Amicon-concentrated Preps were added to the plate, mixed, and then placed in 37° C. incubator.

After 4 days, Rapamycin was added to a final concentration of 10 nM. After 11 days, (15 days after virus) cells were mixed and 200 ul added to wells in a 96 well V-bottom plate. Cells were then washed with 200 ul FACS buffer. The cell pellets were resuspended in 100 ul PBS containing Live/Dead™ Stain (1:1000) and incubate at 4° C. for 20 min followed by another wash in 200 ul FACS buffer. Cells were resuspended in 50 ul of FACS buffer+CD19-FITC conjugate (2 ug/ml), incubated for 20 min at 4° C., washed in 200 ul FACS buffer, resuspended in 100 ul BD Cytofix/Cytoperm™, and incubated at 4° C. for 20 min.

Cells were then washed in 1×BD Perm/Wash™ buffer (“Perm Wash”) buffer, resuspended in 50 ul 1× Perm Wash with anti-2A-af647 (1:100), incubated at 4° C. for 20 min, washed in 1× Perm Wash buffer, resuspended in 100 ul FACS buffer and analyzed by flow cytometry.

### Raji Stimulation for Cytokine Production

13 days after transduction, 250 ul of PBMCs were added to 100,000 Raji cells in a 96 well V-bottom plate. Cells were pelleted and resuspended in 100 ul cell stimulation media containing golgi inhibitors Breldin A (1:1000) and Monensin (1:1000). Cells were briefly centrifuged to pellet, incubated for 5 hours, washed cells with 200 ul FACS buffer, resuspended in 100 ul PBS containing Live/Dead™ Stain (1:1000), incubated at 4° C. for 20 min, washed in 200 ul FACS buffer, resuspended in 50 ul of surface antibody cocktail diluted in FACS buffer, and incubated for 20 min at 4° C.

Diluted surface antibody cocktails used:
a. Anti CD3-Percp 1:100
b. Anti CD4-BV650
c. Anti-CD8-BV605

Following incubation, cells were washed in 200 ul FACS buffer, resuspended in 100 ul of BD Cytofix/Cytoperm™, incubated at 4° C. for 20 min, washed in 1× Perm Wash buffer, resuspended in 50 ul intracellular antibody cocktail diluted in 1× Perm Wash, and incubated at 4° C. for 20 min.

Diluted intracellular antibody cocktails used:
a. Anti-2A-af647
b. Anti-IL-2-PEcy7
c. Anti-IFNg-BV421
d. Anti-GzmB-FITC
e. Anti-TNFa-PE

Following incubation, cells were washed in 1× Perm Wash buffer, resuspended in 100 ul FACS buffer and analyzed by flow cytometry.

### Results and Conclusions

Cells were transduced with following plasmids:
1. VT103 transgene plasmid which expresses mCherry in place of the anti-CD19 scFv CAR with the MND promotor and Cocal surface envelope (SEQ ID NO: 10) protein (VT103 MND-Cocal)
2. Transgene plasmid containing the αCD19 CAR in addition to the RACR component with the MND promotor and Cocal surface envelope (SEQ ID NO: 10) protein (RACR-αCD19 MND-Cocal)
3. Transgene plasmid containing the αCD19 CAR in addition to the RACR component with the CMV promotor and Cocal surface envelope (SEQ ID NO: 10) protein (RACR-αCD19 CMV-Cocal)
4. Transgene plasmid containing the αCD19 CAR in addition to the RACR component with the CMV promotor and αCD3-Cocal surface envelope proteins (SEQ ID NO: 129) (RACR-αCD19 CMV-αCD3-Cocal)
5. Transgene plasmid containing the αCD19 CAR in addition to the RACR component with the CMV promotor and αCD3-Blinded Cocal variant surface envelope proteins (RACR-αCD19 CMV-αCD3-(B)Cocal)

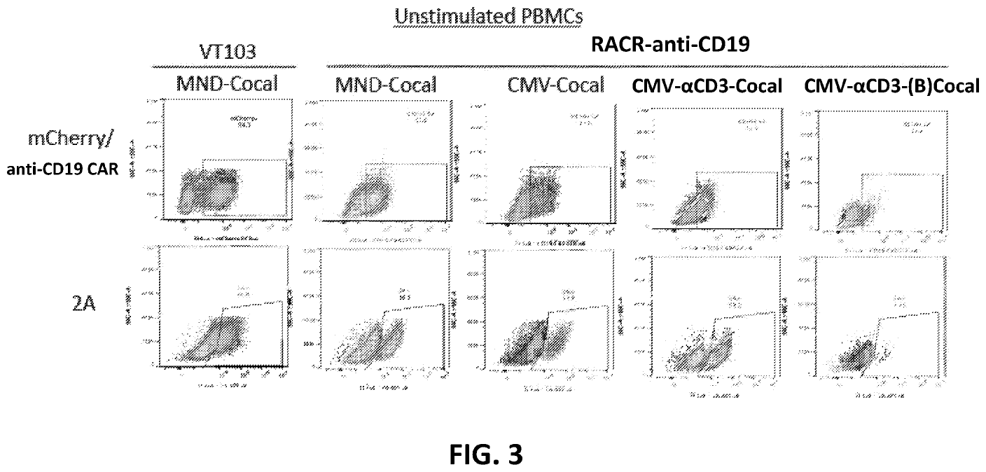

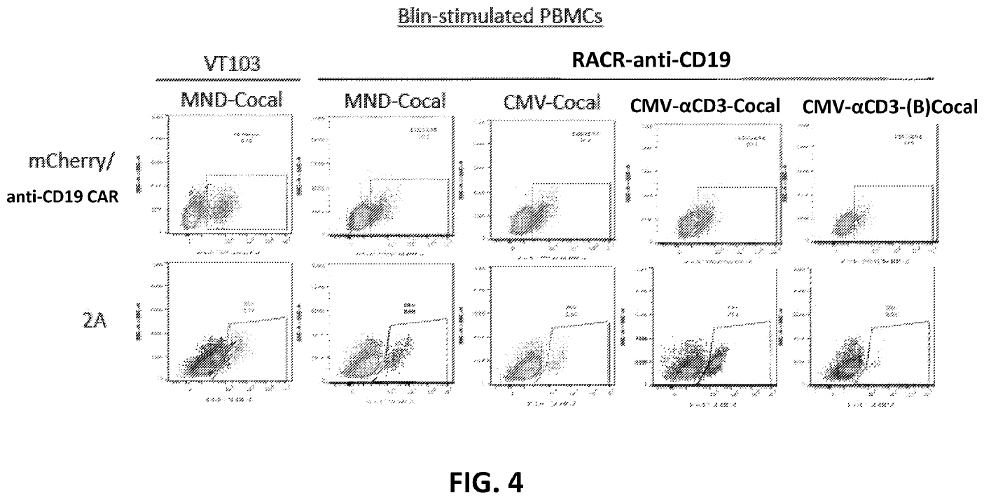

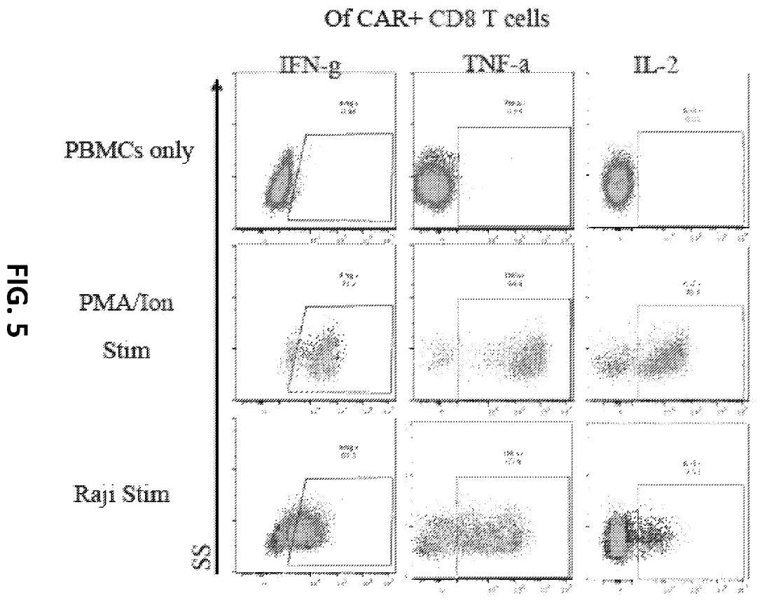

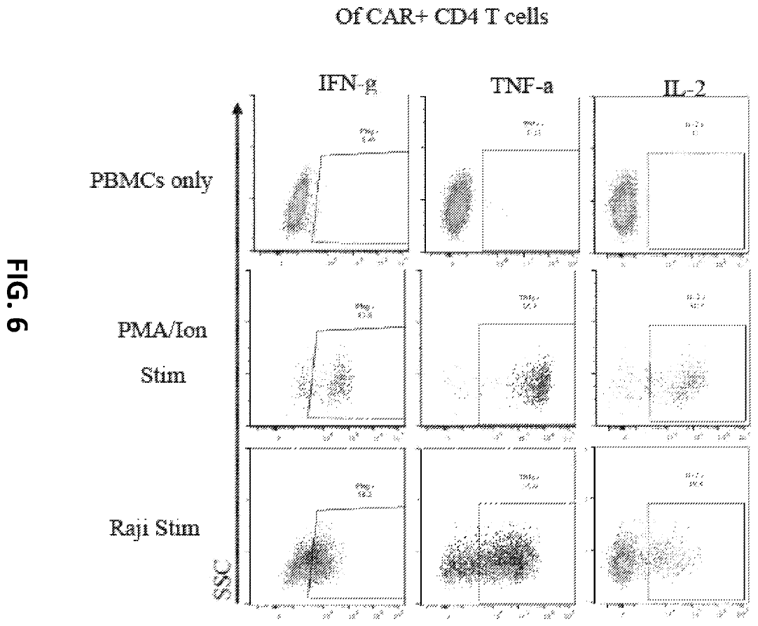

11 days after rapamycin addition (10 nM final concentration), the PBMCs were harvested and analyzed by flow cytometry for αCD19 CAR and intracellular 2A expression. Following culture with rapamycin, both the unstimulated (FIG. 3) and Blinatumomab-stimulated (FIG. 4) PBMC cells showed enhanced expression of the αCD19 CAR. Staining with the anti-2A reagent was successful and gave better separation of positive and negative cells than the CD19-FITC reagent (FIG. 3 and FIG. 4, bottom panels). The data show that staining for the 2A peptide is a viable alternative to staining for surface expression of the CAR.

A stimulation assay to determine CAR T cell functionality was performed. 100,000 PBMCs from the non-stimulated, RACR-αCD19 CAR/αCD3-Cocal-transduced well were cocultured for 5 hours with 100,000 Raji cells in the presence of golgi inhibitors brefeldin A and Monensin. The cells were then harvested, stained for surface markers and intracellular cytokines, and analyzed by flow cytometry. Upon Raji stimulation, both CD8 (FIG. 5) and CD4 (FIG. 6) CAR+ T cells readily produced IFNg, IL-2 and TNFa, to similar levels as PMA+Ionomycin-stimulated controls. As a further control, there was no cytokine production in the PBMC only negative control wells. These data show that αCD3-Cocal-pseudotyped vector particles encoding a RACR-αCD19 CAR payload can successfully transduce non-stimulated PBMCs. Furthermore, culture with rapamycin for ˜2 weeks greatly enhanced surface expression of the αCD19 CAR. The CAR+ cells were still highly functional after rapamycin culture as evidenced by production of IFNg, IL-2, and TNFa upon Raji stimulation. Thus, non-stimulated PBMCs transduced with RACR-αCD19/αCD3-Cocal vectors and cultured in Rapamycin are functional and produce IFNg, IL-2 and TNFa upon Raji cell stimulation.

αCD3-Cocal-pseudotyped viral particles can be packaged with RACR-αCD19 CAR payloads. After extended culture in rapamycin, the particles were capable of transducing non-stimulated PBMCs. Upon stimulation with CD19+ Raji cells, the αCD19 CAR-transduced T cells potently produced IFNg, IL-2, and TNFa cytokines indicating a potent Th1-like phenotype and high degree of functionality.

This study also showed that vector packaging and 293T transduction with envelopes expressed in the MND promoter backbone is greater than with the CMV-driven envelope plasmids.

### Example 3: Comparing Cocal Vs αCD3-Cocal Viral Particle Envelopes with the αCD19 CAR-TGFβ Payload and In Vitro Transduction of T Cells

The aim of this study was to determine the effect of αCD3-Cocal-pseudotyped viral particle payload on the detectability and function of the lentiviral particles comprising of the following payload vectors: αCD19 CAR-TGFβ payload, and RACR-αCD19 CAR payload. Two payload designs were evaluated for their ability to activate and transduce unstimulated human PBMCs as compared to regular Cocal-pseudotyped vector viral particles comprising the same payloads.

### Virus Production

28e6 293T cells were seeded into 16×T175 flasks (8× per vector) with 28e6 293T cells each in a total volume of 25 ml Complete DMEM media. 24 hours later, cells were transfected. (protocol written for 1×T175 flask scale; all reagents should be at 37° C.)

The following DNA was added to 1 ml serum free OptiMEM™ media (without additives): 12 ug transfer plasmid, 6 ug Gag/pol plasmid, 6 ug REV plasmid, 6 ug envelope plasmid. 90 ul (90 ug) PEI was then added to the media/DNA mix. Mixture was mixed well and incubated at room temperature for 20 minutes. The media/DNA/PEI mix was then added to 25 ml fresh Complete DMEM media. The seeding media in 293T-containing well was removed and replaced with fresh media containing the transfection reagents and placed in 37° C. humidified incubator. 48 hours later, the supernatant was collected and stored in the fridge and replaced with fresh DMEM media. The next day, (72 hours) the supernatant was collected and filtered through a 0.45 um PVDF filter. The supernatant was concentrated using Amicon-Ultra 15 100K column and centrifuged at 3000×g for 30 minutes at 4° C. The virus was then stored at 4° C. until use.

List of Virus preps made for study:
1. αCD19 CAR-TGFbDN/MND-Cocal, Titer=4e7 TU/ml
2. αCD19 CAR-TGFbDN/CMV-αCD3-Cocal, Titer=1.8e7 TU/ml

### PBMC Transduction and Staining for Flow Cytometry

50e6 PBMCs were thawed, diluted to 1e6 cells/ml in complete RPMI media. IL-2 was added to a final concentration of 50U/ml. PBMCs were separated into 2× groups for various stimulations:
Group #1—IL-2 only (50U/ml)
Group #2—αCD3/αCD28 Dynabeads added

Each group was incubated at 37° C. overnight. Beads were removed from applicable wells, cells were counted and pelleted. Cells were then resuspended in fresh complete RPMI media at 1e6 cells/ml with IL-2 at a final concentration of 50U/ml. 500 ul (5e5 cells) were added to the wells of a Non-TC-treated 48 well plate. MOI=2, 1, and 0.5 (based on 293T titer) of Amicon-concentrated Preps were added to the plate and then placed in 37° C. incubator.
a. αCD19 CAR-TGFb/αCD3-Cocal (50 ul, 25 ul, and 12.5 ul)
b. αCD19 CAR-TGFb/Cocal (25 ul, 12.5 ul, 6.25 ul)

After 3 days, vector was washed out and replaced with 500 ul fresh RPMI media+IL-2 (50U/ml). Cells were mixed and 100 ul were added to wells in a 96 well V-bottom plate for activation flow cytometry analysis. Cells were then washed with 200 ul FACS buffer. The cell pellets were resuspended in 100 ul PBS containing Live/Dead™ Stain (1:1000) and incubated at 4° C. for 20 min followed by another wash in 200 ul FACS buffer. Cells were resuspended in 50 ul of FACS buffer+surface stain cocktail, incubated for 30 min at 4° C., washed in 200 ul FACS buffer.

Diluted surface stain cocktail used:
a. CD19-FITC 1:50
b. Anti-CD3-Percp 1:100
c. Anti-CD4-BV650 1:200
d Anti CD8-BV605 1:200
e. Anti-CD25-PECy7 1:100

Cells pellets were then resuspended in 100 ul of BD Cytofix/Cytoperm™, incubated at 4° C. for 20 min, washed in 1× Perm Wash buffer, resuspended in 50 ul 1× Perm Wash with anti-2A-af647 (1:100), incubated at 4° C. for 30 min, washed in 1× Perm Wash buffer, resuspended in 100 ul FACS buffer and analyzed by flow cytometry.

3 days later, after CD25 analysis, cells were mixed and 100 ul were added to wells in a 96 well V-bottom plate for transduction flow cytometry analysis. Cells were washed with 200 ul FACS buffer, resuspended in 100 ul PBS containing Live/Dead™ Stain (1:1000), incubated at 4° C. for 20 min, washed in 200 ul FACS buffer, resuspended in 50 ul of FACS buffer+surface stain cocktail and incubated for 30 min at 4° C.

Diluted surface stain cocktail used:
a. CD19-FITC 1:50
b. Anti-CD3-Percp 1:100
c. Anti-CD4-BV650 1:200
d. Anti CD8-BV605 1:200

Following incubation, cells were washed in 200 ul FACS buffer, resuspended in 100 ul of BD Cytofix/Cytoperm™, incubated at 4° C. for 20 min, washed in 1× Perm Wash buffer, resuspended in 50 ul 1× Perm Wash with anti-2A-af647 (1:100), incubated at 4° C. for 30 min, washed in 1× Perm Wash buffer, resuspended in 100 ul FACS buffer and analyzed by flow cytometry.

### Results and Conclusions

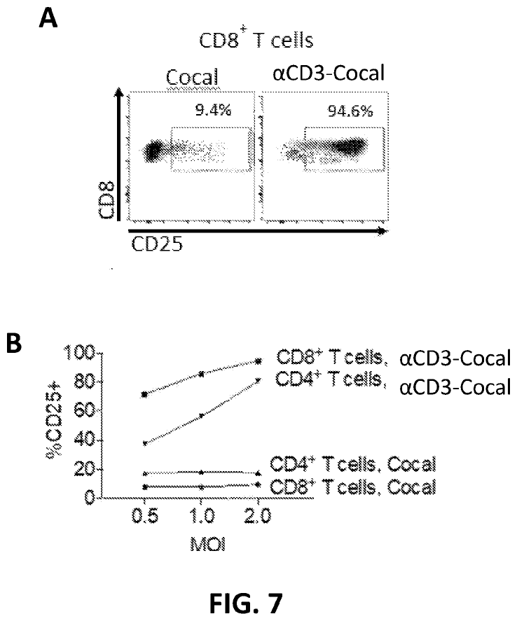

To assess if αCD19 CAR-TGFβDN/αCD3-Cocal viral vector particles can activate human T cells, the vector particles were added to human PBMCs at several MOI's. 3 days later, the virus was removed and the cells were given fresh media and analyzed for the activation marker CD25. αCD19 CAR-TGFβDN/αCD3-Cocal particles potently activated both CD4 and CD8 T cells (FIGS. 7A and 7B). Furthermore, CD25 upregulation was dose-dependent (FIG. 7B). Viral particles pseudotyped with the regular Cocal envelope induced minimal levels of CD25 compared to the αCD3-Cocal particles (FIG. 7A).

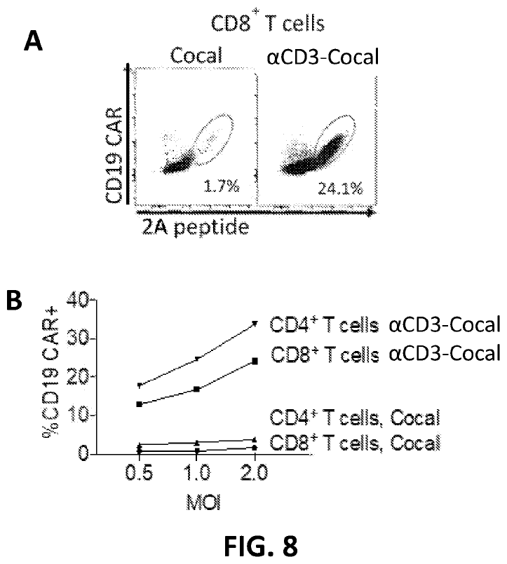

To examine transduction, 6 total days after vector addition, samples were analyzed for αCD19 CAR and 2A expression. Mirroring the CD25 expression on day 3, αCD3-Cocal-pseudotyped particles were capable of transducing unstimulated PBMCs while Cocal-pseudotyped particles were not (FIG. 8A). Furthermore, transduction occurred in a dose-dependent manner for both CD4 and CD8 T cells (FIG. 8B). The data show that αCD19 CAR-TGFβDN/αCD3-Cocal particles efficiently activate and transduce unstimulated PBMCs in vitro.

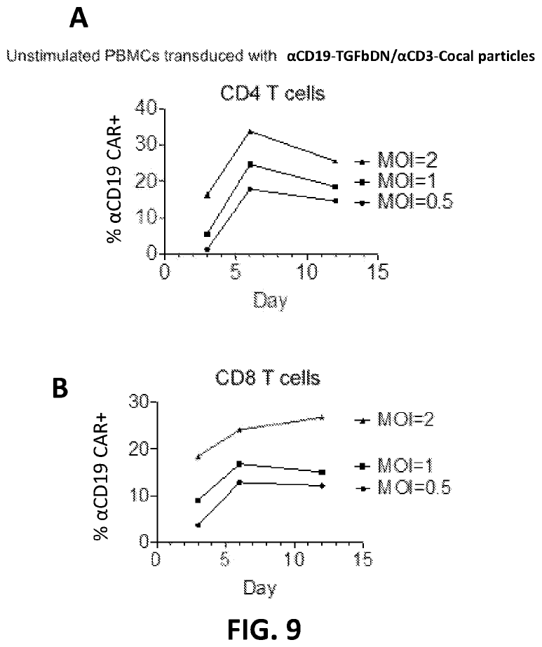

In addition to analyzing αCD19 CAR expression on unstimulated PBMCs at day 6 after transduction, CAR expression on day 3 (when activation was analyzed) and again at day 12 was also analyzed to determine the optimal timing for CAR expression analysis. For both CD4 (FIG. 9A) and CD8 (FIG. 9B) T cells across all MOIs tested, it was found that the percent of T cells expressing the αCD19 CAR peaked at day 6 and remained relatively stable until day 12 (FIG. 9). These data indicate that examining transduction efficiency as measured by CAR surface expression by flow cytometry is optimal at approximately 1 week after transduction.

This study demonstrated the ability of the αCD3-Cocal envelope construct to deliver payloads consisting of a αCD19 CAR to unstimulated PBMCs in vitro. The αCD3-Cocal envelope induced activation of T cells as measured by CD25 expression and this activation correlated with transduction as measured by % of T cells expressing the αCD19 CAR and the 2A peptide. Furthermore, activation and transduction occurred in a dose-dependent manner. In this study, it was also found that αCD19 CAR surface expression, as analyzed by flow cytometry, peaked at approximately day 6 and was similar at day 12. This data further supports the use of αCD3-Cocal-pseudotyped vectors to deliver CAR payloads to unstimulated PBMCs in vitro and in vivo.

### Example 4: Comparing αCD19 CAR-RACR Orientations and In Vitro Transduction of T Cells

The aim of this study was to assess the construct arrangement with the highest αCD19 CAR expression after transduction.
Construct Orientation A) FRB-P2A-RACR-P2A-αCD19 CAR (SEQ ID NO: 81)
Construct Orientation B): αCD19 CAR-P2A-FRB-P2A-RACR (SEQ ID NO: 61)

### Virus Production

28e6 293T cells were seeded into 6×T175 flasks (1× per vector) with 28e6 293T cells each in a total volume of 25 ml Complete DMEM media. 24 hours later, cells were transfected. (protocol written for 1×T175 flask scale; all reagents should be at 37° C.)

The following DNA was added to 2.5 ml serum free DMEM media (without additives): 30 ug transfer plasmid, 15 ug Gag/pol plasmid, 15 ug REV plasmid, and 15 ug envelope plasmid (SEQ ID NO: 130 or 128). 225 ul (225 ug) PEI was then added to the media/DNA mix. The mixture was mixed well and incubated at room temperature for 20 minutes. The media/DNA/PEI mix was then added to 25 ml fresh Complete DMEM media. The seeding media in 293T-containing well was removed and replaced with fresh media containing the transfection reagents and placed in a 37° C. humidified incubator. 48 hours later, the supernatant was collected and stored in the fridge and replaced with fresh DMEM media. The next day, (72 hours) the supernatant was collected and filtered through a 0.45 um PVDF filter. The supernatant was concentrated using an Amicon-Ultra 15 100K column and centrifuged at 3000×g for 30 minutes at 4° C. The virus was then stored at 4° C. until use.

Viral vector particles used in the study:
1. αCD19 CAR-TGFbDN/Cocal (SEQ ID NO: 92), Titer=5.7e7 TU/ml
2. αCD19 CAR-TGFbDN/αCD3-Cocal (SEQ ID NO: 92), Titer=4.1e7 TU/ml
3. RACR-αCD19 CAR/Cocal, Titer=1.3e7 TU/ml
4. RACR-αCD19 CAR/αCD3-Cocal, Titer=4.5e6 TU/ml
5. αCD19 CAR-RACR/Cocal, Titer=2.8e7 TU/ml
6. αCD19 CAR-RACR/αCD3-Cocal, Titer=1e7 TU/ml

### PBMC Transduction and Staining for Flow Cytometry

PBMCs were thawed, diluted to 1e6 cells/ml in complete RPMI media. IL-2 was added to a final concentration of 50U/ml. PBMCs were separated into 2× groups for various stimulations:
Group #1—IL-2 only (50U/ml)
Group #2—+αCD3/αCD28 Dynabeads added

Each group was incubated at 37° C. overnight. Beads were removed from applicable wells, cells were counted and pelleted. Cells were then resuspended in fresh complete RPMI media at 1e6 cells/ml with IL-2 at a final concentration of 50U/ml. 500 ul (5e5 cells) were added to the wells of a Non-TC-treated 48 well plate. MOI=1.5 (based on 293T titer) of Amicon-concentrated Preps were added to the plate and then placed in 37° C. incubator. Transgenes, envelope proteins, 293 titer and ul needed to reach 1.5 MOI are shown in Table L

TABLE 1

| | | | | ul needed |
|---|---|---|---|---|
| | | | | for |
| Group | Transgene | Envelope | 293T titer | MOI = 1.5 |
| 1 | αCD19 CAR TGFb | Cocal | 5.70E+07 | 13.2 |
| 2 | αCD19 CAR TGFb | αCD3-Cocal | 4.10E+07 | 18.3 |
| 3 | RACR-αCD19 CAR | Cocal | 1.30E+07 | 57.7 |
| 4 | RACR-αCD19 CAR | αCD3-Cocal | 4.50E+06 | 166.7 |
| 5 | αCD19 CAR-RACR | Cocal | 2.80E+07 | 26.8 |
| 6 | αCD19 CAR-RACR | αCD3-Cocal | 1.00E+07 | 75.0 |

After 3 days, vector was washed out and replaced with 500 ul fresh RPMI media+IL-2 (50U/ml). Cells were mixed and 100 ul were added to wells in a 96 well V-bottom plate for activation flow cytometry analysis. Cells were then washed with 200 ul FACS buffer. The cell pellets were resuspended in 100 ul PBS containing Live/Dead™ Stain (1:1000) and incubate at 4° C. for 20 min followed by another wash in 200 ul FACS buffer. Cells were resuspended in 50 ul of FACS buffer+surface stain cocktail (see below), incubated for 30 min at 4° C., and washed in 200 ul FACS buffer.

Diluted surface stain cocktail used:
a. CD19-FITC 1:50
b. Anti-CD3-Percp 1:100
c. Anti-CD4-BV650 1:200
d. Anti CD8-BV605 1:200
e. Anti-CD25-PECy7 1:100

Cells pellets were then resuspended in 100 ul of BD Cytofix/Cytoperm™, incubate at 4° C. for 20 min, washed in 1× Perm Wash buffer, resuspended in 50 ul 1× Perm Wash with anti-2A-af647 (1:100), incubated at 4° C. for 30 min, washed in 1× Perm Wash buffer, resuspended in 100 ul FACS buffer and analyzed by flow cytometry.

5 days after CD25 analysis (Day 8 after vector addition), cells were mixed and 100 ul were added to wells in a 96 well V-bottom plate for transduction flow cytometry analysis. Cells were washed with 200 ul FACS buffer, resuspended in 100 ul PBS containing Live/Dead™ Stain (1:1000), incubated at 4° C. for 20 min, washed in 200 ul FACS buffer, resuspended in 50 ul of FACS buffer+surface stain cocktail (see below) and incubated for 30 min at 4° C.

Diluted surface stain cocktail used:
a. CD19-FITC 1:50
b. Anti-CD3-Percp 1:100
c. Anti-CD4-BV650 1:200
d. Anti CD8-BV605 1:200

Following incubation, cells were washed in 200 ul FACS buffer, resuspended in 100 ul of BD Cytofix/Cytoperm™, incubated at 4° C. for 20 min, washed in 1× Perm Wash buffer, resuspended in 50 ul 1× Perm Wash with anti-2A-af647 (1:100), incubated at 4° C. for 30 min, washed in 1× Perm Wash buffer, resuspended in 100 ul FACS buffer and analyzed by flow cytometry.

### Results and Conclusions

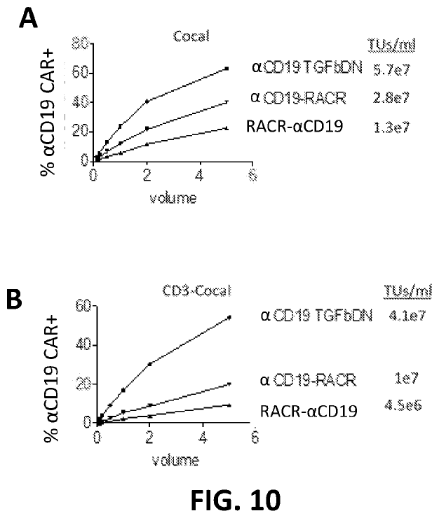

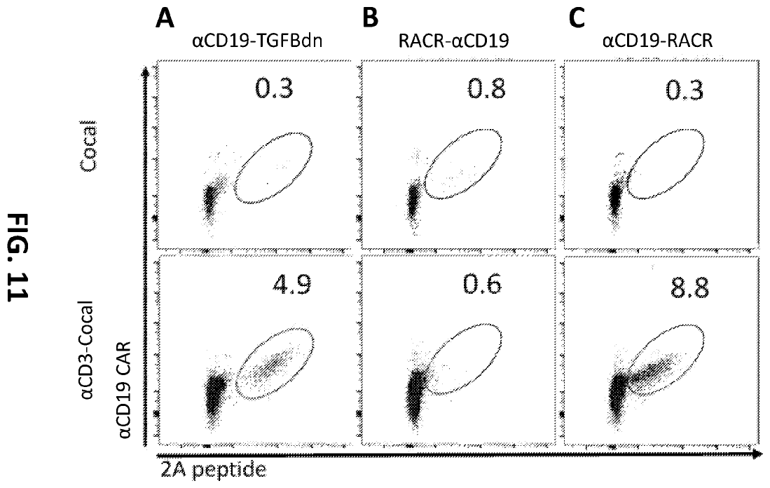

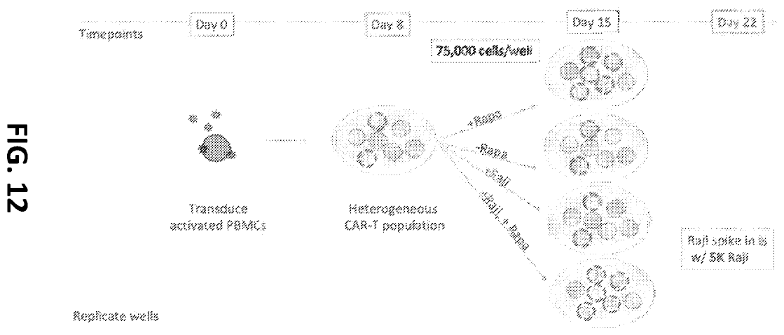

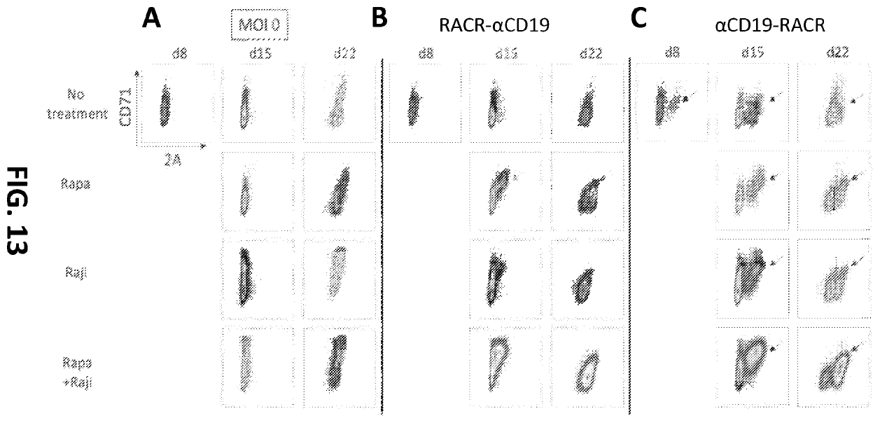

Viral vector particles containing the three transgene plasmids described above were packaged with either Cocal or αCD3-Cocal envelope proteins and preparations were tittered on 293T cells (FIG. 10). Viral vector particles containing αCD19 CAR-TGFbDN transgene exhibited titers higher than preps containing the αCD19 CAR-RACR, in either orientation. When the two RACR-containing constructs were compared, higher titers were achieved when orientating the αCD19 CAR before the RACR components (Construct Orientation B). This was true for both Cocal pseudotyped (FIG. 10A) and αCD3-Cocal-pseudotyped (FIG. 10B) viral particles.

Both analyzed orientations: αCD19 CAR-RACR and RACR-αCD19 CAR particles pseudotyped with αCD3-Cocal envelopes activate unstimulated T cells (data not shown). Concentrated viral vector particles were added to PBMCs in the presence of anti-CD3/anti-CD28 dynabeads+IL-2 or IL-2 alone. 3 days later the PBMCs were washed, the beads were removed, and the PBMCs were resuspended in fresh media containing IL-2. Activation by CD25 expression was then analyzed by flow cytometry. Cocal-pseudotyped vectors did not induce significant upregulation of CD25. In contrast, robust CD25 upregulation was seen on both CD8 and CD4 T cells (data not shown). Similar levels of CD25 were seen in both the RACR-αCD19 CAR and αCD19 CAR-RACR oriented constructs. The data show that both the RACR-αCD19 CAR and the αCD19 CAR-RACR oriented vector particles pseudotyped with αCD3-Cocal potently activate unstimulated T cells.

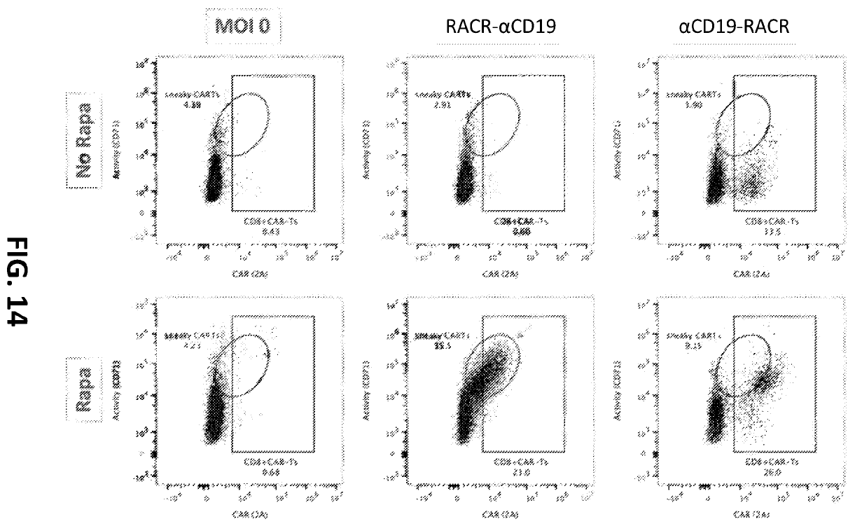

αCD19 CAR and 2A expression in T cells 8 days after vector transduction (5 days after detecting activation) were analyzed to assess T cell transduction. αCD19 CAR surface expression was detected on αCD19 CAR-TGFbDN-transduced cells (FIG. 11A). αCD19 CAR surface expression and intracelluar expression of the 2A peptide were not detected in T cells transduced with the RACR-αCD19 CAR construct (FIG. 11B). In contrast, αCD19 CAR surface expression and intracelluar expression of the 2A peptide were detected in T cells transduced with the αCD19 CAR-RACR construct (FIG. 11C). The lack of transduction seen when using the RACR-αCD19 CAR construct particles was not rescued by the addition of anti-CD3/anti-CD28 stimulation beads prior to viral transduction (data not shown). Similar results were seen for CD4 T cells (data not shown). The data show that αCD19 CAR and RACR orientation are important considerations and that the αCD19-RACR orientation yields higher detectability and manufacturability.

Surprisingly, this data showed that a construct with the CAR 5′ and RACR 3′ both increased the 293T titer of the viral particle and increased the ability to detect the CAR on transduced T cells by flow cytometry. αCD19-RACR transgenes were effectively packaged with the αCD3-Cocal envelope and this particle transduced unstimulated PBMCs in vitro.

### Example 5: Comparing Viral Particle Payload Gene Order and In Vitro Transduction of T Cells

The aim of this study was to determine the effect of order on the detectability and function of a lentiviral payload comprised of the following functional elements: Frb-RACR, and αCD19-CAR. The Frb-RACR element provides a selective advantage to cells when rapamycin is added to the culture. The αCD19-CAR element provides targeting of T cells to CD19+ target cells. The two elements together create CAR-T cells that enrich with rapamycin and are cytotoxic to CD19+ target cells. Two payload designs evaluated differ only in the order in which elements are expressed in the polycistronic transcript. This study tested functional aspects of the payloads in both orientations to see if differences in expression were affecting 1) payload detection by flow cytometry (2A antibody), 2) CAR-T cell rapamycin response, and 3) CAR-T cell CD19+ cell cytotoxicity.

Two payload designs evaluated:
1. MND promoter—FRB-P2A-RACRβ-P2A-RACRγ-P2A-CAR (SEQ ID NO: 81)
2. MND promoter—CAR-P2A-FRB-P2A-RACRβ-P2A-RACRγ (SEQ ID NO: 61)

### Study Protocol

TABLE 2

| Timeline | Activity |
|---|---|
| Day 0 | Thaw and bead-activate PBMCs donor 6094BW |
| | Transduce cells with 0, 0.75, and 1.5 MOIs in a 48-well plate |
| Day 8 | Plate 75,000 cells/well (96-well plate) of from the following |
| | wells: |
| | Bead-activated PBMCs, MOI 1.5 αCD3-cocal RACR- |
| | αCD19-CAR |
| | Bead-activated PBMCs, MOI 1.5 αCD3-cocal αCD19-CAR- |
| | RACR |
| | Bead-activated PBMCs, MOI 0 negative control wells |
| | Apply treatments to wells in duplicate: |
| | No treatment |
| | 10 nM rapamycin |
| | 5,000 Raji cells |
| | 5,000 Rajis + 10 nM rapamycin |
| | Additionally, one extra set of replicate wells +/− Rapa |
| | treatment also received 28,000 counting beads per well. |
| | Flow analysis of representative wells (see flow protocol |
| | below) |
| Day 15 | Count cells with the Vi-Cell Blu |
| | Estimate well volume with a pipet |
| | Flow analysis |
| Day 22 | Count cells with the Vi-Cell Blu |
| | Estimate well volume with a pipet |
| | Flow analysis |

* New media was added to wells as needed, Rapamycin at 10 nM was added to the wells that were treated with Rapamycin. Media was added d10, d14, d15, and d20.

### Results and Conclusions

PBMCs were treated with equal amounts of each of the RACR-αCD19-CAR and αCD19-CAR-RACR vectors. Despite being transduced with the same amount of infection units, a discernable 2A+ population was visible only in the αCD19-CAR-RACR construct on day 8 post-transduction (FIG. 13C, d8: black arrow). Rapamycin selection (10 nM) revealed a 2A+ cell population in cells transfected with the RACR-αCD19-CAR vector at days 15 and 22 (FIG. 13B, red arrows, FIG. 14, red-arrow). In contrast, the αCD19-CAR-RACR T cells formed a well-defined 2A+ population regardless of rapamycin treatment (FIG. 13C, black arrows).

Rapamycin enrichment for αCD19-RACR-CAR-T cells was readily detectable at d15 and continued to enrich through day 22. This enrichment was greatly enhanced when Raji cells were added in addition to rapamycin (data not shown). Enrichment is defined as the increase in the percent of CAR-T cells over time. Enrichment can occur via a decrease in abundance of non-CAR-T cells, and/or an increase in abundance of CAR-T cells.

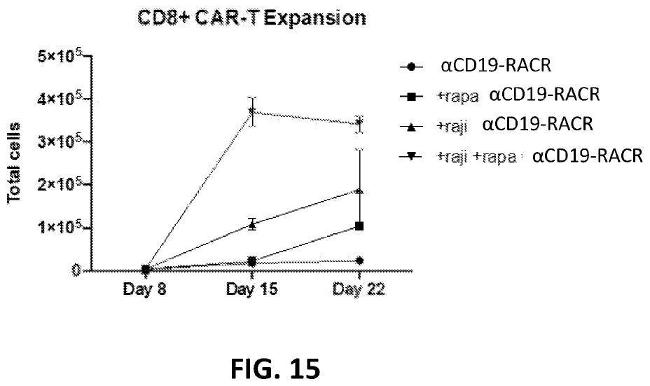

Expansion of CAR-T cells was measured using cell counts, culture volume, and flow cytometry population frequencies to estimate total CAR-T cells. Gating requirements were different for d15 and 22; however, in each case, un-transduced cells were used as a biological negative control. At day 15, there was little rapamycin-driven expansion of cells; however, by day 22 there was significant rapamycin-driven expansion of cells (FIG. 15). Activation through the CAR through the addition of Raji cells produced a larger expansion than the rapamycin-driven expansion, and the largest expansion occurred with both rapamycin addition and Raji cell addition (FIG. 15). This shows that rapamycin is necessary for cellular expansion.

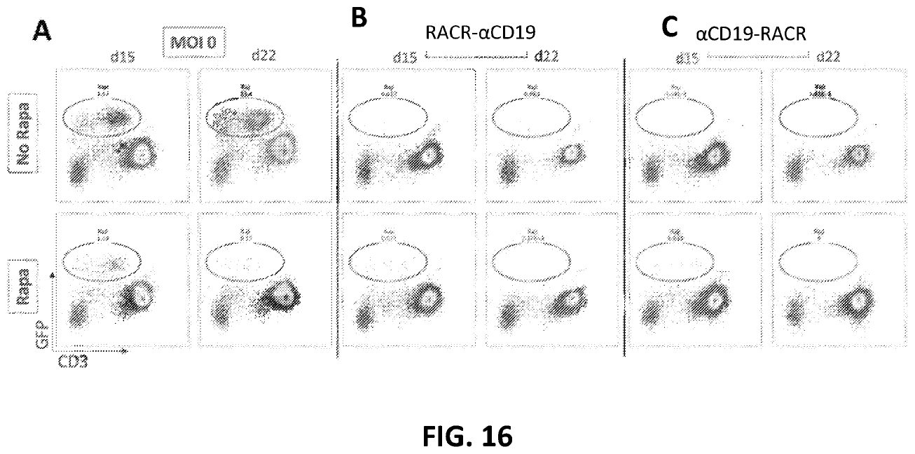

To assess the cytotoxicity of the RACR-αCD19 CAR and αCD19 CAR-RACR vectors against CD19 positive cells, 5,000 Raji GFP::ffluc cells were added to the culture on day 8 post-transduction, with and without rapamycin. The 5,000 Raji cell spike-in corresponds to a 1.3:1 (Effector:T cell) ratio for the αCD19 CAR-RACR. Because RACR-αCD19 CAR was undetectable at d8, the exact E:T were unknown for those samples, but it is likely that was also ˜1.3:1. After 1 week of Raji cell co-culture, Raji cells were eradicated with both αCD19 CAR vectors independent of rapamycin addition (FIGS. 16B and 16C). In the presence of rapamycin, Raji cells were diminished even without the presence of a αCD19-CAR, indicating that rapamycin alone has a profound effect on Raji cell biology (FIG. 16A).

These data showed that detection of the payload by flow cytometry varied with orientation. αCD19 CAR-RACR was detectable across multiple conditions and time points. RACR-αCD19 CAR was not detectable at 8 days after transduction and the vector was most detectable with rapamycin treatment 15 days after transduction/activation. The vector genome ordered with 5′ anti-CD19 CAR and 3′ RACR generates superior cellular expansion and its detectability by flow cytometry is more robust and predictable. Surprisingly, a viral particle whose vector genome has, in 5′ to 3′ order, the polynucleotide sequence encoding the anti-CD19 chimeric antigen receptor and then the polynucleotide sequence encoding the receptor (RACR) results in better transduction efficiency of T cells than a viral particle whose vector genome places to the two polynucleotide sequences in the other order (receptor 5′ to anti-CD19 CAR).

The data also showed that rapamycin enriches for CAR-T cells containing payloads of both orientations. Rapamycin expansion was most pronounced between day 15 and day 22 of the study. Non-rapamycin treated αCD19-CAR-T cells expanded 4.3-fold over non-rapamycin treated cells by day 22 (data not shown). The largest T cell expansion was observed in CAR-T cells treated with Raji cells and rapamycin. The data further showed that both the αCD19 CAR-RACR and RACR-αCD19 CAR payload orientations were cytotoxic to CD19 positive Raji cells, whose growth was negatively impacted by 10 nM rapamycin.

### Example 6: In Vivo Transduction of T Cells by Lentiviral Vector Encoding Anti-CD19 TGFβ-DN CAR

This study assessed in vivo transduction of T cells by a lentiviral particle surface engineered with the Cocal glycoprotein and an anti-CD3 scFv (SEQ ID NO: 129) as described in Example 1. The lentiviral particle contains a polynucleotide encoding an anti-CD19 CAR with a dominant-negative TGFβ receptor designed to provide resistance to TGFβ signaling. The lentiviral particle was delivered via an intraperitoneal or subcutaneous injection into CD34+ humanized mice. The mice used in the study were immune-compromised and contain engrafted human hematopoietic stem cells that generate circulating human T cells and B cells.

### Study Design

### Virus Preparation, Animal Strain, Cell Lines

Regular Cocal and engineered αCD3-Cocal enveloped lentivirus particles carrying an anti-CD19 TGFβ-DN CAR payload (SEQ ID NO: 92) were manufactured by Umoja Biopharma using PEI-mediated transient transfection of adherent 293T cells. These preparations were concentrated using Amicon filters. 19 female CD34+ HSC humanized mice at 19 weeks post-implantation (Jackson laboratory) were housed following institutional guidelines (Fred Hutchinson Cancer Research Center).

### Study Protocol

19 female CD34+ humanized mice were acclimated for one week after receipt. At day −7, blood from all mice was collected for flow cytometry analysis to quantify degree of humanization. Mice were randomized according to their total human CD3 levels into the treatment arms described in Table 3.

TABLE 3
Study Treatment Groups

| | | | Administration | Virus Dose | Virus Dose |
|---|---|---|---|---|---|
| Group | N | Virus type | route* | (Titre Unit) | Volume |
| 1 | 4 | Cocal | IP | 9.1 Million TU | 500 μL |
| | | | | Day 0 | |
| 2 | 4 | Cocal | SC | 9.1 Million TU | 500 μL |
| | | | | Day 0 | |
| 3 | 3 | αCD3 | IP | 9.1 Million TU | 500 μL |
| | | Cocal | | Day 0 | |
| 4 | 4 | αCD3 | SC | 9.1Million TU | 500 μL |
| | | Cocal | | Day 0 | |
| 5 | 3 | Vehicle | SC | Vehicle day 0 | 500 μL |

### Study Timeline

At Study Day 0 (SD0) mice were dosed with virus particles according to the table above and peripheral blood was collected once a week for analysis by flow cytometry for the duration of the study.

At SD28 mice were sacrificed, and peripheral blood, spleen and bone marrow were collected from each mouse for flow cytometry analysis and histology.

### Results and Conclusions

Blood analysis by flow cytometry was used to randomize mice into treatment arms by abundance of human CD3+ cells. To quantify the frequency of human CD3+ cells in the peripheral blood, the following gating strategy was used: Fraction of hCD3+ was multiplied by fraction of humanization (hCD45+) to obtain relative abundance of hCD3+. Human B cells, T cells, and CAR+ cells were quantified by flow cytometry using counting beads.

Some weight loss occurred throughout the study but all groups largely recovered, and no significant trends were observed. At study termination, spleen weights were similar amongst the groups.

T cells, B cells, CD71+ T cells (a marker of activation) and CAR+ cells were quantified in the peripheral blood throughout the study. T cell numbers fluctuated throughout the study as did activation levels without significant trends observed (data not shown). While the abundance of human B cells gradually dropped during the study in all groups due to drift in the CD34-humanized mouse model, total B cell depletion was observed in the αCD3-Cocal and cocal IP-dosed groups by day 7 (data not shown). On day 14, CAR+ T cells were detected above background levels only in the blood of mice treated with αCD3-Cocal via the IP route (data not shown).

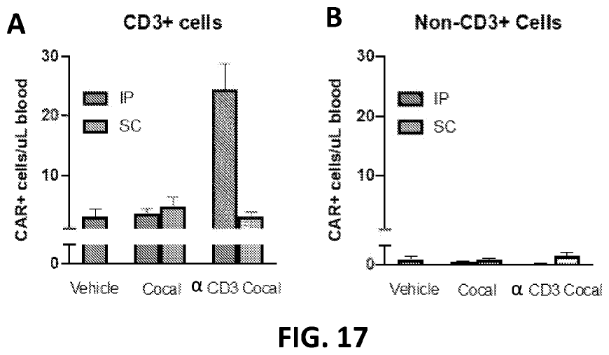

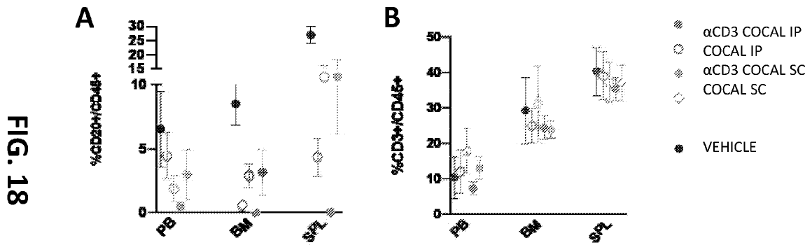

CAR+ cells were detected by flow cytometry only in CD3+ T cells from the αCD3-Cocal IP-dosed mice (FIG. 17A) and not in any non-CD3+ cells (FIG. 17B) suggesting selectivity of CAR transduction to CD3+ T cells. Day 14 flow cytometry data from peripheral blood showed that the CAR+ population of T cells in the αCD3-Cocal IP dosed groups was strongly enriched for CD8+ cells while the non-CAR+ population has approximately equal proportions of CD4 and CD8 (data not shown).

On Day 14 of the study, ddPCR for WPRE in blood pellets confirmed that the CAR was only detected in αCD3-Cocal IP-dosed mice (data not shown). At the termination of the study, B cells (FIG. 18A) and T cells (FIG. 18B) were assessed by flow cytometry in the peripheral blood, bone marrow, and spleen of mice. Complete B cell depletion was observed in mice treated with αCD3-cocal particles via the IP route suggesting a strong killing potency of the αCD3-Cocal engineered lentivirus particle transduced T cells. In contrast, incomplete depletion of B cells observed in mice treated with Cocal particles (non-αCD3) via the IP route, which was most pronounced in the bone marrow.

In summary, when delivered intra-peritoneally, 9 million TU of αCD3-Cocal engineered lentivirus particles successfully transduced T cells in vivo and caused rapid and complete B cell depletion in all tissues analyzed.

Summary: Off-target transduction was not observed (FIG. 17). In mice that received αCD3-Cocal engineered particles by IP, B cells were completely undetectable in blood starting on Day 7 post injection. In blood, bone marrow, and spleen, B cells were undetectable 4 weeks later at the termination of the study. CAR positive cells from the blood of mice treated with αCD3-Cocal engineered particles via IP at day 14 post injection were enriched for CD8+ cells compared to CAR negative, indicating the expansion and enrichment of cytotoxic effector cells. In vivo transduction of CD3+ T cells in the blood of mice treated with αCD3-Cocal particles by IP was confirmed with ddPCR to detect the WPRE sequence. Peripheral blood B cell depletion was observed also in mice that received Cocal particles via IP (FIG. 3A) though no CAR+ cells were detected by flow cytometry or ddPCR.

### Example 7: In Vivo Transduction of T Cells by Lentiviral Vector Encoding Anti-CD19 CAR and Anti-B Cell Activity

The aim of this study was to assess in vivo transduction of anti-CD19 CAR T cells in CD34 humanized NSG mice using an αCD19 CAR-RACR payload (SEQ ID NO: 121) packaged in a αCD3-Cocal envelop (SEQ ID NO: 128) with helper plasmids comprising gag/pol (SEQ ID NO: 131) and Rev proteins (SEQ ID NO: 132). In this study, αCD19 CAR-RACR αCD3-Cocal lentivirus particles were assessed for their ability to deplete B cells in a CD34 humanized model. Another study aim was to determine if rapamycin administration changed the course of B cell depletion, or the expansion of CART cells.

### Virus Preparation and QC Data

Lentiviruses were concentrated by ultracentrifugation, titered on 293T cells, cryopreserved, and stored at −80° C. until use. All preparations used in animal studies were tested for Mycoplasma and certified as negative for contamination. Endotoxin activity was less than 1 EU/mL for all lots.

αCD19 CAR-TGFB αCD3-Cocal viral particles with titer 1.6×10\hat{ }8/mL was thawed at room temperature or in hand. 290 uL of virus was diluted with 1 mL of Hanks' Balanced Salt Solution (HBSS) and kept on ice. Each mouse was given 250 uL per injection, intraperitoneally (4 mice total). 4.7 mL αCD19 CAR-RACR αCD3-Cocal viral particle with titre 4×10\hat{ }7 TU/mL was thawed, diluted with 0.5 mL HBSS, and kept on ice. Each mouse was given 250 uL per injection, intraperitoneally (16 mice total).

### Study Protocol

Female HuNSG mice 21 weeks of age and 16 weeks post CD34+ HSC implantation were used for this study. Mice rested for 1 week after arrival at the facility prior to beginning the study. The mice were bled and randomized into the treatment groups described in Table 4 based on engraftment parameters:

TABLE 4

| | | | Rapamycin 1 mg/kg IP |
|---|---|---|---|
| | | Virus | Monday/Wednesday/Friday |
| Group | N | (9 million TU IP day 0) | beginning Day 2 |
| 1 | 6 | Vehicle | Vehicle |
| 2 | 6 | Vehicle | Rapamycin |
| 3 | 8 | αCD19 CAR-RACR transgene, anti-CD3 Cocal | Vehicle |
| | | envelope | |
| 4 | 8 | αCD19 CAR-RACR transgene, anti-CD3 Cocal | Rapamycin |
| | | envelope | |
| 5 | 4 | αCD19 CAR-TGFB DNRII transgene, anti- | Vehicle |
| | | CD3 cocal | |

### Treatment Schedule:

Mice in all study arms were treated on the same schedule with either virus or vehicle (on study day 0), followed by injections of rapamycin or vehicle beginning on study day 2.

### Tissue Collection Schedule:

Mice in study arms 1-4 were divided into two equal endpoint groups, A and B, to allow for more frequent blood draws and two terminal harvests. Mice in study arm 5 were assigned the same end point schedule as the “B” endpoint groups.

### Group A Endpoint Schedule:

Blood was collected from the A endpoint groups on study day 3 and the mice were euthanized on study day 10. Approximately 75% of the spleen and 1 femur were collected from all mice and were fixed in 10% neutral buffered formalin (NBF) for 72 hours at ˜20× volume of the tissue, transferred to 70% ethanol, and kept at 4° C. for processing and paraffin embedding. Terminal blood, a small section of spleen and 1 femur were placed in PBS on ice for flow cytometry.

### Group B Endpoint Schedule:

Blood was collected from the B endpoint groups once a week. Body weight was measured twice a week for the length of the study. Group B mice were harvested on day 29. Approximately 75% of the spleen and 1 femur were collected from all mice and were fixed in 10% neutral buffered formalin (NBF) for 72 hours at ˜20× volume of the tissue, transferred to 70% ethanol, kept at 4° C. for processing and paraffin embedding. Terminal blood, a small section of spleen and 1 femur were placed in PBS on ice for flow cytometry. The study timeline is shown in Table 5.

TABLE 5

| | | | Rapamycin | | |
|---|---|---|---|---|---|
| | | 9 Million TU | IP (MWF | | |
| | | Virus IP | schedule | | Har- |
| Group | N | (day 0) | starting day 2) | Bleed | vest |
| 1A | 3 | none | none | Day 3 | Day 10 |
| 1B | 3 | none | none | Day 7, | Day 29 |
| | | | | 14, 23 | |
| 2A | 3 | none | 1 mg/kg | Day 3 | Day 10 |
| 2B | 3 | none | 1 mg/kg | Day 7, | Day 29 |
| | | | | 14, 23 | |
| 3A | 4 | aCD19-CAR RACR | none | Day 3 | Day 10 |
| 3B | 4 | aCD19-CAR RACR | none | Day 7, | Day 29 |
| | | | | 14, 23 | |
| 4A | 4 | aCD19-CAR RACR | 1 mg/kg | Day 3 | Day 10 |
| 4B | 4 | aCD19-CAR RACR | 1 mg/kg | Day 7, | Day 29 |
| | | | | 14, 23 | |
| 5 | 5 | aCD19 CAR TGFβ | none | Day 7, | Day 29 |
| | | | | 14, 23 | |

### Results and Conclusions

For the analysis by flow cytometry, all cells were gated by debris exclusion, singlet discrimination, live discrimination, and expression of human CD45. B cells were defined as human CD20+CD3−. T cells were defined as human CD3+CD20−. CAR+ events were defined as CD19-FITC positive or anti-2A peptide APC positive. Negative gates were set by stained samples from mice that received no virus. Positive staining was verified using cultured CAR T cells. T cells were further analyzed for expression of CD71 as the primary marker for activation.

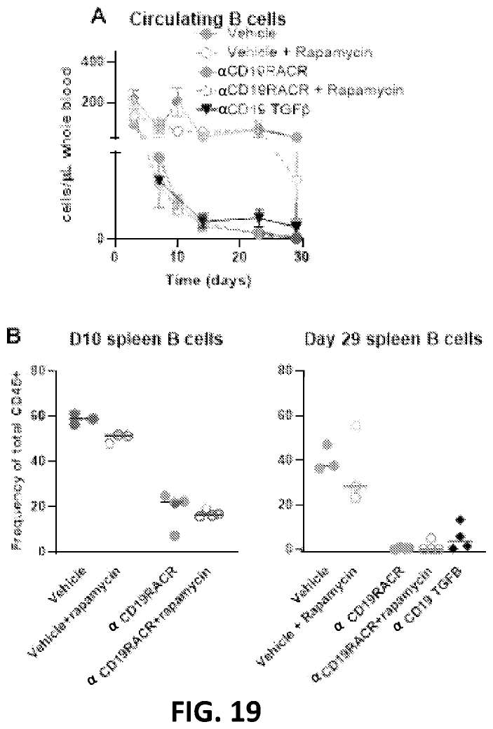

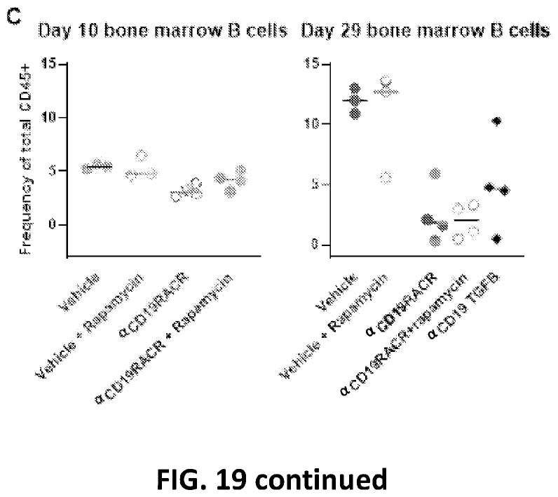

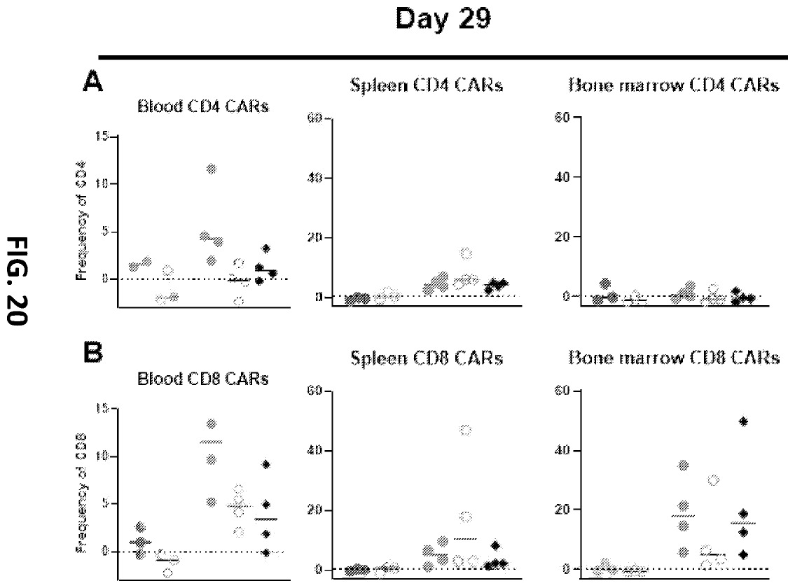

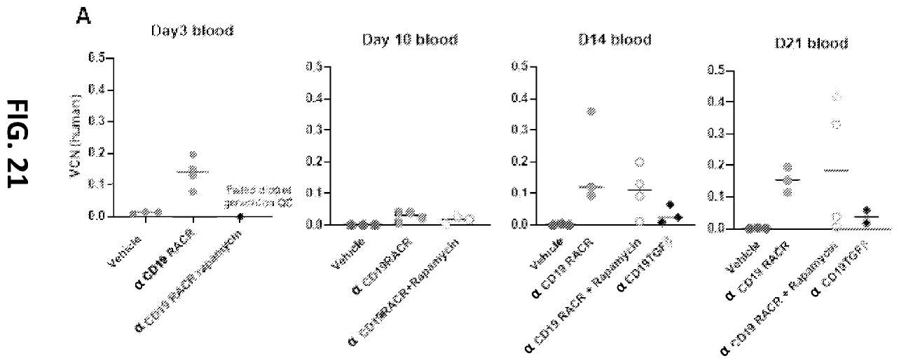

Surprisingly, mice treated with αCD19 CAR-RACR and αCD19 CAR-TGFβ displayed profound B cell depletion beginning 7 days post virus administration and reaching a nadir of almost no detectable B cells by study termination. In contrast, mice treated with vehicle displayed a gradual reduction of circulating B cells over time, as has been reported in the CD34-humanization model (FIG. 19A). The spleen (FIG. 19B) and bone marrow (FIG. 19C) were harvested from mice on study days 10 and 29 and B cell populations were assessed. On study day 10, B cell populations in the spleen of mice treated with lentivirus were reduced by approximately 70% compared to mice treated with vehicle (FIG. 19B, left panel). On study day 29, spleen B cell populations in lentivirus-treated mice were reduced by >95% compared to those of mice treated with vehicle (FIG. 19B, right panel). B cell populations in the bone marrow were equal between all study arms on day 10 and reduced by 70% in lentivirus-treated arms on study day 29 (FIG. 19C, right panel). B cells were notably depleted first from the blood beginning day 7, then from the spleen on day 10, and from the bone marrow on day 29. B cell depletion was consistent with anti-CD19 CAR activity. Rapamycin did not affect the course of B cell depletion in lentivirus treated mice or vehicle treated mice.

Humanization rates remained relatively constant during the length of the study and were not different between study arms. Little change was observed in CD4 and CD8 T cell numbers in all groups across the study (data not shown). T cell activation was assessed over the course of the study by CD71 expression (data not shown). CD4 T cells in mice treated with rapamycin exhibited lower CD71 expression than mice that were not treated with rapamycin on study days 10 and 14. Lentivirus administration did not affect CD71 expression in CD4 T cells. CD71 expression was elevated in CD8 T cells of mice treated with lentivirus on day 3 post lentivirus administration compared to mice treated with vehicle, which is consistent with αCD3-Cocal dependent T cell activation.

Plasma was collected by centrifugation from mice at the indicated time points and analyzed human cytokine production to determine if T cell activation and B cell depletion were associated with cytokine release. Low levels of human cytokines, close to the detection threshold, were observed and were not different between study arms. The cytokines IL-13, IL-1β, and IL-4 were below the detection threshold. The cytokines IFNγ, IL-10, IL-12p70, IL-2, IL-6, IL-8, and TNFα were detectable at low levels and equivalent between groups (data not shown).

CAR expression in T cells was assessed in the blood on study days 3, 7, 10, 14, 23, and 29, and in the spleen and bone marrow on study days 10 and 29. CAR+ populations were only observed on study day 29 in the CD8+ fractions of the blood, spleen and bone marrow. Background noise from the P2A+CAR+ was subtracted by using the average value of vehicle-treated mice as the baseline. With background subtraction, 5-10% of CD8 T cells were CAR+ in the blood, 5-20% were CAR+ in the spleen, and 10-40% were CAR+ in the bone marrow (FIG. 20). While putative CAR populations were observed at other time points in all lentivirus-treated arms, they were not sufficiently distinct from background events to be represented as bona fide CAR T cells.

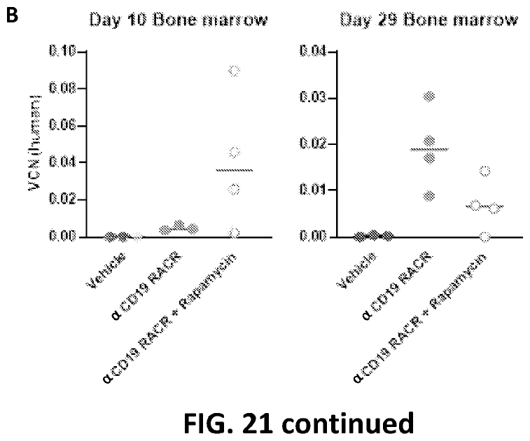

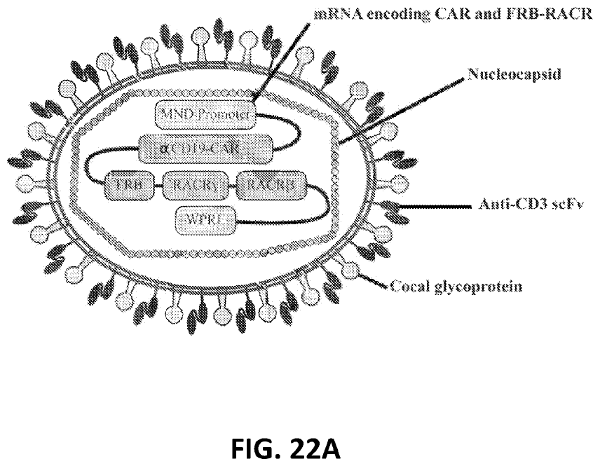

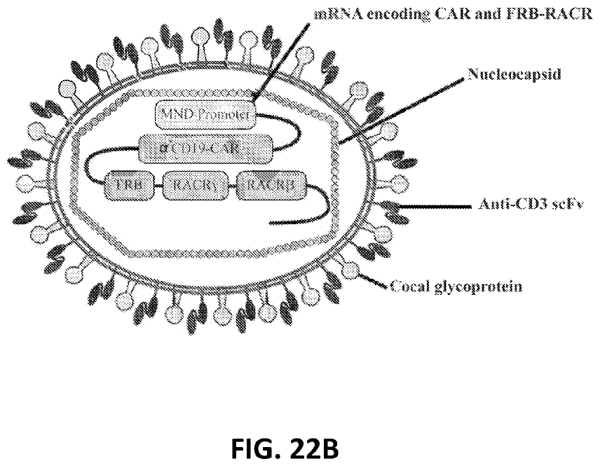

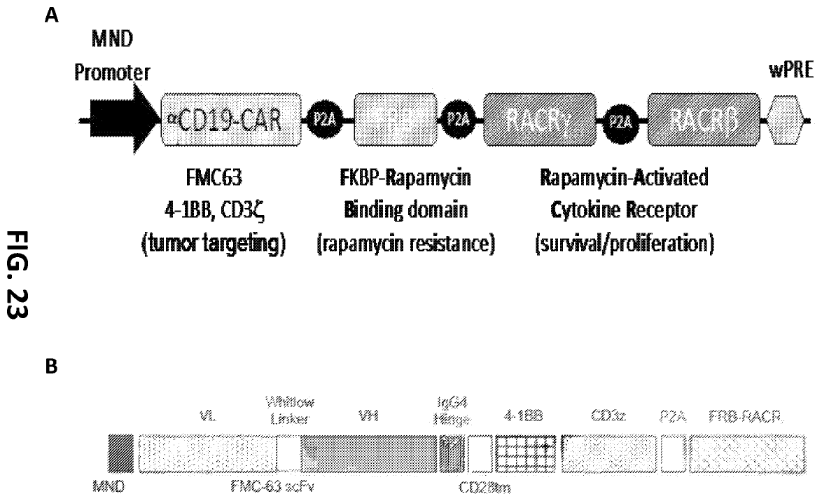

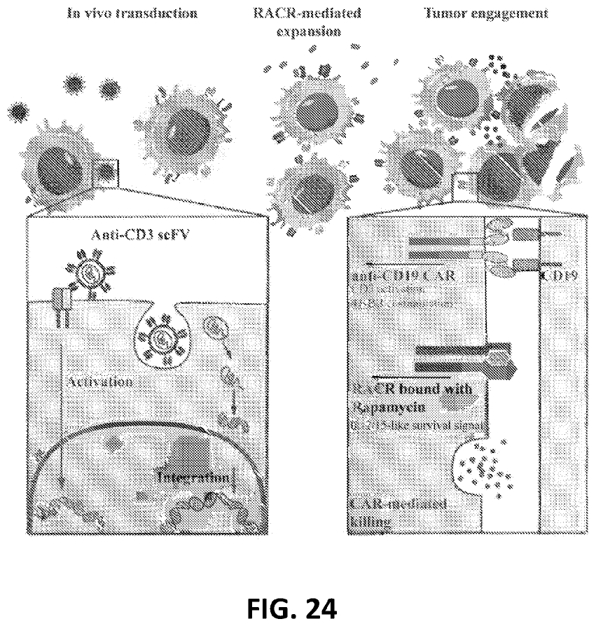

Digital droplet PCR (ddPCR) was used to assess blood, spleen, and bone marrow populations for vector integration relative to the human genome by targeting the WPRE sequence. Detectable levels of vector were found in the blood on days 3, 10, 14, and 21 (FIG. 21A). Vector integration was detectable in the bone marrow on days 10 and 29 (FIG. 21B). Vector copy number was approximately 10-fold higher in the blood as compared to the bone marrow, because T cells constitute a large fraction of human leukocytes in the blood, but a very small fraction of human leukocytes in the bone marrow.

The data show that in the CD34 humanization model with endogenous B cells as the sole source of antigen, 9 million transducing units (TUs) of αCD19 CAR αCD3-Cocal envelope lentivirus administered intraperitoneally caused rapid and complete B cell depletion. B cell depletion was similar in αCD19 CAR αCD3-Cocal envelope lentivirus-treated mice as compared to mice treated with the αCD19-TGFβ αCD3-Cocal lentivirus.

### Example 8: Clinical Study for Safety, Efficacy, and Pharmacokinetics

### Study Objectives

The primary objectives of the study were to evaluate and characterize the tolerability and safety profile of the drug product and rapamycin in adult and pediatric patients with B-ALL and B-lineage lymphomas and to evaluate the antitumor activity of the drug product and rapamycin in adult and pediatric patients with B-lineage hematologic malignancies

The secondary objectives of the study were to evaluate the complete response rate and durability of response of the drug product in B-ALL and aggressive B-lineage lymphomas, to evaluate the progression-free and overall survival of adult and pediatric patients with B-lineage hematologic malignancies treated with the drug product, and to assess the pharmacokinetics of the drug product

The exploratory objectives of the study were to explore biomarkers of response and toxicity to the drug product, to explore the immunogenicity of the drug product, to explore the pharmacodynamics of the drug product, to assess insertion site and frequency on safety/efficacy/PK attributes of the drug product, and to assess the impact of tumor microenvironment on anti-tumor activity and PK/PD.

A qualified strength test for the measurement of functional virions in the drug product (DP) will performed to determine the dose. The strength of the drug product will be reported in transducing units per milliliter (TU/mL) derived from transduction of HOS cells (a human osteosarcoma cell line) and measured via PCR performed on genomic DNA to quantify integration of a payload component (e.g., viral packaging sequences). The measurement of TU/mL (referred to as “functional titer”) using a molecular readout for viral integration into a susceptible recipient cell line is a routinely used measurement of strength for a virus product as it determines the concentration of functional units of the virus present in the preparation. The most accurate and quantitative measurement of strength at this stage of drug product development is the ability of viral particles to transduce human cells (as measured by the proposed assay) since transduction implies functional virus particles.

To understand the biological effect of the drug product, each lot of the drug product will be qualitatively evaluated to measure expression and/or functionality of all elements that contribute to the biological activity of this product. The proposed drug product characterization plan will include measurements of expression and/or functionality of the αCD3-cocal surface engineering, the anti-CD19 CAR, and the RACR-FRB system. Particularly, this product characterization effort will involve transduction of primary human unstimulated PBMCs in vitro and measurement of: T cell activation, CAR expression, RACR expression and/or RACR function, and CAR activity in response to the antigen. The relationship of these functional readouts to the quantified dose in TU will be evaluated in both nonclinical pharmacology studies and during clinical development.

TABLE 6
Synopsis of Phase 1 Clinical Study

| | |
|---|---|
| Protocol Title | A phase I/II, open-label, multicenter study of the drug product and |
| | rapamycin in patients with relapsed/R/R\B cell leukemia and |
| | lymphoma |
| Investigational Phase | I/II |
| Therapeutic Products | the drug product for injection, provided as frozen vials, and |
| | Rapamycin tablets (0.5-2 mg) or oral solution (1 mg/mL)- |
| | e.g., Rapamune ® (sirolimus) |
| Study Sites | Multicenter in the United States |
| Study Duration | Each subject is expected to participate at least 15 years as follows: |
| | Screening: up to 4 weeks |
| | Treatment and follow-up: |
| | Treatment |
| | DLT evaluation (during dose escalation) |
| | Post-treatment follow-up: for preliminary efficacy, until |
| | disease progression, unacceptable toxicity, death or study |
| | termination |
| | Long term follow-up: up to 15 years following drug |
| | product administration |
| Study Population | Adult (and potentially pediatric patients, pending final indication |
| | studied) with relapsed/refractory CD19-expressing malignancies |
| | which could include but is not limited to diffuse large B-cell |
| | lymphoma, including Burkitt's type (B-LBL), follicular |
| | lymphoma (FL), chronic lymphocytic leukemia (CLL), acute |
| | lymphocytic leukemia (ALL), and/or mantle cell lymphoma |
| | (MCL) |
| Study Design | This is an open-label, multicenter, Phase I/II study of the safety, |
| | efficacy, and PK of the drug product in combination with |
| | rapamycin in adult (and possibly pediatric) patients with R/R B- |
| | lineage hematologic malignancies. Specific histologies to be |
| | included in the trial remain to be determined but will be studied in |
| | disease-specific cohorts (e.g., B-LBL, CLL, etc.) |
| | The dose-finding (DF) groups in this study will evaluate safety |
| | profile of the drug product at various dose levels and those |
| | groups, along with dose expansion (DE) groups will further define |
| | the safety profile and preliminary efficacy in the disease cohorts. |
| | The study will use a modified toxicity probability interval (mTPI) |
| | method to allocate patients to various dose levels to minimize |
| | exposure to subtherapeutic dose levels while maintaining |
| | appropriate safety parameters. |
| | Patients will be screened for the trial and upon meeting all |
| | inclusion/exclusion criteria, will give written consent and be |
| | enrolled on the trial. |
| | The subjects will receive a dose at the assigned Dose Level on |
| | Study Day 1 (Day 1). Based on preclinical data, commercially |
| | available rapamycin will be started at a time that optimizes its |
| | activity while minimizing safety concerns, targeting commercially |
| | approved blood levels. Maximum duration will be determined |
| | based on further nonclinical studies to optimize magnitude and |
| | duration of response by limiting any observed toxicity. |
| | Per the mTPI method, after the probability of DLT (p[DLT]) and |
| | probability of complete response (p[CR]) are established, a |
| | minimum of 6 subjects will be enrolled in each Dose Group for |
| | each disease-specific cohort (DSC) to determine to assess whether |
| | the next Dose Group can be opened for each DSC. |
| | After each Dose Group is fully enrolled, all subjects will be |
| | assessed for DLT and CR determine if the next highest Dose |
| | Group can be opened for DF. Enrollment into DF of the higher |
| | dose level is prioritized over that already tested which will remain |
| | open for dose expansion to take patient “overflow” and collect |
| | more data at that Dose Level |
| | The same procedure for subsequent dosing levels/groups will be |
| | followed until a recommended Phase 2 dose is reached, as |
| | determined by the mTPI algorithm and final decision by the |
| | Sponsor. |
| | This process will be completed independently for each DSC, as |
| | toxicity, efficacy, and PK profiles may vary for each. |
| | Safety will be monitored throughout the study by a safety |
| | monitoring committee (SMC) consisting at least of the medical |
| | monitor, Sponsor, and study investigators. The SMC will meet |
| | regularly and ad hoc to review emerging data and provide |
| | recommendations to the sponsor regarding dose escalation and |
| | dose expansion. Additionally, an independent Data Safety |
| | Monitoring Board (DSMB) will be convened to monitor trial |
| | conduct for safety events. |
| Therapeutic Products, | the drug product, by interstitial/intranodal injection, |
| Dose, Mode of | Dose levels of the drug product to be evaluated (to be provided in |
| Administration | TU/kg). |
| | Rapamycin to be dosed orally per approved USPI |

Study Endpoints

| | |
|---|---|
| Safety | Safety monitoring periods will include: |
| | From informed consent to administration of the drug |
| | product |
| | From administration the drug product to administration |
| | of rapamycin |
| | From administration of either the drug product or |
| | rapamycin for 90 days |
| | From 90 days to end of study or administration of |
| | additional anticancer therapy |
| | Adverse events (AEs), serious adverse events (SAEs), adverse |
| | events of special interest (AESIs), along with specified |
| | laboratory values will be collected. |
| | DLT Definitions to be determined during clinical |
| | development after evaluation of nonclinical in vitro/in vivo |
| | data |
| | Recombinant lentiviral (RCL) testing will be performed at |
| | regular intervals at baseline and after administration of the |
| | drug product |
| Efficacy | Efficacy evaluations will depend on the final indication(s) to be |
| | studied, with appropriate modalities/timing depending. |
| PK/PD/ | Peripheral blood will be obtained to assess PK/PD, including |
| Immunogenicity | peripheral blood maximum CAR T-cell concentration (Cmax), |
| | time of maximum concentration (Tmax), total exposure as |
| | measured by total area under the curve from Day 1 to Day 29 |
| | (AUC₀₋₂₈), and B-cell aplasia, at Days 1 (prior to administration of |
| | the drug product), 2, 4, 8, 15, 22, and 29, as well as at all |
| | subsequent study visits to analyze persistence. Bone marrow and |
| | lymph node samples may be requested if obtained for other |
| | clinical reasons to assess PK/PD in those tissues as well. |
| | Immunogenicity evaluations will be obtained at all prescribed |
| | clinical assessments to evaluate antibodies to the drug product and |
| | its protein products as well as prior to administration as baseline. |
| Inclusion Criteria | Specific inclusion criteria will be provided in later protocol drafts |
| Exclusion Criteria | Specific exclusion criteria will be provided in later protocol drafts |

### The Drug Product

### Description and Proposed Mechanism of Action of the Drug Product

The drug product is an investigational, replication incompetent, self-inactivating (SIN), lentiviral vector (LVV) that is designed to transduce T cells in vivo to express an anti-CD19 CAR and target CD19-expressing tumor cells.

The drug product has a multi-step mechanism of action:
The LVV binds to T cells in vivo via an anti-CD3 scFv, which activates T cells and facilitates lentivirus internalization through interaction with the Cocal glycoprotein
The payload RNA genome, αCD19 CAR-FRB-RACR, is reverse-transcribed into DNA, shuttled to the nucleus, and integrated into the genome
Transduced T cells express the anti-CD19 CAR and target CD19-expressing cells, while also expressing the FRB and RACR system for rapamycin-controlled cytokine signaling

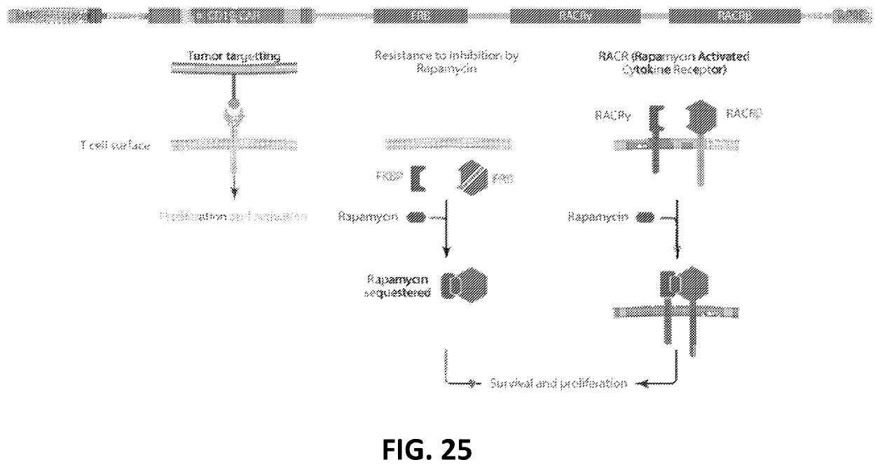

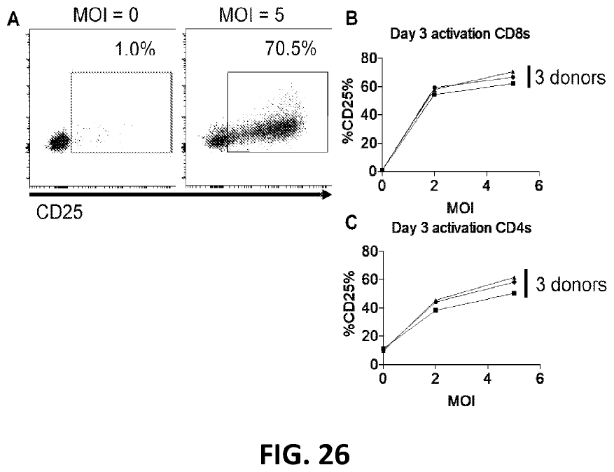

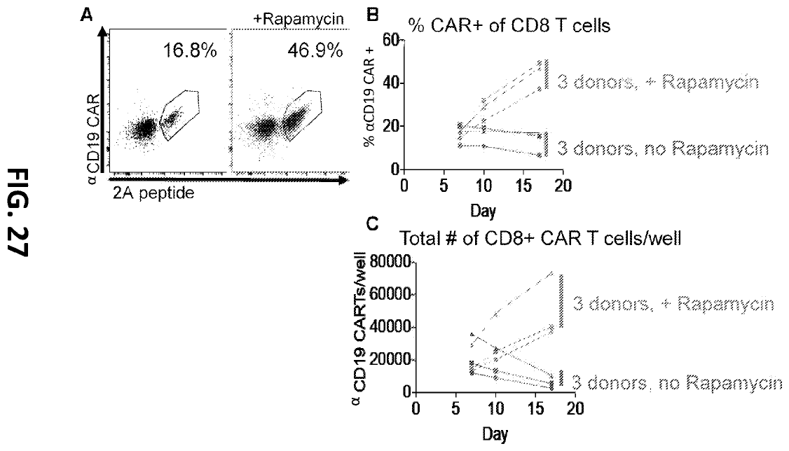

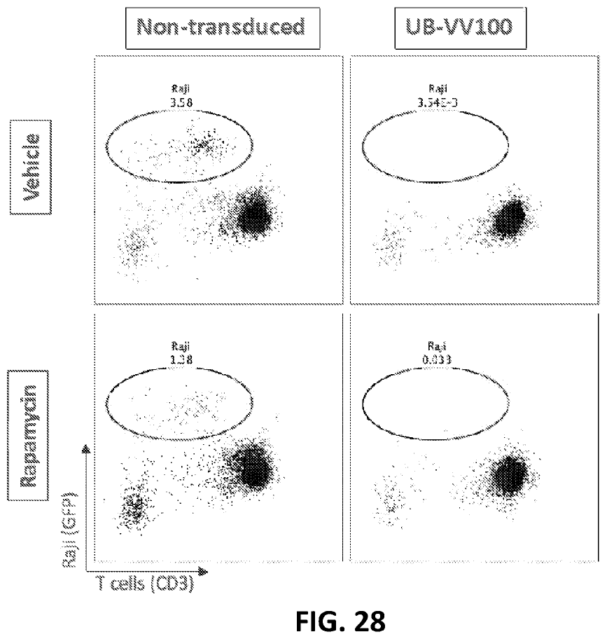

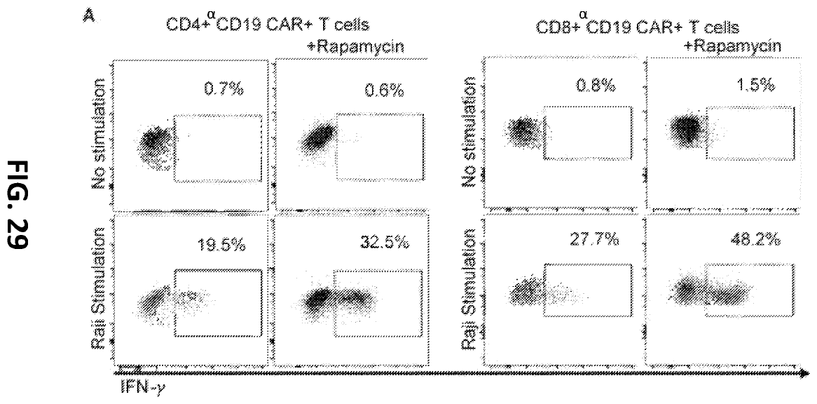

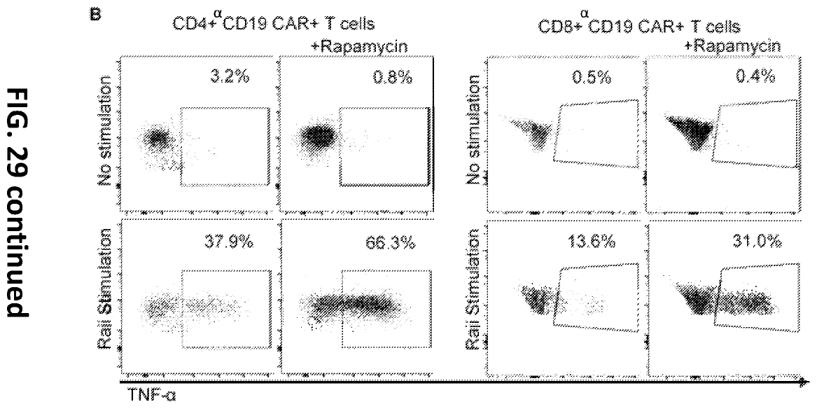

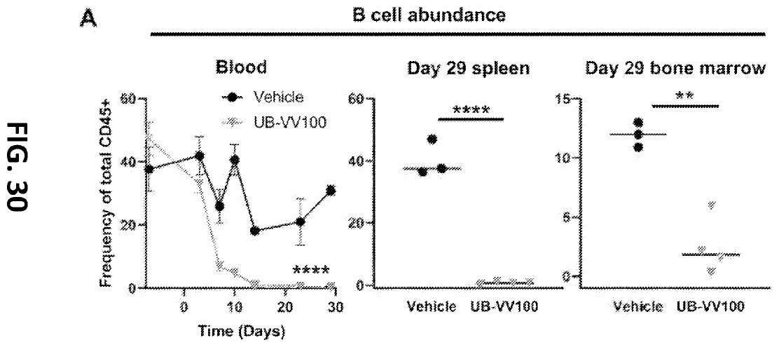

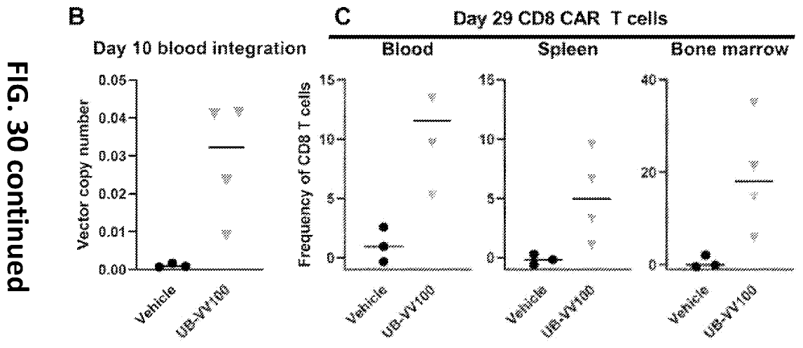

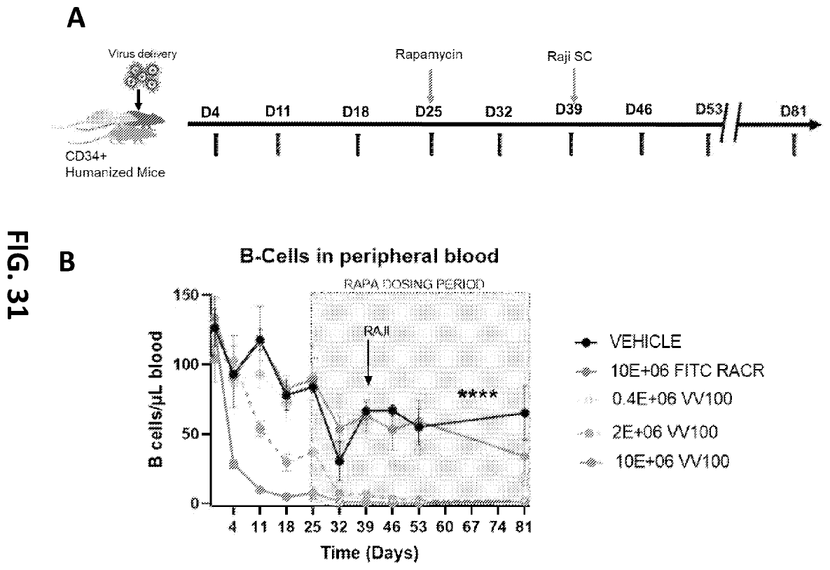

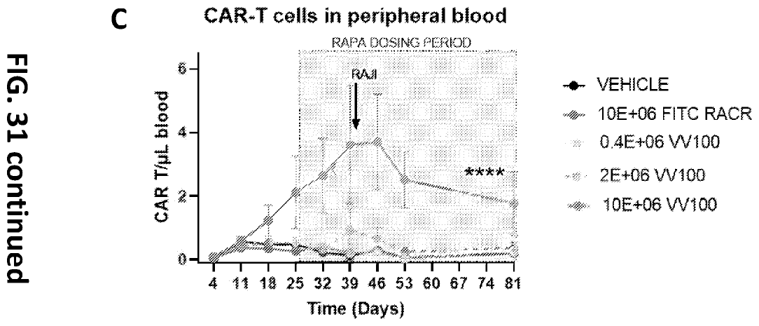

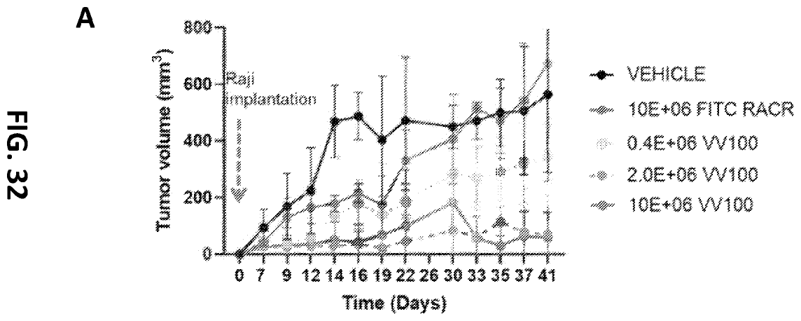

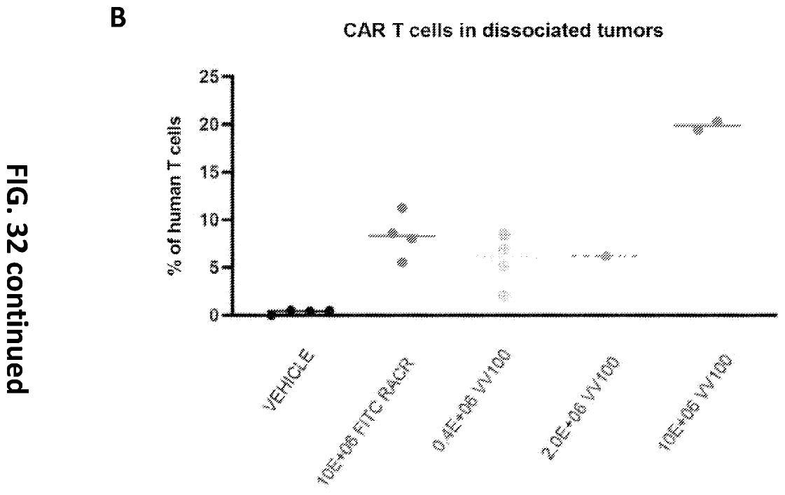

The five plasmids used in the manufacture of drug product LVV include:
1) Gag-Pol helper plasmid: Expression of viral gag and pol genes (SEQ ID NO: 124). These encode the viral structural proteins and proteins necessary for reverse transcription and integration into the target genome.
2) Rev helper plasmid: Encodes the viral protein Rev, which is necessary for the nuclear export of the un-spliced packageable RNA genome in the producer cell line (SEQ ID NO: 125).
3) αCD3 scFv plasmid: Encodes the anti-CD3 scFv (SEQ ID NO: 127)
4) Cocal helper plasmid: Encodes the Cocal envelope glycoprotein (SEQ ID NO: 123).
5) αCD19 CAR-FRB-RACR: Encodes the transgene to be delivered to activated T cells, which includes two chimeric receptor systems: 1) a second generation anti-CD19 CAR comprised of the FMC63 scFv fused to the 4-1BB and CD3zeta intracellular signaling domains, and 2) the RACR-FRB system (SEQ ID NO: 122) (FIG. 25).

### Lentiviral Particle Delivery and In Vivo T Cell Interaction

The lentiviral particle delivers a genetic payload to T cells either by intranodal injection or delivery to an interstitial space (e.g., SC or IP injection), which drains to local lymph nodes. Like other LVVs, the drug product viral particles are expected to be 80-120 nm in size, and thus, following administration, are taken up from interstitial fluid into lymph, allowing their direct transit into secondary lymphoid tissue (i.e., lymphatic vessels and lymph nodes). It is anticipated that either administration route will result in the drug product engaging and transducing CD3+ T-cells in the lymph nodes.

The drug product's capacity to deliver a genetic payload efficiently to in vivo T-cells is enabled through the surface engineering of lentiviral particles, which includes the expression of the anti-CD3 scFv and the Cocal glycoprotein. Specifically:
The anti-CD3 scFv on the surface of the LVV particles is designed to bind and activate T cells via CD3. CD3 stimulation activates T cells by initiating a signal cascade within the cell resulting in cell cycle progression and transcriptional/metabolic changes associated with activation. This state renders T cells permissive to LVV transduction.
Cocal is a fusion glycoprotein that after receptor binding and virus particle internalization acts to fuse the virus membrane to the endosomal membrane following acidification of the endosome, releasing the viral capsid into the cytosol. Thus, Cocal complements the action of CD3, as CD3-mediated T-cell activation leads to the upregulation of one of the Cocal receptors, the low-density lipoprotein receptor (LDLR), and supports internalization of the LVV.

### In Vivo Transduction

Following capsid delivery to the T-cell cytoplasm:
1) The lentiviral particle capsid traffics to the T-cell nuclear envelope/membrane. During this transport, reverse transcription of the viral genome and formation of a pre-integration complex (PIC) occurs within the capsid. The increase in cellular nucleotide pools generated by drug product-mediated T-cell activation through binding to CD3 enables efficient reverse transcription of the viral particle payload. During the process of nuclear membrane transit, disassembly of the capsid occurs, freeing the PIC to bind the host cell ubiquitous endogenous cellular chromatin regulator, LEDGF.
2) The binding of the PIC to LEDGF tethers the PIC to host cell active chromatin regions, and results in integration of the reverse-transcribed payload cassette into the genome of the transduced cell through the activity of the lentiviral INT protein on the viral long terminal repeat (LTR) sequences. The use of lentiviral machinery to affect the integration tends toward active genes and thus generally safer than other non-directed integration methods.

### Surface Expression of Payload

The drug product payload comprises an approximately 7 kb RNA genome that is reverse transcribed into a gene expression cassette to drive expression of a αCD19 CAR, FRB, and RACR components that provide drug-regulated immune cell activation, expansion, and targeting signals (FIG. 23).

Expression of the polycistronic transgene payload is driven by the MND promoter (SEQ ID NO: 35). The MND promoter (myeloproliferative sarcoma virus enhancer, negative control region deleted, d1587rev primer-binding site substituted) is a viral-derived synthetic promoter that contains the U3 region of a modified Moloney murine leukemia virus (MoMuLV) LTR with myeloproliferative sarcoma virus enhancer13 and has high expression in human CD34+ stem cells, lymphocytes, and other tissues. Four separate proteins are expressed, separated by 2A peptide sequences that induce ribosomal skipping and cleavage during translation. The CAR is a second-generation CAR comprised of the FMC63 mouse anti-human CD19 scFv linked to the 4-1BB costimulatory domain and the CD3zeta intracellular signaling domain.

Following integration of the drug product transgene (FIG. 23) into the genome of the cell, the transgene promoter (MND) drives expression of the payload open reading frame, which encodes four distinct proteins separated by 2A ribosomal skip peptide sequences.

The CAR and RACR receptors drive transmission of complementary signals that regulate T-cell survival, proliferation and “activation” of anti-tumor effector properties (FIG. 24). Expression and binding of the anti-CD19 CAR to CD19 on normal and malignant B cells in the lymphatic tissue and peripheral blood will generate signals that replicates both TCR and co-stimulatory receptor engagement. This is due to the CAR construct including both CD3 (“signal 1”) and 4-1BB (“signal 2”) co-stimulatory signaling domains, which are necessary for T-cell immune responses.

Uniquely for the drug product, administration of rapamycin acts as a ligand for the RACR subunits to dimerize and provide an IL-2/15 cytokine-like pro-survival/proliferative signal (“signal 3”) to viral particle transduced T-cells. Together, the signals mediated by the CAR and RACR receptors result in enhancement of in vivo expansion and maintenance of the CD19-targeting CAR T-cell population.

### RACR Function

The RACR components, RACR gamma and RACR beta are distinct fusion proteins that are expressed as membrane spanning receptor proteins. RACR gamma comprises a fusion between an extracellularly located FK Binding Protein (FKBP) and the common cytokine receptor gamma subunit transmembrane and cytoplasmic tail, and RACR beta comprises a fusion between an extracellularly located FKBP Rapamycin Binding (FRB) domain and the IL2RB transmembrane and cytoplasmic tail.

Rapamycin is an FDA approved mTOR inhibitor immunosuppressive agent, for use in a number of clinical settings. Rapamycin induces dimerization of the two RACR components, triggering IL-2/IL-15-like signaling in the transduced cells. The transgene includes a naked FRB domain, an approximately 100 amino acid domain extracted from the mTOR protein kinase. It is expressed in the cytosol as a freely diffusible soluble protein. The purpose of the FRB domain is to reduce the inhibitory effects of rapamycin on mTOR in the transduced cells, which allows for consistent activation of transduced T cells and gives them a proliferative advantage over native T cells (FIG. 25). Thus, the RACR system provides a survival/proliferative advantage to transduced T cells by providing IL2/15 signaling while non-transduced T cells are subject to the inhibitory effects of rapamycin mTOR inhibition.

### In Vivo Pharmacology

To assess the ability of the drug product LVV to transduce T cells in vivo, a humanized mouse model was employed. NSG mice engrafted with human cord blood CD34+ stem cells were obtained. These mice have approximately 25-50% human CD45+ immune cells in circulation as well as active generation of human B and T cells from bone marrow. At about 20 weeks post engraftment, the human CD45+ fraction in these humanized mice typically contains about 60-80% B cells and 20-40% T cells; therefore, these mice were considered an appropriate model for transduction of human T cells in vivo with depletion of B cells as a readout for the anti-CD19 CAR activity.

As shown in FIG. 30, to assess the function of the drug product in vivo, female CD34 humanized mice (n=3 or 4 per group) were treated with approximately 10 million TU of the drug product via intraperitoneal injection and the B cell populations via serial bleed were quantified by flow cytometry. Rapid depletion of B cells in the peripheral blood was observed, reaching a nadir of virtually no detectable B cells 14 days post injection, suggesting anti-CD19 CAR activity. Mice sacrificed 29 days post drug product administration displayed depleted splenic and bone marrow B cell populations by flow cytometry analysis. The drug product payload integration was detected in leukocytes collected from the peripheral blood by ddPCR analysis, and expression of the anti-CD19 CAR by flow cytometry.

These in vivo pharmacology studies have demonstrated that the drug product LVV is able to transduce a detectable population of CAR T cells in vivo and cause near complete elimination of B cells.

### Example 9: Dose Exploration for Lentiviral Vector Encoding Anti-CD19 CAR in CD34+ Humanized Mice Study Overview

Lentivirus was prepared using adherent production and titered on 293T cells by flow cytometry. Raji GFP FFLUC cells expressing luciferase and GFP were obtained from the Jensen lab, SCRI, cultured by Umoja Biopharma, and delivered in PBS on ice to Fred Hutch for injection.

20 human-NSG CD34+ females were randomized into 5 groups according to their human B cell levels. At Study D0 different doses of UB-VV100 Viral particles, FITC RACR particles as control or vehicle were injected IP according to Table 7 below.

TABLE 7
Study Groups

| Group | N | Dose (IP) | Virus IP | Rapamycin IP | Raji cells SC |
|---|---|---|---|---|---|
| 1 | 4 | 10 million | UB-VV100 | 1 mg/kg | 2 million |
| 2 | 4 | 2 million | UB-VV100 | 1 mg/kg | 2 million |
| 3 | 4 | 0.4 million | UB-VV100 | 1 mg/kg | 2 million |
| 4 | 4 | 10 million | FITC RACR | 1 mg/kg | 2 million |
| 5 | 4 | 0 | Vehicle | n/a | 2 million |

The UB-VV100 Viral particles comprised SEQ ID NOs: 121, 128, 131, and 132.

Starting at D4 mice were bleed retro-orbitally once a week until D53, their blood was analyzed by flow cytometry. At day 26 all groups except Vehicle group started receiving a dose of 1 mg/kg of rapamycin 3 times a week. At day 40 all mice were implanted subcutaneously with 100 ul of a mixture of 2 million RAJI GFP ffLUC and Matrigel at a 1:1 ratio. Tumors were measured with digital calipers 3 times a week to monitor their growth, the formula (W\hat{ }2×L)/2 was used to calculate tumor volume. From day 59 to day 70 tumors were measured 2 times a week. From day 59 to day to day 70 mice were switched to twice a week rapamycin schedule then back to a tree times a week from day 73 to receiving their last dose on day 77. All mice were sacrificed on Study day 81. Bone marrow, spleen, peripheral blood and tumor were collected, processed into a single cell suspension, and analyzed by flow cytometry. Schematics of the study timeline is shown in FIG. 31A.

### Results

Blood B cell populations were monitored by flow cytometry as a surrogate for anti-CD19 CAR T cell activity. Animals treated with 10 million TU of UB-VV100 exhibited 95% B cell depletion as relative to vehicle-treated controls by study day 11, sustaining that level of depletion up until study day 25, after which B cells further declined to where circulating B cells were virtually undetectable by flow cytometry. This second phase of B cell decline correlated with rapamycin dosing which began on study day 26. Animals treated with 2 million TU of UB-VV100 exhibited a ˜75% B cell depletion relative to vehicle-treated controls by study day 18, which was sustained at study day 25, then followed by a decline to nearly undetectable levels by study day 32. This second phase of B cell decline also correlated with rapamycin dosing.

Animals treated with vehicle only exhibited a gradual decline of circulating B cells, which is typically seen over time in CD34-humanized mice and was observed in our previous studies. In the vehicle-treated mice, a sharp decline in B cells was observed at study day 32, which correlates with rapamycin dosing. However, vehicle-treated mice did not receive rapamycin, so this decline cannot be explained by rapamycin effects, and since by study day 32 B cell levels in vehicle-treated mice were elevated once more, this decline is not significant. Animals treated with 0.4 million TU of UB-VV100 did not exhibit B cell depletion at early time points, but had a trend towards decreased B cells at later time points as compared to vehicle-treated animals. Animals treated with anti-CD3 scFv surface engineered cocal-pseudotyped lentivirus particles encoding an anti-FITC CAR showed no B cell depletion relative to vehicle, suggesting that B cell depletion is dependent on expression of the anti-CD19 CAR (FIG. 31B).

Circulating CAR T cells were measured throughout the study by flow cytometry. Circulating CART cells were not observed in animals treated with 0.4 million TU UB-VV100 particles, or 10 million TU of lentivirus particles encoding the anti-FITC CAR. Circulating CAR T cells were observed in animals treated with 10 million TU UB-VV100 starting on day 18 and increasing up to day 46, after which a gradual decline in total CAR T cell numbers occurred. In the 2 million TU dosed mice, CAR T cells were detectable only after rapamycin dosing; numbers peaked at day 39 and declined back to about baseline by day 53 (FIG. 31C).

Since complete depletion of endogenous B cells was observed in animals treated with 2 and 10 million TU of UB-VV100, animals were implanted with subcutaneous Raji tumor to assess the ability of potential circulating CART cells to clear malignant CD19-expressing cells. Tumor growth was monitored using the formula (W\hat{ }2×L)/2 and it was found that groups receiving 10E⁶ or 2E⁶ doses of UB-VV100 showed reduced tumor engraftment and proliferation as compared to animals treated with vehicle or lentivirus particles encoding an anti-FITC CAR (FIG. 32A).

Upon study termination, tumor CAR T cell infiltration into tumors was assessed by flow cytometry and it was found that CAR T cells were more abundant in tumors from mice treated with 10E⁶ UB-VV100 particles (FIG. 32B).

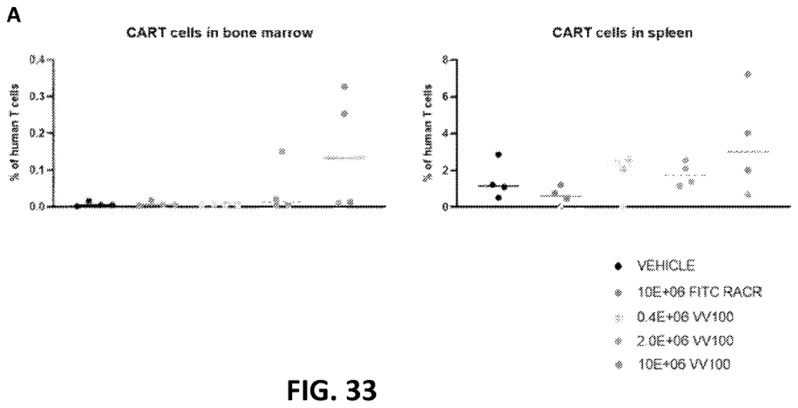

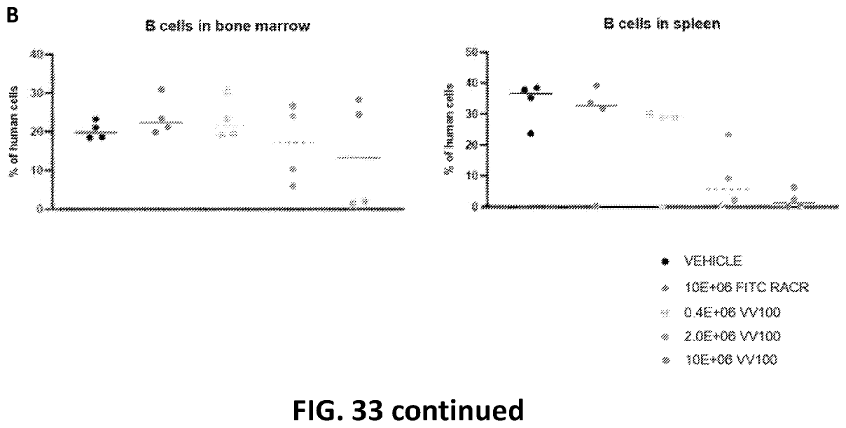

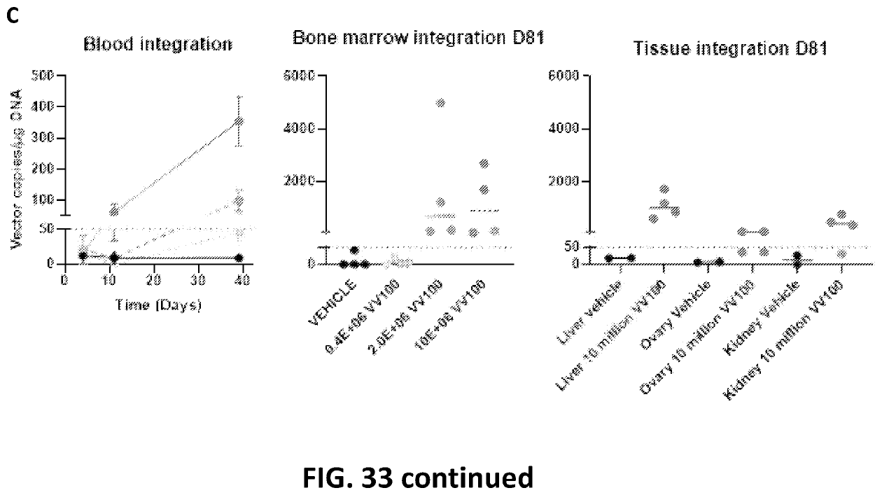

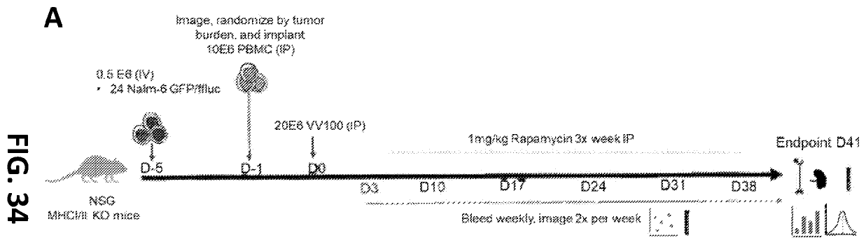

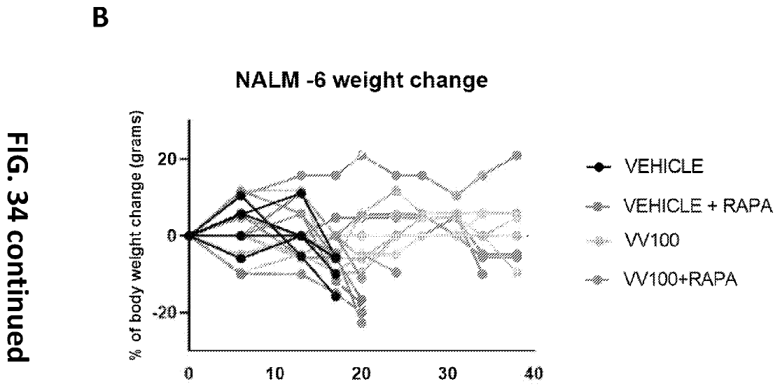

At day 81 all mice in the study were sacrificed, their bone marrow and spleen were analyzed by flowcytometry, we measured the percentages of CAR T positive cells present in the human T cell population of the bone marrow and spleen. Partial B cell depletion in the bone marrow and a significant B cell depletion was observed in the spleens of mice dosed with 10 million TU UB-VV100 compared to mice treated with Vehicle (FIG. 33).

We weighted all mice throughout the study once a week and calculate the percentage of weight change compared to their weight upon arrival. We did not observe weight loss after UB-VV100 treatment, throughout rapamycin dosing and after Raji tumor implantation in all different treatment groups (data not shown).

Transduction events were analyzed by ddPCR in the blood, bone marrow, spleen, liver, ovary, and kidney. While transgene integration was detected in the bone marrow and spleen of mice treated with 2 million or 10 million TU 81 days post VV100 administration, no transgene was detected in the bone marrow or spleen of the animals treated with 0.4 million TU, suggesting that 0.4 million TU is below the minimum efficacious dose in the current study design model (FIG. 33C). VV100 transgene was detected in the liver, kidney, and possibly ovary in mice treated with 10 million TU that were sacrificed on day 81 of the study.

Non-T cell transduction events were also assessed in this study. Flow cytometry was used to detect FMC63+ populations in the CD3− human fraction, the mouse CD45+ fraction, and the human CD45− mouse CD45− fractions in the spleen, blood, and bone marrow were assessed for FMC63 expression. No definitive non-T-cell transduced populations were observed in the analyzed organs, except perhaps in the mouse CD45+ fraction of the spleen (data not shown).

### Conclusions

Dose-dependent B cell depletion was observed in the peripheral blood of UB-VV100 treated CD34-humanized mice. This B cell depletion was sustained over 81 days and partially sustained in the bone marrow and spleen.

A sharp decline of B cells occurred in mice treated with 10 million TU UB-VV100 or 2 million TU UB-VV100, as compared to only gradual B cell depletion in mice treated with vehicle or control particles carrying an anti-FITC CAR (typically seen overtime in the CD34+ model), confirming that B cell depletion is CAR mediated and specific to CD19 antigen (FIG. 31).

Addition of rapamycin enhanced UB-VV100 mediated B-cell depletion and expanded the CAR-T cell population. Before the addition of rapamycin, CAR T cells were only evident at the 10 million TU UB-VV100 group, whereas after rapamycin addition an evident population of CART cells appeared in the blood of mice treated with 2 million TU UB-VV100 (FIG. 31C).

The results further show that circulation of large numbers of CAR T cells is not necessary for anti-B cell effector activity and treatment with VV100 inhibited SC Raji tumor growth in a dose dependent manner (FIG. 32).

No obvious off-target event was detected in the blood, bone marrow, or spleen by flow cytometry.

### Example 10: Efficacy of Lentiviral Vector Encoding Anti-CD19 CAR in Nalm-6 Systemic Tumor Models in PBMC-Humanized Mice

The aim of this study is to evaluate the efficacy of UB-VV100 and the effects of rapamycin dosing after UB-VV100 administration in a Nalm-6 systemic tumor model in PBMC-humanized mice. The UB-VV100 Viral particles comprised SEQ ID NOs: 121, 128, 131, and 132.

### Study Overview

Lentivirus was prepared using adherent production and tiered on 293T cells by flow cytometry. Nalm-6 GFP FFLUC cells expressing luciferase and GFP were obtained from the Jensen lab, SCRI, cultured by Umoja Biopharma, and delivered in PBS for injection.

24 MHC I/II KO NSG female mice were used in the study. On study day −5 (D-5) mice were implanted with 0.5 million Nalm-6 cells via retroorbital injection.

At study day −1 mice where humanized with 10 million PBMC via IP injection. The same day all mice were imaged with an IVIS imager 15 minutes after d-Luciferin injection (15 mg/kg) to detect Nalm-6 disease burden. All mice from each tumor group were randomized according to their tumor bioluminescence (Total Flux) levels into 4 cohorts according to the Table 8 below.

TABLE 8
Study Groups

| | | Tumor | | | |
|---|---|---|---|---|---|
| | | (day −5 | Human- | UB-VV100 | |
| | | RO) 0.5 | ization | (20 million | Rapamycin |
| Group | N | million | (Day −1) | TU IP) Day 0 | (Day 4) |
| 1 | 6 | NALM-6 | 10 million | Vehicle | no |
| | | | PBMC IP | | |
| 2 | 6 | NALM-6 | 10 million | Vehicle | 1 mg/kg MWF |
| | | | PBMC IP | | |
| 3 | 6 | NALM-6 | 10 million | UB-VV100 | no |
| | | | PBMC IP | | |
| 4 | 6 | NALM-6 | 10 million | UB-VV100 | 1 mg/kg MWF |
| | | | PBMC IP | | |

At study day 0 mice from groups 3 and 4 were treated IP with 20 million viral particles of UB-VV100 in 500 ul of PBS. Groups 1 and 2 received vehicle (PBS) IP injection. Starting on Study day 3, all mice were imaged twice a week (Tuesday, Friday) for the remainder of the study. At study day 4 mice in groups 2 and 4 started receiving 1 mg/kg rapamycin treatment via IP injection 3 times a week (Monday, Wednesday, Friday). The study timeline is shown in FIG. 34A. At Study days 10,13,17, and 20 mice with high disease burden had to be euthanized due to weight loss and signs of distress.

Percentage of body weight loss was monitored during the study (FIG. 34B). Only mice in the vehicle and vehicle+Rapamycin group had significant weight loss due to rapid expansion of tumor cells. All mice that survived to the end of the study were sacrificed on study day 41. Bone marrow, spleen, peripheral blood and tumor were collected, processed into a single cell suspension, and analyzed by flow cytometry.

### Results

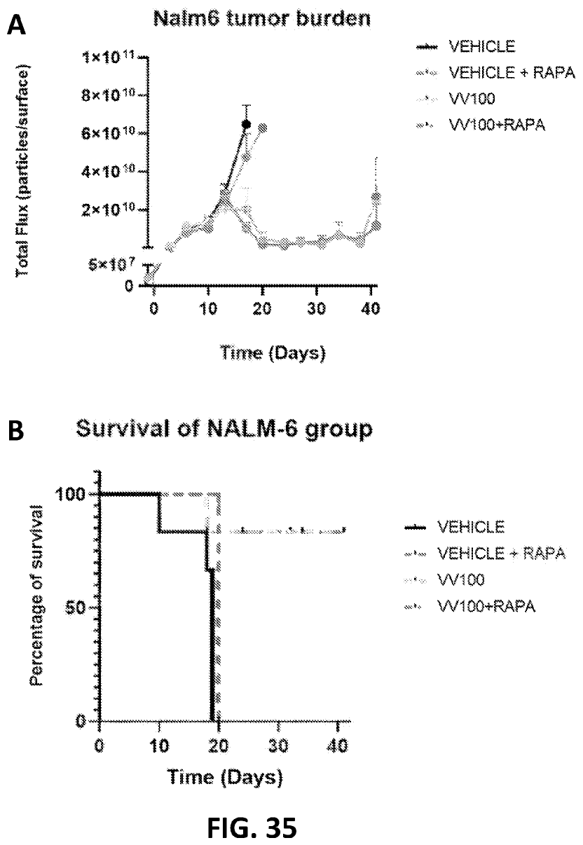

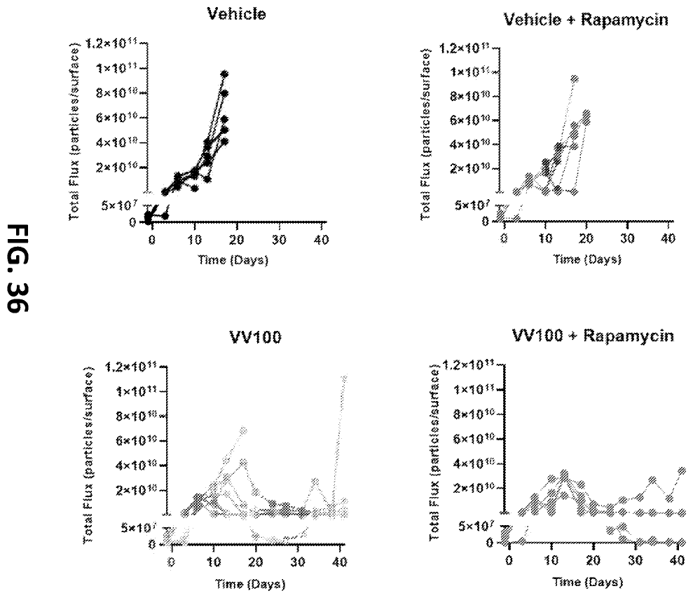

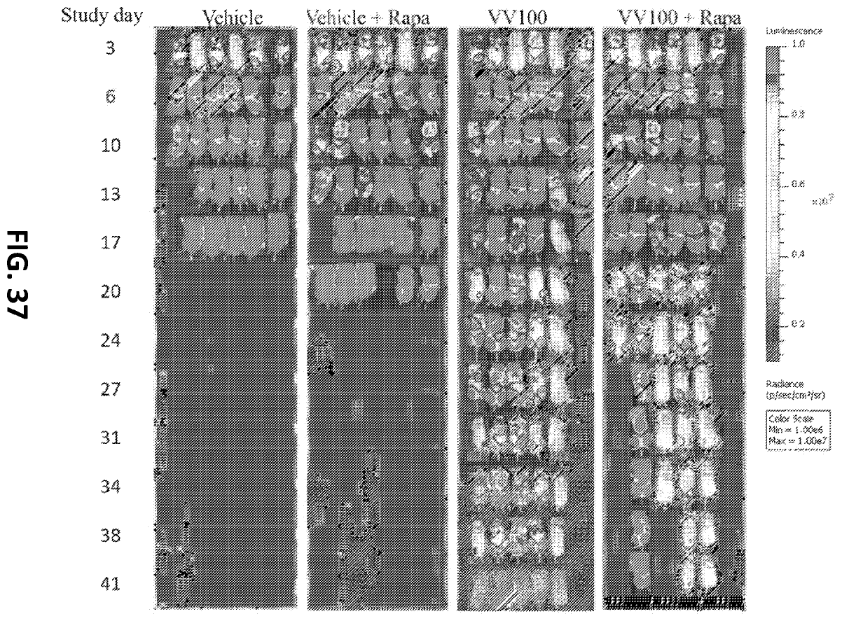

UB-VV100 treatment significantly decreased tumor burden measured by tumor bioluminescence (photons/second) (FIG. 35A) and increased survival (FIG. 35B) in the Nalm-6 tumor model. Mice that received UB-VV100 treatment extended their survival up to study day 41. In contrast, all mice in the Vehicle and Vehicle+rapamycin groups succumbed to disease between study days 17 and 20.

As shown in FIG. 36, mice in the Vehicle group (upper left) and Vehicle+ rapamycin (upper right) groups had an elevated disease burden starting at study day 10. Mice in these groups had to be euthanized by day 17. Mice in UB-VV100 group (lower left) had a decrease of disease burden starting at day 17; however, the effects of UB-VV100 in this group were temporary. All mice in the UB-VV100+rapamycin group (lower right) had a significant decrease in disease burden starting at day 17, and low disease burden remained in two mice. Only one mouse from this group had tumor burden increase after the initial regression.

As shown in FIG. 37, mice in the Vehicle group succumb to Nalm-6 disease at day 17; mice in Vehicle+Rapamycin group succumb to disease by day 20. Most of the mice in the UB-VV100 treatment group had a temporary decrease in disease burden; and mice in the UB-VV100+Rapamycin group had a significant decrease of disease burden that stayed low to undetectable in most of the mice (only one mouse had a partial reduction in tumor burden that then increased).

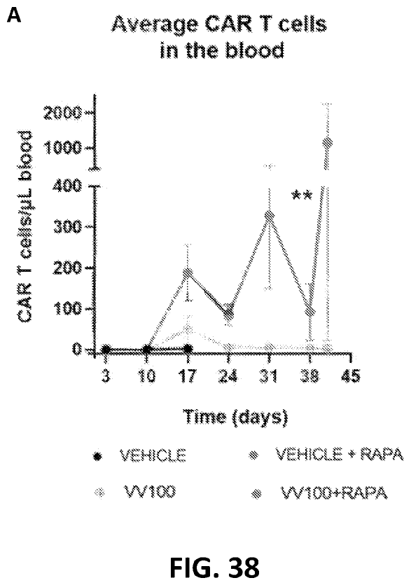

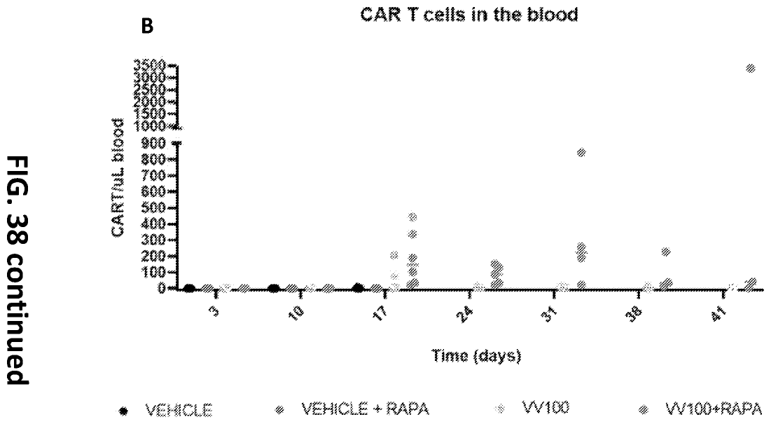

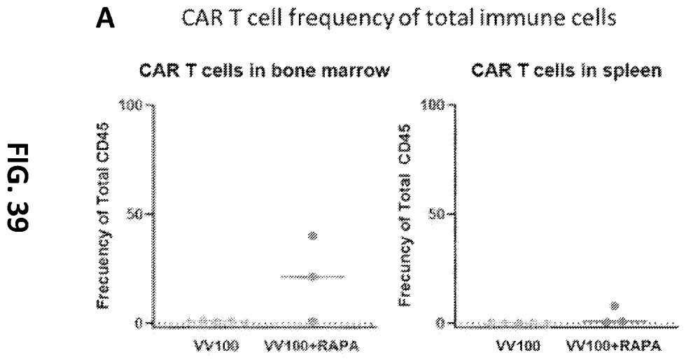

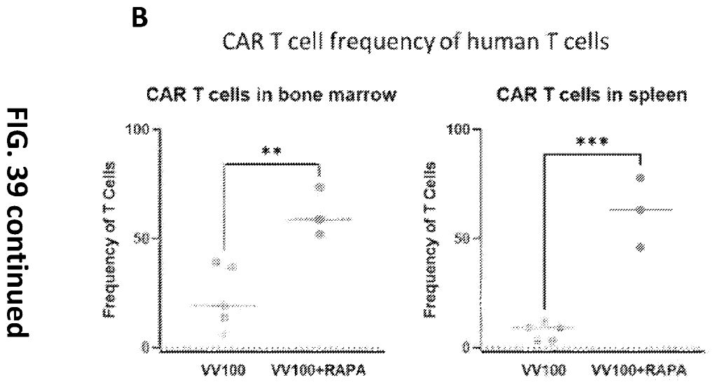

As shown in FIG. 38, CAR T cells significantly expanded overtime in the peripheral blood of mice treated with UB-VV100+Rapamycin. In comparison, in mice treated with only UB-VV100, CAR T cells showed a small increase that peaked at day 17 then decreased and remained stable for the remainder of the study.

At study day 17, tumor regression was observed in the VV100 group, however after day 20, tumor burden begins to increase. Mice in the VV100+Rapamycin group had tumor regression starting at day 17, two mice had apparent clearance of tumor burden and one had a temporary reduction followed by an increase of tumor burden detected by bioluminescence imaging (FIG. 37). CAR T cells significantly expanded overtime in the peripheral blood of mice treated with VV100+Rapamycin. In the mice treated with only VV100, CAR T cells showed a small increase that peaked at day 17 then decreased and remained stable for the remainder of the study (FIG. 38).

As shown in FIG. 39, CAR T cell frequency in the total immune cell population is higher in the UB-VV100+Rapamycin treatment group and higher in the bone marrow than in the spleen at day 41 (FIG. 39A). In addition, the frequency of CAR T cells in the T cell population in bone marrow and spleen were significantly higher in the UB-VV100+Rapamycin treatment group than in the UB-VV100 treatment group (FIG. 39B). Therefore, rapamycin treatment results in enrichment of CAR+ T cells in this study.

The total CAR T population was higher in bone marrow than in spleen and the percentage of CAR T cells present in the total T cell population was higher in VV100+Rapamycin group than in the VV100 Alone group (FIG. 39).

Bone marrow stained with a panel that includes P2A and CD19 confirmed that Nalm-6 tumor cells were not transduced by the UB-VV100 lentiviral vector (data not shown).

### Conclusions

This data shows that VV100 significantly reduces NALM-6 tumor burden and prolongs survival. Additionally, Rapamycin expands the CAR T cell population in the NALM-6 group mice and this expansion is inversely proportional to tumor burden.

### Example 11: In Vitro Pharmacology Studies

Results of additional in vitro pharmacology studies are summarized here. The UB-VV100 Viral particles comprised SEQ ID NOs: 122, 123, 127, 101, and 103.

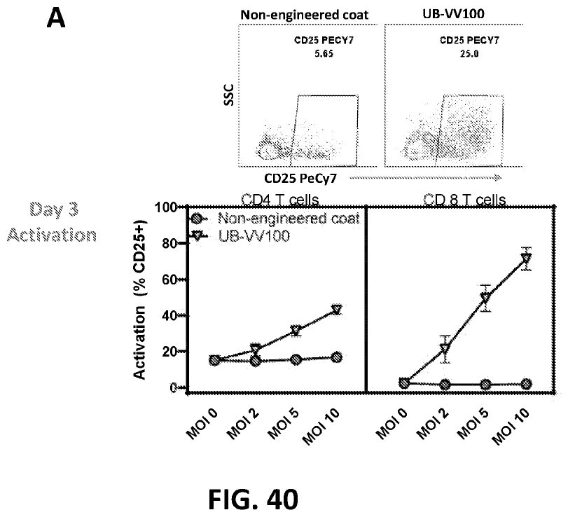

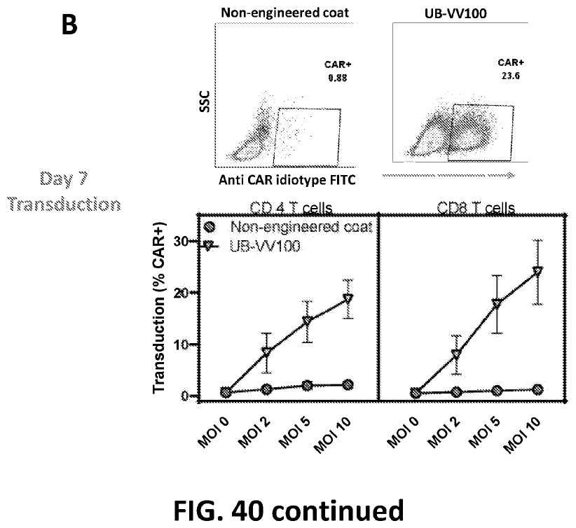

As shown in FIG. 40A and FIG. 40B, PBMCs from 3 donors were transduced with UB-VV100 or Cocal-pseudotyped lentivirus without anti-CD3 scFv. Activation was assessed after 3 days by flow cytometry for CD25 (FIG. 40A). Transduction was assessed after 7 days by flow cytometry for CAR expression (FIG. 40B). Plots showing data for CD25 are representative of all activation markers measured (i.e CD71 and CD69; data not shown). Plots are from a multiplicity of infection (MOI) of 5, gated on CD3+ live singlets. Summarized plots are combined data from 3 donors, error bars indicate 1 SEM. The results demonstrate that anti-CD3 scFv facilitates activation and transduction of T cells.

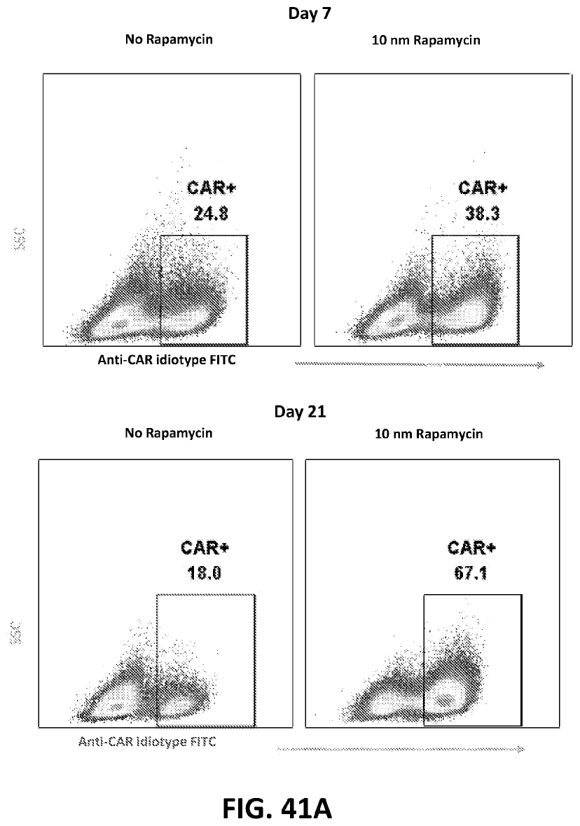

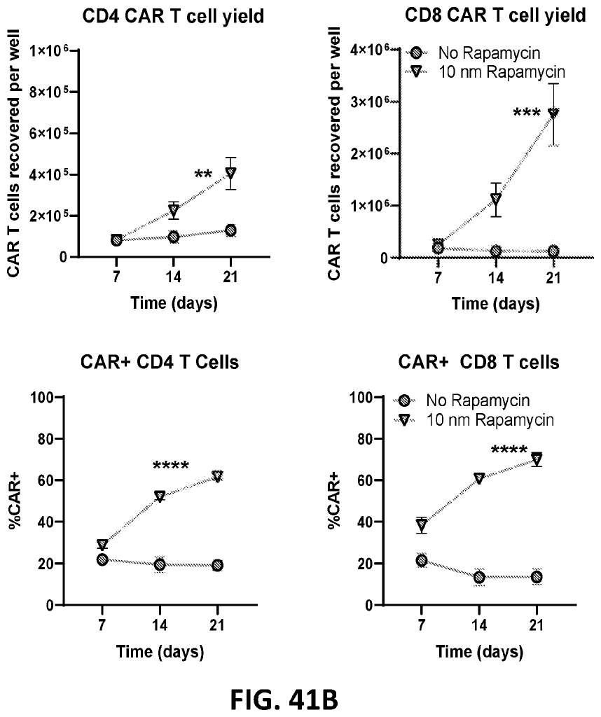

As shown in FIG. 41A and FIG. 41B, PBMCs were transduced with UB-VV100 at a multiplicity of infection of 10. Beginning on day 3, cells were split into separate cultures and treated with or without 10 nM rapamycin. CAR T cell transduction and expansion were assessed by flow cytometry and enumeration of cells. Representative flow plots are gated on live CD3+ singlets. The results demonstrate that RACR engine and rapamycin drives enrichment and proliferation of CAR T cells in vitro.

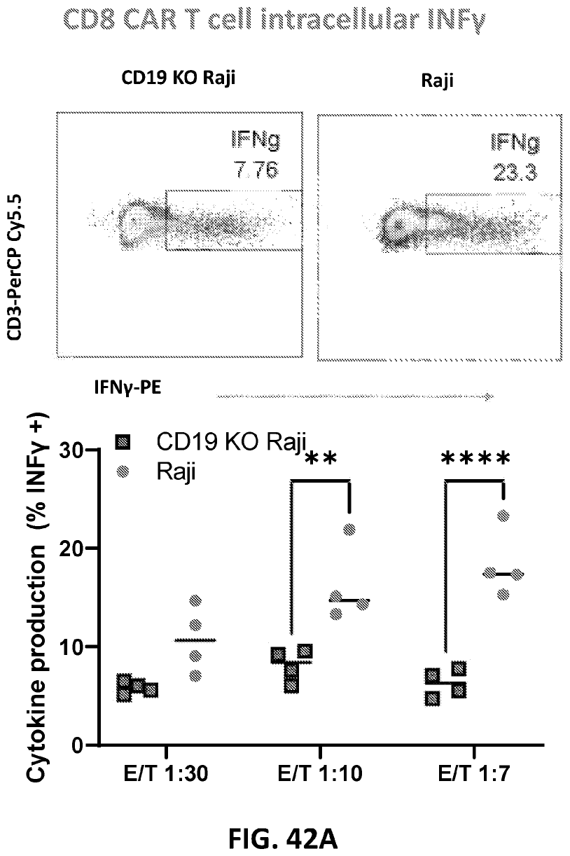

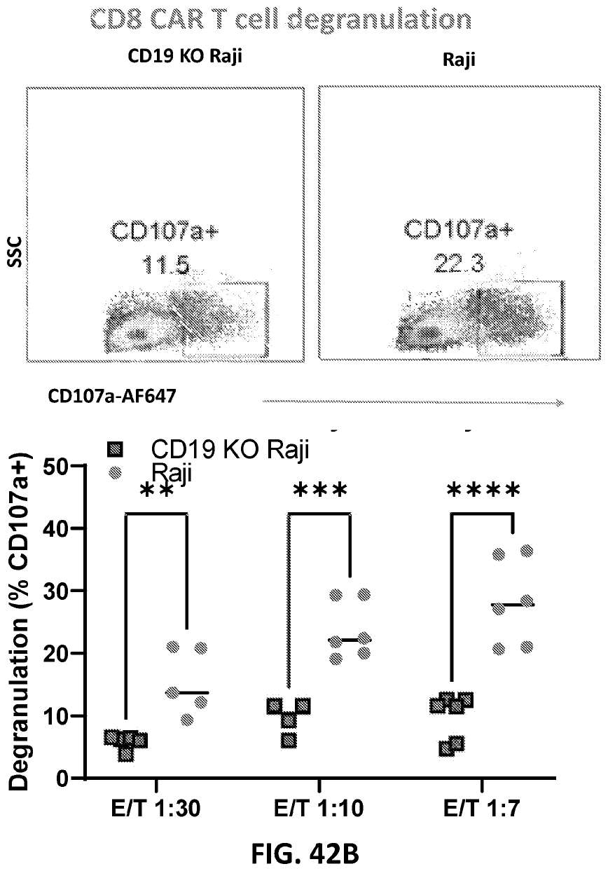

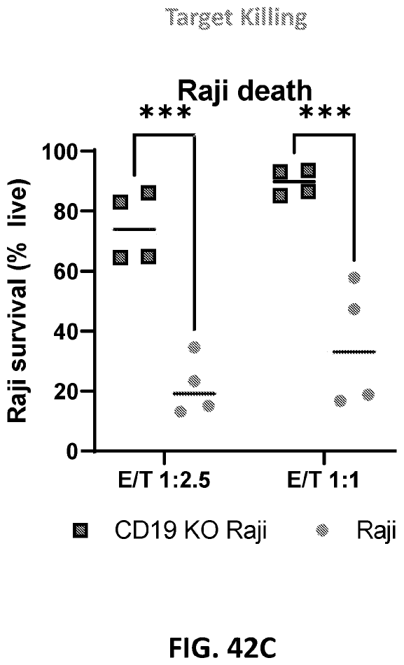

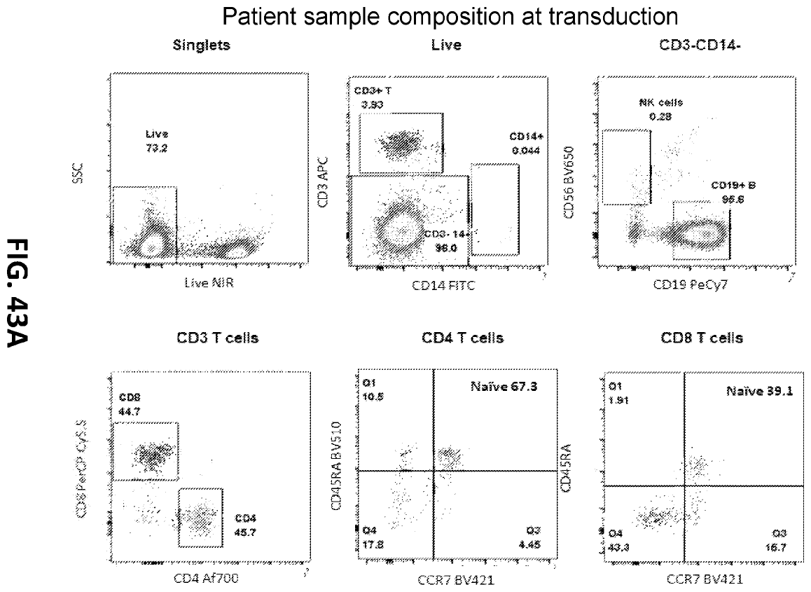

The cytotoxicity of transduced CAR T cells against Raji cells was assessed. PBMCs from 2 donors transduced with UB-VV100 were co-cultured with CD19-knock out (KO) or CD19-expressing Raji-GFP tumor cells for 5 hours with 2 mM of Monensin, 5 mg/mL of Brefeldin A, and 2 mg/mL of CD107a Ab. The cytotoxicity of CD8 T cells was assessed by intracellular staining of INFγ (FIG. 42A) and surface CD107a (FIG. 42B). In another set of experiments (FIG. 42C), UB-VV100-transduced PBMCs were co-cultured with CD19-KO or CD19-expressing Raji-GFP tumor cells for 48 hours, and killing potency was assessed by (Viable Raj i-GFP/Total Raji-GFP). For results analysis, representative flow plots are gated on CD8+CD3+ live singlets. **, ***, and **** indicate p values of <0.01, 0.001, and 0.0001 by 2-way ANOVA multiple comparisons test. The results demonstrate that UB-VV100 transduced CAR T cells display CD19-dependent cytotoxicity against Raji cells in vitro.

Transduction of T Cells from Patient

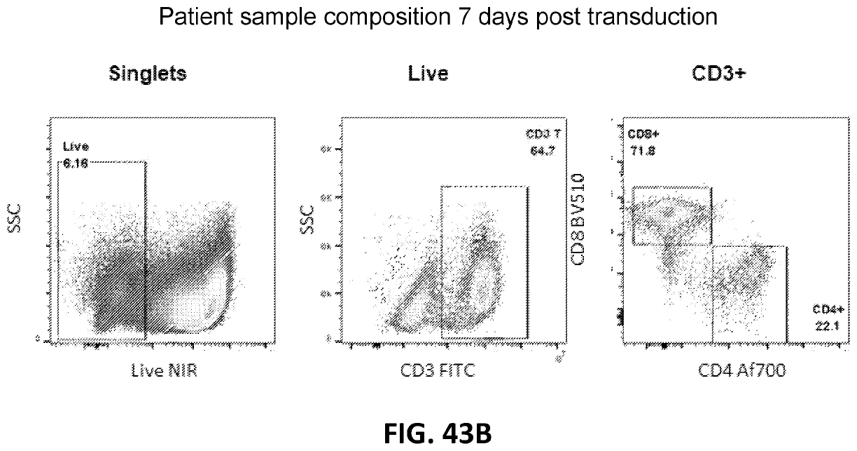

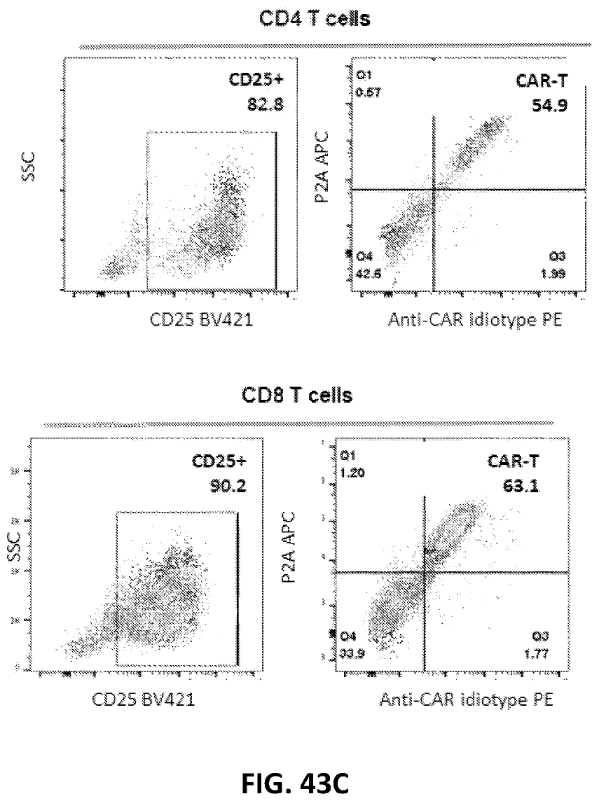

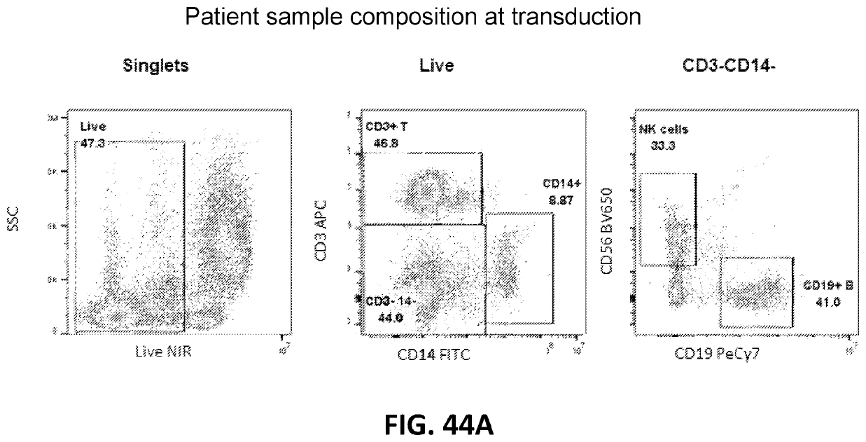

A PBMC sample from a B-ALL patient (male, 23-year old) was collected upon diagnosis and prior to initiating treatment. Hematology reports indicated the patient was in blast phase with 62% blasts in the blood, 95% blasts in the bone marrow, and a white blood cell count of 205.7×10⁹ cells/L. T cells comprised <4% of total live PBMCs. (FIG. 43A) Cells were transduced with two daily treatments of 2.5 UB-VV100 transducing units per live PBMC. T cell activation and transduction were assessed on day 7 by flow cytometry. (FIG. 43B) The results demonstrate that UB-VV100 effectively transduces T cells even when the T cell fraction is <4% of total PBMCs.

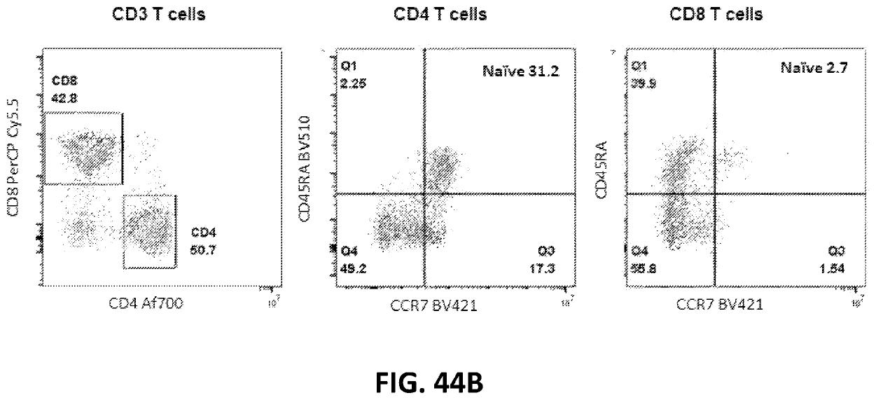

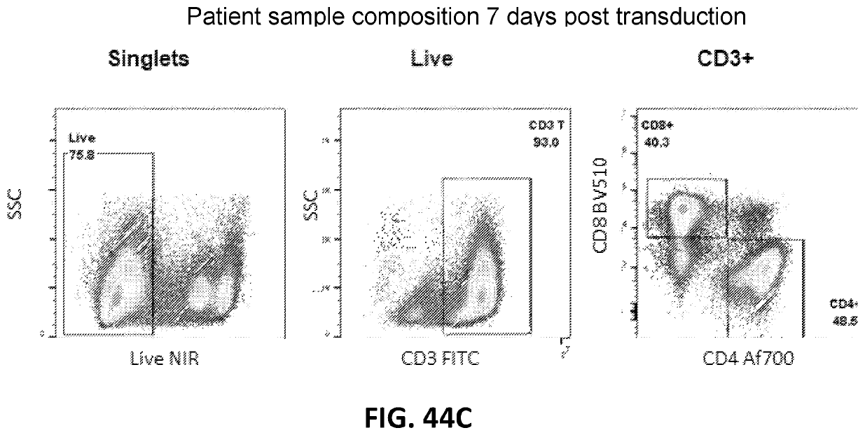

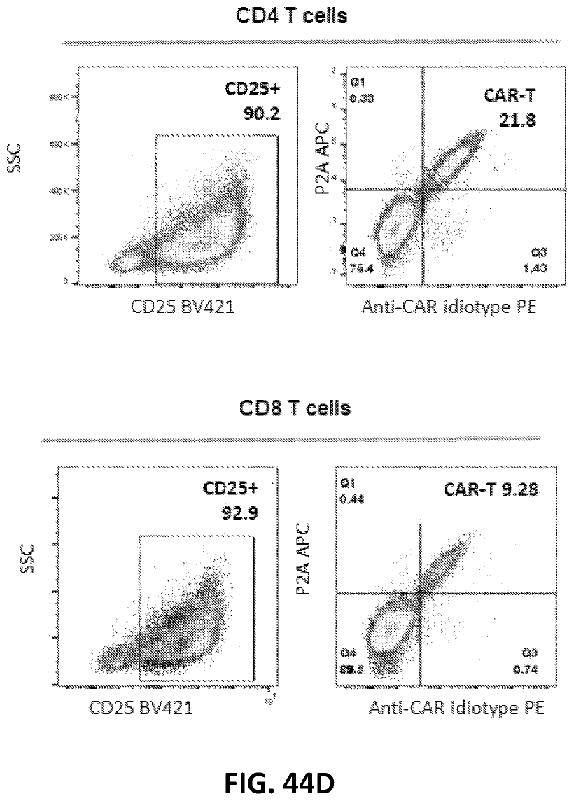

A PBMC sample from a DLBCL patient (male, 70-year old) was collected upon diagnosis and prior to initiating treatment. At the time of collection, white blood cell count was 11.3×10⁹ cells/L (FIG. 44A). Cells were transduced with two daily treatments of 2.5 UB-VV100 transducing units (TU) per live PBMC. T cell activation and transduction were assessed on day 7 by flow cytometry (FIG. 44B). The results demonstrate that UB-VV100 effectively transduces T cells in a PBMC population derived from a DLBCL patient.

### Conclusions

Results in this example demonstrate that 1) UB-VV100 viral particles activate and transduce T cells from healthy human PBMCs in a surface-engineering dependent fashion; 2) UB-VV100 viral particles transduce T cells from the PBMCs of patients with B cell malignancies; and 3) UB-VV100 transduced CAR T cells display CD19-specific anti-tumor activity in vitro.

### Example 12: Safety and Biodistribution of UB-VV100 in Mice

### Study Overview

The objective of this study was to assess UB-VV100 tolerability and compare the effect of time and dose on vector biodistribution in three mouse models (wild-type C57BL/6J mice, NSG CD34-humanized mice [strain NOD/SCID/IL2Rγnull (NSG)—humanized with CD34+ cord blood], and NSG MHCI/II DKO mice [strain NOD.Cg-Prkdc^{scid} H2-K1^{tm1Bpe} H2-Ab1^{em1Mvw} H2-D1^{tm1Bpe} Il2rg^{tm1wjl}/SzJ] with PBMC humanization). The NSG MHCI/II DKO mice have NOD SCID-IL-2 receptor gamma deleted (immunodeficient model lacking mature B, T and NK (Natural Killer), monocyte cells and complements). These mice also lack endogenous MHC I and II expression. The CD34-NSG mice have NOD SCID-IL-2 receptor gamma deleted (immunodeficient model lacking mature B, T and NK (Natural Killer), monocyte cells and complements) and were engrafted with CD34+ cells for reconstitution of a human immune system.

The UB-VV100 Viral particles comprised SEQ ID NOs: 122, 123, 126, 101, and 103. The presence and quantity of CD19-directed CAR+ T cells was measured in peripheral blood, spleen tissue, and bone marrow by flow cytometry. B-cell aplasia, an indicator of UB-VV100 activity and an early surrogate measurement for the presence of CAR+ T cells, was assessed in the blood by flow cytometry. Vector integration was determined by ddPCR analysis of genomic DNA extracted from mouse tissues. Multiplex RNA in situ hybridization (ISH) was performed to assess overall expression of transgene RNA levels and to determine the identity of transduced cells within each tissue.

UB-VV100 vector particles were manufactured using suspension HEK293T clone A3 cells. After viral harvest, material was purified using the Mustang Q XT5 system, concentrated, and sterile filtered through a 0.22 uM filter. Viral preparations were formulated in CTS™ OpTmizer™ (ThermoFisher Scientific) and aliquoted and stored at −80° C. in single use volumes. All viral preparations were tested for mycoplasma and endotoxin before being administered to subjects. All animals were administered Rapamycin three times weekly (M, W, F), starting on Study Day 5 and continuing through study end.

Ultra-pure grade phosphate buffered saline (PBS), USP, pH 7.4, was administered to subjects as the Vehicle control for both UB-VV100 and rapamycin dosing. All mice were randomized into cohorts according to the Study group shown in Table 9.

TABLE 9
Study Groups
Study Design

| | | | UB-VV100 | Rapamycin IP | | | |
|---|---|---|---|---|---|---|---|
| | | PBMC | TU | (M, W, F | | | |
| | | IP | IP | starting | Necropsy N (M/F) | | |
| Group | Mouse Strain | (Day −1) | (Day 0) | Day 5) | Week 1 | Week 4 | Week 12 |
| 1 | C57BL/6J | N/A | vehicle | 1 mg/kg | 0 | 2 (1/1) | 2 (1/1) |
| 2 | C57BL/6J | N/A | 20 million | 1 mg/kg | 0 | 4 (2/2) | 4 (2/2) |
| 3 | NSG MHC I/II DKO | 20 million | vehicle | 1 mg/kg | 0 | 2 (1/1) | 2 (1/1) |
| 4 | NSG MHC I/II DKO | 20 million | 20 million | 1 mg/kg | 0 | 4 (2/2) | 4 (2/2) |
| 5 | NSG MHC I/II DKO | 20 million | 20 million | 0 mg/kg | 0 | 0 | 4 (2/2) |
| 6 | NSG MHC I/II DKO | PBS | 20 million | 1 mg/kg | 0 | 4 (2/2) | 0 |
| 7 | CD34-NSG | N/A | vehicle | 1 mg/kg | 2 (0/2) | 2 (0/2) | 2 (0/2) |
| 8 | CD34-NSG | N/A | 20 million | 1 mg/kg | 4 (0/4) | 4 (0/4) | 4 (0/4) |
| 9 | CD34-NSG | N/A | 20 million | 0 mg/kg | 0 | 0 | 4 (0/4) |
| 10 | CD34-NSG | N/A | 100 million | 1 mg/kg | 0 | 4 (0/4) | 0 |

| | | | |
|---|---|---|---|
| TOTAL | 6 (0/6) | 26 (8/18) | 26 (8/18) |

TU = transducing units;
IP = intraperitoneal;
M = male;
F = female;
M = Monday;
W = Wednesday;
F = Friday

### Results

UB-V100 was administered to CD34-humanized NSG mice, which harbor both human T cells and human B cells, to assess its ability to generate CD19 targeting CAR-T cells in vivo across a range of two doses: a Low Dose of 20 million TU/animal and a High Dose of 100 million TU/animal. Mice were treated with either Vehicle and rapamycin (Group 7), Low Dose UB-VV100 (20 million TU/mouse) and rapamycin (Group 8), Low Dose UB-VV100 (20 million TU/mouse) without rapamycin (Group 9), or High Dose UB-VV100 (100 million TU/mouse) and rapamycin (Group 10). The ability of resultant anti-CD19 CAR-T cells to cause endogenous B cell depletion was then assessed to confirm on-target activity. A subset of the first 4 weeks post UB-V100 administration was analyzed for activity since humanization levels were greatly decreased after this time period.

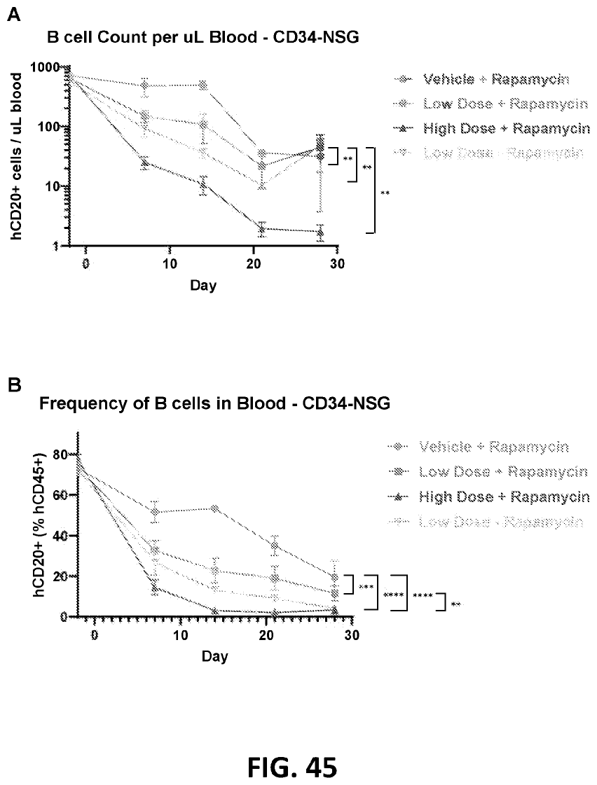

UB-VV100 resulted in a dose-dependent depletion of human CD20+ B cells in the blood of CD34-NSG mice treated with either Low Dose or High Dose UB-VV100, as compared to Vehicle treated controls (FIG. 45). B cell levels (hCD20+ cell population gated on single cells, live cells, and hCD45+) were measured over time in blood taken prior to dosing, as well as weekly after UB-VV100 administration, starting on Study Day 7. While human B cells levels naturally depleted over time due to loss of humanization in Vehicle treated mice (Group 7), a significant, dose-dependent depletion of human B cell frequency occurred in UB-VV100 treated mice (FIG. 45).

No depletion of endogenous circulating mouse B cells was observed in wild type mice treated with a Low Dose of UB-VV100 and rapamycin (20 million TU/mouse; Group 2), as compared to Vehicle and rapamycin treated controls (Group 1) (data not shown). These findings are consistent with the fact that the FMC63 binder on the anti-human CD19 CAR construct expressed by UB-VV100 is not predicted to cross-react with mouse CD19.

Detection of UB-VV100-generated anti-CD19 CAR-T cells was achieved using an anti-FMC63 antibody in a human immune cell flow cytometry panel. CAR-T cell levels (CAR+ population gated on single cells, live cells, hCD45+ cells, and hCD3+ cells) were measured in the blood from Week 1 to Week 12 and in the spleen and bone marrow during scheduled necropsy timepoints. The anti-FMC63 antibody was also used with a mouse immune cell flow cytometry panel to assess CAR+ immune cell populations in wild type mice (Group 1-2) and in the spleen and bone marrow of humanized mice during scheduled necropsy timepoints (Group 3-10).

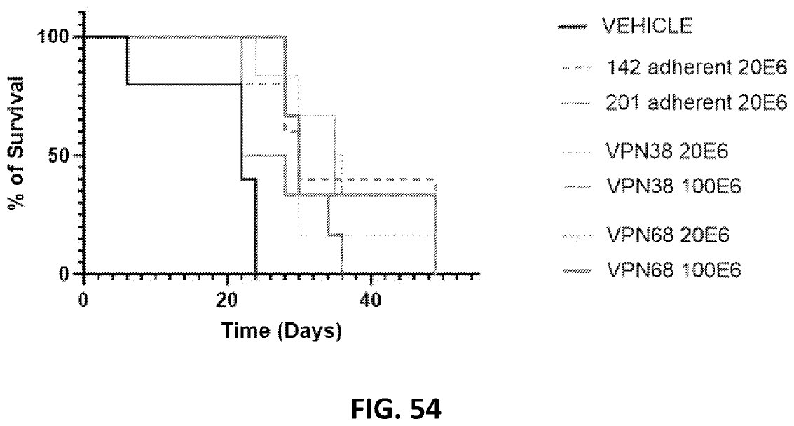

There was a significant increase CAR-T cells detected in the blood of CD34-NSG mice treated with a High Dose (100 million TU/animal) UB-VV100 and rapamycin (Group 10) as compared to Vehicle controls (Group 7), with the highest levels occurring on Week 4 (FIG. 54A). CAR-T cells levels were also significantly higher in the spleens of High Dose UB-VV100 CD34-NSG mice and detected in the spleens of some mice treated with Low Dose (20 million TU/animal) UB-VV100 and rapamycin (Group 8).

No CAR+ mouse T cell (mCD3+ cell) staining above levels in Vehicle treated animals was detected in the blood, bone marrow, or spleen of wild-type C57BL/6J mice treated with a Low Dose (20 million TU/animal) of UB-VV100 at any timepoint. These findings are consistent with predictions that the αCD3 scFv molecule present on the surface of UB-VV100 viral particles does not bind to mouse CD3, and therefore, would not activate or transduce mouse T cells.

To determine which cell types within each ddPCR positive tissue have integrated UB-VV100 payload, an RNAscope™ LS Multiplex Fluorescent ISH assay was performed on a multi-tissue array containing spleen, ovary, liver, kidney, and lung. The assay was performed on tissue from CD34-NSG mice; one Vehicle treated (Group 7) as a negative control and four treated with High Dose UB-VV100 (Group 10). The 4-multiplex stain contained a custom RNA ISH probe targeting the RACR region of UB-VV100, an RNA ISH probe targeting human T cells (hCD3), an RNA ISH probe targeting mouse macrophage/monocytes (mCD68), and an RNA ISH probe targeting mouse endothelial cells (mPecam). The cell markers used in this assessment were selected based on a pathologist's review of RACR+ cell morphology in test tissue during initial assay development of the RACR RNA ISH probe. Visual scoring was performed by a qualified scientist to assign a single score to each sample based on the predominant staining pattern throughout the entire sample. Percentage of cells positive for each marker, as well as the percent of RACR+ cells that were dual positive for other cell type markers, was scored visually based on number of cells with >1 dot/cell and binned into categories (0%, 1-10%, etc).

All samples had negative control stains pass quality control, and the Vehicle treated mouse tissue (TOX001_39) was negative for RACR mRNA expression in all tissues. Across tissues from the UB-VV100 High Dose group, RACR positive staining was highest in the spleen (11-20% for all mice) and CD80 (1-10% for all mice). No RACR positive cells were observed in the heart. None to very rare RACR positivity (0%, <5 cells, or <1%) occurred in the ovary, kidney, and lung. These results correlate excellently with the levels of vector genomes detected by ddPCR in the same tissues (data not shown).

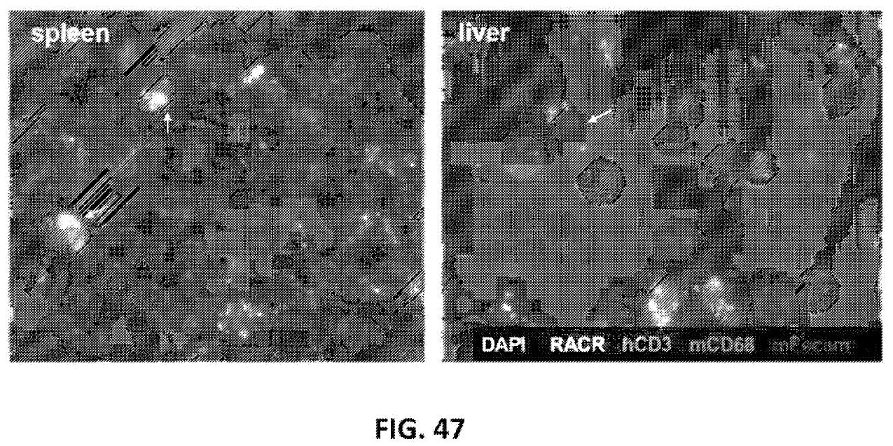

An analysis of dual positivity of RACR+ cells in each tissue was also performed to classify the cell type. In all mice, a vast majority of RACR+ cells were co-positive for either mCD68 or hCD3, indicating that the vector genomes detected are due to transduction of immune cells (macrophages or T cells) (FIG. 47). In the spleen, overlap between staining of the three cell type markers was noted (likely due to the high cellular density of the tissue); CAR-T cells were detected in the spleen of all UB-VV100 treated mice, though CAR+ macrophages were more common. In the liver, the vast majority of RACR+ cells were macrophages with some rarer RACR+ endothelial cells, though overlap between the Pecam and CD68 signal was noted (FIG. 47). Rare RACR+ endothelial cell transduction was also observed in some tissues. Between 1 to 3 RACR+ cells that were negative for all markers were identified on the outer ovarian lining or uterine lining of some mice, which is likely a result of the route of administration (intraperitoneal) leading to direct exposure. With the exception of the cells lining the uterus/ovary and a few cells in the kidney of one mouse, no unclassified RACR positive cell types were observed.

To further enhance the function of UB-VV100 lentiviral particles, T cell costimulatory ligands were incorporated into the particles' surfaces to initiate co-stimulation in conjunction with particle binding, T cell activation, and transduction. In vitro, incorporation of one or more co-stimulatory ligands on the particle surface enhanced lentiviral particle binding to and activation of T cells, resulting in enhanced proliferation and activation of transduced CAR+ T cells. Furthermore, CAR T cells generated with co-stimulatory ligand-displaying UB-VV100 lentiviral particles exhibited a less-differentiated, central memory-like phenotype, and enhanced CAR-mediated proliferation and tumor killing in vitro compared to CAR-T cells generated without co-stimulatory ligands. It was observed that co-stimulatory ligand surface-engineered UB-VV100 lentiviral particles generated CAR T-cells in vivo with enhanced antitumor activity in a humanized NSG mouse model of B cell malignancies. For example, UB-VV100 lentiviral particles were optimized by incorporating the T cell costimulatory ligand, CD80, which triggers CD28 co-stimulation during T cell activation and transduction. The presence of CD80 on the particle surface enhances lentiviral particle binding to T cells and activation of T cells resulting in enhanced proliferation and activation of CAR+ T cells in vitro. CAR T cells generated with CD80-containing lentiviral particles lead to enhanced antitumor activity in a humanized NSG mouse model of B cell malignancies.

The results suggest that the collective mechanism of action of UB-VV100 lentiviral particles to initiate anti-tumor immune responses can be augmented through expression of combinations of surface displayed ligands that engage T-cell activation and co-stimulatory pathways necessary to both render T-cells competent for transduction while optimizing their immunophenotype and function.

UB-VV100 toxicology studies further demonstrated a favorable safety and biodistribution profile using intranodal administration to canines. The study design is depicted in Table 10.

TABLE 10
Study Groups

| | | Dose | Dose Volume | Dose | | |
|---|---|---|---|---|---|---|
| | | Level | (uL/ | Volume | | |
| Group | Test | (TU/ | injection | (uL/ | DoseConc. | |
| No. | Material | animal) | site) | animal) | (TU/mL) | Adm. Route |
| 1 | Vehicle | 0 | 300 | 600 | 0 | Bilateralinguinal |
| | | | | | | LN |
| 2 | αCD3- | 3.98e8 | 300 | 600 | 6.63e8 | Bilateralinguinal |
| | Cocal- | | | | | LN |
| | EGFP | | | | | |
| 3 | αCD3- | 3.98e9 | 6000 | 6000 | 6.63e8 | IP |
| | Cocal- | | | | | |
| | EGFP | | | | | |
| 4 | αCD3- | 3.98e8 | 300 | 600 | 6.63e8 | Bilateralinguinal |
| | Cocal- | | | | | LN |
| | EGFP | | | | | |

Conc. = Concentration; No. = Number; TU = transducing units; LN = lymph nodes; IP = intraperitoneal; Adm = Administration.

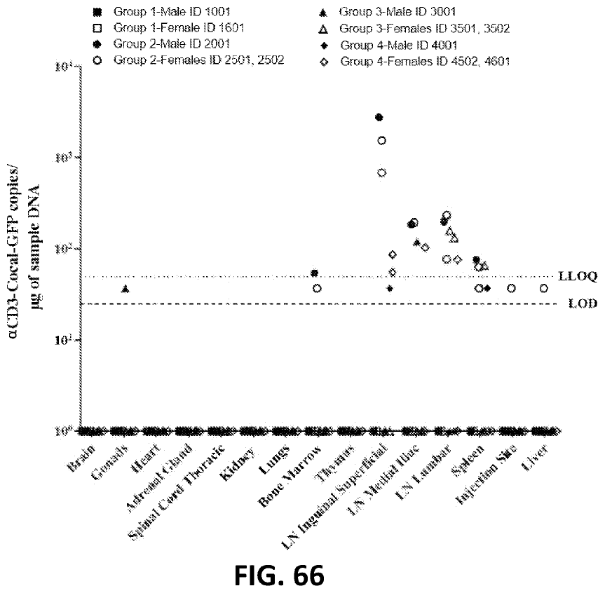

All tissues and blood samples of control animals in Group 1 were negative for αCD3-Cocal-GFP. Groups 2, 3 and 4 were negative or below the lower limit of quantification for brain, ovary, testis, heart, adrenal gland, spinal cord thoracic, kidney, lung, thymus, injection site and liver (FIG. 66). Administration of αCD3-Cocal-EGFP once by intranodal and intraperitoneal at 3.98e8 TU/mL and 3.98e9 TU/mL, respectively, was well tolerated in canines. There were no effects on in-life toxicology endpoints (clinical signs, body weights, food consumption and clinical pathology) and no post-mortem macroscopic and microscopic findings. Quantification of αCD3-Cocal-GFP by qPCR showed detection in the blood (via intraperitoneal), superficial inguinal, medial iliac, and lumbar lymph nodes (via intranodal), and spleen (both intraperitoneal and intranodal).

Intranodal administration of UB-VV100 lentiviral particles to canines was well tolerated and resulted in transduction that was largely restricted to the injected lymph nodes, with ˜90% lower transduction in the downstream draining lymph node, and no transduction in non-immune organs.

### Conclusions

In this study, three mouse models (wild-type C57BL/6J mice, CD34-humanized NSG mice, and NSG MHCI/II DKO mice with PBMC humanization) were evaluated. UB-VV100 was well tolerated at a dose of 20 million TU per animal in all three mouse models and at a dose of 100 million TU per animal in CD34-NSG mice. Importantly, histopathological tissue evaluation by a board-certified pathologist did not find any microscopic findings definitively related to UB-VV100 treatment.

UB-VV100 treatment resulted in in vivo generation of human CAR-T cells in the blood and spleen of CD34 and PBMC humanized NSG mice. No evidence of any UB-VV100 biological activity (CAR-T cell generation, B cell depletion) was detected in wild-type C57BL/6J mice, which lack human T cells, the target of UB-VV100. Full UB-VV100 biological activity could only be evaluated in CD34-humanized NSG mice since PBMC humanized mice lack human B cells, the target of the viral αCD19 CAR payload. A significant, dose-dependent depletion of circulating B cells was observed over time during the first 28 days post intraperitoneal injection of UB-VV100 into CD34-NSG mice (FIG. 45).

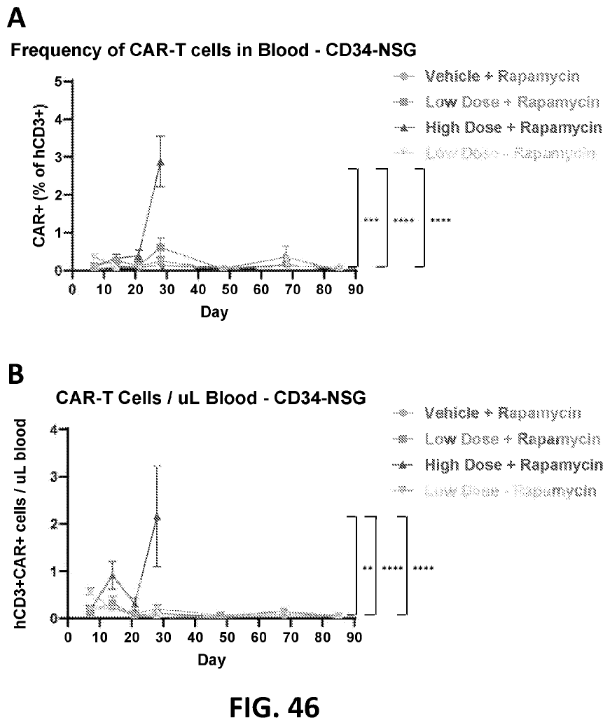

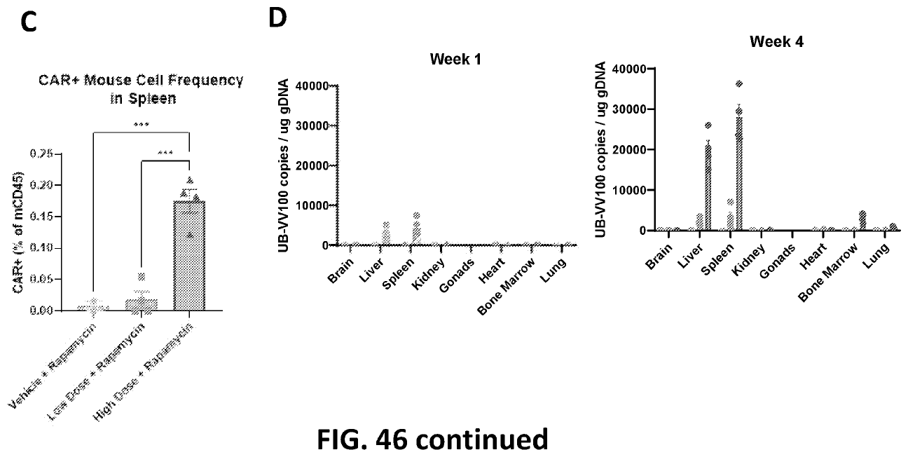

Biodistribution of UB-VV100 was first assessed using ddPCR on tissue-extracted genomic DNA due to the high sensitivity of the assay, followed by multiplex RNA ISH to identify the cell type of transduced cells in ddPCR positive tissues. In all three mouse models dosed with a single intraperitoneal injection of 20 million TU UB-VV100, the presence of detectable vector copies was largely restricted to the liver and spleen (FIGS. 46C and 46D), and the predominant transduced cell types observed were human T cells and mouse macrophages. Rare transduction of tissue epithelial cells was observed in some tissues. Vector integration occurred in a dose-dependent manner in CD34-NSG mice (the only model treated at two dose levels). No transduction above Vehicle controls was observed in the heart, brain, kidney, and lung at the 20 million TU UB-VV100 dose (FIG. 46C).

In conclusion, in this study the predominant tissues in which transduction events were detected were liver and spleen, and the predominant transduced cell types observed were human T cells and mouse macrophages (FIG. 47). Rare transduced non-immune cells were identifiable in these tissues and in the external ovarian lining, attributed to the IP route of administration. At the low dose, no transduction above Vehicle control levels was measured in the heart, brain, kidney, and lung. Importantly, no histopathological findings definitively related to UB-VV100 administration were observed.

The data shows that the CD34-humanized NSG mouse model transduced with UB-V100 has full biologic activity (CAR-T cell generation and B cell aplasia) and the mice displayed higher and more stable humanization levels.

### Example 13: Transducing CD19+ B Cells with VV100

VV100 is a lentiviral drug product engineered to selectively activate and transduce T cells in vivo. The VV100 lentiviral drug product comprised SEQ ID NOs: 122, 123, 127, 124, and 125. Selective T cell activation is achieved by the expression of anti-CD3-scFv on the viral envelope. The lentivirus encodes an anti-CD19-CAR with a FMC63 scFv, a short IgG4 hinge (SEQ ID NO: 95), and a 4-1BB/CD3ζ signaling domain for tumor targeting. It also encodes a rapamycin activated cytokine receptor cassette (FRB-RACR) to promote CAR-T cell survival and proliferation in the presence of rapamycin (FIG. 23B). The objective of this study was to assess the therapeutic application of VV100 to treat CD19-expressing B cell malignancies via an intranodal injection.

Epitope masking occurs when tumor B cells are unintentionally transduced to express an anti-CD19 CAR and the expression of the CAR renders the CD19 epitope invisible, or ‘masked’ to recognition by CAR T cells. The underlying mechanism driving epitope masking is the anti-CD19 CAR binding in cis to CD19 on the tumor cell surface. This phenomenon was first reported in a pediatric B-ALL patient treated with the anti-CD19 CAR T cell product CTL019 (Ruella M, et al. Nat Med. 2018; 24(10):1499-1503). CTL019 encodes a CD8a hinge which is 45 amino acids long. It is hypothesized that the longer length of the CD8a hinge enables epitope masking by providing the flexibility needed to access the CAR-binding epitope. To minimize the risk of epitope masking with VV100, the anti-CD19 CAR was intentionally designed to encode a short IgG4 hinge (14 amino acids). Structural analysis of CD19 demonstrates a membrane distal location of the CAR epitope, and thus is not readily accessible for cis-binding by a CAR with a short hinge. To assess if epitope masking occurs with VV100, Nalm6 cells, a B-ALL tumor cell line, were transduced with VV100 to model a transduced CD19+ tumor cell scenario. The study sought to answer two questions: (1) Do CAR+ Nalm6 cells exhibit an apparent loss in surface CD19 detection by flow cytometry? and (2) Can anti-CD19 CAR-T cells kill CAR+ Nalm6 cells?

### Results

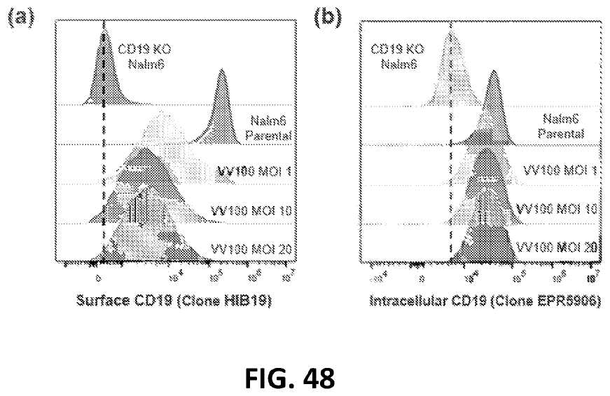

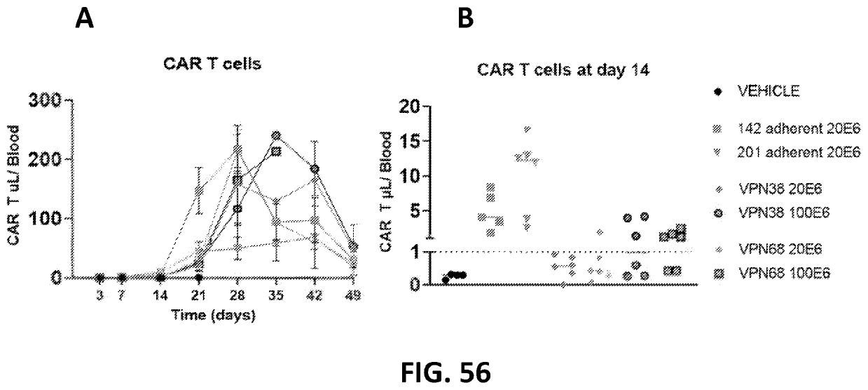

To determine the effects of transducing CD19+ B cells with VV100, Nalm6, a B-ALL tumor cell line, was used as a model. Transduction with VV100 at MOI 1 generated 37.6% CAR+ Nalm6 cells while transduction with MOIs 10 and 20 led to even higher transduction efficiencies with 70% CAR+ Nalm6 cells by day 10. Next, CAR+ Nalm6 cells were stained with an anti-CD19 antibody (clone HIB19) to assess surface CD19 levels by flow cytometry (FIG. 48A). The HIB19 antibody binds to the same CD19 epitope as the anti-CD19 CAR FMC63 scFv. Using this antibody, it was observed that CAR+ Nalm6 cells had reduced surface CD19 detection compared to untransduced Nalm6 parental cells and transduction with higher MOIs led to lower surface CD19 MFIs (FIG. 56A). When CAR+ Nalm6 cells were stained with a different anti-CD19 antibody that recognizes an intracellular CD19 epitope (clone EPR5906), it was observed that overall CD19 protein levels were similar to untransduced Nalm6 cells, confirming that CD19 protein was still expressed in CAR+ Nalm6 cells (FIG. 48B). The data show that CAR+ Nalm6 cells have reduced surface CD19 detection because the CD19 epitope recognized by the HIB19 antibody is blocked by the anti-CD19 CAR binding in cis to CD19. However, surface CD19 levels on CAR+ Nalm6 cells remain detectable and above the level of the CD19 knockout Nalm6 cells.

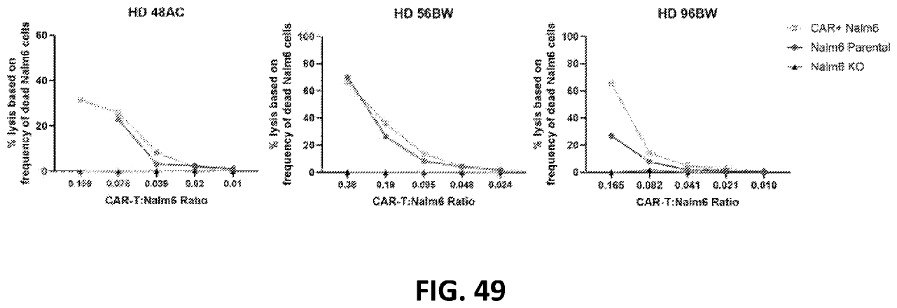

To determine if anti-CD19 CAR-T cells can kill CAR+ Nalm6 cells with reduced surface CD19 detection, an in vitro killing assay was established. Nalm6 GFP cells were transduced with VV100 at a MOI of 10 and generated 88% CAR+ Nalm6 cells with reduced surface CD19 detection as observed in the studies described above. PBMCs from 3 healthy donors were transduced with VV100, producing 7.8% to 19% CAR-T cells. The transduced Nalm6 cells were then cocultured with the transduced PBMCs at different CAR-T to Nalm6 ratios. To normalize for background non-specific killing, transduced Nalm6 cells were also cocultured with mock transduced PBMCs. After 24 hours, transduced Nalm6 cells were gated as CAR+ Nalm6 cells based on intracellular transgene expression (detected with an anti-P2A antibody) by flow cytometry. The percentage of lysis was calculated as the frequency of dead CAR+ Nalm6 cells when cocultured with CAR-T cells minus the frequency of dead CAR+ Nalm6 cells when cocultured with mock-transduced PBMCs. As seen in FIG. 49, anti-CD19 CAR-T cells were able to kill CAR+ Nalm6 cells. Importantly, the percentage of lysis was similar between CAR+ Nalm6 cells and untransduced Nalm6 parental cells, which were higher than that of CD19 knockout Nalm6 cells in all 3 healthy donors. The data demonstrate that epitope masking and subsequent antigen escape does not occur when Nalm6 cells are transduced with VV100. However, despite reduced surface CD19 detection, anti-CD19 CAR-T cells can still mediate CD19 antigen specific cytolytic activity against CAR+ Nalm6 cells in vitro.

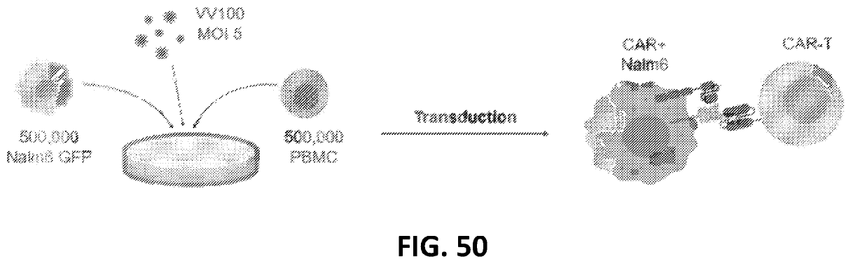

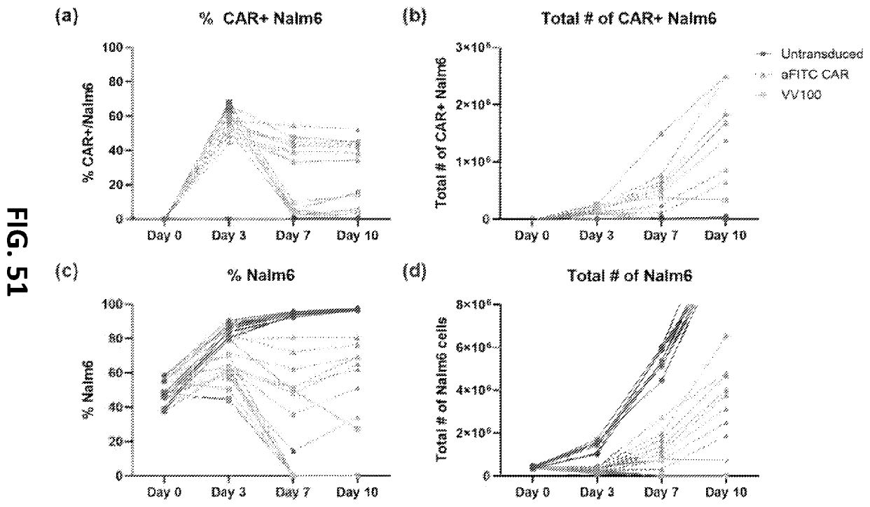

When VV100 is injected into a patient, tumor cells may be exposed to viral particles. To model a scenario in which a high number of tumor B cells are present at the time of transduction, 5e5 Nalm6 GFP cells and 5e5 healthy donor PBMCs were mixed and transduced with VV100 at a MOI of 5 (FIG. 50). In this model, transduction with VV100 generates anti-CD19 CAR-T cells and CAR+ Nalm6 cells in the same well. It can then be assessed whether anti-CD19 CAR-T cells eliminate CAR+ Nalm6 cells or if CAR+ Nalm6 escape detection and grow unchecked. To understand if anti-CD19 CAR-T cells from multiple healthy donors can kill CAR+ Nalm6 cells, PBMCs from 8 healthy donors were studied in a single experiment. The healthy donors represent a wide age range with varying percentages of naïve and memory T cell subsets within the PBMC samples.

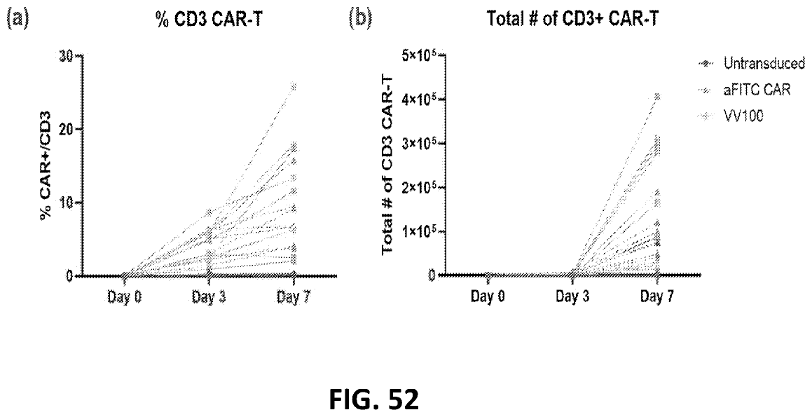

In all 8 healthy donors, transducing a mixed population of Nalm6 cells and PBMCs with either VV100 or an irrelevant anti-FITC CAR generated both CAR+ Nalm6 cells and CAR-T cells (FIGS. 51 and 52). On day 3, transduction with VV100 generated 51.7%-67.9% anti-CD19 CAR+ Nalm6 cells with reduced surface CD19 detection. By day 7, the frequency and total number of anti-CD19 CAR+ Nalm6 cells had declined until all anti-CD19 CAR+ Nalm6 cells were eliminated in 7 out of 8 healthy donors (FIG. 51B). In contrast, transduction with an irrelevant CAR did not eliminate any Nalm6 cells, demonstrating that killing of CAR+ Nalm6 cells is mediated by anti-CD19 CAR-T cells binding to the CD19 epitope.

Healthy donor 66BW did not eliminate Nalm6 cells by day 7, however, this donor did exhibit control over CAR- and CAR+ Nalm6 cells (FIG. 51). We hypothesize this occurred because 66BW did not generate sufficient CAR-T cell numbers to eliminate the rapidly dividing Nalm6 cells. On day 7, 66BW had the lowest frequency (2.03%) and total number of CAR-T cells (1.39e4) compared to other healthy donors (FIG. 52). Transduction with VV100 in this donor still resulted in fewer Nalm6 cells (7.31e5 cells) on day 10 compared to transduction with anti-FITC CAR (4.78e6 cells), and proportions of CAR+ and CAR− Nalm6 cells remained constant over time. This indicates that donor 66BW had similar levels of control over both CAR+ and CAR− Nalm6 and that any apparent reduction in killing was not due to CAR expression by Nalm6 cells but an overall deficit in T cell effector function specific to this donor (FIGS. 51A and 51B). Overall, this data confirms that anti-CD19 CAR-T cells can target and kill CAR+ Nalm6 cells following VV100 treatment, demonstrating that epitope masking does not occur when CD19+ B cells are transduced with VV100.

### Conclusions

Nalm6 cells transduced with VV100 have reduced surface CD19 detection, but the reduced level is higher than that of CD19 knockout Nalm6 cells. In an in vitro killing assay, anti-CD19 CAR-T cells can kill CAR+ Nalm6 with similar percentage of lysis between CAR+ and CAR− Nalm6 cells. When a mixed population of Nalm6 and PBMCs are transduced with VV100, anti-CD19 CAR-T cells can eliminate CAR+ Nalm6 cells in the same well.

### Example 14: Comparison of Different Versions of UB-VV100 Against Nalm-6 Systemic Tumor Model

To assess the efficacy of different versions of UB-VV100 in a humanized NSG mouse model, UB-VV100 produced by suspension vs adherent methods, produced with different payload versions, and produced with different anti-CD3 scFv versions were compared and analyzed.

This study directly compared the efficacy of adherent UB-VV100 particles to suspension UB-VV100 particles. To perform this experiment, 7 different treatment groups were analyzed: two groups of mice were treated with 20E+06 TU of UB-VV100 produced with 293T adherent cells using a 4-plasmid system comparing the WPRE-containing payload (142) (SEQ ID NO: 121) or the payload without WPRE (201) (SEQ ID NO: 122). Two groups of mice were treated with 20E+06 or 100E+06 TU of VPN38, a suspension vector produced with a 5-plasmid system using transgene SEQ ID NO: 122 and the αCD3 scFv (SEQ ID NO: 126). Two groups of mice were treated with 20E+06 or 100E+06 TU of VPN68, a 5-plasmid suspension vector containing the αCD3 scFv binder (SEQ ID NO: 127).

The goals of the study were to:
1) Determine if adherent vector manufactured with transgene SEQ ID NO: 121 was comparable to adherent vector manufactured with transgene SEQ ID NO: 122.
2) Determine if there was reduced efficacy during the manufacturing change from adherent to suspension vector.
3) Assess whether dose escalation to 100E+06 TU could overcome potential loss of activity/PK shift observed with suspension lots relative to adherent lots.
4) Compare the anti-CD3 scFv binder (SEQ ID NO: 127) to version (SEQ ID NO: 126).

### Study Overview

48 Female 6-8-week-old mice NSG MHC Class I and II KO mice were used in the study. Lentivirus was prepared using 293T adherent production (142 (SEQ ID NO: 121), 201 (SEQ ID NO: 122)) titrated on 293T adherent cells. Suspension lots were produced in 293T suspension cells (VPN38 and VPN68) and titrated on SUPT1 cells by ddPCR. UB-VV100 Virus preparations were negative for mycoplasma with an endotoxin activity of less than 2 EU/ml respectively.

The morning of infection, virus was thawed at room temperature and diluted in PBS. Virus preparations were kept on ice and allowed to equilibrate to room temperature before injecting 500 ul per mouse (IP).

### Study Protocol

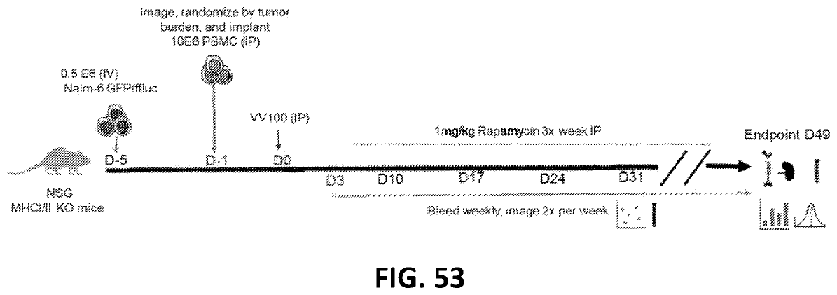

On study day −5, 40 mice were engrafted with 5E05 Nalm-6 GFP FFLUC tumor cells via retroorbital injection. The morning of study day −1, tumor burden was assessed with IVIS imaging to ensure successful engraftment, the same day mice were humanized with 10 million human PBMCs via IP injection. Mice were assigned study arms at this time and distributed into 7 treatment groups based on tumor burden. On study day 0 mice were treated with UB-VV100 or vehicle via IP (Table 11). Starting on study day 4 all mice were treated with 1 mg/kg of rapamycin on a Monday/Wednesday/Friday schedule via intraperitoneal injection. A study timeline is depicted in FIG. 53.

TABLE 11
Study Groups

| | | Tumor (day −5 RO) | Humanization | | Rapamycin |
|---|---|---|---|---|---|
| Group | N | 0.5 million | (Day −1) | UB-VV100 Day 0 | (Day 4) |
| 1 | 5 | Nalm6 | 10 million PBMC IP | Vehicle | 1 mg/kg MWF |
| 2 | 5 | Nalm6 | 10 million PBMC IP | 20e6 VV100 | 1 mg/kg MWF |
| | | | | adherent 142 payload | |
| | | | | (SEQ ID NO: 121) | |
| 3 | 5 | Nalm6 | 10 million PBMC IP | 20e6 VV100 | 1 mg/kg MWF |
| | | | | adherent 201 payload | |
| | | | | (SEQ ID NO: 122) | |
| 4 | 5 | Nalm6 | 10 million PBMC IP | 20e6 VV100 | 1 mg/kg MWF |
| | | | | suspension VPN38 | |
| 5 | 5 | Nalm6 | 10 million PBMC IP | 100e6 VV100 | 1 mg/kg MWF |
| | | | | suspension VPN38 | |
| 6 | 5 | Nalm6 | 10 million PBMC IP | 20e6 VV100 | 1 mg/kg MWF |
| | | | | suspension VPN68 | |
| 7 | 5 | Nalm6 | 10 million PBMC IP | 100e6 VV100 | 1 mg/kg MWF |
| | | | | suspension VPN68 | |

### Results

Animals were monitored for survival during the study. It was found that all animals treated with vehicle died by study day 22, whereas animals treated with either adherent or suspension lots of UB-VV100 showed survival past this time point (FIG. 54). 40% of animals had to be euthanized during the study due to development of retroorbital tumors. Additionally, 5 animals that received 100 million TU of VPN68 were euthanized due to acute weight loss soon after a flooding event.

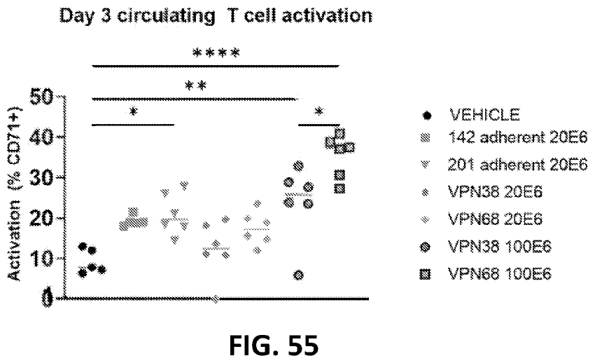

T cell populations on day 3 were analyzed to assess T cell activation shortly after vector administration. It was found that CD25 expression was low in all study arms (data not shown). In animals treated with the various tested lots of UB-VV100 T cells, east test lot had higher CD71 expression than T cells of animals treated with vehicle (FIG. 55). It was found that animals treated with 100E06 TU of VPN68 showed higher CD71 expression than animals treated with 100E6 TU VPN38. There was a trend towards higher CD71 expression in the T cells of animals treated with 100E06 TU as compared to those treated with 20E06 TU of the suspension lots. Taken together, these findings suggest that both adherent and suspension particles promote activation as measured by upregulation of CD71, and that upregulation is dose dependent. Animals treated with VPN68 showed the highest activation (highest % CD71 expression) (FIG. 55).

Serial weekly blood draws were taken to assess CAR T cell expansion during the study. It was found that animals treated with all preparations of UB-VV100 generated CAR T cells which expanded between study days 14-42, and then contracted over days 42-49 in the remaining surviving animals (FIG. 56A). Animals treated with 20E06 TU adherent material with 142 payload (SEQ ID NO: 121) showed CART cell expansion between days 14-28, which contracted thereafter. Animals treated with adherent preparation 142 (SEQ ID NO: 121), VPN38 20E06 TU, VPN38 100E06 TU, VPN68 20E06 TU, and VPN68 100E06 TU developed peak circulating CAR T cell concentrations of >150 CAR T cells/μL blood, whereas animals treated with adherent prep 201 reached peaks of <100 CAR T cells μL blood. Animals treated with both adherent preps, and animals treated with 100E06 TU of VPN68 and VPN38 had readily detectable CAR T cells by study day 14; in contrast, animals treated with 20E06 TU of VPN38 and VPN68 had no detectable CAR T cells at this early time point. In summary, all preparations of UB-VV100 promoted upregulation of CD71 on study day 3 and generated detectable CAR T cells by study day 21, but animals treated with 20E6 TU of suspension particles displayed a 1 week delay of CAR T cell detection compared to animals treated with either adherent preparation or at higher dose (FIG. 56B).

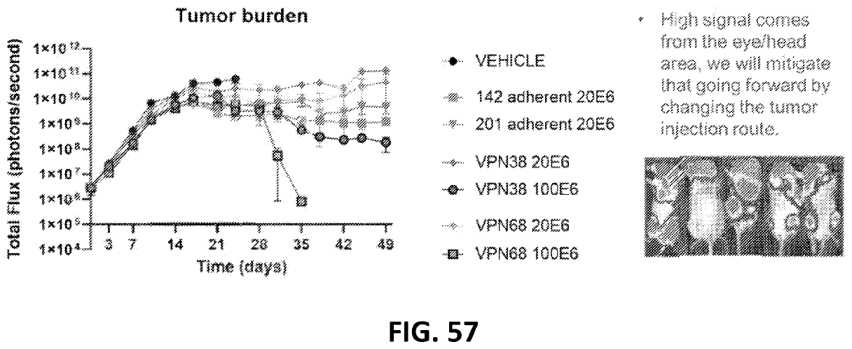

Disease progression was monitored by bioluminescence imaging after d-Luciferin injections via the IP route. Bioluminescence Imaging was performed twice a week throughout the study. Mice in the control group reach humane endpoint around day 25 and had to be euthanized due to disease burden and weight loss. About 40 percent of mice developed eye tumors and had to be euthanized prematurely. Mice treated with 20E6 of UB-VV100 VPN38 and VPN68 had controlled tumor burden that extended their life compared to vehicle, however tumor reduction was not observed. Mice treated with 100E6 UB-VV100 VPN38 and VPN68 had increased tumor reduction compared to mice treated with 20E6 UB-VV100. The greatest tumor reduction was observed in mice treated with 100E6 of VPN68 (FIG. 57). Mice treated with 20E6 adherent lots of UB-VV100 (either 142 (SEQ ID NO: 121) or 201 (SEQ ID NO: 122) payloads) had greater tumor reduction than mice treated with 20E6 TU of suspension lots (FIG. 57).

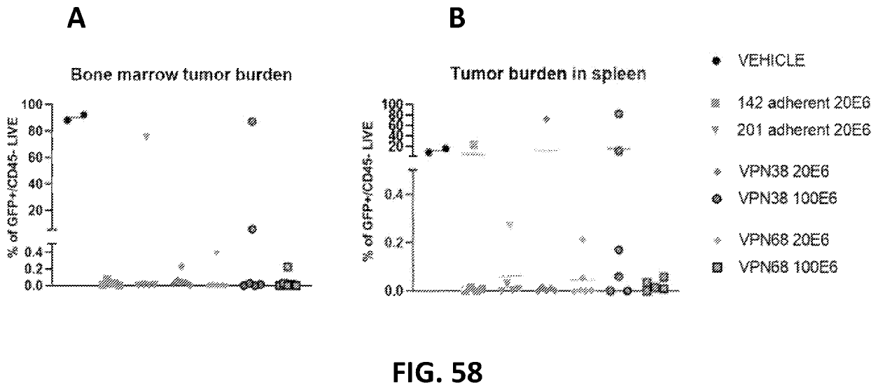

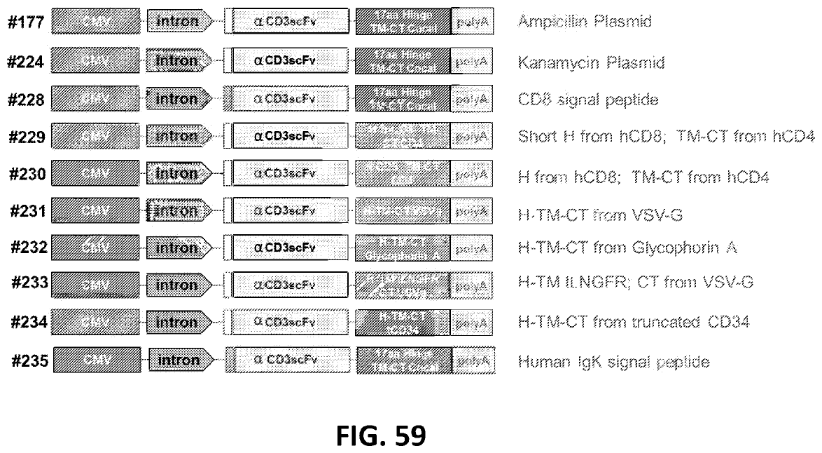

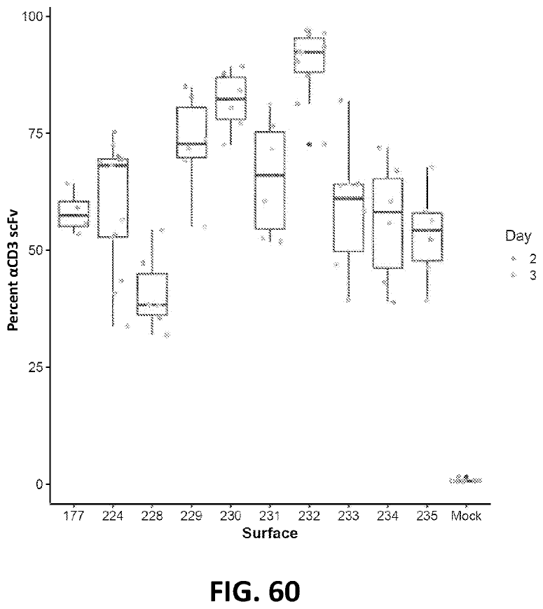

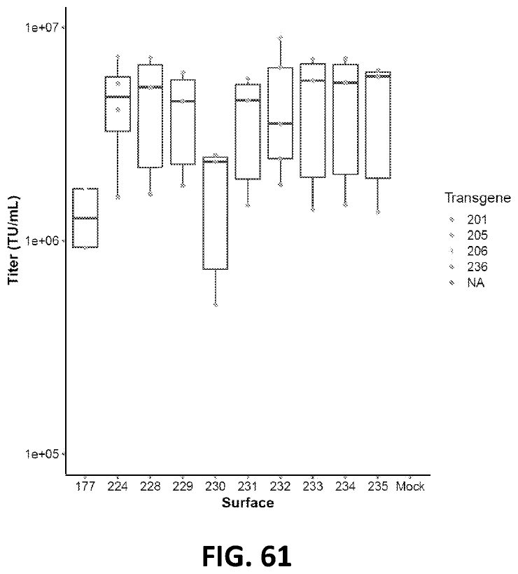

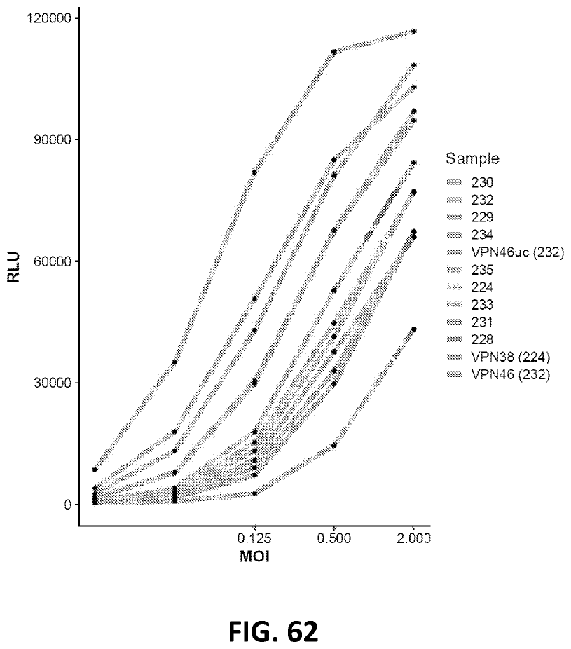

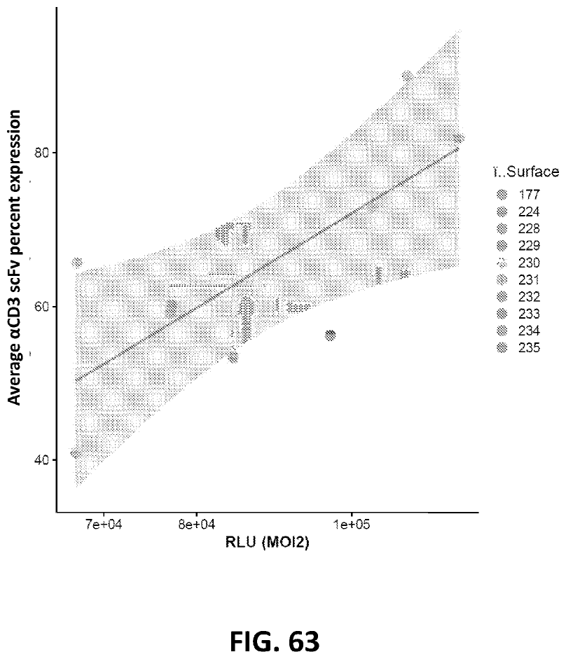

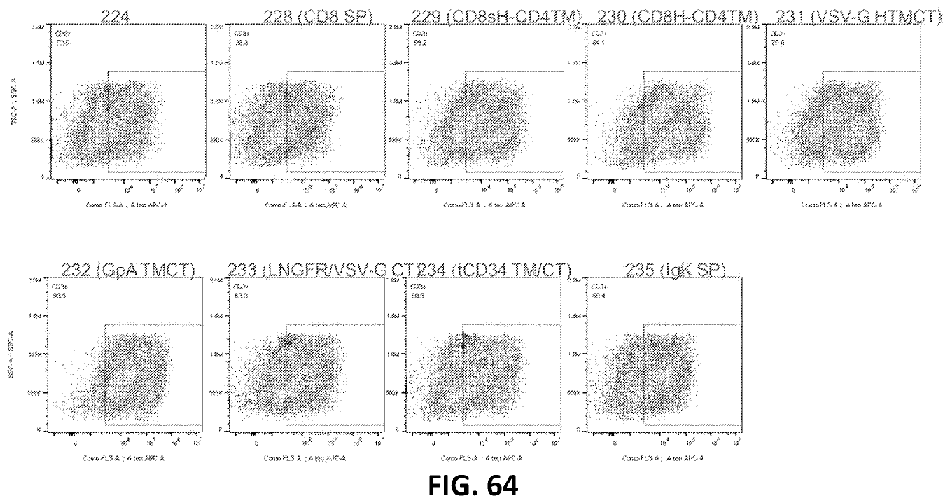

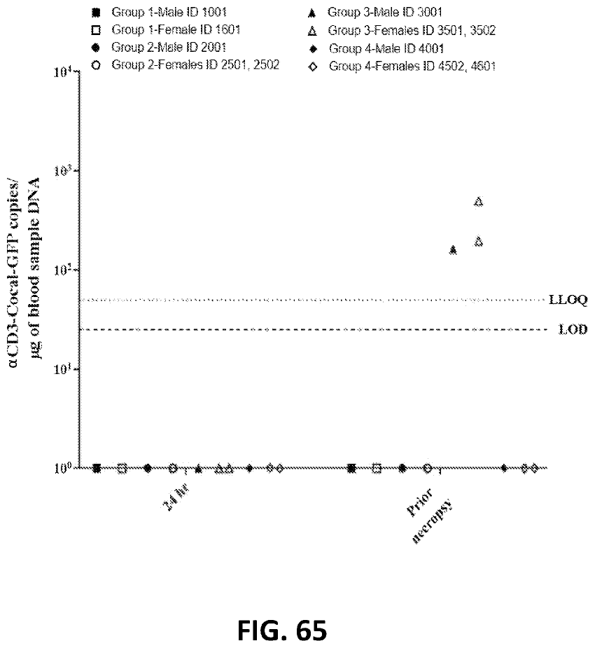

When mice reached humane endpoint, spleen and bone marrow were collected and processed for analysis including tumor burden by flow cytometry. To evaluate tumor burden the percentage of NALM-6 cells (Live, CD45−, GFP+) was measured. It was observed that mice in the vehicle control group had NALM-6 cells representing over 20% of cells in their spleen and close to 80% in their bone marrow. Tumor was greatly reduced in spleen and bone marrow after UB-VV100 treatment (FIG. 58). The greatest tumor reduction in bone marrow and spleen was observed in mice treated with 100E6 of VPN68 (FIG. 58).

### Conclusions

Increased survival was observed in mice treated with UB-VV100 relative to controls. During the study, 40% of animals developed retroorbital tumors and many of these animals had to be euthanized despite low tumor burden in peripheral organs and absence of other euthanasia criteria, according to the standards of the attending veterinarian recommendation. Mice treated with 100E6 TU UB-VV100 VPN68 had a cage-flooding event and had to be euthanized shortly after due to weight loss.

All versions of UB-VV100 tested showed anti-tumor activity, extending survival as compared to mice treated with vehicle alone, however, mice treated with VPN68 showed the most significant tumor control. At sacrifice mice treated with UB-VV100 presented tumor reduction in spleen and bone marrow compared to vehicle treated mice. Taken together, these results demonstrate that that all preparations of UB-VV100 had some activity. The most significant tumor reduction/control in non-retroorbital sites was observed with treatment of 20E+06 TU of adherent lots and 100E+06 TU of suspension lots.

The activation of T cells (using activation markers CD25 and CD71) after UB-VV100 administration was examined to examine differences in efficacy. On study day 3 no differences in CD25 expression were found in any of the groups. However, all mice treated with UB-VV100 showed higher expression levels of CD71 on T cells compared to vehicle treated mice. Specifically, at the 20E6 UB-VV100 dose higher expression of CD71 was observed in mice treated with adherent particles compared to suspension particles. This finding suggests that 20E+06 TU of adherent vector is more activating than 20E+06 TU of the suspension vector. When examining VPN38 vs VPN68, it was observed that the construct for αCD3 scFv expression present in VPN68 (SEQ ID NO: 127) is associated with significantly higher levels of activation measured by CD71 at the higher dose level. In addition, higher levels of CD71 were found in mice treated with 100E6 UB-VV100 compared to 20E6 suspension particles, suggesting that upregulation of CD71 is dose dependent.

All preparations of UB-VV100 generated detectable CAR T cells that expanded between study days 14-21 and contracted over days 42 to 49. Consistent with the lower expression of CD71 detected at study day 3, animals treated with 20E+06 TU of suspension particles displayed a 1-week delay of CAR T cell detection compared to animals treated with either adherent prep, which generated detectable CAR T cells by study day 14.

In summary, it was found that the switch from plasmid SEQ ID NO: 121 to plasmid SEQ ID NO: 122 had minimal effect on in vivo efficacy, that suspension particles appear to be less potent and less activating than adherent particles and that this difference can be overcome by increasing dose. In addition, switch from the SEQ ID NO: 126 plasmid to SEQ ID NO: 127 plasmid was associated with higher activation of T cells on day 3 and better tumor clearance in animals treated with 100E+06 TU. Dose escalation to 100E+06 TU will likely be necessary to generate consistent and predictable efficacy in a systemic Nalm-6 tumor model.

### Abbreviations

| | |
|---|---|
| AAV | Adeno-associated virus |
| AE | Adverse event |
| ALL | Acute lymphoblastic leukemia |
| AUC | Area under curve |
| B-LBL | B-lineage lymphoblastic lymphoma |
| BCS | Body conditioning score |
| BH | Bulk harvest |
| CAR | Chimeric Antigen Receptor |
| CD19 | Cluster of Differentiation 19, B-lymphocyte antigen |
| CD3 | Cluster of Differentiation 3 |
| CDMO | Contract development and manufacturing organization |
| CLL | Chronic lymphocytic leukemia |
| CMC | Chemical Manufacturing and Controls |
| ddPCR | Digital droplet polymerase chain reaction |
| DLT | Dose limiting toxicity |
| DNA | Deoxyribonucleic acid |
| DP | Drug product |
| DS | Drug substance |
| DSC | Disease-specific cohort |
| DSMB | Data Safety Monitoring Board |
| ELISA | Enzyme-linked immunosorbent assay |
| FL | Follicular lymphoma |
| FRB | FKBP-rapamycin binding (domain) |
| GLP | Good laboratory practice |
| GMP | Good Manufacturing Practice |
| IFNγ | Interferon gamma |
| IL-15 | Interleukin 15 |
| IL-2 | Interleukin 2 |
| IN | Intra-nodal |
| IP | Intraperitoneal |
| ISH | in situ hybridization |
| LAL | Limulus amebocyte lysate |
| LDLR | Low-density lipoprotein receptor |
| LEDGF | Lens epithelium-derived growth factor |
| LVV | Lentiviral vector |
| MCL | Mantle cell lymphoma |
| MEK | Mitogen-activated protein kinase |
| MOI | Multiplicity of infection |
| mTOR | Mammalian target of rapamycin |
| NHL | Non-Hodgkin's lymphoma |
| NSG | Non-obese-diabetic SCID Gamma mouse |
| PBMC | Peripheral blood mononuclear cells |
| PCR | Polymerase chain reaction |
| PD | Pharmacodynamics |
| PK | Pharmacokinetic |
| POC | Proof of concept |
| RACR | Rapamycin activated cytokine receptor |
| RAF | Rapidly Accelerated Fibrosarcoma |
| RAS | Rat sarcoma virus oncogene |
| RCL | Replication competent lentivirus |
| RNA | Ribonucleic acid |
| SAE | Serious adverse event |
| SC | Subcutaneous |
| scFv | Single chain variable fragment |
| SEM | Standard error of the mean |
| SIN | Self-inactivating |
| SMC | Safety monitoring committee |
| STDEV | Standard deviation |
| TCR | T cell receptor |
| TNFα | Tumor necrosis factor alpha |
| TU | Transducing units |
| VCN | Vector copy number |
| WPRE | Woodchuck (Hepatitis Virus) Posttranscriptional Regulatory |
| | Element |

What is claimed is:

1. A viral particle comprising a vector genome comprising a polynucleotide sequence encoding an anti-CD19 chimeric antigen receptor, wherein the viral particle transduces immune cells in vivo.

2. The particle of claim 1, wherein the viral particle is a lentiviral particle.

3. The particle of claim 1, wherein the immune cells are T cells.

4. The particle of claim 1, wherein the vector genome comprises a polynucleotide sequence encoding a multipartite cell-surface receptor.

5. The particle of claim 4, wherein the multipartite cell-surface receptor is a chemically inducible cell-surface receptor.

6. The particle of claim 4, wherein the vector genome comprises a polynucleotide sequence encoding a multipartite cell-surface receptor comprising a FKBP-rapamycin complex binding domain (FRB domain) or a functional variant thereof; and the polynucleotide comprises a polynucleotide sequence encoding a FK506 binding protein domain (FKBP) or a functional variant thereof.

7. The particle of claim 5, wherein the multipartite cell-surface receptor is a rapamycin-activated cell-surface receptor.

8. The particle of claim 1, wherein the vector genome comprises a polynucleotide sequence that confers resistance to an immunosuppressive agent.

9. The particle of claim 8, wherein the vector genome sequence that confers resistance to an immunosuppressive agent encodes a polypeptide that binds rapamycin, wherein optionally, the polypeptide is an FRB.

10. The particle of claim 4, wherein the vector genome comprises, in 5′ to 3′ order on a polycistronic transcript: the polynucleotide sequence encoding the multipartite cell-surface receptor and the polynucleotide sequence encoding the anti-CD19 chimeric antigen receptor.

11. The particle of claim 4, wherein the vector genome comprises, in 5′ to 3′ order on a polycistronic transcript: the polynucleotide sequence encoding the anti-CD19 chimeric antigen receptor and the polynucleotide sequence encoding the multipartite cell-surface receptor, and/or wherein the anti-CD19 chimeric antigen receptor shares at least 80%, 90%, 95%, or 100% identity to SEQ ID NO: 51, 79, 89, 121, or 122.

12. The particle of claim 1, wherein the polynucleotide encoding the anti-CD19 chimeric antigen receptor and/or the polynucleotide encoding the multipartite cell-surface receptor is operatively linked to one or more promoters.

13. The particle of claim 12, wherein at least one of the one or more promoters is an inducible promoter.

14. The particle of claim 1, wherein the viral particle comprises a viral envelope comprising one or more immune cell-activating proteins exposed on the surface and/or conjugated to the surface of the viral envelope.

15. The particle of claim 14, wherein the viral envelope comprises an anti-CD3 single-chain variable fragment exposed on the surface and/or conjugated to the surface of the viral envelope.

16. The particle of claim 14, wherein the viral envelope comprises a Cocal glycoprotein exposed on the surface and/or conjugated to the surface of the viral envelope, optionally wherein the Cocal glycoprotein comprises the R354Q mutation compared to a reference sequence according to SEQ ID NO: 5.

17. The particle of claim 14, wherein the viral envelope comprises an anti-CD3 single-chain variable fragment and a Cocal glycoprotein exposed on the surface and/or conjugated to the surface of the viral envelope.

18. The particle of claim 14, wherein the viral envelope comprises an anti-CD3 single-chain variable fragment sequence that shares at least 80%, at least 85%, at least 90%, at least 95%, at least 99%, or 100% identity to SEQ ID NO: 2 or 12.

19. The particle of claim 14, wherein the viral envelope comprises a Cocal glycoprotein sequence that shares at least 80%, at least 85%, at least 90%, at least 95%, at least 99%, or 100% identity to SEQ ID NO: 5, 13, 19, 123, 128, 129, or 130.

20. The particle of claim 13, wherein the promoter is an MND promoter.

21. The particle of claim 14, wherein the vector genome shares at least 80%, at least 85%, at least 90%, at least 95%, at least 99%, or 100% identity to SEQ ID NO: 49.

22. The particle of claim 14, wherein the vector genome shares at least 80%, at least 85%, at least 90%, at least 95%, at least 99%, or 100% identity to SEQ ID NO: 75.

23. The particle of claim 14, wherein the vector genome shares at least 80%, at least 85%, at least 90%, at least 95%, at least 99%, or 100% identity to SEQ ID NO: 87.

24. A method of treating a disease or disorder associated with malignant CD19+ cells, transducing immune cells in vivo, and/or generating an immune cell expressing an anti-CD19 chimeric antigen receptor in a subject in need thereof, comprising administering the viral particle of any one of claims 1 to 23 to the subject.

25. The method of claim 24, wherein the viral particle is administered by intraperitoneal, subcutaneous, or intranodal injection.

26. A method of treating a disease or disorder associated with malignant CD19+ cells in a subject in need thereof, comprising administering immune cells transduced with the viral particle of any one of claims 1 to 23 to the subject.

27. A method of treating a disease or disorder in a subject in need thereof, comprising administering a therapeutically effective amount of a viral particle to the subject by intraperitoneal, subcutaneous, or intranodal injection, wherein the viral particle transduces immune cells in vivo.

28. The method of claim 27, wherein the viral particle is administered by intra-nodal injection, via inguinal lymph node.

29. The method of claim 27, wherein the viral particle is administered by intraperitoneal injection.

30. A viral particle for use in transducing immune cells in vivo, comprising a polynucleotide comprising a polynucleotide sequence encoding a chimeric antigen receptor.

31. The particle of any one of claims 1-23 or claim 30, wherein the viral particle further comprises a polynucleotide sequence encoding a dominant-negative TGFβ receptor.

32. The particle of any one of claim 1 or 30, wherein expression of the chimeric antigen receptor is modulated by a FRB-degron fusion polypeptide and wherein suppression of the FRB-degron fusion polypeptide is chemically inducible by a ligand.

33. The particle of claim 32, wherein the ligand is rapamycin.

34. The particle of any one of claim 1 or 30, wherein expression of the chimeric antigen receptor is modulated by a degron fusion polypeptide and wherein suppression of the degron fusion polypeptide is chemically inducible by a ligand.

35. The method of any one of claim 24 or 27, where the disease or disorder comprises B-cell malignancy, relapsed/refractory CD19-expressing malignancy, diffuse large B-cell lymphoma (DLBCL), Burkitt's type large B-cell lymphoma (B-LBL), follicular lymphoma (FL), chronic lymphocytic leukemia (CLL), acute lymphocytic leukemia (ALL), mantle cell lymphoma (MCL), hematological malignancy, colon cancer, lung cancer, liver cancer, breast cancer, renal cancer, prostate cancer, ovarian cancer, skin cancer, melanoma, bone cancer, brain cancer, squamous cell carcinoma, leukemia, myeloma, B cell lymphoma, kidney cancer, uterine cancer, adenocarcinoma, pancreatic cancer, chronic myelogenous leukemia, glioblastoma, neuroblastoma, medulloblastoma, sarcoma, and any combination thereof.

36. The method of any one of claim 24 or 27, where the disease or disorder comprises diffuse large B-cell lymphoma (DLBCL).

37. The method of any one of claim 24 or 27, where the disease or disorder comprises Burkitt's type large B-cell lymphoma (B-LBL).

38. The method of any one of claim 24 or 27, where the disease or disorder comprises follicular lymphoma (FL).

39. The method of any one of claim 24 or 27, where the disease or disorder comprises chronic lymphocytic leukemia (CLL).

40. The method of any one of claim 24 or 27, where the disease or disorder comprises acute lymphocytic leukemia (ALL).

41. The method of any one of claim 24 or 27, where the disease or disorder comprises mantle cell lymphoma (MCL).

42. A pharmaceutical composition comprising the viral particle of any one of claims 1 to 23.

43. A kit comprising the pharmaceutical composition of claim 42 and optionally a composition comprising a ligand, optionally rapamycin.

44. A viral particle for use in a method according to any one of claim 24-29 or 35-41.

45. The use of a viral particle in a method according to any one of claim 24-29 or 35-41.

46. The method of claim 24, wherein CD19+ B cells in the subject are depleted by at least 80%, at least 85%, at least 90%, or at least 95% as compared to the subject without viral particles.

47. The method of claim 46 wherein the CD19+ B cells are depleted in peripheral blood of the subject.

48. The method of claim 46, wherein B cells depletion is sustained in the subject for at least 7, at least 10, at least 20, at least 30 days, at least 40 days, at least 50 days, at least 60 days, at least 70 days, or at least 80 days after administering the viral particle.

49. The method of claim 24, wherein at least 2 million, at least 4 million, at least 6 million, at least 8 million or at least 10 million transducing units of viral particle are administered to the subject.

50. The method of claim 32, wherein contacting the immune cells with the ligand increases the number of immune cells expressing an anti-CD19 chimeric antigen receptor in a subject by at least 10-fold, at least 50-fold, at least 100-fold, at least 200-fold, at least 500-fold, or at least 1000-fold.

51. A polypeptide comprising a single-chain variable fragment that specifically binds CD3 (anti-CD3 scFv) and a glycophorin A transmembrane fragment.

52. The polypeptide of claim 51, wherein the glycophorin A transmembrane fragment shares at least 80%, at least 85%, at least 90%, at least 95%, at least 99%, or 100% identity to
(SEQ ID NO: 105)
HFSEPEITLIIFGVMAGVIGTILLISYGIRRLIKKSPSDVKPLPS
PDTDVPLSSVEIENPETSDQ.

53. The polypeptide of claim 51, wherein the anti-CD3 scFv shares at least 80%, at least 85%, at least 90%, at least 95%, at least 99%, or 100% identity to SEQ ID NO: 2 or 12.

54. The polypeptide of any one of claims 51 to 53, wherein the polypeptide shares at least 80%, at least 85%, at least 90%, at least 95%, at least 99%, or 100% identity to SEQ ID NO: 119.

55. The polypeptide of claim 51, wherein the transmembrane fragment shares at least 80%, at least 85%, at least 90%, at least 95%, at least 99%, or 100% identity to SEQ ID NO 13, 19, 25, 31, 37, 43, or 105.

56. A surface-engineered lentiviral particle comprising the polypeptide of any one of claims 51 to 54 displayed on the surface of the lentiviral particle.

57. A method of transducing cells, comprising contacting the viral particle of any one of claims 1-23 with an immune cell in vivo.

58. A polynucleotide comprising an anti-CD3 scFv and a glycophorin A transmembrane fragment.

59. The polynucleotide of claim 58, wherein the glycophorin A transmembrane fragment shares at least 80%, at least 85%, at least 90%, at least 95%, at least 99%, or 100% identity to SEQ ID NO: 106.

60. The polynucleotide of claim 58, wherein the anti-CD3 scFv shares at least 80%, at least 85%, at least 90%, at least 95%, at least 99%, or 100% identity to SEQ ID NO: 7 or 15.

61. The polynucleotide of any one of claims 58 to 60, wherein the polypeptide shares at least 80%, at least 85%, at least 90%, at least 95%, at least 99%, or 100% identity to SEQ ID NO: 120.

62. The polynucleotide of claim 58, wherein the transmembrane fragment shares at least 80%, at least 85%, at least 90%, at least 95%, at least 99%, or 100% identity to SEQ ID NO 16, 22, 25, 28, 34, 40, 47, or 106.

63. A method of making a viral particle comprising:
a) providing a cell in a culture medium; and
b) transfecting the cell with the vector genome of any one of claims 1 to 23, a transfer plasmid, and a packaging plasmid, simultaneously or sequentially; whereby the cell expresses a surface-engineered viral particle.

64. A method of treating a disease or disorder associated with malignant CD19+ cells comprising transducing immune cells in vivo, and/or generating a viral particle expressing an anti-CD3 single-chain variable fragment exposed on the surface and/or conjugated to the surface of the viral envelope that shares at least 80%, at least 85%, at least 90%, at least 95%, at least 99%, or 100% identity to SEQ ID NO: 2 or 12 and administering the viral particle to a subject.

65. The method of claim 64, wherein the viral particle is administered by intraperitoneal, subcutaneous, or intranodal injection.
